(19) United States
(12) Patent Application Publication
(10) Pub. No.: US 2009/0047216 A1
(43) Pub. Date: Feb. 19, 2009

(54) Tumor vasculature markers and methods of use thereof

(76) Inventors: George Coukos, Yardley, PA (US); Ronald J. Buckanovich, Ann Arbor, MI (US); Phyllis A. Gimotty, Wallingford, PA (US)

Correspondence Address: Pearl Cohen Zedek Latzer, LLP, 1500 Broadway, 12th Floor, New York, NY, 10036, US

(21) Appl. No.: 11/657,719

(22) Filed: Jan. 25, 2007

Related U.S. Application Data
(60) Provisional application No. 60/762,787, filed on Jan. 26, 2006.
(60) Provisional application No. 60/791,212, filed on Apr. 12, 2006.
(60) Provisional application No. 60/844,347, filed on Sep. 14, 2006.

(51) Int. Cl. A61K 49/00; A61B 18/18

(52) U.S. Cl. 424/9.1; 606/13

(57) ABSTRACT

This invention provides methods of detecting and localizing tumor vasculature cells (TVC) and solid tumors; and treating, impeding vascularization of, and determining the stage of solid tumors in a subject, comprising the step of contacting a subject or a TVC with a ligand that binds to a nucleic acid molecule of the present invention, or binds to a protein encoded by the nucleic acid molecule.

### CROSS REFERENCE TO RELATED APPLICATIONS

This application claims priority of U.S. Provisional Application Ser. No. 60/762,787, filed Jan. 26, 2006, U.S. Provisional Application Ser. No. 60/791,212, filed Apr. 12, 2006, and U.S. Provisional Application Ser. No. 60/844,347, filed Sep. 14, 2006. These applications are hereby incorporated in their entirety by reference herein.

### STATEMENT REGARDING FEDERALLY SPONSORED RESEARCH OR DEVELOPMENT

The invention described herein was supported in whole or in part by grants from The National Institutes of Health (Grant No. CA098951, P50-CA083638, K12-HD43459, and D43-TW00671). The government has certain rights in the invention.

### FIELD OF INVENTION

This invention provides methods of detecting and localizing tumor vasculature cells (TVC) and solid tumors; and treating, impeding vascularization of, and determining the stage of solid tumors in a subject, comprising the step of contacting a subject or a TVC with a ligand that binds to a nucleic acid molecule of the present invention, or binds to a protein encoded by the nucleic acid molecule.

### BACKGROUND OF THE INVENTION

Markers of solid tumors and their vasculature have been difficult to identify because of difficulty in isolating these cells in contaminant-free preparations. Such markers are urgently need for use in a variety of therapeutic and diagnostic applications.

### SUMMARY OF THE INVENTION

This invention provides methods of detecting and localizing tumor vasculature cells (TVC) and solid tumors; and treating, impeding vascularization of, and determining the stage of solid tumors in a subject, comprising the step of contacting a subject or a TVC with a ligand that binds to a nucleic acid molecule of the present invention, or binds to a protein encoded by the nucleic acid molecule.

In one embodiment, the present invention provides a method of localizing a solid tumor vasculature in a subject, the method comprising the steps of: (a) contacting the subject with a ligand that binds a DR6-encoding nucleic acid molecule or that binds a DR6 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds an ADAM12-encoding nucleic acid molecule or that binds an ADAM12 protein; and (b) localizing the ligand, thereby localizing a solid tumor vasculature in a subject. In another embodiment, the solid tumor is an ovarian tumor. In another embodiment, the solid tumor is any other type of solid tumor known in the art. In another embodiment, the ADAM12-encoding nucleic acid molecule or ADAM12 protein is a short isoform thereof. In another embodiment, the nucleotide or protein is any other isoform known in the art. Each possibility represents a separate embodiment of the present invention.

In another embodiment, the present invention provides a method of localizing a solid tumor vasculature in a subject, the method comprising the steps of: (a) contacting the subject with a ligand that binds a long isoform of an ADAM12-encoding nucleic acid molecule or an ADAM12 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a CDCP1-CUB-encoding nucleic acid molecule or a CDCP1-CUB protein; and (b) localizing the ligand, thereby localizing a solid tumor vasculature in a subject. In another embodiment, the solid tumor is an ovarian tumor. In another embodiment, the solid tumor is any other type of solid tumor known in the art. In another embodiment, the CDCP1-CUB-encoding nucleic acid molecule or CDCP1-CUB protein is a long isoform thereof. In another embodiment, the nucleotide or protein is any other isoform known in the art. Each possibility represents a separate embodiment of the present invention.

In another embodiment, the present invention provides a method of localizing a solid tumor vasculature in a subject, the method comprising the steps of: (a) contacting the subject with a ligand that binds a short isoform of a CDCP1-CUB-encoding nucleic acid molecule or a CDCP1-CUB protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a SLC11A1-NRAMP-encoding nucleic acid molecule or that binds a SLC11A1-NRAMP protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a BLAME/SLAMF8-encoding nucleic acid molecule or that binds a BLAME/SLAMF8 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds an ESM1-encoding nucleic acid molecule or that binds an ESM1 protein; and (b) localizing the ligand, thereby localizing a solid tumor vasculature in a subject. In another embodiment, the solid tumor is a renal tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds an EGFL6-encoding nucleic acid molecule or that binds an EGFL6 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds an FZD10-encoding nucleic acid molecule or that binds an FZD10 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a COL11A1-encoding nucleic acid molecule or that binds a COL11A1 protein; and (b) localizing the ligand, thereby localizing a solid tumor vasculature in a subject. In another embodiment, the solid tumor is a breast cancer tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a TNFAIP6-encoding nucleic acid molecule or that binds a TNFAIP6 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds an Adlican-encoding nucleic acid molecule or that binds an Adlican protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a nucleic acid molecule selected from DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a nucleic acid molecule that encodes a protein selected from DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a protein selected from DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of treating a solid tumor in a subject, the method comprising the step of contacting the subject with an antibody or ligand that is conjugated to a cytotoxic agent or cytotoxic drug and that binds a nucleic acid molecule selected from DR6, ADAM12, CDCP1-CUB, SLC11A1-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, thereby treating a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of treating a solid tumor in a subject, the method comprising the step of contacting the subject with an antibody or ligand that is conjugated to a cytotoxic agent or cytotoxic drug and that binds a nucleic acid molecule encoding a protein selected from DR6, ADAM12, CDCP1-CUB, SLC11AI-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, thereby treating a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of treating a solid tumor in a subject, the method comprising the step of contacting the subject with an antibody or ligand that is conjugated to a cytotoxic agent or cytotoxic drug and that binds a protein selected from DR6, ADAM12, CDCP1-CUB, SLC11A1-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, thereby treating a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of treating a solid tumor in a subject, the method comprising the step of contacting the subject with an antibody or ligand that is conjugated to a cytotoxic agent or cytotoxic drug and that binds an ESM1-encoding nucleic acid molecule or an ESM1 protein, thereby treating a solid tumor in a subject. In another embodiment, the solid tumor is a renal tumor. In another embodiment, the solid tumor is any other type of solid tumor known in the art. Each possibility represents a separate embodiment of the present invention.

In another embodiment, the present invention provides a method of treating a solid tumor in a subject, the method comprising the step of contacting the subject with an antibody or ligand that is conjugated to a cytotoxic agent or cytotoxic drug and that binds a COL11A1-encoding nucleic acid molecule or a COL11A1 protein, thereby treating a solid tumor in a subject. In another embodiment, the solid tumor is a breast cancer tumor. In another embodiment, the solid tumor is any other type of solid tumor known in the art. Each possibility represents a separate embodiment of the present invention.

In another embodiment, the present invention provides a method of impeding a vascularization of a solid tumor in a subject, the method comprising the step of contacting the subject with a ligand capable of inhibiting an activity of a nucleic acid molecule selected from DR6, ADAM12, CDCP1-CUB, SLC11A1-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, whereby the ligand is taken up by a vasculature cell of the solid tumor, thereby impeding a vascularization of a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of impeding a vascularization of a solid tumor in a subject, the method comprising the step of contacting the subject with a ligand capable of inhibiting an activity of a nucleic acid molecule encoding a protein selected from DR6, ADAM12, CDCP1-CUB, SLC11A1-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, whereby the ligand is taken up by a vasculature cell of the solid tumor, thereby impeding a vascularization of a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of impeding a vascularization of a solid tumor in a subject, the method comprising the step of contacting the subject with a ligand capable of inhibiting an activity of a protein selected from DR6, ADAM12, CDCP1-CUB, SLC11A1-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, whereby the ligand is taken up by a vasculature cell of the solid tumor, thereby impeding a vascularization of a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of impeding a vascularization of a solid tumor in a subject, the method comprising the step of contacting the subject with a ligand capable of inhibiting an activity of an ESM1-encoding nucleic acid molecule or an ESM1 protein, whereby the ligand is taken up by a vasculature cell of the solid tumor, thereby impeding a vascularization of a solid tumor in a subject. In another embodiment, the solid tumor is a renal tumor.

In another embodiment, the solid tumor is any other type of solid tumor known in the art. Each possibility represents a separate embodiment of the present invention.

In another embodiment, the present invention provides a method of impeding a vascularization of a solid tumor in a subject, the method comprising the step of contacting the subject with a ligand capable of inhibiting an activity of a COL11A1-encoding nucleic acid molecule or a COL11A1 protein, whereby the ligand is taken up by a vasculature cell of the solid tumor, thereby impeding a vascularization of a solid tumor in a subject. In another embodiment, the solid tumor is a breast cancer tumor. In another embodiment, the solid tumor is any other type of solid tumor known in the art. Each possibility represents a separate embodiment of the present invention.

In another embodiment, the present invention provides a method of detecting a presence of a solid tumor in a subject, the method comprising the step of detecting a presence of an Adlican protein in a body fluid of the subject, whereby the presence in a body fluid of the protein indicates the presence of a solid tumor in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor in a subject, the method comprising the step of detecting a presence of an Adlican-encoding nucleotide molecule in a body fluid of the subject, whereby the presence in a body fluid of the nucleotide molecule indicates the presence of a solid tumor in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor in a subject, the method comprising the step of detecting a presence of a DR6 protein in a body fluid of the subject, whereby the presence in a body fluid of the protein indicates the presence of a solid tumor in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor in a subject, the method comprising the step of detecting a presence of a DR6-encoding nucleotide molecule in a body fluid of the subject, whereby the presence in a body fluid of the nucleotide molecule indicates the presence of a solid tumor in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor vasculature cell in a subject, the method comprising the step of detecting a presence, in a body fluid of the subject, of a protein selected from FLJ46072, IVNS1ABP, SPP1, TNPAIP6, DKFZp762e1312, WFDC2, KIAA1892, C6orf69, and KIBRA, whereby the presence in a body fluid of the protein indicates the presence of a solid tumor vasculature cell in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor vasculature cell in a subject, the method comprising the step of detecting a presence, in a body fluid of the subject, of a nucleotide molecule that encodes a protein selected from FLJ46072, IVNS1ABP, SPP1, TNPAIP6, DKFZp762e1312, WFDC2, KIAA1892, C6orf69, and KIBRA, whereby the presence in a body fluid of the nucleotide molecule indicates the presence of a solid tumor vasculature cell in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor vasculature cell in a subject, the method comprising the step of detecting a presence of a COL11A1 protein in a body fluid of the subject, whereby the presence in a body fluid of the protein indicates the presence of a solid tumor vasculature cell in the subject. In another embodiment, the solid tumor is a breast cancer tumor. In another embodiment, the solid tumor is any other type of solid tumor known in the art. Each possibility represents a separate embodiment of the present invention.

In another embodiment, the present invention provides a method of detecting a presence of a solid tumor vasculature cell in a subject, the method comprising the step of detecting a presence, in a body fluid of the subject, of a nucleotide molecule encoding a COL11A1 protein, whereby the presence in a body fluid of the nucleotide molecule indicates the presence of a solid tumor vasculature cell in the subject. In another embodiment, the solid tumor is a breast cancer tumor. In another embodiment, the solid tumor is any other type of solid tumor known in the art. Each possibility represents a separate embodiment of the present invention.

In another embodiment, the present invention provides a peptide having the amino acid sequence CPGAKALSRVREDIVEDE (SEQ ID No: 88).

In another embodiment, the present invention provides an antibody that recognizes a peptide of the present invention.

In another embodiment, the present invention provides a method of treating a solid tumor in a subject, the method comprising the steps of localizing the solid tumor by a method of the present invention, and irradiating the solid tumor, thereby treating a solid tumor in a subject. In another embodiment, the solid tumor is an ovarian tumor. In another embodiment, the solid tumor is a breast cancer tumor. In another embodiment, the solid tumor is a renal tumor. In another embodiment, the solid tumor is any other type of solid tumor known in the art. Each possibility represents a separate embodiment of the present invention.

In another embodiment, the present invention provides a method of treating a solid tumor in a subject, the method comprising the steps of: (a) contacting the subject with a photo-activatable cytotoxic drug or pharmaceutical composition; (b) localizing the solid tumor by a method of the present invention; and (c) contacting the solid tumor with a concentrated light source, thereby treating a solid tumor in a subject. In another embodiment, the solid tumor is an ovarian tumor. In another embodiment, the solid tumor is a breast cancer tumor. In another embodiment, the solid tumor is a renal tumor. In another embodiment, the solid tumor is any other type of solid tumor known in the art. Each possibility represents a separate embodiment of the present invention.

### BRIEF DESCRIPTION OF THE FIGURES

FIG. 1: A. Immuno-LCM steps. Left panel: Rapid IHC for CD31 allows prompt identification of vasculature in ovarian cancer frozen sections. Middle panel: Tissue section after LCM of CD31⁺ cells. Right panel: captured tumor vascular cells. B. RT-PCR analysis of lineage-specific markers in RNA from tumor vascular cells (TVC) isolated with immuno-LCM, whole tumor (Tum) or a no template control (NTC). C. Scatter plot and correlation value of amplified RNA from unstained tissue (Pre IHC) versus RNA amplified after IHC optimized as in Table 1 (Post IHC). D. Part 1. Heat map condition tree developed using a hierarchical clustering algorithm, excluding all genes where the difference between the means of the tumor and normal vascular samples was less than its standard error. D, part 2: Red color shown alone. D, part 3: Green color shown alone. E. Archived Gene Expression Datasets. Data used for expression of the TVM in normal and tumor tissue samples; also available in the Gene Expression Omnibus (GEO; National Center for Biotechnology Information [NCBI]) with series numbers GSE3526 and GSE2109, respectively. All CEL-files were similarly processed using the Robust Multi-array Average (RMA) algorithm.

FIG. 2. Quality of total RNA isolated from 8 μm frozen ovarian cancer tissue sections through different methodologies and analyzed by Agilent Bioanalyzer (A-E), quantitative real-time PCR (F) or Affymetrix U133A arrays (G). A. RNA distribution profiles following fixation with different fixatives. (Lanes: 1, ethanol; 2, methanol; 3, acetone; 4, acetic acid+ethanol; 5, paraformaldehyde). B. RNA profiles after isolation without immunostaining (Lane 1) or following IHC with or without RNAse inhibitor. (Lanes: 2, RNA Protector; 3, Placental RNAse inhibitor; 4, SuperRNASin; 5, RNA Protector+SuperRNASin; 6, no RNAse inhibitor. C. RNA profiles after different immunostaining procedures. (Lanes: 1, IHC using DAB; 2, IHC using AEC; 3, immunofluorescence). D. Time course demonstrating RNA stability after IHC performed with procedure optimized as in Table 1. (Lanes: 1, 0 min; 2, 30 min; 3, one hr; 4, two hrs; 5, three hrs). E. RNA profiles following different RNA isolation methods. (Lanes: 1, Arcturus kit; 2, Stratagene kit; 3, modified Trizol; 4, Zymo kit). F. qRT-PCR for β-actin transcripts with RNA purified with the indicated RNA purification protocols. G. Scatter plots and correlation values of amplified RNA (y-axis) to unamplified total tumor RNA (x-axis).

FIG. 3: Expression of novel TVM. A. qRT-PCR analysis of tumor vascular markers (TVM) in whole epithelial ovarian cancer (Ovarian Ca, n=20) and normal ovaries (Nml Ovary, n=5). Results are expressed as relative expression units against β-actin. B. Expression of select TVM by qRT-PCR in a panel of normal human tissues (Sm Intest, small intestine; Sk Muscle, skeletal muscle); expression in ovarian cancer (Ov Ca) is defined as 100%. C. Expression of select TVM by qRT-PCR in different tumor types; expression in ovarian cancer is defined as 100%. Data were normalized against CD31. D. Expression of TVM in tissues with physiologic angiogenesis including corpus luteum (Corp Lut); proliferative endometrium (Prolif Endomet); and placenta, as well as ovarian cancer (Ov Ca). Data were normalized against CD31. E. Expression of TVM in normal human tissues by digital northern derived from the NIH SAGE expression database. Sp Ch (spinal cord), Kid (kidney, Ov (ovary), Lym (lymph node), BM (bone marrow), Vasc (vasculature). Squares with 32-63 tags are indicated by a double arrow to the left. Squares with 16-31 tags have a single arrow to the left. The remainder of the light-colored squares had 15 or fewer tags. “EPB41L3” refers to EPB41L3. This figure also includes additional markers that do not appear in Table 5; selection of these additional markers is described below in Example 11.

FIG. 4. Part 1. Immuno-localization of identified markers to tumor vasculature. IHC is shown for CD24, endothelial lipase (Endo Lipase), FolH1 and AML-1. Immunofluorescent localization of SPON-1 to vascular structures and co-staining of PAR2, STC2 and AML-1 (red) with CD31 (green) in microvascular structures in human ovarian tumors. Merge includes nuclear staining with DAPI. Red fluorescence is shown alone in part 2. Green fluorescence is shown alone in part 3. Blue fluorescence from DAPI is shown alone in part 4.

FIG. 5. A. In-situ hybridization demonstrates expression of mRNA for the indicated tumor vascular markers in association with vascular structures in ovarian cancer. Negative controls included hybridization of tumor section with sense strand (Sense), hybridization of normal ovary with antisense probe (Nml Ov) and hybridization of RNAse-pretreated tumor section with antisense probe (RNAse Tx) for all TVM. Representative controls are shown for adlican, but were identical for all TVM. Merge is shown with DAPI nuclear staining. Red fluorescence is shown alone in part 2. Blue fluorescence from DAPI is shown alone in part 3.

FIG. 6. Expression of select TVM by qRT-PCR in FACS-purified VE-cadherin⁺ CD146⁺ CD45⁻ tumor endothelial cells (TEC) and VE-cadherin⁺ CD146⁺ CD45⁺ vascular leukocytes (VLC) compared with peripheral blood mononuclear cells (PBMC). Data were normalized against β-actin.

FIG. 7, part 1. Expression of novel human TVM by myeloid monocytic cells. A. Flow cytometry analysis; Top, Expression of CD45 (x-axis) and VE-cadherin (y-axis) in DC derived from PBMC prior to exposure to rhVEGF₁₆₅ (Pre VEGF-A) and following 7-day treatment with rhVEGF₁₆₅ (Post VEGF-A); VE-cadherin⁺ CD45⁺ cells appear after VEGF165 treatment. Middle, Expression of CD45 and VE-cadherin in DC derived from enriched monocytes prior to (Pre VEGF-A) and following 7-day treatment with rhVEGF₁₆₅ (Post VEGF-A); 91% of output cells after VEGF₁₆₅ treatment were VE-cadherin⁺ CD45⁺, indicating that double positive cells derived from the monocyte fraction. Bottom, No significant CFSE dilution was noted in monocyte-derived DC after VEGF₁₆₅ treatment, indicating no cell proliferation in these cultures. B. Expression of markers by PBMC-derived DC at baseline (Day 0) and following exposure to rhVEGF₁₆₅, with or without antibody against VEGF-A for 2 weeks. Induction of several tumor vascular markers and suppression of AML-1 and LZTS1 are observed after VEGF₁₆₅. This was abrogated by VEGF blockade, except for AML-1 and LTZS1. Data were normalized against β-actin. C. Expression of TVM in human 2008 ovarian cancer xenografts injected alone (2008) or enriched with human PBMC-derived DC pretreated with VEGF₁₆₅ ex vivo (DC), FACS-purified human tumor endothelial cells (TEC) or FACS-purified human tumor vascular leukocytes (VLC). TVM are expressed by xenografts enriched with human myeloid cells or TEC but not by xenografts generated with human tumor cells only. Data were normalized against human β-actin. D. Immunofluorescent detection of human CD34⁺ vascular structures within 2008 tumors supplemented with human PBMC-derived DC pretreated with VEGF₁₆₅: 1: One CD34-PE positive vessel was noted in the early tumor xenograft. 2 and 3: Merge of CD34-PE and DAPI depicts vascular structures at higher magnification. 4: Control tumor generated with 2008 cells alone without VEGF-pretreated DC, TEC or VLC shows no expression of human CD34. Similar results were obtained with immunostaining for human CD31. E. Raw data-described in legend for FIG. 7C.

FIG. 7, parts 2-3. Red fluorescence is shown alone in part 2. Blue fluorescence from DAPI is shown alone in part 3.

FIG. 8, part 1. Secreted (sec) and transmembrane (TM) TVM. Column 1: expression rank (described in Example 2). Column 2: “Scnt”: The number of normal samples that ranked in the bottom 4 positions. (e.g. if snt=4, the 4 lowest expressing samples are all normal samples). Column 3: Gene symbol. Column 4: Number of normal samples (4 total) in which the transcript was classified as present. Column 5: Number of tumor samples (21 total) in which the transcript was classified as present. Column 6: Mean signal from the tumor samples. Column 7: Mean signal from the normal samples. Column 8: Fold increase of expression in tumor samples. Column 9: foldc3: Value of 1 means that the fold change was (>3). 2 fold change was (<3). Column 10: srank25. 1 means that the sum of ranks <25. Criteria for this list was a value of “1” in at least one of column 9 and column 10.

FIG. 8, part 2. Column 1: Gene symbol. Column 2: Localization. Column 3: Description. Column 4: Available antibodies. Abbreviations: Pred: predicted; Mb: membrane; EC: extracellular; ECM extracellular matrix; PM: plasma membrane.

FIG. 9. A. Real-time PCR analysis of Adlican in normal tissues and ovarian cancer. B. In situ hybridization of adlican in ovarian cancer. C. Real-time PCR analysis of adlican in ovarian tumors. D. Western blot shows adlican protein in tumor tissue lysate (T1-3) but not normal ovary (Nml) (left) and in serum (S1-2) and ascites (A1-2) of patients with ovarian cancer but not normal human serum (NHS).

FIG. 10. Model of step-wise vascular differentiation of monocyte-derived DC precursors in tumors. VEGF-A alone induces in monocytes downregulation of transcriptional regulators AML-1 and LZTS1, while newly identified markers (collagen 11α1, DR6, FZD10, GPM6B, SCP1 and SPON-1) as well as known endothelial markers (VE-cadherin, TEM-1) are upregulated, yielding VLC. Additional factors from the tumor microenvironment cooperate with VEGF-A to induce additional TVM found in TEC, as myeloid cells structurally participate in the assembly of vasculature.

FIG. 11. Additional TVM.

FIG. 12. List of additional TVM from another analysis.

FIG. 13. A. DR6 mRNA expression by qPCR in normal tissues and ovarian tumor. B-D. IHC of DR6 in ovarian tumor (B), high-magnification image thereof (C), and normal ovary tissue (D).

FIG. 14. DR6 protein is detected at higher levels in the serum of patients with ovarian tumors compared to healthy individuals. A—Western. B—quantitation.

FIG. 15. Quantitation of ADAM12 mRNA relative expression by qPCR in normal tissues and ov tumor. A. Long isoform. B. Short isoform.

FIG. 16. Staining with anti-ADAM12 Ab in frozen ovarian tumor sections

FIG. 17. Double fluorescent staining with anti-ADAM12 Ab (red in original) and anti-CD31 Ab (green in original) in frozen ovarian tumor sections. Nuclei are stained blue. Top panel: green color alone (appears as white). Bottom: red color alone (appears as white).

FIG. 18. Double fluorescent staining with anti-ADAM12 Ab (red in original) and anti-CD45 Ab (green in original) in frozen ovarian tumor sections. Nuclei are stained blue. Top panel: green color alone (appears as white). Bottom: orange color alone, representing overlap of green and red staining (appears as white).

FIG. 19. CDCP1 mRNA relative expression in normal tissues and ovarian tumor (qPCR). A. Long isoform. B. Short isoform. Bars represent, from left to right: Ovarian tumor, bladder, esophagus, colon, liver, lung, brain, cervix, kidney, heart, adipose, testes, thymus, skeletal muscle, small intestine, spleen, trachea, ovary, placenta, thyroid, prostate.

FIG. 20. Staining with anti-CDCP1 Ab in frozen ovarian tumor sections.

FIG. 21. SLC11A1 mRNA expression by qPCR in normal tissues and ovarian tumor.

FIG. 22. Staining with anti-SLC11A1 Ab in frozen ovarian tumor sections. Left panels: sample from first patient, at low (top) and high (bottom) magnification. Right panels: sample from second patient, at low (top) and high (bottom) magnification.

FIG. 23. Double fluorescent staining with anti-SLC11A1 Ab (red in original) and anti-CD31 Ab (green in original) in frozen ovarian tumor sections. Nuclei are stained blue. A: green color alone (appears as white). B: red color alone (appears as white).

FIG. 24. Double fluorescent staining with anti-SLC11A1 Ab (red in original) and anti-CD45 Ab (green in original) in frozen ovarian tumor sections. Nuclei are stained blue. A: green color alone (appears as white). B: orange color alone, representing overlap of green and red staining (appears as white).

FIG. 25. BLAME mRNA expression by qPCR in normal tissues and ovarian tumor.

FIG. 26. Staining with anti-BLAME Ab in frozen ovarian tumor sections. A: samples from 2 patients at low magnification (40×). B: samples from 2 patients at high magnification (60×).

FIG. 27. A-B. Double fluorescent staining with anti-BLAME Ab (red in original) and anti-CD31 Ab (green in original) in frozen ovarian tumor sections. Nuclei are stained blue. A: green color alone. B: red color alone. C-E. Double fluorescent staining with anti-BLAME Ab (red in original) and anti-CD45 Ab (green in original) in frozen ovarian tumor sections. Nuclei are stained blue. C: green color alone. D: red color alone. E. orange color alone, representing overlap of green and red staining. Selected color (green, red, or orange) appears as white in each panel.

### DETAILED DESCRIPTION OF THE INVENTION

This invention provides methods of detecting and localizing tumor vasculature cells (TVC) and solid tumors; and treating, impeding vascularization of, and determining the stage of solid tumors in a subject, comprising the step of contacting a subject or a TVC with a ligand that binds to a nucleic acid molecule of the present invention, or binds to a protein encoded by the nucleic acid molecule.

In one embodiment, the present invention provides a method of localizing a solid tumor vasculature in a subject, the method comprising the steps of: (a) contacting the subject with a ligand that binds a DR6-encoding nucleic acid molecule or that binds a DR6 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds an ADAM12-encoding nucleic acid molecule or that binds an ADAM12 protein; and (b) localizing the ligand, thereby localizing a solid tumor vasculature in a subject. In another embodiment, the solid tumor is an ovarian tumor. In another embodiment, the solid tumor is any other type of solid tumor known in the art. In another embodiment, the ADAM12-encoding nucleic acid molecule or ADAM12 protein is a short isoform thereof. In another embodiment, the nucleotide or protein is any other isoform known in the art. Each possibility represents a separate embodiment of the present invention.

In another embodiment, the present invention provides a method of localizing a solid tumor vasculature in a subject, the method comprising the steps of: (a) contacting the subject with a ligand that binds a long isoform of an ADAM12-encoding nucleic acid molecule or an ADAM12 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a CDCP1-CUB-encoding nucleic acid molecule or that binds a CDCP1-CUB protein; and (b) localizing the ligand, thereby localizing a solid tumor vasculature in a subject. In another embodiment, the solid tumor is an ovarian tumor. In another embodiment, the solid tumor is any other type of solid tumor known in the art. In another embodiment, the CDCP1-CUB-encoding nucleic acid molecule or CDCP1-CUB protein is a long isoform thereof. In another embodiment, the nucleotide or protein is any other isoform known in the art. Each possibility represents a separate embodiment of the present invention.

In another embodiment, the present invention provides a method of localizing a solid tumor vasculature in a subject, the method comprising the steps of: (a) contacting the subject with a ligand that binds a short isoform of a CDCP1-CUB-encoding nucleic acid molecule or a CDCP1-CUB protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a SLC11A1-NRAMP-encoding nucleic acid molecule or that binds a SLC11A1-NRAMP protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a BLAME/SLAMF8-encoding nucleic acid molecule or that binds a BLAME/SLAMF8 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds an ESM1-encoding nucleic acid molecule or that binds an ESM1 protein; and (b) localizing the ligand, thereby localizing a solid tumor vasculature in a subject. In another embodiment, the solid tumor is a renal tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds an EGFL6-encoding nucleic acid molecule or that binds an EGFL6 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds an FZD10-encoding nucleic acid molecule or that binds an FZD10 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a COL11A1-encoding nucleic acid molecule or that binds a COL11A1 protein; and (b) localizing the ligand, thereby localizing a solid tumor vasculature in a subject. In another embodiment, the solid tumor is a breast cancer tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a TNFAIP6-encoding nucleic acid molecule or that binds a TNFAIP6 protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds an Adlican-encoding nucleic acid molecule or that binds an Adlican protein; and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a nucleic acid molecule selected from DSG2, c6orf55, SCGB2AI, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a nucleic acid molecule that encodes a protein selected from DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of localizing a solid tumor vasculature in a subject, the method comprising the steps of: (a) contacting the subject with a ligand that binds a protein selected from DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, and (b) localizing the ligand, thereby localizing a solid tumor vasculature in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of treating a solid tumor in a subject, the method comprising the step of contacting the subject with an antibody or ligand that is conjugated to a cytotoxic agent or cytotoxic drug and that binds a nucleic acid molecule produced by the solid tumor, wherein the nucleic acid molecule is selected from DR6, ADAM12, CDCP1-CUB, SLC11A1-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, thereby treating a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of treating a solid tumor in a subject, the method comprising the step of contacting the subject with an antibody or ligand that is conjugated to a cytotoxic agent or cytotoxic drug and that binds a nucleic acid molecule produced by the solid tumor, wherein the nucleic acid molecule encodes a protein selected from DR6, ADAM12, CDCP1-CUB, SLC11A1-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, thereby treating a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of treating a solid tumor in a subject, the method comprising the step of contacting the subject with an antibody or ligand that is conjugated to a cytotoxic agent or cytotoxic drug and that binds a protein produced by the solid tumor, wherein the protein is selected from DR6, ADAM12, CDCP1-CUB, SLC11AI-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, thereby treating a solid tumor in a subject. In another embodiment, the solid tumor is an ovarian tumor. In another embodiment, the solid tumor is any other type of solid tumor known in the art. Each possibility represents a separate embodiment of the present invention.

“Ligand” refers, in another embodiment, to any molecule or structure capable of binding the target molecule. In another embodiment, “ligand” includes antibodies. In another embodiment, the term includes nucleotide molecules that hybridize to a target of interest. In another embodiment, the term includes small molecules with an affinity for the target. Each possibility represents a separate embodiment of the present invention.

In another embodiment, the present invention provides a method of treating a solid tumor in a subject, the method comprising the step of contacting the subject with an antibody or ligand that is conjugated to a cytotoxic agent or cytotoxic drug and that binds an ESM1-encoding nucleic acid molecule or ESM1 protein expressed by the tumor, thereby treating a solid tumor in a subject. In another embodiment, the solid tumor is a renal tumor. In another embodiment, the solid tumor is any other type of solid tumor known in the art. Each possibility represents a separate embodiment of the present invention.

In another embodiment, the present invention provides a method of treating a solid tumor in a subject, the method comprising the step of contacting the subject with an antibody or ligand that is conjugated to a cytotoxic agent or cytotoxic drug and that binds a COL11A1-encoding nucleic acid molecule or COL11A1 protein expressed by the tumor, thereby treating a solid tumor in a subject. In another embodiment, the solid tumor is a breast cancer tumor. In another embodiment, the solid tumor is any other type of solid tumor known in the art. Each possibility represents a separate embodiment of the present invention.

In another embodiment, the present invention provides a method of impeding a vascularization of a solid tumor in a subject, the method comprising the step of contacting the subject with a ligand capable of inhibiting an activity of a nucleic acid molecule selected from DR6, ADAM12, CDCP1-CUB, SLC11A1-NRAMP, BLAME/SLAMF8, EGFL6, FZD10, TNFAIP6, Adlican, DSG2, c6orf55, SCGB2A 1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, whereby the ligand is taken up by a vasculature cell of the solid tumor, thereby impeding a vascularization of a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of impeding a vascularization of a solid tumor in a subject, the method comprising the step of contacting the subject with a ligand capable of inhibiting an activity of a nucleic acid molecule encoding a protein selected from DR6, ADAM12, CDCP1-CUB, SLC11A1-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, whereby the ligand is taken up by a vasculature cell of the solid tumor, thereby impeding a vascularization of a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of impeding a vascularization of a solid tumor in a subject, the method comprising the step of contacting the subject with a ligand capable of inhibiting an activity of a protein selected from DR6, ADAM12, CDCP1-CUB, SLC11A1-NRAMP, BLAME/SLAMF8, EGFL6, FZD10, TNFAIP6, Adlican, DSG2, c6orf55, SCGB2A1, EPSTI1, SEC23B, MS4A6A, LOC51136, KCNE3, KCNE4, c14orf100, C2orf6, KCNK5, C14orf28, PCDHB2, ST14, OLFML2B, GPR105, SDC1, FLJ46072, IVNS1ABP, SPP1, DKFZp762e1312, WFDC2, KIAA1892, C6orf69, and KIBRA, whereby the ligand is taken up by a vasculature cell of the solid tumor, thereby impeding a vascularization of a solid tumor in a subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of impeding a vascularization of a solid tumor in a subject, the method comprising the step of contacting the subject with a ligand capable of inhibiting an activity of an ESM1-encoding nucleic acid molecule or an ESM1 protein, whereby the ligand is taken up by a vasculature cell of the solid tumor, thereby impeding a vascularization of a solid tumor in a subject. In another embodiment, the solid tumor is a renal tumor. In another embodiment, the solid tumor is any other type of solid tumor known in the art. Each possibility represents a separate embodiment of the present invention.

In another embodiment, the present invention provides a method of impeding a vascularization of a solid tumor in a subject, the method comprising the step of contacting the subject with a ligand capable of inhibiting an activity of a COL11A1-encoding nucleic acid molecule or a COL11A1 protein, whereby the ligand is taken up by a vasculature cell of the solid tumor, thereby impeding a vascularization of a solid tumor in a subject. In another embodiment, the solid tumor is a breast cancer tumor. In another embodiment, the solid tumor is any other type of solid tumor known in the art. Each possibility represents a separate embodiment of the present invention.

In another embodiment, the present invention provides a method of detecting a presence of a solid tumor in a subject, the method comprising the step of detecting a presence in a body fluid of the subject of an Adlican protein, whereby the presence in a body fluid of the protein indicates the presence of a solid tumor in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor in a subject, the method comprising the step of detecting a presence in a body fluid of the subject of an Adlican-encoding nucleotide molecule, whereby the presence in a body fluid of the nucleotide molecule indicates the presence of a solid tumor in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor in a subject, the method comprising the step of detecting a presence in a body fluid of the subject of a DR6 protein, whereby the presence in a body fluid of the protein indicates the presence of a solid tumor in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor in a subject, the method comprising the step of detecting a presence in a body fluid of the subject of a DR6-encoding nucleotide molecule, whereby the presence in a body fluid of the nucleotide molecule indicates the presence of a solid tumor in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor vasculature cell in a subject, the method comprising the step of detecting a presence in a body fluid of the subject of a protein selected from FLJ46072, IVNS1ABP, SPP1, TNPAIP6, DKFZp762e1312, WFDC2, KIAA1892, C6orf69, and KIBRA, whereby the presence in a body fluid of the protein indicates the presence of a solid tumor vasculature cell in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor vasculature cell in a subject, the method comprising the step of detecting a presence in a body fluid of the subject of a nucleotide molecule that encodes a protein selected from FLJ46072, IVNS1ABP, SPP1, TNPAIP6, DKFZp762e1312, WFDC2, KIAA1892, C6orf69, and KIBRA, whereby the presence in a body fluid of the nucleotide molecule indicates the presence of a solid tumor vasculature cell in the subject. In another embodiment, the solid tumor is an ovarian tumor. In another embodiment, the solid tumor is any other type of solid tumor known in the art. Each possibility represents a separate embodiment of the present invention.

In another embodiment, the present invention provides a method of detecting a presence of a solid tumor vasculature cell in a subject, the method comprising the step of detecting a presence in a body fluid of the subject of a COL11A1 protein, whereby the presence in a body fluid of the protein indicates the presence of a solid tumor vasculature cell in the subject. In another embodiment, the solid tumor is a breast cancer tumor. In another embodiment, the solid tumor is any other type of solid tumor known in the art. Each possibility represents a separate embodiment of the present invention.

In another embodiment, the present invention provides a method of detecting a presence of a solid tumor vasculature in a subject, the method comprising the step of detecting a presence in a body fluid of the subject of a nucleotide molecule encoding a COL11A1 protein, whereby the presence in a body fluid of the nucleotide molecule indicates the presence of a solid tumor vasculature in the subject. In another embodiment, the solid tumor is a breast cancer tumor. In another embodiment, the solid tumor is any other type of solid tumor known in the art. Each possibility represents a separate embodiment of the present invention.

In another embodiment, the present invention provides a method of localizing an ovarian tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 69; and (b) localizing the ligand, thereby localizing an ovarian tumor vasculature in a subject.

In another embodiment, the present invention provides a method of localizing an ovarian tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 1; and (b) localizing the ligand, thereby localizing an ovarian tumor vasculature in a subject.

In another embodiment, the present invention provides a method of localizing an ovarian tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 119; and (b) localizing the ligand, thereby localizing an ovarian tumor vasculature in a subject.

In another embodiment, the present invention provides a method of localizing an ovarian tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 106; and (b) localizing the ligand, thereby localizing an ovarian tumor vasculature in a subject.

In another embodiment, the present invention provides a method of localizing an ovarian tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 58; and (b) localizing the ligand, thereby localizing an ovarian tumor vasculature in a subject.

In another embodiment, the present invention provides a method of localizing a renal tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 30; and (b) localizing the ligand, thereby localizing a renal tumor vasculature in a subject.

In another embodiment, the present invention provides a method of localizing an ovarian tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 27; and (b) localizing the ligand, thereby localizing an ovarian tumor vasculature in a subject.

In another embodiment, the present invention provides a method of localizing an ovarian tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 37; and (b) localizing the ligand, thereby localizing an ovarian tumor vasculature in a subject.

In another embodiment, the present invention provides a method of localizing a breast cancer tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 21; and (b) localizing the ligand, thereby localizing a breast cancer tumor vasculature in a subject.

In another embodiment, the present invention provides a method of localizing an ovarian tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 68; and (b) localizing the ligand, thereby localizing an ovarian tumor vasculature in a subject.

In another embodiment, the present invention provides a method of localizing an ovarian tumor vasculature in a subject, the method comprising the steps of: (a) contacting the subject with a ligand that binds a nucleic acid molecule, or that binds a protein encoded by the nucleic acid molecule, wherein the sequence of the nucleic acid molecule is set forth in SEQ ID No: 2; and (b) localizing the ligand, thereby localizing an ovarian tumor vasculature in a subject.

In another embodiment, the present invention provides a method of localizing an ovarian tumor vasculature in a subject, the method comprising the steps of: (a) contacting the subject with a ligand that binds a nucleic acid molecule, or that binds a protein encoded by the nucleic acid molecule, the nucleic acid molecule having a sequence selected from the sequences set forth in SEQ ID No: 13, 14, 26, 35, 39, 42, 43, 47, 104, 108, 111, 113, 116, 118, 121, 127, 130, 132, 134, 146, 152, 165, 169, 170, 179, and 181; and (b) localizing the ligand, thereby localizing an ovarian tumor vasculature in a subject.

In another embodiment, the present invention provides a method of treating an ovarian tumor in a subject, the method comprising the step of contacting the subject with an antibody or ligand that is conjugated to a cytotoxic agent or cytotoxic drug and that binds a nucleic acid molecule or a protein encoded by the nucleic acid molecule, wherein the sequence of the nucleic acid molecule is selected from the sequences set forth in SEQ ID No: 1, 2, 13, 14, 26, 27, 35, 37, 39, 42, 43, 47, 58, 68, 69, 104, 106, 108, 111, 113, 116, 118, 119, 121, 127, 130, 132, 134, 146, 152, 165, 169, 170, 179, and 181, thereby treating an ovarian tumor in a subject.

In another embodiment, the present invention provides a method of treating a renal tumor in a subject, the method comprising the step of contacting the subject with an antibody or ligand that is conjugated to a cytotoxic agent or cytotoxic drug and that binds a nucleic acid molecule or a protein encoded by the nucleic acid molecule, wherein the sequence of the nucleic acid molecule is set forth in SEQ ID No: 30, thereby treating a renal tumor in a subject.

In another embodiment, the present invention provides a method of treating a breast cancer tumor in a subject, the method comprising the step of contacting the subject with an antibody or ligand that is conjugated to a cytotoxic agent or cytotoxic drug and that binds a nucleic acid molecule or a protein encoded by the nucleic acid molecule, wherein the sequence of the nucleic acid molecule is set forth in SEQ ID No: 21, thereby treating a breast cancer tumor in a subject.

In another embodiment, the present invention provides a method of impeding a vascularization of an ovarian tumor in a subject, the method comprising the step of contacting the subject with a ligand capable of inhibiting an activity of a nucleic acid molecule or a protein encoded by the nucleic acid molecule wherein the sequence of the nucleic acid molecule is selected from the sequences set forth in SEQ ID No: 1, 2, 13, 14, 26, 27, 35, 37, 39, 42, 43, 47, 58, 68, 69, 104, 106, 108, 111, 113, 116, 118, 119, 121, 127, 130, 132, 134, 146, 152, 165, 169, 170, 179, and 181, whereby the ligand is taken up by a vasculature cell of the ovarian tumor, thereby impeding a vascularization of an ovarian tumor in a subject.

In another embodiment, the present invention provides a method of impeding a vascularization of a renal tumor in a subject, the method comprising the step of contacting the subject with a ligand capable of inhibiting an activity of a nucleic acid molecule or a protein encoded by the nucleic acid molecule wherein the sequence of the nucleic acid molecule is set forth in SEQ ID No: 30, whereby the ligand is taken up by a vasculature cell of the renal tumor, thereby impeding a vascularization of a solid tumor in a subject.

In another embodiment, the present invention provides a method of impeding a vascularization of a breast cancer tumor in a subject, the method comprising the step of contacting the subject with a ligand capable of inhibiting an activity of a nucleic acid molecule or a protein encoded by the nucleic acid molecule wherein the sequence of the nucleic acid molecule is set forth in SEQ ID No: 21, whereby the ligand is taken up by a vasculature cell of the breast cancer tumor, thereby impeding a vascularization of a solid tumor in a subject.

In another embodiment, the present invention provides a method of detecting a presence of an ovarian tumor in a subject, the method comprising the step of detecting a presence in a body fluid of the subject of a protein encoded by a nucleotide molecule, wherein the sequence of the nucleotide molecule is set forth in SEQ ID No: 2, whereby the presence in a body fluid of the protein indicates the presence of an ovarian tumor in the subject.

In another embodiment, the present invention provides a method of detecting a presence of an ovarian tumor in a subject, the method comprising the step of detecting a presence in a body fluid of the subject of a nucleotide molecule, wherein the sequence of the nucleotide molecule is set forth in SEQ ID No: 2, whereby the presence in a body fluid of the nucleotide molecule indicates the presence of an ovarian tumor in the subject.

In another embodiment, the present invention provides a method of detecting a presence of an ovarian tumor in a subject, the method comprising the step of detecting a presence in a body fluid of the subject of a protein encoded by a nucleotide molecule, wherein the sequence of the nucleotide molecule is set forth in SEQ ID No: 69, whereby the presence in a body fluid of the protein indicates the presence of an ovarian tumor in the subject.

In another embodiment, the present invention provides a method of detecting a presence of an ovarian tumor in a subject, the method comprising the step of detecting a presence in a body fluid of the subject of a nucleotide molecule, wherein the sequence of the nucleotide molecule is set forth in SEQ ID No: 69, whereby the presence in a body fluid of the nucleotide molecule indicates the presence of an ovarian tumor in the subject.

In another embodiment, the present invention provides a method of detecting a presence of an ovarian tumor vasculature cell in a subject, the method comprising the step of detecting a presence in a body fluid of the subject of a protein encoded by a nucleotide molecule selected from SEQ ID No: 14, 35, 42, 68, 121, 127, 169, 179, and 181, whereby the presence in a body fluid of the protein indicates the presence of an ovarian tumor vasculature cell in the subject.

In another embodiment, the present invention provides a method of detecting a presence of an ovarian tumor vasculature cell in a subject, the method comprising the step of detecting a presence in a body fluid of the subject of a nucleotide molecule selected from SEQ ID No: 14, 35, 42, 68, 121, 127, 169, 179, and 181, whereby the presence in a body fluid of the nucleotide molecule indicates the presence of an ovarian tumor vasculature cell in the subject.

In another embodiment, the present invention provides a method of detecting a presence of a breast cancer vasculature in a subject, the method comprising the step of detecting a presence in a body fluid of the subject of a protein encoded by a SEQ ID No: 21, whereby the presence in a body fluid of the protein indicates the presence of a breast cancer vasculature in the subject.

In another embodiment, the present invention provides a method of detecting a presence of a breast cancer vasculature cell in a subject, the method comprising the step of detecting a presence in a body fluid of the subject of a nucleotide molecule, wherein the sequence of the nucleotide molecule is set forth in SEQ ID No: 21, whereby the presence in a body fluid of the nucleotide molecule indicates the presence of a breast cancer vasculature cell in the subject.

In another embodiment, the present invention provides a method of detecting the presence of a TVC in a subject, the method comprising the steps of (a) contacting the subject with a ligand that binds to a nucleic acid molecule, the nucleic acid molecule encoding a protein selected from an Adlican protein; a collagen, type XI, alpha 1 (COL11A1) protein; a Defensin, beta 1 (DEFB1) protein; an EPB41L3 protein; a Coagulation factor II (thrombin) receptor-like 1 (F2RL1) protein; a Frizzled 10 (FZD10) protein; a Glycoprotein M6B (GPM6B) protein; a B lymphocyte activator macrophage expressed (BLAME) protein; a Spondin 1 (SPON-1) protein; a Stanniocalcin 2 (STC2) protein; a Tumor necrosis factor, alpha-induced protein 6 (TNFAIP6) protein; and a Tumor necrosis factor receptor superfamily, member 21 (TNFRSF21) protein; and (b) imaging the ligand in the subject, whereby, if the ligand is concentrated in an area of a tissue of the subject, then the subject has a TVC in the area.

“Nucleic acid molecule” and “nucleotide” refer, in another embodiment, to an RNA molecule. In another embodiment, the terms refer to a DNA molecule. In another embodiment, the terms refer to any other type of nucleic acid molecule enumerated herein. In another embodiment, the terms refer to any other type of nucleic acid molecule known in the art. Each possibility represents a separate embodiment of the present invention.

In another embodiment, the present invention provides a method of detecting the presence of a TVC in a subject, the method comprising the steps of (a) contacting the subject with a ligand that binds to a protein selected from an Adlican protein, a COL11A1 protein, a DEFB1 protein, an EPB41L3 protein, a F2RL1 protein, an FZD10 protein, a GPM6B protein, a BLAME protein, a SPON1 protein, a STC2 protein, a TNFAIP6 protein, and a TNFRSF21 protein; and (b) imaging the ligand in the subject, whereby, if the ligand is concentrated in an area of a tissue of the subject, then the subject has a TVC in the area.

In another embodiment, the present invention provides a method of detecting the presence of a TVC in a subject, the method comprising the steps of (a) contacting the subject with a ligand that binds to a nucleic acid molecule, the nucleic acid molecule encoding a protein selected from an AML-1 protein and an LZTS1 protein; and (b) imaging the ligand in the subject, whereby, if the ligand is concentrated in an area of a tissue of the subject, then the subject has a TVC in the area.

In another embodiment, the present invention provides a method of detecting the presence of a TVC in a subject, the method comprising the steps of (a) contacting the subject with a ligand that binds to a protein selected from an AML-1 protein and an LZTS1 protein; and (b) imaging the ligand in the subject, whereby, if the ligand is concentrated in an area of a tissue of the subject, then the subject has a TVC in the area.

In another embodiment, the present invention provides a method of detecting the presence of a TVC in a subject, the method comprising the steps of (a) contacting the subject with a ligand that binds to a nucleic acid molecule, the nucleic acid molecule encoding a protein selected from selected from FAD104; WARP; BCAP29; CDH1; FLJ10826; OPN3; HIATL2; IL28RA; TMEM19; C10orf69; FRAP1; CKLFSF6; MPHOSPH9; CLST11240; MS4A6A; SGPP2; SLC11A1; SLCO3A1; LOC51136; DKFZp56411922; KCNE3; CALM3; KCNE4; MGC34647; MUC1; SDC1; SLC30A6; ST14; CDCP1; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; GPR105; IL7R; ARHGAP18; GRM1; PREX1; MUC3A; EPSTI1; and UBE2J1; and (b) imaging the ligand in the subject, whereby, if the ligand is concentrated in an area of a tissue of the subject, then the subject has a TVC in the area.

In another embodiment, the present invention provides a method of detecting the presence of a TVC in a subject, the method comprising the steps of (a) contacting the subject with a ligand that binds to a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; MS4A6A; SGPP2; SLC11A1; SLCO3A1; LOC51136; DKFZp56411922; KCNE3; CALM3; KCNE4; MGC34647; MUC1; SDC1; SLC30A6; ST14; CDCP1; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; GPR105; IL7R; ARHGAP18; GRM1; PREX1; MUC3A; EPSTI1; and UBE2J1; and (b) imaging the ligand in the subject, whereby, if the ligand is concentrated in an area of a tissue of the subject, then the subject has a TVC in the area.

“Imaging” refers, in another embodiment, to localizing a ligand of interest using an imaging or scanning technology. In another embodiment, the ligand is a fluorescent ligand. In another embodiment, the ligand is radioactive. In another embodiment, the ligand is bound by a molecule (e.g. an antibody) that is detectable by the imaging or scanning technology. In another embodiment, any suitable imaging or scanning technology known in the art may be utilized. Each possibility represents a separate embodiment of the present invention.

In another embodiment of methods of the present invention, the TVM is expressed at detectable levels only in the TVC, but not in the surrounding tissue. In another embodiment, the TVC is expressed at significantly higher levels in the TVC, relative to the surrounding tissue. In another embodiment, the TVM is expressed at detectable levels only in the TVC, but not in other body tissues. In another embodiment, the TVC is expressed at significantly higher levels in the TVC, relative to other body tissues. Each possibility represents a separate embodiment of the present invention.

As provided herein, a rapid protocol was developed and optimized for immuno-LCM of TVC, followed by extraction and amplification of RNA for array analysis of tumor vascular cells (Example 1). This enabled identification of the novel tumor vasculature markers (TVM) of the present invention. The identified transcripts and proteins encoded thereby were validated as TVM by a number of independent lines of evidence, including enrichment in independent tumor samples, relative to normal vascular samples; enrichment in tumor tissue relative to a variety of tissue samples; and comparison of expression levels between tumor tissue and tissues with physiologic angiogenesis (Example 3).

In another embodiment, the present invention provides a method of detecting a solid tumor in a subject, the method comprising the steps of (a) contacting the subject with a ligand that binds to a nucleic acid molecule, the nucleic acid molecule encoding a protein selected from an Adlican protein, a COL11A1 protein, a DEFB1 protein, an EPB41L3 protein, a F2RL1 protein, an FZD10 protein, a GPM6B protein, a BLAME protein, a SPON1 protein, a STC2 protein, a TNFAIP6 protein, and a TNFRSF21 protein; and (b) imaging the ligand in the subject, whereby, if the ligand is concentrated in an area of a tissue of the subject, then the subject has a solid tumor in the area.

In another embodiment, the present invention provides a method of detecting a solid tumor in a subject, the method comprising the steps of (a) contacting the subject with a ligand that binds to a protein selected from an Adlican protein, a COL11A1 protein, a DEFB1 protein, an EPB41L3 protein, a F2RL1 protein, an FZD10 protein, a GPM6B protein, a BLAME protein, a SPON1 protein, a STC2 protein, a TNFAIP6 protein, and a TNFRSF21 protein; and (b) imaging the ligand in the subject, whereby, if the ligand is concentrated in an area of a tissue of the subject, then the subject has a solid tumor in the area.

In another embodiment, the present invention provides a method of detecting a solid tumor in a subject, the method comprising the steps of (a) contacting the subject with a ligand that binds to a nucleic acid molecule, the nucleic acid molecule encoding a protein selected from an AML-1 protein and an LZTS1 protein; and (b) imaging the ligand in the subject, whereby, if the ligand is concentrated in an area of a tissue of the subject, then the subject has a solid tumor in the area.

In another embodiment, the present invention provides a method of detecting a solid tumor in a subject, the method comprising the steps of (a) contacting the subject with a ligand that binds to a protein selected from an AML-1 protein and an LZTS1 protein; and (b) imaging the ligand in the subject, whereby, if the ligand is concentrated in an area of a tissue of the subject, then the subject has a solid tumor in the area.

In another embodiment, the present invention provides a method of detecting a solid tumor in a subject, the method comprising the steps of (a) contacting the subject with a ligand that binds to a nucleic acid molecule, the nucleic acid molecule encoding a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; MS4A6A; SGPP2; SLC11A1; SLCO3A1; LOC51136; DKFZp56411922; KCNE3; CALM3; KCNE4; MGC34647; MUC1; SDC1; SLC30A6; ST14; CDCP1; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; GPR105; IL7R; ARHGAP18; GRM1; PREX1; MUC3A; EPSTI1; and UBE2A1; and (b) imaging the ligand in the subject, whereby, if the ligand is concentrated in an area of a tissue of the subject, then the subject has a solid tumor in the area.

In another embodiment, the present invention provides a method of detecting a solid tumor in a subject, the method comprising the steps of (a) contacting the subject with a ligand that binds to a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; MS4A6A; SGPP2; SLC1A1; SLCO3A1; LOC51136; DKFZp56411922; KCNE3; CALM3; KCNE4; MGC34647; MUC1; SDC1; SLC30A6; ST14; CDCP1; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; GPR105; IL7R; ARHGAP18; GRM1; PREX1; MUC3A; EPSTI1; and UBE2J1; and (b) imaging the ligand in the subject, whereby, if the ligand is concentrated in an area of a tissue of the subject, then the subject has a solid tumor in the area.

In another embodiment, the present invention provides a method of localizing a tumor vasculature in a subject, the method comprising the steps of (a) contacting the tumor vasculature with a ligand that binds to a nucleic acid molecule, the nucleic acid molecule encoding a protein selected from a DEFB1 protein, an EPB41L3 protein, a F2RL1 protein, a GPM6B protein, a SPON1 protein, and a STC2 protein; and (b) localizing the ligand, thereby localizing a tumor vasculature.

As provided herein, certain TVM transcripts of the present invention and the proteins encoded thereby are efficacious in localizing solid tumors and vasculature thereof (Examples 4 and 5).

In another embodiment, the present invention provides a method of localizing a tumor vasculature in a subject, the method comprising the steps of (a) contacting the tumor vasculature with a ligand that binds to a protein selected from a DEFB1 protein, an EPB41L3 protein, a F2RL1 protein, a GPM6B protein, a SPON1 protein, and a STC2 protein; and (b) localizing the ligand, thereby localizing a tumor vasculature in a subject.

In another embodiment, the present invention provides a method of localizing a tumor vasculature in a subject, the method comprising the steps of (a) contacting the tumor vasculature with a ligand that binds to a nucleic acid molecule, the nucleic acid molecule encoding a protein selected from an AML-1 protein and an LZTS1 protein; and (b) localizing the ligand, thereby localizing a tumor vasculature in a subject.

In another embodiment, the present invention provides a method of localizing a tumor vasculature in a subject, the method comprising the steps of (a) contacting the tumor vasculature with a ligand that binds to a protein selected from an AML-1 protein and an LZTS1 protein; and (b) localizing the ligand, thereby localizing a tumor vasculature in a subject.

In another embodiment, the present invention provides a method of localizing a tumor vasculature in a subject, the method comprising the steps of (a) contacting the tumor vasculature with a ligand that binds to a nucleic acid molecule, the nucleic acid molecule encoding a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; SGPP2; SLCO3A1; DKFZp56411922; CALM3; MGC34647; MUC1; SLC30A6; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; IL7R; ARHGAP18; GRM1; PREX1; MUC3A; and UBE2J1; and (b) localizing the ligand, thereby localizing a tumor vasculature in a subject.

In another embodiment, the present invention provides a method of localizing a tumor vasculature in a subject, the method comprising the steps of (a) contacting the tumor vasculature with a ligand that binds to a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; SGPP2; SLCO3A1; DKFZp56411922; CALM3; MGC34647; MUC1; SLC30A6; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; IL7R; ARHGAP18; GRM1; PREX1; MUC3A; and UBE2J1; and (b) localizing the ligand, thereby localizing a tumor vasculature in a subject.

In another embodiment, the present invention provides a method of localizing a solid tumor in a subject, the method comprising the steps of (a) contacting the subject with a ligand that binds to a nucleic acid molecule, the nucleic acid molecule encoding a protein selected from an Adlican protein, a COL11A1 protein, a DEFB1 protein, an EPB41L3 protein, a F2RL1 protein, an FZD10 protein, a GPM6B protein, a BLAME protein, a SPON1 protein, a STC2 protein, a TNFAIP6 protein, and a TNFRSF21 protein; and (b) localizing the ligand, thereby localizing a solid tumor in a subject.

In another embodiment, the present invention provides a method of localizing a solid tumor in a subject, the method comprising the steps of (a) contacting the subject with a ligand that binds to a protein selected from an Adlican protein, a COL11A1 protein, a DEFB1 protein, an EPB41L3 protein, a F2RL1 protein, an FZD10 protein, a GPM6B protein, a BLAME protein, a SPON1 protein, a STC2 protein, a TNFAIP6 protein, and a TNFRSF21 protein; and (b) localizing the ligand, thereby localizing a solid tumor in a subject.

In another embodiment, the present invention provides a method of localizing a solid tumor in a subject, the method comprising the steps of (a) contacting the subject with a ligand that binds to a nucleic acid molecule, the nucleic acid molecule encoding a protein selected from an AML-1 protein and an LZTS1 protein; and (b) localizing the ligand, thereby localizing a solid tumor in a subject.

In another embodiment, the present invention provides a method of localizing a solid tumor in a subject, the method comprising the steps of (a) contacting the subject with a ligand that binds to a protein selected from an AML-1 protein and an LZTS1 protein; and (b) localizing the ligand, thereby localizing a solid tumor in a subject.

In another embodiment, the present invention provides a method of localizing a solid tumor in a subject, the method comprising the steps of (a) contacting the subject with a ligand that binds to a nucleic acid molecule, the nucleic acid molecule encoding a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; MS4A6A; SGPP2; SLC11A1; SLC03A1; LOC51136; DKFZp56411922; KCNE3; CALM3; KCNE4; MGC34647; MUC1; SDC1; SLC30A6; ST14; CDCP1; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; GPR105; IL7R; ARHGAP18; GRM1; PREX1; MUC3A; EPSTI1; and UBE2J1; and (b) localizing the ligand, thereby localizing a solid tumor in a subject.

In another embodiment, the present invention provides a method of localizing a solid tumor in a subject, the method comprising the steps of (a) contacting the subject with a ligand that binds to a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; MS4A6A; SGPP2; SLC11A1; SLCO3A1; LOC51136; DKFZp56411922; KCNE3; CALM3; KCNE4; MGC34647; MUC1; SDC1; SLC30A6; ST14; CDCP1; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; GPR105; IL7R; ARHGAP18; GRM1; PREX1; MUC3A; EPSTI1; and UBE2J1; and (b) localizing the ligand, thereby localizing a solid tumor in a subject.

In another embodiment, a method of present invention of localizing or detecting a tumor, TVC, or tumor vasculature utilizes positron-emission tomography (PET) scanning. In another embodiment, the method utilizing any other imaging technique known in the art. Each possibility represents a separate embodiment of the present invention.

In another embodiment, the present invention provides a method of treating a solid tumor in a subject, the method comprising the step of contacting the subject with an antibody or ligand that is conjugated to a anti-cancer agent and that binds to a nucleic acid molecule, the nucleic acid molecule encoding a protein selected from a DEFB1 protein, an EPB41L3 protein, a F2RL1 protein, a GPM6B protein, a SPON1 protein, and a STC2 protein, thereby treating a solid tumor in a subject.

In another embodiment, the present invention provides a method of treating a solid tumor in a subject, the method comprising the step of contacting the subject with an antibody or ligand that is conjugated to a anti-cancer agent and that binds to a protein selected from a DEFB1 protein, an EPB41L3 protein, a F2RL1 protein, a GPM6B protein, a SPON1 protein, and a STC2 protein, thereby treating a solid tumor in a subject.

In another embodiment, the present invention provides a method of treating a solid tumor in a subject, the method comprising the step of contacting the subject with an antibody or ligand that is conjugated to a anti-cancer agent and that binds to a nucleic acid molecule, the nucleic acid molecule encoding a protein selected from an AML-1 protein and an LZTS1 protein, thereby treating a solid tumor in a subject.

In another embodiment, the present invention provides a method of treating a solid tumor in a subject, the method comprising the step of contacting the subject with an antibody or ligand that is conjugated to a anti-cancer agent and that binds to a protein selected from an AML-1 protein and an LZTS1 protein, thereby treating a solid tumor in a subject.

In another embodiment, the present invention provides a method of treating a solid tumor in a subject, the method comprising the step of contacting the subject with an antibody or ligand that is conjugated to a anti-cancer agent and that binds to a nucleic acid molecule, the nucleic acid molecule encoding a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; SGPP2; SLCO3A1; DKFZp56411922; CALM3; MGC34647; MUC1; SLC30A6; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; IL7R; ARHGAP18; GRM1; PREX1; MUC3A; and UBE2J1, thereby treating a solid tumor in a subject.

In another embodiment, the present invention provides a method of treating a solid tumor in a subject, the method comprising the step of contacting the subject with an antibody or ligand that is conjugated to a anti-cancer agent and that binds to a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; SGPP2; SLCO3A1; DKFZp56411922; CALM3; MGC34647; MUC1; SLC30A6; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; IL7R; ARHGAP18; GRM1; PREX1; MUC3A; and UBE2J1, thereby treating a solid tumor in a subject.

As provided in the Examples herein, certain TVM of the present invention are expressed at detectable levels only by TVC. In another embodiment, the TVM are expressed at higher levels by TVC than by healthy tissue. Thus, TVM provide a means of specifically targeting therapeutic modalities to solid tumors and their vasculature.

In another embodiment, the present invention provides a method of inducing a regression of a solid tumor in a subject, the method comprising the step of contacting the subject with an antibody or ligand that is conjugated to a anti-cancer agent and that binds to a nucleic acid molecule, the nucleic acid molecule encoding a protein selected from an Adlican protein, a COL11A1 protein, a DEFB1 protein, an EPB41L3 protein, a F2RL1 protein, an FZD10 protein, a GPM6B protein, a BLAME protein, a SPON1 protein, a STC2 protein, a TNFAIP6 protein, and a TNFRSF21 protein, thereby inducing a regression of a solid tumor in a subject.

In another embodiment, the present invention provides a method of inducing a regression of a solid tumor in a subject, the method comprising the step of contacting the subject with an antibody or ligand that is conjugated to a anti-cancer agent and that binds to a protein selected from an Adlican protein, a COL11A1 protein, a DEFB1 protein, an EPB41L3 protein, a F2RL1 protein, an FZD10 protein, a GPM6B protein, a BLAME protein, a SPON1 protein, a STC2 protein, a TNFAIP6 protein, and a TNFRSF21 protein, thereby inducing a regression of a solid tumor in a subject.

The anti-cancer agent utilized in methods and compositions of the present invention is, in another embodiment, a radioactive isotope. In another embodiment, the anti-cancer agent is a cytotoxic agent. In another embodiment, the anti-cancer agent is a cytotoxic drug. In another embodiment, the anti-cancer agent is a nucleic acid molecule. In another embodiment, the anti-cancer agent is an antisense molecule. In another embodiment, the anti-cancer agent is an RNA inhibitory molecule. In another embodiment, the anti-cancer agent is an anti-tumor agent. In another embodiment, the anti-cancer agent is a cytotoxic virus. In another embodiment, the anti-cancer agent is a cytotoxic pathogen. In another embodiment, the anti-cancer agent is a nanosphere. In another embodiment, the nanosphere is loaded with a cytotoxic compound. In another embodiment, the nanosphere is loaded with a chemotherapy drug. In another embodiment, the nanosphere is loaded with a toxin. In another embodiment, the nanosphere is loaded with an anti-cancer compound. In another embodiment, the anti-cancer agent is a nanoparticle. In another embodiment, the anti-cancer agent is an engineered T cell. In another embodiment, the anti-cancer agent is an engineered cytotoxic cell. In another embodiment, the anti-cancer agent is any other type of engineered molecule known in the art. In another embodiment, the anti-cancer agent is any other agent used in cancer therapy. In another embodiment, the anti-cancer agent is any other type of anti-cancer agent known in the art. Each possibility represents a separate embodiment of the present invention.

“Engineered T cell” refers, in another embodiment, to a T cell designed to recognize a cell containing or expressing a molecule of interest. In another embodiment, the molecule of interest is a TVM of the present invention. In another embodiment, the term refers to a T cell with redirected specificity (T-bodies) for a TVM. In another embodiment, an engineered T cell of the present invention expresses a ligand that binds to or interacts with a TVM. In another embodiment, the engineered T cell exhibits specific activity against a TVC.

In another embodiment, an engineered T cell of the present invention expresses a chimeric immunoreceptor (CIR) directed against a TVM. In another embodiment, the CIR contains a bi-partite signaling module. In another embodiment, the extracellular module of the CIR is a single chain variable fragment (scFv) antibody that binds or interacts with a TVM. In another embodiment, the intracellular module of the CIR contains a costimulatory domain. In another embodiment, the costimulatory domain is a 4-1BB domain. In another embodiment, the costimulatory domain is a TCRζ domain. In another embodiment, the CIR contains both a 4-1BB domain and a TCRζ domain.

In another embodiment, an engineered T cell of the present invention is expanded in culture. In another embodiment, an engineered T cell of the present invention is activated in culture.

Each type of engineered T cell represents a separate embodiment of the present invention.

“Cytotoxic virus” refers, in another embodiment, to a virus capable of lysing a cell. In another embodiment, the term refers to a virus capable of lysing a tumor cell. In another embodiment, the virus is a recombinant virus that has been engineered to exhibit a characteristic favorable for anti-tumor activity. In another embodiment, the virus is wild-type, other than is conjugation to an antibody or ligand of the present invention. In another embodiment, the virus is an attenuated virus. Each possibility represents a separate embodiment of the present invention.

In another embodiment, the cytotoxic agent or anti-tumor agent is concentrated in the solid tumor. In another embodiment, the cytotoxic agent or anti-tumor agent is targeted to the solid tumor. In another embodiment, concentration of the cytotoxic agent or anti-tumor agent induces cytotoxicity in a tumor cell of the solid tumor. Each possibility represents a separate embodiment of the present invention.

In another embodiment, the present invention provides a method of impeding a vascularization of a solid tumor, the method comprising the step of contacting the subject with a ligand capable of binding to or inhibiting an activity of a nucleic acid molecule, the nucleic acid molecule encoding a protein selected from a DEFB1 protein, an EPB41L3 protein, a F2RL1 protein, a GPM6B protein, a SPON1 protein, and a STC2 protein, whereby the ligand is taken up by a vasculature cell of the solid tumor, thereby impeding a vascularization of a solid tumor.

In another embodiment, the present invention provides a method of impeding a vascularization of a solid tumor, the method comprising the step of contacting the subject with an antibody or ligand that binds to or inhibits an activity of a protein selected from a DEFB1 protein, an EPB41L3 protein, a F2RL1 protein, a GPM6B protein, a SPON1 protein, and a STC2 protein, whereby the ligand inhibits an activity of a vasculature cell of the solid tumor, thereby impeding a vascularization of a solid tumor.

In another embodiment, the present invention provides a method of impeding a vascularization of a solid tumor, the method comprising the step of contacting the subject with a ligand capable of binding to or inhibiting an activity of a nucleic acid molecule, the nucleic acid molecule encoding a protein selected from an AML-1 protein and an LZTS1 protein, whereby the ligand is taken up by a vasculature cell of the solid tumor, thereby impeding a vascularization of a solid tumor.

In another embodiment, the present invention provides a method of impeding a vascularization of a solid tumor, the method comprising the step of contacting the subject with an antibody or ligand that binds to or inhibits an activity of a protein selected from an AML-1 protein and an LZTS1 protein, whereby the ligand inhibits an activity of a vasculature cell of the solid tumor, thereby impeding a vascularization of a solid tumor.

In another embodiment, the present invention provides a method of impeding a vascularization of a solid tumor, the method comprising the step of contacting the subject with a ligand capable of binding to or inhibiting an activity of a nucleic acid molecule, the nucleic acid molecule encoding a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; SGPP2; SLCO3A1; DKFZp56411922; CALM3; MGC34647; MUC1; SLC30A6; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; IL7R; ARHGAP18; GRM1; PREX1; MUC3A; and UBE2J1, whereby the ligand is taken up by a vasculature cell of the solid tumor, thereby impeding a vascularization of a solid tumor.

In another embodiment, the present invention provides a method of impeding a vascularization of a solid tumor, the method comprising the step of contacting the subject with an antibody or ligand that binds to or inhibits an activity of a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; SGPP2; SLCO3A1; DKFZp56411922; CALM3; MGC34647; MUC1; SLC30A6; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; IL7R; ARHGAP18; GRM1; PREX1; MUC3A; and UBE2J1, whereby the ligand inhibits an activity of a vasculature cell of the solid tumor, thereby impeding a vascularization of a solid tumor.

In another embodiment, the present invention provides a method of impeding an angiogenesis in a subject, the method comprising the step of contacting the subject with a ligand capable of binding to a nucleic acid molecule, wherein the nucleic acid molecule is a TVM of the present invention, whereby the ligand is taken up by a vasculature cell of the solid tumor or a precursor of the vasculature cell, thereby impeding an angiogenesis in a subject.

In another embodiment, the present invention provides a method of impeding an angiogenesis in a subject, the method comprising the step of contacting the subject with an antibody or ligand that binds to a protein that is a TVM of the present invention, whereby the ligand inhibits an activity of a vasculature cell of the solid tumor or a precursor of the vasculature cell, thereby impeding an angiogenesis in a subject.

“Vasculature cell” refers, in another embodiment, to a cell capable of forming a tumor vasculature. In another embodiment, the term refers to a cell of a tumor vasculature. In another embodiment, “precursor of the vasculature cell” refers to a cell capable of differentiating into a cell capable of forming a tumor vasculature. In another embodiment, the term refers to a cell capable of differentiating into a cell of a tumor vasculature. Each possibility represents a separate embodiment of the present invention.

“Activity” refers, in another embodiment, to an angiogenic activity. In another embodiment, the term refers to production of a protein of the present invention. In another embodiment, the term refers to any other tumorigenic activity known in the art. Each possibility represents a separate embodiment of the present invention.

In another embodiment, the ligand reduces the expression of the nucleic acid to which it binds, or the expression of the protein encoded by the nucleic acid. In another embodiment, the ligand reduces the activity of the protein to which it binds. In another embodiment, binding to the ligand results in degradation of the nucleic acid molecule or protein to which it binds. In another embodiment, the ligand antagonizes the nucleic acid molecule or protein to which it binds via any other mechanism known in the art. Each possibility represents a separate embodiment of the present invention.

As provided herein, certain TVM of the present invention are up-regulated upon differentiation of precursor cells into TVC. Thus, these TVM (both the nucleic acid molecules and the proteins encoded thereby) play important roles in the function of TVC in angiogenesis, and thus in the pathogenesis of solid tumors. Accordingly, targeting the expression or activity of the nucleic acids and proteins represents an efficacious means of impeding vascularization of solid tumors.

The ligand utilized in methods and compositions of the present invention is, in another embodiment, an antisense molecule. In another embodiment, the ligand is an RNA molecule. In another embodiment, the ligand is an inhibitory RNA (RNAi) molecule. In another embodiment, the RNA molecule is a short hairpin RNA (shRNA). In another embodiment, the RNA molecule is a small inhibitory RNA (siRNA). In another embodiment, the RNA molecule is a microRNA (miRNA). The use of siRNA and miRNA has been described, inter alia, in (Caudy A A et al, Genes & Devel 16: 2491-96 and references cited therein). In another embodiment, the RNA molecule is an anti-sense locked-nucleic acid (LNA) oligonucleotide. In another embodiment, the RNA molecule is any type of inhibitory RNA enumerated or described in Banan M et al (The ins and outs of RNAi in mammalian cells. Curr Pharm Biotechnol. 2004 October; 5(5):441-50. In another embodiment, the RNA molecule is any type of RNAi known in the art.

In another embodiment, an RNA molecule utilized in methods of the present invention comprises a string of at least two base-sugar-phosphate combinations. The term includes, in another embodiment, compounds comprising nucleotides in which the sugar moiety is ribose. In another embodiment, the term includes both RNA and RNA derivates in which the backbone is modified. “Nucleotides” refers, in one embodiment, to the monomeric units of nucleic acid polymers. RNA may be, in another embodiment, in the form of a tRNA (transfer RNA), snRNA (small nuclear RNA), rRNA (ribosomal RNA), mRNA (messenger RNA), and ribozymes. In addition, these forms of RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E (Curr Opin Struct Biol 9:353-57); and Raz N K et al (Biochem Biophys Res Commun. 297:1075-84). The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention.

In another embodiment, the ligand induces degradation of the target nucleic acid molecule. In another embodiment, the ligand inhibits transcription of the nucleic acid molecule. In another embodiment, the ligand kills the TVC. In another embodiment, the ligand induces apoptosis of the TVC. In another embodiment, the ligand induces necrosis of the TVC. In another embodiment, the ligand inhibits proliferation of the TVC. In another embodiment, the ligand inhibits division of the TVC. In another embodiment, the ligand inhibits growth of the solid tumor. In another embodiment, the ligand induces regression of the solid tumor. Each possibility represents a separate embodiment of the present invention.

The activity of a vasculature cell that is inhibited by methods of the present invention, is, another embodiment, a catalytic activity. In another embodiment, the activity is interaction with another cell. In another embodiment, the activity is cell-cell adhesion. In another embodiment, the activity is contact repulsion. In another embodiment, the activity is DNA binding. In another embodiment, the activity is transcriptional regulation. In another embodiment, the activity is cartilage condensation. In another embodiment, the activity is protein binding. In another embodiment, the activity is extracellular structure organization and/or biogenesis. In another embodiment, the activity is phosphate transport. In another embodiment, the activity is G-protein coupled receptor protein signaling. In another embodiment, the activity is intracellular calcium signaling. In another embodiment, the activity is rhodopsin-like receptor activity. In another embodiment, the activity is thrombin receptor activity. In another embodiment, the activity is cell cycle modulation. In another embodiment, the activity is cell cycle inhibition. In another embodiment, the activity is cell surface receptor-linked signal transduction. In another embodiment, the activity is inflammatory signaling. In another embodiment, the activity is anti-inflammatory signaling. In another embodiment, the activity is hyaluronic acid binding. In another embodiment, the activity is any other activity performed by a protein encoded by a nucleic acid molecule having a sequence selected from the sequences set forth in SEQ ID No: 2, 5, 21, 25, 29, 36-38, 44, 58, 60, 62, and 68-69. Each possibility represents a separate embodiment of the present invention.

In another embodiment, the present invention provides a method of determining a stage of a solid tumor in a subject, the method comprising the steps of (a) contacting the subject with a ligand capable of binding to a nucleic acid molecule, the nucleic acid molecule encoding a protein selected from an Adlican protein, a COL11A1 protein, a DEFB1 protein, an EPB41L3 protein, a F2RL1 protein, an FZD10 protein, a GPM6B protein, a BLAME protein, a SPON1 protein, a STC2 protein, a TNFAIP6 protein, and a TNFRSF21 protein; (b) imaging the ligand in the subject, whereby if the ligand is concentrated in the solid tumor, then the solid tumor has a tumor vasculature.

As provided herein, TVM of the present invention are upregulated upon differentiation to TVC, both in vitro and in vivo (Examples 6-7), showing that expression levels of these proteins, and nucleotides encoding same can be used to determine the sate of a solid tumor.

In another embodiment, the present invention provides a method of determining a stage of a solid tumor, the method comprising the steps of (a) contacting the solid tumor with a ligand capable of binding to a protein selected from an Adlican protein, a COL11A1 protein, a DEFB1 protein, an EPB41L3 protein, a F2RL1 protein, an FZD10 protein, a GPM6B protein, a BLAME protein, a SPON1 protein, a STC2 protein, a TNFAIP6 protein, and a TNFRSF21 protein; (b) imaging the ligand in the subject, whereby if the ligand is concentrated in the solid tumor, then the solid tumor has a tumor vasculature.

In another embodiment, the present invention provides a method of determining a stage of a solid tumor, the method comprising the steps of (a) contacting the solid tumor with a ligand capable of binding to a nucleic acid molecule, the nucleic acid molecule encoding a protein selected from an AML-1 protein and an LZTS1 protein; (b) imaging the ligand in the subject. In another embodiment if the ligand is concentrated in the solid tumor, then the solid tumor has a tumor vasculature. In another embodiment, the solid tumor is forming a tumor vasculature. In another embodiment, the solid tumor is committed to forming a tumor vasculature. Each possibility represents a separate embodiment of the present invention.

In another embodiment, the present invention provides a method of determining a stage of a solid tumor, the method comprising the steps of (a) contacting the solid tumor with a ligand capable of binding to a protein selected from an AML-1 protein and an LZTS1 protein; (b) imaging the ligand in the subject. In another embodiment if the ligand is concentrated in the solid tumor, then the solid tumor has a tumor vasculature. In another embodiment, the solid tumor is forming a tumor vasculature. In another embodiment, the solid tumor is committed to forming a tumor vasculature. Each possibility represents a separate embodiment of the present invention.

In another embodiment, the present invention provides a method of determining a stage of a solid tumor, the method comprising the steps of (a) contacting the solid tumor with a ligand capable of binding to a nucleic acid molecule, the nucleic acid molecule encoding a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; MS4A6A; SGPP2; SLC11A1; SLCO3A1; LOC51136; DKFZp56411922; KCNE3; CALM3; KCNE4; MGC34647; MUC1; SDC1; SLC30A6; ST14; CDCP1; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA 703; C1orf10; POLYDOM; TUBAL3; GPR105; IL7R; ARHGAP18; GRM1; PREX1; MUC3A; EPSTI1; and UBE2J1; (b) imaging the ligand in the subject, whereby if the ligand is concentrated in the solid tumor, then the solid tumor has a tumor vasculature.

In another embodiment, the present invention provides a method of determining a stage of a solid tumor, the method comprising the steps of (a) contacting the solid tumor with a ligand capable of binding to a protein selected from FAD104; WARP; BCAP29; CDH1; FLJ10826; OPN3; HIATL2; IL28RA; TMEM19; C10orf69; FRAP1; CKLFSF6; MPHOSPH9; CLST11240; MS4A6A; SGPP2; SLC11A1; SLCO3A1; LOC51136; DKFZp56411922; KCNE3; CALM3; KCNE4; MGC34647; MUC1; SDC1; SLC30A6; ST14; CDCP1; TLCD1; SPTB; FNDC3; SPRY1; MME; INSR; LPPR4; C14orf100; SLC9A5; SCGB2A1; FLT1; MOBK1B; TMEM2; TMEM8; SLC5A4; MEST; CHODL; TRIO; IL10RA; LGALS3BP; STK4; ERBB3; C14orf28; KIAA1024; KIAA1906; F3; PCDHB2; KIAA0703; C1orf10; POLYDOM; TUBAL3; GPR105; IL7R; ARHGAP18; GRM1; PREX1; MUC3A; EPSTI1; and UBE2J1; (b) imaging the ligand in the subject, whereby if the ligand is concentrated in the solid tumor, then the solid tumor has a tumor vasculature.

In another embodiment, the present invention provides a method of localizing or detecting a presence of a TEC (Tumor Endothelial Cell) in a subject, the method comprising the steps of (a) contacting the subject with a ligand that binds to a nucleic acid molecule, the nucleic acid molecule encoding a protein selected from an Adlican protein; an EGF-like-domain, multiple 6 (EGFL6) protein; a Frizzled 10 (FZD10) protein; a Glycoprotein M6B (GPM6B) protein; and a Spondin 1 (SPON-1) protein; and (b) imaging the ligand in the subject, whereby, if the ligand is concentrated in an area of a tissue of the subject, then the subject has a TEC in the area.

In another embodiment, the present invention provides a method of detecting a presence of a solid tumor in a subject, the method comprising the step of detecting a presence in a body fluid of the subject of a protein selected from TNFAIP6; STC2; ESM1; IBSP; CKLFSF6; HAPLN1; WFDC2; SPP1; FLT1; LGALS3BP; CCL15; PLA2G2D; MUC3A; and LTBP2 (Latent transforming growth factor beta binding protein 2), wherein the presence in a body fluid of the subject of the protein indicates the presence of a solid tumor in the subject.

In another embodiment, the present invention provides a method of detecting a presence of a tumor vasculature cell in a subject, the method comprising the step of detecting a presence in a body fluid of the subject of a protein selected from TNFAIP6; STC2; ESM1; IBSP; CKLFSF6; HAPLN1; WFDC2; SPP1; FLT1; LGALS3BP; CCL15; PLA2G2D; MUC3A; and LTBP2, wherein the presence in a body fluid of the subject of the protein indicates the presence of a tumor vasculature cell in the subject.

In another embodiment, the present invention provides a method of detecting a presence of a solid tumor in a subject, the method comprising the step of detecting a presence in a body fluid of the subject of a nucleotide molecule encoding a protein selected from TNFAIP6 (Tumor necrosis factor, alpha-induced protein 6); STC2 (Stanniocalcin); ESM1 (Endothelial cell-specific molecule 1); IBSP (Integrin-binding sialoprotein); CKLFSF6 (CKLF-like MARVEL transmembrane domain containing 6); HAPLN1 (Hyaluronan and proteoglycan link protein 1); WFDC2 (WAP four-disulfide core domain 2); SPP1 (Secreted phosphoprotein 1 (osteopontin, bone sialoprotein I, early T-lymphocyte activation 1); FLT1 (Fms-related tyrosine kinase 1 (vascular endothelial growth factor/vascular permeability factor receptor); LGALS3BP (Lectin, galactoside-binding, soluble, 3 binding protein); CCL15 (chemokine (C-C motif) ligand 15); PLA2G2D (Phospholipase A2, group IID); MUC3A (Mucin 3A, intestinal); and LTBP2 (Latent transforming growth factor beta binding protein 2); wherein the presence in a body fluid of the subject of the nucleotide molecule indicates the presence of a solid tumor in the subject.

In another embodiment, the present invention provides a method of detecting a presence of a TVC in a subject, the method comprising the step of detecting a presence in a body fluid of the subject of a nucleotide molecule encoding a protein selected from TNFAIP6; STC2; ESM1; IBSP; CKLFSF6; HAPLN1; WFDC2; SPP1; FLT1; LGALS3BP; CCL15; PLA2G2D; MUC3A; and LTBP2; wherein the presence in a body fluid of the subject of the nucleotide molecule indicates the presence of a TVC in the subject.

In another embodiment, the present invention provides a method of detecting a presence of a solid tumor in a subject, the method comprising the step of detecting a presence in a body fluid of the subject of an Adlican protein, wherein the presence in a body fluid of the subject of the Adlican protein indicates the presence of a solid tumor in the subject.

In another embodiment, the present invention provides a method of detecting a presence of a tumor vasculature cell in a subject, the method comprising the step of detecting a presence in a body fluid of the subject of an Adlican protein, wherein the presence in a body fluid of the subject of the Adlican protein indicates the presence of a tumor vasculature cell in the subject.

In another embodiment, the present invention provides a method of detecting a presence of a solid tumor in a subject, the method comprising the step of detecting a presence in a body fluid of the subject of a nucleotide molecule encoding an Adlican protein, wherein the presence in a body fluid of the subject of the nucleotide molecule indicates the presence of a solid tumor in the subject.

In another embodiment, the present invention provides a method of detecting a presence of a tumor vasculature cell in a subject, the method comprising the step of detecting a presence in a body fluid of the subject of a nucleotide molecule encoding an Adlican protein, wherein the presence in a body fluid of the subject of the nucleotide molecule indicates the presence of a tumor vasculature cell in the subject.

In another embodiment, the present invention provides a peptide having the amino acid sequence CPGAKALSRVREDIVEDE (SEQ ID No: 88). In another embodiment, the sequence of the peptide consists of SEQ ID No: 88. Each possibility represents a separate embodiment of the present invention.

In another embodiment, the present invention provides an antibody that recognizes a peptide of the present invention. In another embodiment, the present invention provides an antibody that binds to a peptide of the present invention. In another embodiment, the present invention provides an antibody raised against a peptide of the present invention. Each possibility represents a separate embodiment of the present invention.

In another embodiment, the present invention provides the use of the peptide of the present invention in the detection of a solid tumor or tumor vasculature.

In another embodiment, the present invention provides the use of the peptide of the present invention in the localization of a solid tumor or tumor vasculature.

In another embodiment, the present invention provides the use of the peptide of the present invention in the treatment of a solid tumor or tumor vasculature.

As provided herein (Example 10), Adlican was detected in serum and ascites of patients with stage III ovarian cancer, but not control subjects. Thus, TVM of the present invention are efficacious for detection of tumors, by detecting their presence in bodily fluids of a subject. In another embodiment, a secreted TVM of the present invention is used. In another embodiment, a TVM of the present invention localized to the ECM is used. Each possibility represents a separate embodiment of the present invention.

“Presence in a body fluid” refers, in another embodiment, to a detectable presence. In another embodiment, the term refers to an amount that can be detected by a method used to for detection of proteins or antigens in body fluids. In another embodiment, the term refers to an amount that generates a signal over the background in a method used to for detection of proteins or antigens in body fluids.

In another embodiment, the method is ELISA. In another embodiment, the method is Western blot. In another embodiment, the method is any other method known in the art. Each possibility represents a separate embodiment of the present invention.

In another embodiment, the present invention provides a method of localizing or detecting a presence of a TEC (Tumor Endothelial Cell) in a subject, the method comprising the steps of (a) contacting the subject with a ligand that binds to a protein encoded by a nucleic acid molecule, the nucleic acid molecule encoding a protein selected from an Adlican protein; an EGFL6 protein, an FZD10 protein, a GPM6B protein, and a SPON-1 protein; and (b) imaging the ligand in the subject, whereby, if the ligand is concentrated in an area of a tissue of the subject, then the subject has a TEC in the area.

In another embodiment, the present invention provides a method of localizing or detecting a presence of a VLC (Vascular Leukocyte) in a subject, the method comprising the steps of (a) contacting the subject with a ligand that binds to a nucleic acid molecule that encodes a B lymphocyte activator macrophage expressed (BLAME) protein; and (b) imaging the ligand in the subject, whereby, if the ligand is concentrated in an area of a tissue of the subject, then the subject has a VLC in the area.

In another embodiment, the present invention provides a method of early diagnosis of a tumor, comprising a method of the present invention. In another embodiment, the present invention provides a method of early diagnosis of a cancer, comprising a method of the present invention. In another embodiment, the present invention provides a method of early diagnosis of a tumor, comprising contacting subject with a ligand that binds a TVM of the present invention. In another embodiment, the present invention provides a method of early diagnosis of a cancer, comprising contacting subject with a ligand that binds a TVM of the present invention. In another embodiment, the present invention provides a method of diagnosing or detecting a circulating TVC, comprising a method of the present invention. In another embodiment, the present invention provides a method of diagnosing or detecting a circulating TVC, comprising contacting subject with a ligand that binds a TVM of the present invention. Each possibility represents a separate embodiment of the present invention.

In another embodiment, one of the above methods utilizes a BLAME protein. As provided herein, BLAME is a marker for VLC of solid tumors and is an efficacious marker for localizing, detecting, and treating TVC and solid tumors.

In another embodiment, the present invention provides a method of inhibiting general angiogenesis, analogous to one of the above methods, comprising contacting a subject with a ligand that binds to EGFL6. In another embodiment, the present invention provides a method of detecting general angiogenesis, analogous to one of the above methods, comprising contacting a subject with a ligand that binds to EGFL6. In another embodiment, the present invention provides a method of inhibiting general angiogenesis, analogous to one of the above methods, comprising contacting a subject with a ligand that binds to OLFML2B. In another embodiment, the present invention provides a method of detecting general angiogenesis, analogous to one of the above methods, comprising contacting a subject with a ligand that binds to OLFML2B. As provided herein, EGFL6 and OLFML2B are markers of general angiogenesis.

In another embodiment, the present invention provides a method of localizing or detecting a presence of a VLC (Vascular Leukocyte) in a subject, the method comprising the steps of (a) contacting the subject with a ligand that binds to a BLAME protein; and (b) imaging the ligand in the subject, whereby, if the ligand is concentrated in an area of a tissue of the subject, then the subject has a VLC in the area.

The TVM, nucleic acid molecule, or protein encoded thereby that is detected, bound, inhibited, or targeted by a method of the present invention is, in another embodiment, in the target cell (e.g. the vasculature cell, solid tumor cell, or the like). In another embodiment, the nucleic acid molecule or protein is associated with the target cell. In another embodiment, the nucleic acid molecule or protein is expressed by the target cell. Each possibility represents a separate embodiment of the present invention.

In another embodiment, the present invention provides a method of treating a solid tumor in a subject, the method comprising the steps of localizing the solid tumor by a method of the present invention, and irradiating the solid tumor, thereby treating a solid tumor in a subject.

In another embodiment, the present invention provides a method of treating a solid tumor in a subject, the method comprising the steps of: (a) contacting the subject with a photo-activatable cytotoxic drug or pharmaceutical composition; (b) localizing the solid tumor by a method of the present invention; and (c) contacting the solid tumor with a concentrated light source, thereby treating a solid tumor in a subject.

In another embodiment, the present invention provides a method of treating an ovarian tumor in a subject, the method comprising the steps of localizing the ovarian tumor by a method of the present invention, and irradiating the ovarian tumor, thereby treating an ovarian tumor in a subject.

In another embodiment, the present invention provides a method of treating an ovarian tumor in a subject, the method comprising the steps of: (a) contacting the subject with a photo-activatable cytotoxic drug or pharmaceutical composition; (b) localizing the ovarian tumor by a method of the present invention; and (c) contacting the ovarian tumor with a concentrated light source, thereby treating an ovarian tumor in a subject.

In another embodiment, the present invention provides a method of treating a breast tumor in a subject, the method comprising the steps of localizing the breast tumor by a method of the present invention, and irradiating the breast tumor, thereby treating a breast tumor in a subject.

In another embodiment, the present invention provides a method of treating a breast tumor in a subject, the method comprising the steps of: (a) contacting the subject with a photo-activatable cytotoxic drug or pharmaceutical composition; (b) localizing the breast tumor by a method of the present invention; and (c) contacting the breast tumor with a concentrated light source, thereby treating a breast tumor in a subject.

In another embodiment, the present invention provides a method of treating a renal tumor in a subject, the method comprising the steps of localizing the renal tumor by a method of the present invention, and irradiating the renal tumor, thereby treating a renal tumor in a subject.

In another embodiment, the present invention provides a method of treating a renal tumor in a subject, the method comprising the steps of: (a) contacting the subject with a photo-activatable cytotoxic drug or pharmaceutical composition; (b) localizing the renal tumor by a method of the present invention; and (c) contacting the renal tumor with a concentrated light source, thereby treating a renal tumor in a subject.

Methods for isolation of VLC are well known in the art, and are described, for example, in Conejo-Garcia, J. R., Buckanovich, R. J., Benencia, F., Courreges, M. C., Rubin, S. C., Carroll, R. G. & Coukos, G. (2005) Blood 105: 679-81. In another embodiment, “VLC” refers to VE-cadherin⁺ CD146⁺CD45⁺ cells. In another embodiment, the term refers to human myeloid vascular cells with endothelial-like behavior.

In another embodiment, a VLC of the present invention is a precursor of a TEC of the present invention. In another embodiment, a VLC of the present invention is a separate lineage from of a TEC of the present invention. In another embodiment, VLC of the present invention cooperate with TEC of the present invention in neo-vessel formation. Each possibility represents a separate embodiment of the present invention.

In another embodiment, a TVM of the present invention is expressed by pericytes, in addition to TVC. In another embodiment, the TVM is expressed by a subset of pericytes. In another embodiment, the TVM is not expressed on pericytes.

A TVC of the present invention is, in another embodiment, an endothelial cell. In another embodiment, the TVC is a perivascular cell. In another embodiment, the TVC derives from a myeloid DC. In another embodiment, the TVC derives from a myeloid monocytic precursor. Each possibility represents a separate embodiment of the present invention.

In another embodiment, the present invention provides a method for isolation of a TVC, as exemplified in the Examples herein. Each method exemplified or described herein represents a separate embodiment of the present invention.

In another embodiment, a TVM of the present invention is particularly efficacious for treating, localizing, or diagnosing a particular tumor type. In another embodiment, a TVM of the present invention is efficacious for treating, localizing, or diagnosing multiple tumor types. In another embodiment, collagen 11α1 is particularly useful for breast tumors. In another embodiment, collagen 11α1 is particularly useful for lung tumors. In another embodiment, LZTS1 is particularly useful for melanoma. In another embodiment, LZTS1 is particularly useful for ovarian cancer. In another embodiment, FZD10 is particularly useful for ovarian tumors. In another embodiment, EMBPL1 is particularly useful for ovarian tumors. In another embodiment, BLAME is particularly useful for a tumor selected from ovarian, adrenal, and testis tumors. In another embodiment, ESM1 is particularly useful for a tumor selected from ovarian, adrenal, and renal tumors. In another embodiment, DSG2 is particularly useful for a tumor selected from colon and recto-sigmoid. In another embodiment, EPSTI1 is particularly useful for a tumor selected from adrenal and testes. In another embodiment, MS4A6A is particularly useful for a tumor selected from adrenal and testes. In another embodiment, LOC51136 is particularly useful for a tumor selected from adrenal, breast, and liver. In another embodiment, EGFL6 is particularly useful for a tumor selected from uterine corpus, lung and omentum. In another embodiment, KCNE3 is particularly useful for a tumor selected from recto-sigmoid, stomach, kidney, and adrenal. In another embodiment, KCNE4 is particularly useful for a tumor selected from breast, pancreas, and adrenal. In another embodiment, c14orf100 is particularly useful for adrenal tumors. In another embodiment, KCNK5 is particularly useful for a tumor selected from In another embodiment, BLAME is particularly useful for a tumor selected from recto-sigmoid and adrenal. In another embodiment, FZD10 is particularly useful for a corpus uteri malignancy. In another embodiment, ST14 is particularly useful for a tumor selected from colon, liver, recto-sigmoid, and adrenal. In another embodiment, PCDHB2 is particularly useful for a tumor selected from adrenal, brain, renal, lung, pancreas, and stomach. In another embodiment, OLFML2B is particularly useful for a tumor selected from adrenal and corpus uteri. In another embodiment, GPR105 is particularly useful for a tumor selected from stomach and testes. In another embodiment, IVNS1ABP is particularly useful for a tumor selected from adrenal, kidney, and testes. In another embodiment, SPP1 is particularly useful for a tumor selected from adrenal, kidney, and liver. In another embodiment, KIAA1892 is particularly useful for a testicular tumor. In another embodiment, C6orf69 is particularly useful for an adrenal malignancy. In another embodiment, KIBRA is particularly useful for a tumor selected from kidney and prostate. Each possibility represents a separate embodiment of the present invention.

In another embodiment, a TVM of the present invention has a sequence selected from the sequences set forth in SEQ ID No: 1-16, 18-23, 25-26, 28-32, 34-46, 48-58, 60-66, 68-70, and 85-211). In another embodiment, the TVM has an AA sequence encoded by a nucleotide sequence set forth in Table 6, or in a GenBank entry whose Accession Number appears therein. In another embodiment, the TVM has an AA sequence comprising a nucleotide sequence set forth in Table 6, or in a GenBank entry whose Accession Number appears therein. Each possibility represents a separate embodiment of the present invention.

The nucleic acid molecule that is targeted by methods of the present invention, has, in another embodiment, a sequence selected from the sequences set forth in SEQ ID No: 2, 21, 25, 29, 36-38, 58, 60, 62, and 68-69. In another embodiment, the nucleic acid molecule has a sequence selected from the sequences set forth in SEQ ID No: 2, 27, 37, 38, and 60. In another embodiment, the nucleic acid molecule has the sequence set forth in SEQ ID No: 58. Each possibility represents a separate embodiment of the present invention.

In another embodiment, the TVM has a sequence set forth in Table 6. In another embodiment, the TVM has a sequence comprising a sequence set forth in Table 6. In another embodiment, the TVM has a sequence comprising a partial gene sequence set forth in Table 6. In another embodiment, the TVM has a sequence comprising a partial transcript sequence set forth in Table 6. In another embodiment, the TVM has a sequence set forth in a GenBank entry whose Accession Number appears in Table 6. In another embodiment, the TVM has a sequence comprising a sequence set forth a GenBank entry whose Accession Number appears in Table 6. In another embodiment, the TVM has a sequence comprising a partial gene sequence set forth in a GenBank entry whose Accession Number appears in Table 6. In another embodiment, the TVM has a sequence comprising a partial transcript sequence set forth in a GenBank entry whose Accession Number appears in Table 6.

In another embodiment, the TVM has a sequence set forth in Table 7. In another embodiment, the TVM has a sequence comprising a sequence set forth in Table 7. In another embodiment, the TVM has a sequence comprising a partial gene sequence set forth in Table 7. In another embodiment, the TVM has a sequence comprising a partial transcript sequence set forth in Table 7. In another embodiment, the TVM has a sequence set forth in a GenBank entry whose Accession Number appears in Table 7. In another embodiment, the TVM has a sequence comprising a sequence set forth a GenBank entry whose Accession Number appears in Table 7. In another embodiment, the TVM has a sequence comprising a partial gene sequence set forth in a GenBank entry whose Accession Number appears in Table 7. In another embodiment, the TVM has a sequence comprising a partial transcript sequence set forth in a GenBank entry whose Accession Number appears in Table 7.

In another embodiment, a nucleic acid molecule of the present invention encodes a TVM. In another embodiment, the nucleic acid molecule is a TVM. Each possibility represents a separate embodiment of the present invention.

The protein that is targeted by methods of the present invention, is, in another embodiment, encoded by a nucleic acid molecule having a sequence selected from the sequences set forth in SEQ ID No: 2, 5, 21, 25, 29, 36-38, 44, 58, 60, 62, and 68-69. In another embodiment, the protein is encoded by a nucleic acid molecule having a sequence selected from the sequences set forth in SEQ ID No: 2, 27, 37, 38, and 60. In another embodiment, the protein is encoded by a nucleic acid molecule having the sequence set forth in SEQ ID No: 58. In another embodiment, the protein is a tumor vasculature marker. In another embodiment, the protein has one of the sequences set forth below. Each possibility represents a separate embodiment of the present invention.

In another embodiment, the tumor vasculature marker (TVM) is an Adlican protein. In another embodiment, the marker is a nucleic acid molecule encoding an Adlican protein. In another embodiment, the Adlican protein is encoded by a nucleic acid molecule having the sequence set forth in SEQ ID No: 2. In another embodiment, the Adlican protein is encoded by a nucleic acid molecule having a sequence set forth in GenBank Accession No. AF245505. In another embodiment, the Adlican protein has an amino acid (AA) sequence set forth in GenBank Accession No. AF245505. In another embodiment, the Adlican protein is an MXRA5 protein. In another embodiment, the Adlican protein is encoded by any other Adlican gene sequence known in the art. In another embodiment, the Adlican protein is any other Adlican protein known in the art. In another embodiment, the TVM is an isoform of an Adlican protein. In another embodiment, the TVM is a homologue of an Adlican protein. In another embodiment, the TVM is a variant of an Adlican protein. In another embodiment, the TVM is a fragment of an Adlican protein. In another embodiment, the TVM is a fragment of an isoform, homologue, or variant of an Adlican protein. Each possibility represents a separate embodiment of the present invention.

In another embodiment, the TVM is an AML1 protein. In another embodiment, the marker is a nucleic acid molecule encoding an AML1 protein. In another embodiment, the AML1 protein is encoded by a nucleic acid molecule having the sequence set forth in SEQ ID No: 5. In another embodiment, the AML1 protein is encoded by a nucleic acid molecule having a sequence set forth in GenBank Accession No. NM_{—}001001890. In another embodiment, the AML1 protein is encoded by a nucleic acid molecule having a sequence selected from those set forth in GenBank Accession No. NM_{—}001754 and NM_{—}001987. In another embodiment, the AML1 protein has an AA sequence set forth in one of the above GenBank entries. In another embodiment, the AML1 protein is encoded by a nucleic acid molecule comprising a sequence set forth in DQ224380, DQ224379, DQ224378, DQ207762, DQ207763, DQ207764, DQ207765, DQ207766, DQ207767, DQ207768, DQ207769, DQ207770, DQ100455, DQ100456, DQ100457, AJ888032, AJ888033, AJ888034, AJ888035, AJ888036, AJ888037, AJ888038, AJ888039, AJ888040, or AJ888041. In another embodiment, the AML1 protein has an AA sequence comprising an AA sequence set forth in one of the above GenBank entries. In another embodiment, the AML1 protein is encoded by any other AML1 gene sequence known in the art. In another embodiment, the AML1 protein is any other AML1 protein known in the art. In another embodiment, the TVM is an isoform of an AML1 protein. In another embodiment, the TVM is a homologue of an AML1 protein. In another embodiment, the TVM is a variant of an AML1 protein. In another embodiment, a TEL/AML1 protein is utilized in methods and compositions of the present invention. In another embodiment, the TEL/AML1 protein is encoded by any TEL/AML1 gene sequence known in the art. In another embodiment, the TEL/AML1 protein is any TEL/AML1 protein known in the art. In another embodiment, the TVM is an isoform of a TEL/AML1 protein. In another embodiment, the TVM is a homologue of a TEL/AML1 protein. In another embodiment, an ETV6/RUNX1 protein is utilized in methods and compositions of the present invention. In another embodiment, the ETV6/RUNX1 protein is encoded by any ETV6/RUNX1 gene sequence known in the art. In another embodiment, the ETV6/RUNX1 protein is any ETV6/RUNX1 protein known in the art In another embodiment, the TVM is an isoform of an ETV6/RUNX1 protein. In another embodiment, the TVM is a homologue of an ETV6/RUNX1 protein. In another embodiment, the TVM is a fragment of an AML1, TEL/AML1, or ETV6/RUNX1 protein. In another embodiment, the TVM is a fragment of an isoform, homologue, or variant of an AML1, TEL/AML1, or ETV6/RUNX1 protein. Each possibility represents another embodiment of the present invention.

In another embodiment, the TVM is a COL11A1 protein. In another embodiment, the marker is a nucleic acid molecule encoding a COL11A1 protein. In another embodiment, the COL11A1 protein is encoded by a nucleic acid molecule having the sequence set forth in SEQ ID No: 21. In another embodiment, the COL11A1 protein is encoded by a nucleic acid molecule with the following sequence:

(SEQ ID No: 21)

acacagtactctcagcttgttggtggaagcccctcatctgccttcattct
gaaggcagggcccggcagaggaaggatcagagggtcgcggccggagggtc
ccggccggtggggccaactcagagggagaggaaagggctagagacacgaa
gaacgcaaaccatcaaatttagaagaaaaagccctttgactttttccccc
tctccctccccaatggctgtgtagcaaacatccctggcgataccttggaa
aggacgaagttggtctgcagtcgcaatttcgtgggttgagttcacagttg
tgagtgcggggctcggagatggagccgtggtcctctaggtggaaaacgaa
acggtggctctgggatttcaccgtaacaaccctcgcattgaccttcctct
tccaagctagagaggtcagaggagctgctccagttgatgtactaaaagca
ctagattttcacaattctccagagggaatatcaaaaacaacgggattttg
cacaaacagaaagaattctaaaggctcagatactgcttacagagtttcaa
agcaagcacaactcagtgccccaacaaaacagttatttccaggtggaact
ttcccagaagacttttcaatactatttacagtaaaaccaaaaaaaggaat
tcagtctttccttttatctatatataatgagcatggtattcagcaaattg
gtgttgaggttgggagatcacctgtttttctgtttgaagaccacactgga
aaacctgccccagaagactatcccctcttcagaactgttaacatcgctga
cgggaagtggcatcgggtagcaatcagcgtggagaagaaaactgtgacaa
tgattgttgattgtaagaagaaaaccacgaaaccacttgatagaagtgag
agagcaattgttgataccaatggaatcacggtttttggaacaaggatttt
ggatgaagaagtttttgagggggacattcagcagtttttgatcacaggtg
atcccaaggcagcatatgactactgtgagcattatagtccagactgtgac
tcttcagcacccaaggctgctcaagctcaggaacctcagatagatgagta
tgcaccagaggatataatcgaatatgactatgagtatggggaagcagagt
ataaagaggctgaaagtgtaacagagggacccactgtaactgaggagaca
atagcacagacggaggcaaacatcgttgatgattttcaagaatacaacta
tggaacaatggaaagttaccagacagaagctcctaggcatgtttctggga
caaatgagccaaatccagttgaagaaatatttactgaagaatatctaacg
ggagaggattatgattcccagaggaaaaattctgaggatacactatatga
aaacaaagaaatagacggcagggattctgatcttctggtagatggagatt
taggcgaatatgatttttatgaatataaagaatatgaagataaaccaaca
agcccccctaatgaagaatttggtccaggtgtaccagcagaaactgatat
tacagaaacaagcataaatggccatggtgcatatggagagaaaggacaga
aaggagaaccagcagtggttgagcctggtatgcttgtcgaaggaccacca
ggaccagcaggacctgcaggtattatgggtcctccaggtctacaaggccc
cactggaccccctggtgaccctggcgataggggccccccaggacgtcctg
gcttaccaggggctgatggtctacctggtcctcctggtactatgttgatg
ttaccgttccgttatggtggtgatggttccaaaggaccaaccatctctgc
tcaggaagctcaggctcaagctattcttcagcaggctcggattgctctga
gaggcccacctggcccaatgggtctaactggaagaccaggtcctgtgggg
gggcctggttcatctggggccaaaggtgagagtggtgatccaggtcctca
gggccctcgaggcgtccagggtccccctggtccaacgggaaaacctggaa
aaaggggtcgtccaggtgcagatggaggaagaggaatgccaggagaacct
ggggcaaagggagatcgagggtttgatggacttccgggtctgccaggtga
caaaggtcacaggggtgaacgaggtcctcaaggtcctccaggtcctcctg
gtgatgatggaatgaggggagaagatggagaaattggaccaagaggtctt
ccaggtgaagctggcccacgaggtttgctgggtccaaggggaactccagg
agctccagggcagcctggtatggcaggtgtagatggccccccaggaccaa
aagggaacatgggtccccaaggggagcctgggcctccaggtcaacaaggg
aatccaggacctcagggtcttcctggtccacaaggtccaattggtcctcc
tggtgaaaaaggaccacaaggaaaaccaggacttgctggacttcctggtg
ctgatgggcctcctggtcatcctgggaaagaaggccagtctggagaaaag
ggggctctgggtccccctggtccacaaggtcctattggatacccgggccc
ccggggagtaaagggagcagatggtgtcagaggtctcaagggatctaaag
gtgaaaagggtgaagatggttttccaggattcaaaggtgacatgggtcta
aaaggtgacagaggagaagttggtcaaattggcccaagaggggaagatgg
ccctgaaggacccaaaggtcgagcaggcccaactggagacccaggtcctt
caggtcaagcaggagaaaagggaaaacttggagttccaggattaccagga
tatccaggaagacaaggtccaaagggttccactggattccctgggtttcc
aggtgccaatggagagaaaggtgcacggggagtagctggcaaaccaggcc
ctcggggtcagcgtggtccaacgggtcctcgaggttcaagaggtgcaaga
ggtcccactgggaaacctgggccaaagggcacttcaggtggcgatggccc
tcctggccctccaggtgaaagaggtcctcaaggacctcagggtccagttg
gattccctggaccaaaaggccctcctggaccacctgggaaggatgggctg
ccaggacaccctgggcaacgtggggagactggatttcaaggcaagaccgg
ccctcctgggccagggggagtggttggaccacagggaccaaccggtgaga
ctggtccaataggggaacgtgggcatcctggccctcctggccctcctggt
gagcaaggtcttcctggtgctgcaggaaaagaaggtgcaaagggtgatcc
aggtcctcaaggtatctcagggaaagatggaccagcaggattacgtggtt
tcccaggggaaagaggtcttcctggagctcagggtgcacctggactgaaa
ggaggggaaggtccccagggcccaccaggtccagttggctcaccaggaga
acgtgggtcagcaggtacagctggcccaattggtttaccagggcgcccgg
gacctcagggtcctcctggtccagctggagagaaaggtgctcctggagaa
aaaggtccccaagggcctgcagggagagatggagttcaaggtcctgttgg
tctcccagggccagctggtcctgccggctcccctggggaagacggagaca
agggtgaaattggtgagccgggacaaaaaggcagcaagggtgacaaggga
gaaaatggccctcccggtcccccaggtcttcaaggaccagttggtgcccc
tggaattgctggaggtgatggtgaaccaggtcctagaggacagcagggga
tgtttgggcaaaaaggtgatgagggtgccagaggcttccctggacctcct
ggtccaataggtcttcagggtctgccaggcccacctggtgaaaaaggtga
aaatggggatgttggtcccatggggccacctggtcctccaggcccaagag
gccctcaaggtcccaatggagctgatggaccacaaggacccccagggtct
gttggttcagttggtggtgttggagaaaagggtgaacctggagaagcagg
gaacccagggcctcctggggaagcaggtgtaggcggtcccaaaggagaaa
gaggagagaaaggggaagctggtccacctggagctgctggacctccaggt
gccaaggggccaccaggtgatgatggccctaagggtaacccgggtcctgt
tggttttcctggagatcctggtcctcctggggaacctggccctgcaggtc
aagatggtgttggtggtgacaagggtgaagatggagatcctggtcaaccg
ggtcctcctggcccatctggtgaggctggcccaccaggtcctcctggaaa
acgaggtcctcctggagctgcaggtgcagagggaagacaaggtgaaaaag
gtgctaagggggaagcaggtgcagaaggtcctcctggaaaaaccggccca
gtcggtcctcagggacctgcaggaaagcctggtccagaaggtcttcgggg
catccctggtcctgtgggagaacaaggtctccctggagctgcaggccaag
atggaccacctggtcctatgggacctcctggcttacctggtctcaaaggt
gaccctggctccaagggtgaaaagggacatcctggtttaattggcctgat
tggtcctccaggagaacaaggggaaaaaggtgaccgagggctccctggaa
ctcaaggatctccaggagcaaaaggggatgggggaattcctggtcctgct
ggtcccttaggtccacctggtcctccaggtttaccaggtcctcaaggccc
aaagggtaacaaaggctctactggacccgctggccagaaaggtgacagtg
gtcttccagggcctcctgggtctccaggtccacctggtgaagtcattcag
cctttaccaatcttgtcctccaaaaaaacgagaagacatactgaaggcat
gcaagcagatgcagatgataatattcttgattactcggatggaatggaag
aaatatttggttccctcaattccctgaaacaagacattgagcatatgaaa
tttccaatgggtactcagaccaatccagcccgaacttgtaaagacctgca
actcagccatcctgacttcccagatggtgaatattggattgatcctaacc
aaggttgctcaggagattccttcaaagtttactgtaatttcacatctggt
ggtgagacttgcatttatccagacaaaaaatctgagggagtaagaatttc
atcatggccaaaggagaaaccaggaagttggtttagtgaatttaagaggg
gaaaactgctttcatacttagatgttgaaggaaattccatcaatatggtg
caaatgacattcctgaaacttctgactgcctctgctcggcaaaatttcac
ctaccactgtcatcagtcagcagcctggtatgatgtgtcatcaggaagtt
atgacaaagcacttcgcttcctgggatcaaatgatgaggagatgtcctat
gacaataatccttttatcaaaacactgtatgatggttgtgcgtccagaaa
aggctatgaaaagactgtcattgaaatcaatacaccaaaaattgatcaag
tacctattgttgatgtcatgatcaatgactttggtgatcagaatcagaag
ttcggatttgaagttggtcctgtttgttttcttggctaagattaagacaa
agaacatatcaaatcaacagaaaatataccttggtgccaccaacccattt
tgtgccacatgcaagttttgaataaggatggtatagaaaacaacgctgca
tatacaggtaccatttaggaaataccgatgcctttgtgggggcagaatca
catggcaaaagctttgaaaatcataaagatataagttggtgtggctaaga
tggaaacagggctgattcttgattcccaattctcaactctccttttccta
tttgaatttctttggtgctgtagaaaacaaaaaaagaaaaatatatattc
ataaaaaatatggtgctcattctcatccatccaggatgtactaaaacagt
gtgtttaataaattgtaattattttgtgtacagttctatactgttatctg
tgtccatttccaaaacttgcacgtgtccctgaattccatctgactctaat
tttatgagaattgcagaactctgatggcaataaatatatgtattatgaaa
aaataaagttgtaatttctgatgactctaagtccctttctttgttaataa
taaaatgcctttgtatatattgatgttgaagagttcaattatttgatgtc
gccaacaaaattctcagagggcaaaaatctggaagacttttggaagcaca
ctctgatcaactcttctctgccgacagtcattttgctgaatttcagccaa
aaatattatgcattttgatgctttattcaaggctatacctcaaacttttt
cttctcagaatccaggatttcacaggatacttgtatatatggaaaacaag
caagtttatatttttggacagggaaatgtgtgtaagaaagtatattaaca
aatcaatgcctccgtcaagcaaacaatcatatgtatactttttttctacg
ttatctcatctccttgttttcagtgtgcttcaataatgcaggttaatatt
aaagatggaaattaagcaattatttatgaatttgtgcaatgttagatttt
cttatcaatcaagttcttgaatttgattctaagttgcatattataacagt
ctcgaaaattattttacttgcccaacaaatattacttttttcctttcaag
ataattttataaatcatttgacctacctaattgctaaatgaataacatat
ggtggactgttattaagagtatttgttttaagtcattcaggaaaatctaa
acttttttttccactaaggtatttactttaaggtagcttgaaatagcaat
acaatttaaaaattaaaaactgaattttgtatctattttaagtaatatat
gtaagacttgaaaataaatgttttatttcttatataaagtgttaaattaa
ttgataccagatttcactggaacagtttcaactgataatttatgacaaaa
gaacatacctgtaatattgaaattaaaaagtgaaatttgtcataaagaat
ttcttttatttttgaaatcgagtttgtaaatgtccttttaagaagggaga
tatgaatccaataaataaactcaagtcttggctacctgga.

In another embodiment, the COL11A1 protein is encoded by a nucleic acid molecule having a sequence set forth in GenBank Accession No. NM_{—}001854. In another embodiment, the COL11A1 protein is encoded by a nucleic acid molecule having a sequence selected from those set forth in GenBank Accession No. NM_{—}080629, NM_{—}080630, J04177, AB208844, and AB208844. In another embodiment, the COL11A1 protein has an AA sequence set forth in one of the above GenBank entries. In another embodiment, the COL11A1 protein has an AA sequence set forth in GenBank Accession No. NP_{—}542196, NP_{—}542197, AAA51891, or BAD92081. In another embodiment, the COL11A1 protein is encoded by a COL11A transcript variant A. In another embodiment, the COL11A1 protein is encoded by a COL11A transcript variant B. In another embodiment, the COL11A1 protein is encoded by a COL11A transcript variant C. In another embodiment, the COL11A1 protein is a COL11A isoform A. In another embodiment, the COL11A1 protein is a COL11A isoform B. In another embodiment, the COL11A1 protein is a COL11A isoform C. In another embodiment, the COL11A1 protein is encoded by any other COL11A1 gene sequence known in the art. In another embodiment, the COL11A1 protein is any other COL11A1 protein known in the art. In another embodiment, the TVM is an isoform of a COL11A1 protein. In another embodiment, the TVM is a homologue of a COL11A1 protein. In another embodiment, the TVM is a variant of a COL11A1 protein. In another embodiment, the TVM is a fragment of a COL11A1 protein. In another embodiment, the TVM is a fragment of an isoform, homologue, or variant of a COL11A1 protein. Each possibility represents another embodiment of the present invention.

In another embodiment, the TVM is a DEFB1 protein. In another embodiment, the marker is a nucleic acid molecule encoding a DEFB1 protein. In another embodiment, the DEFB1 protein is encoded by a nucleic acid molecule having the sequence set forth in SEQ ID No: 25. In another embodiment, the DEFB1 protein is encoded by a nucleic acid molecule having a sequence set forth in GenBank Accession No. BC033298. In another embodiment, the DEFB1 protein is encoded by a nucleic acid molecule having a sequence selected from those set forth in GenBank Accession No. BC047677, NM_{—}005218, U73945, Z50788, and X92744. In another embodiment, the DEFB1 protein has an AA sequence set forth in one of the above GenBank entries. In another embodiment, the DEFB1 protein has an AA sequence selected from the sequences set forth in GenBank Accession No. NP_{—}005209, AAH33298, AAH47677, CAA63405, and CAA90650. In another embodiment, the DEFB1 protein is encoded by any other DEFB1 gene sequence known in the art. In another embodiment, the DEFB1 protein is any other DEFB1 protein known in the art. In another embodiment, the TVM is an isoform of a DEFB1 protein. In another embodiment, the TVM is a homologue of a DEFB1 protein. In another embodiment, the TVM is a variant of a DEFB1 protein. In another embodiment, the TVM is a fragment of a DEFB1 protein. In another embodiment, the TVM is a fragment of an isoform, homologue, or variant of a DEFB1 protein. Each possibility represents another embodiment of the present invention.

In another embodiment, the TVM is an EPB41L3 protein. In another embodiment, the marker is a nucleic acid molecule encoding an EPB41L3 protein. In another embodiment, the TVM is a homologue of an EPB41L3 precursor protein. In another embodiment, the TVM is a variant of an EPB41L3 precursor protein. In another embodiment, the TVM is an isoform of an EPB41L3 precursor protein. In another embodiment, the TVM is a fragment of an EPB41L3 protein. In another embodiment, the TVM is a fragment of an isoform, homologue, or variant of an EPB41L3 protein. Each possibility represents another embodiment of the present invention.

In another embodiment, the TVM is an F2RL1 protein. In another embodiment, the marker is a nucleic acid molecule encoding an F2RL1 protein. In another embodiment, the F2RL1 protein is encoded by a nucleic acid molecule having the sequence set forth in SEQ ID No: 36. In another embodiment, the F2RL1 protein is encoded by a nucleic acid molecule having a sequence set forth in GenBank Accession No. BC012453. In another embodiment, the F2RL1 protein is encoded by a nucleic acid molecule having a sequence selected from those set forth in GenBank Accession No. BC018130, U34038, BC012453, BC018130, BT009856, AY336105, and NM_{—}005242. In another embodiment, the F2RL1 protein has an AA sequence set forth in one of the above GenBank entries. In another embodiment, the F2RL1 protein has an AA sequence selected from the sequences set forth in GenBank Accession No. NP_{—}005233, AAB47871, AAH12453, AAH18130, AAP88858, and AAP97012. In another embodiment, the F2RL1 protein is encoded by any other F2RL1 gene sequence known in the art. In another embodiment, the F2RL1 protein is any other F2RL1 protein known in the art. In another embodiment, the TVM is an isoform of an F2RL1 protein. In another embodiment, the TVM is a homologue of an F2RL1 protein. In another embodiment, the TVM is a variant of an F2RL1 protein. In another embodiment, a coagulation factor II (thrombin) receptor-like 1 (F2RL1) precursor protein is utilized in methods and compositions of the present invention. In another embodiment, the F2RL1 precursor protein is encoded by a gene having a sequence set forth in GenBank Accession No. NP_{—}005233. In another embodiment, the F2RL1 precursor protein is encoded by any F2RL1 precursor gene sequence known in the art. In another embodiment, the F2RL1 precursor protein is any F2RL1 precursor protein known in the art. In another embodiment, the TVM is an isoform of a F2RL1 precursor protein. In another embodiment, the TVM is a fragment of an F2RL1 protein or precursor thereof. In another embodiment, the TVM is a fragment of an isoform, homologue, or variant of an F2RL1 protein or precursor thereof. Each possibility represents another embodiment of the present invention.

In another embodiment, the TVM is a GPM6B protein. In another embodiment, the marker is a nucleic acid molecule encoding a GPM6B protein. In another embodiment, the GPM6B protein is encoded by a nucleic acid molecule having the sequence set forth in SEQ ID No: 38. In another embodiment, the GPM6B protein is encoded by a nucleic acid molecule having a sequence set forth in GenBank Accession No. BC008151. In another embodiment, the GPM6B protein is encoded by a nucleic acid molecule having a sequence selected from those set forth in GenBank Accession No. BC047295, NM_{—}005278, NM_{—}001001994, NM_{—}001001995, NM_{—}001001996, AK095657, AB209525, and U45955. In another embodiment, the GPM6B protein has an AA sequence set forth in one of the above GenBank entries. In another embodiment, the GPM6B protein has an AA sequence selected from the sequences set forth in GenBank Accession No. NP_{—}005269, AAH08151, BAC04600, BAD92762, and AAB16888. In another embodiment, the GPM6B protein is encoded by a transcript variant 1 of a GPM6B-encoding RNA. In another embodiment, the GPM6B protein is encoded by a transcript variant 2 of a GPM6B-encoding RNA. In another embodiment, the GPM6B protein is encoded by a transcript variant 3 of a GPM6B-encoding RNA. In another embodiment, the GPM6B protein is encoded by a transcript variant 4 of a GPM6B-encoding RNA. In another embodiment, the GPM6B protein is encoded by any other GPM6B gene sequence known in the art. In another embodiment, the GPM6B protein is a GPM6B isoform 1. In another embodiment, the GPM6B protein is a GPM6B isoform 2. In another embodiment, the GPM6B protein is an M6b-2. In another embodiment, the GPM6B protein is a GPM6B isoform 3. In another embodiment, the TVM is another isoform of a GPM6B protein. In another embodiment, the GPM6B protein is any other GPM6B protein known in the art. In another embodiment, the TVM is a homologue of a GPM6B protein. In another embodiment, the TVM is a variant of a GPM6B protein. In another embodiment, the TVM is a fragment of a GPM6B protein. In another embodiment, the TVM is a fragment of an isoform, homologue, or variant of a GPM6B protein. Each possibility represents another embodiment of the present invention.

In another embodiment, the TVM is an LZTS1 protein. In another embodiment, the marker is a nucleic acid molecule encoding a LZTS1 protein. In another embodiment, the LZTS1 protein is encoded by a nucleic acid molecule having the sequence set forth in SEQ ID No: 44. In another embodiment, the LZTS1 protein is encoded by a nucleic acid molecule having a sequence set forth in GenBank Accession No. NM_{—}021020. In another embodiment, the LZTS1 protein is encoded by a nucleic acid molecule having a sequence selected from those set forth in GenBank Accession No. AF123659, BC075006, AF123654, AF123655, AF123656, AF123657, AF123658, BC075006, BC075007, and BC075007. In another embodiment, the LZTS1 protein has an AA sequence set forth in one of the above GenBank entries. In another embodiment, the LZTS1 protein has an AA sequence selected from the sequences set forth in NP_{—}066300, AAD23833, AAD23835, AAD23836, AAD23837, AAD23838, AAD23839, AAD23840, AAH75006 and AAH75007. In another embodiment, the LZTS1 protein is encoded by any other LZTS1 gene sequence known in the art. In another embodiment, the LZTS1 protein is any other LZTS1 protein known in the art. In another embodiment, the TVM is an isoform of a LZTS1 protein. In another embodiment, the TVM is a homologue of a LZTS1 protein. In another embodiment, the TVM is a variant of a LZTS1 protein. In another embodiment, an E16T8 FEZ1 or a fasciculation and elongation protein zeta 1 (FEZ1) protein is utilized in methods and compositions of the present invention. In another embodiment, the FEZ1 protein is encoded by any FEZ1 gene sequence known in the art. In another embodiment, the FEZ1 protein is any FEZ1 protein known in the art. In another embodiment, the TVM is an isoform of a FEZ1 protein. In another embodiment, the TVM is a homologue of a FEZ1 protein. In another embodiment, a zygin I protein is utilized in methods and compositions of the present invention. In another embodiment, the zygin I protein is encoded by any zygin I gene sequence known in the art. In another embodiment, the zygin I protein is any zygin I protein known in the art. In another embodiment, the TVM is an isoform of a zygin I protein. In another embodiment, the TVM is a homologue of a zygin I protein. In another embodiment, a LAPSER1 protein is utilized in methods and compositions of the present invention. In another embodiment, the LAPSER1 protein is encoded by any LAPSER1 gene sequence known in the art. In another embodiment, the LAPSER1 protein is any LAPSER1 protein known in the art. In another embodiment, the TVM is an isoform of a LAPSER1 protein. In another embodiment, the TVM is a homologue of a LAPSER1 protein. In another embodiment, the TVM is a fragment of a LZTS1, FEZ1, zygin I, or LAPSER1 protein. In another embodiment, the TVM is a fragment of an isoform, homologue, or variant of a LZTS1, FEZ1, zygin I, or LAPSER1 protein. Each possibility represents another embodiment of the present invention.

In another embodiment, the TVM is a BLAME protein. In another embodiment, the marker is a nucleic acid molecule encoding a BLAME protein. In another embodiment, the BLAME protein is encoded by a nucleic acid molecule having the sequence set forth in SEQ ID No: 58. In another embodiment, the BLAME protein is encoded by a nucleic acid molecule having a sequence set forth in GenBank Accession No. AK074669. In another embodiment, the BLAME protein is encoded by a nucleic acid molecule having a sequence selected from those set forth in GenBank Accession No. BC109194, NM_{—}020125, AF144235, or AF146761. In another embodiment, the BLAME protein is encoded by a FLJ90188 cDNA. In another embodiment, the BLAME protein has an AA sequence set forth in one of the above GenBank entries. In another embodiment, the BLAME protein has an AA sequence selected from the sequences set forth in GenBank Accession No. NP_{—}064510, AAD33923, AAF67470, AA109195, and BAC11123. In another embodiment, the BLAME protein is referred to as “SLAMF8.” In another embodiment, the BLAME protein is encoded by any other BLAME gene sequence known in the art. In another embodiment, the BLAME protein is any other BLAME protein known in the art. In another embodiment, the TVM is an isoform of a BLAME protein. In another embodiment, the TVM is a homologue of a BLAME protein. In another embodiment, the TVM is a variant of a BLAME protein. In another embodiment, a BCM-like membrane protein precursor or IgSF protein is utilized in methods and compositions of the present invention. In another embodiment, the protein is encoded by any BCM-like membrane protein precursor or IgSF protein gene sequence known in the art. In another embodiment, the protein is any BCM-like membrane protein precursor or IgSF protein known in the art. In another embodiment, the TVM is an isoform of a BCM-like membrane protein precursor or IgSF protein. In another embodiment, the TVM is a homologue of a BCM-like membrane protein precursor or IgSF protein. In another embodiment, an FLJ20442 protein is utilized in methods and compositions of the present invention. In another embodiment, the FLJ20442 protein is encoded by any FLJ20442 gene sequence known in the art. In another embodiment, the FLJ20442 protein is any FLJ20442 protein known in the art. In another embodiment, the TVM is an isoform of an FLJ20442 protein. In another embodiment, the TVM is a homologue of an FLJ20442 protein. In another embodiment, the TVM is a fragment of a BLAME, IgSF, or FLJ20442 protein. In another embodiment, the TVM is a fragment of an isoform, homologue, or variant of a BLAME, IgSF, or FLJ20442 protein. Each possibility represents another embodiment of the present invention.

In another embodiment, the TVM is a SPON1 protein. In another embodiment, the marker is a nucleic acid molecule encoding a SPON1 protein. In another embodiment, the SPON1 protein is encoded by a nucleic acid molecule having the sequence set forth in SEQ ID No: 60. In another embodiment, the SPON1 protein is encoded by a nucleic acid molecule having a sequence set forth in GenBank Accession No. NM_{—}006108. In another embodiment, the SPON1 protein is encoded by a nucleic acid molecule having a sequence selected from those set forth in GenBank Accession No. NM_{—}006108, AB051390, AK074803, AK074803, NP_{—}006099, and BC041974. In another embodiment, the SPON1 protein is encoded by a FLJ90322 cDNA. In another embodiment, the SPON1 protein has an AA sequence set forth in one of the above GenBank entries. In another embodiment, the SPON1 protein has an AA sequence selected from the sequences set forth in GenBank Accession No. BAA34482, BAB18461, BAC11217, AAH19825, and AAH41974. In another embodiment, the SPON1 protein is encoded by a nucleic acid molecule comprising a sequence set forth in BC019825, BC041974, and AB018305. In another embodiment, the SPON1 protein has an AA sequence comprising an AA sequence set forth in one of the above GenBank entries. In another embodiment, the SPON1 protein is encoded by any other SPON1 gene sequence known in the art. In another embodiment, the SPON1 protein is any other SPON1 protein known in the art. In another embodiment, the TVM is an isoform of a SPON1 protein. In another embodiment, the TVM is a homologue of a SPON1 protein. In another embodiment, the TVM is a variant of a SPON1 protein. In another embodiment, a VSGP/F-spondin protein is utilized in methods and compositions of the present invention. In another embodiment, the protein is encoded by any VSGP/F-spondin gene sequence known in the art. In another embodiment, the protein is any VSGP/F-spondin protein known in the art. In another embodiment, the TVM is an isoform of a VSGP/F-spondin protein. In another embodiment, the TVM is a homologue of a VSGP/F-spondin protein. In another embodiment, a KIAA0762 protein is utilized in methods and compositions of the present invention. In another embodiment, the protein is encoded by any KIAA0762 gene sequence known in the art. In another embodiment, the protein is any KIAA0762 protein known in the art. In another embodiment, the TVM is an isoform of a KIAA0762 protein. In another embodiment, the TVM is a homologue of a KIAA0762 protein. In another embodiment, the TVM is a fragment of a SPON1, VSGP/F-spondin, or KIAA0762 protein. In another embodiment, the TVM is a fragment of an isoform, homologue, or variant of a SPON1, VSGP/F-spondin, or KIAA0762 protein. Each possibility represents another embodiment of the present invention.

In another embodiment, the TVM is an STC2 protein. In another embodiment, the marker is a nucleic acid molecule encoding an STC2 protein. In another embodiment, the STC2 protein is encoded by a nucleic acid molecule having the sequence set forth in SEQ ID No: 62. In another embodiment, the STC2 protein is encoded by a nucleic acid molecule having a sequence set forth in GenBank Accession No. BC000658. In another embodiment, the STC2 protein is encoded by a nucleic acid molecule having a sequence selected from those set forth in GenBank Accession No. BC006352, BC013958, AF055460, AB012664, AK027390, AK075406, AF098462, AF031036, BT019591, CR541825, NP_{—}003705, and AK095891. In another embodiment, the STC2 protein is encoded by a cDNA selected from FLJ14484 fis, PSEC0097 fis, and FLJ38572 fis. In another embodiment, the STC2 protein has an AA sequence set forth in one of the above GenBank entries. In another embodiment, the STC2 protein has an AA sequence set forth in GenBank Accession No. AAC27036, AAC97948, AAD01922, AAH00658, AAH06352, AAH13958, AAV38398, BAA33489, and CAG46624. In another embodiment, the STC2 protein is encoded by any other STC2 gene sequence known in the art. In another embodiment, the STC2 protein is any other STC2 protein known in the art. In another embodiment, the TVM is an isoform of an STC2 protein. In another embodiment, the TVM is a homologue of an STC2 protein. In another embodiment, a STC2 precursor protein is utilized in methods and compositions of the present invention. In another embodiment, the protein is encoded by any STC2 precursor gene sequence known in the art. In another embodiment, the protein is any STC2 precursor protein known in the art. In another embodiment, the TVM is an isoform of an STC2 precursor protein. In another embodiment, the TVM is a homologue of an STC2 precursor protein. In another embodiment, the TVM is a variant of an STC2 protein. In another embodiment, the TVM is a fragment of a STC2 protein or precursor thereof. In another embodiment, the TVM is a fragment of an isoform, homologue, or variant of a STC2 protein or precursor thereof. Each possibility represents another embodiment of the present invention.

In another embodiment, the TVM is a TNFAIP6 protein. In another embodiment, the marker is a nucleic acid molecule encoding a TNFAIP6 protein. In another embodiment, the TNFAIP6 protein is encoded by a nucleic acid molecule having the sequence set forth in SEQ ID No: 68. In another embodiment, the TNFAIP6 protein is encoded by a nucleic acid molecule having a sequence set forth in GenBank Accession No. BC030205. In another embodiment, the TNFAIP6 protein is encoded by a nucleic acid molecule having a sequence selected from those set forth in GenBank Accession No. NM_{—}007115, M31165, AJ421518, and AJ419936. In another embodiment, the TNFAIP6 protein has an AA sequence set forth in one of the above GenBank entries. In another embodiment, the TNFAIP6 protein has an AA sequence selected from the sequences set forth in GenBank entries NP_{—}009046, AAB00792, AAH30205, CAD12353, and CAD13434. In another embodiment, the TNFAIP6 protein is encoded by a nucleic acid molecule comprising a sequence set forth in GenBank entry BC039384. In another embodiment, the TNFAIP6 protein has an AA sequence comprising an AA sequence set forth in GenBank entry BC039384. In another embodiment, the TNFAIP6 protein is encoded by any other TNFAIP6 gene sequence known in the art. In another embodiment, the TNFAIP6 protein is any other TNFAIP6 protein known in the art. In another embodiment, the TVM is an isoform of a TNFAIP6 protein. In another embodiment, the TVM is a homologue of a TNFAIP6 protein. In another embodiment, the TVM is a variant of a TNFAIP6 protein. In another embodiment, a TNFAIP6 precursor protein is utilized in methods and compositions of the present invention. In another embodiment, the protein is encoded by any TNFAIP6 precursor gene sequence known in the art. In another embodiment, the protein is any TNFAIP6 precursor protein known in the art. In another embodiment, the TVM is an isoform of a TNFAIP6 precursor protein. In another embodiment, the TVM is a homologue of a TNFAIP6 precursor protein. In another embodiment, a tumor necrosis factor-stimulated gene 6 (TSG-6) protein is utilized in methods and compositions of the present invention. In another embodiment, the protein is encoded by any TSG-6 gene sequence known in the art. In another embodiment, the protein is any TSG-6 protein known in the art. In another embodiment, the TVM is an isoform of a TSG-6 protein. In another embodiment, the TVM is a homologue of a TSG-6 protein. In another embodiment, the TVM is a fragment of a TNFAIP6 or TSG-6 protein. In another embodiment, the TVM is a fragment of an isoform, homologue, or variant of a TNFAIP6 or TSG-6 protein. Each possibility represents another embodiment of the present invention.

In another embodiment, the TVM is a TNFRSF21 protein. In another embodiment, the marker is a nucleic acid molecule encoding a TNFRSF21 protein. In another embodiment, the TNFRSF21 protein is encoded by a nucleic acid molecule having the sequence set forth in SEQ ID No: 69. In another embodiment, the TNFRSF21 protein is encoded by a nucleic acid molecule having a sequence set forth in GenBank Accession No. BC010241. In another embodiment, the TNFRSF21 protein is encoded by a nucleic acid molecule having a sequence selected from those set forth in GenBank Accession No. BC017730, NM_{—}014452, AY358304, BC005192, BC015466, AB209394, AJ420531, AF068868, AF208860, BC010241, BT007420, NP_{—}055267, or CR457190. In another embodiment, the TNFRSF21 protein has an AA sequence set forth in one of the above GenBank entries. In another embodiment, the TNFRSF21 protein is encoded by any other TNFRSF21 gene sequence known in the art. In another embodiment, the TNFRSF21 protein is any other TNFRSF21 protein known in the art. In another embodiment, the TVM is an isoform of a TNFRSF21 protein. In another embodiment, the TVM is a homologue of a TNFRSF21 protein. In another embodiment, the TVM is a variant of a TNFRSF21 protein. In another embodiment, a TNFR-related death receptor-6 (DR6) protein is utilized in methods and compositions of the present invention. In another embodiment, the protein is encoded by any DR6 gene sequence known in the art. In another embodiment, the protein is any DR6 protein known in the art. In another embodiment, the TVM is an isoform of a DR6 protein. In another embodiment, the TVM is a homologue of a DR6 protein. In another embodiment, a TNFRSF21 precursor protein is utilized in methods and compositions of the present invention. In another embodiment, the protein is encoded by any TNFRSF21 precursor gene sequence known in the art. In another embodiment, the protein is any TNFRSF21 precursor protein known in the art. In another embodiment, the TVM is an isoform of a TNFRSF21 precursor protein. In another embodiment, the TVM is a homologue of a TNFRSF21 precursor protein. In another embodiment, the TVM is a fragment of a TNFRSF21 protein, DR6 protein, or precursor thereof. In another embodiment, the TVM is a fragment of an isoform, homologue, or variant of a TNFRSF21 protein, DR6 protein, or precursor thereof. Each possibility represents another embodiment of the present invention.

In another embodiment, the TVM is an FZD1 protein. In another embodiment, the marker is a nucleic acid molecule encoding an FZD10 protein. In another embodiment, the FZD10 protein is encoded by a nucleic acid molecule having the sequence set forth in SEQ ID No: 37. In another embodiment, the FZD10 protein is encoded by a nucleic acid molecule having a sequence set forth in GenBank Accession No. AB027464. In another embodiment, the FZD10 protein is encoded by a nucleic acid molecule having a sequence selected from those set forth in GenBank Accession No. BC070037, BC074997, BC074998, NP_{—}009128, and NM_{—}007197. In another embodiment, the FZD10 protein has an AA sequence set forth in one of the above GenBank entries. In another embodiment, the FZD10 protein is encoded by any other FZD10 gene sequence known in the art. In another embodiment, the FZD10 protein is any other FZD10 protein known in the art. In another embodiment, the TVM is an isoform of an FZD10 protein. In another embodiment, the TVM is a homologue of an FZD10 protein. In another embodiment, the TVM is a variant of an FZD10 protein. In another embodiment, the TVM is a fragment of an FZD10 protein. In another embodiment, the TVM is a fragment of an isoform, homologue, or variant of an FZD10 protein. Each possibility represents another embodiment of the present invention.

In another embodiment, the TVM is an HOXA9 protein. In another embodiment, the marker is a nucleic acid molecule encoding an HOXA9 protein. In another embodiment, the HOXA9 protein is encoded by a nucleic acid molecule having the sequence:

(SEQ ID No: 85)

agtttcataatttccgtgggtcgggccgggcgggccaggcgctgggcacg
gtgatggccaccactggggccctgggcaactactacgtggactcgttcct
gctgggcgccgacgccgcggatgagctgagcgttggccgctatgcgccgg
ggaccctgggccagcctccccggcaggcggcgacgctggccgagcacccc
gacttcagcccgtgcagcttccagtccaaggcgacggtgtttggcgcctc
gtggaacccagtgcacgcggcgggcgccaacgctgtacccgctgcggtgt
accaccaccatcaccaccacccctacgtgcacccccaggcgcccgtggcg
gcggcggcgccggacggcaggtacatgcgctcctggctggagcccacgcc
cggtgcgctctccttcgcgggcttgccctccagccggccttatggcatta
aacctgaaccgctgtcggccagaaggggtgactgtcccacgcttgacact
cacactttgtccctgactgactatgcttgtggttctcctccagttgatag
agaaaaacaacccagcgaaggcgccttctctgaaaacaatgctgagaatg
agagcggcggagacaagccccccatcgatcccaataacccagcagccaac
tggcttcatgcgcgctccactcggaaaaagcggtgcccctatacaaaaca
ccagaccctggaactggagaaagagtttctgttcaacatgtacctcacca
gggaccgcaggtacgaggtggctcgactgctcaacctcaccgagaggcag
gtcaagatctggttccagaaccgcaggatgaaaatgaagaaaatcaacaa
agaccgagcaaaagacgagtgatgccatttgggcttatttagaaaaaagg
gtaagctagagagaaaaagaaagaactgtccgtcccccttccgccttctc
ccttttctcacccccaccctagcctccaccatccccgcacaaagcggctc
taaacctcaggccacatcttttccaaggcaaaccctgttcaggctggctc
gtaggcctgccgctttgatggaggaggtattgtaagctttccattttcta
taagaaaaaggaaaagttgaggggggggcattagtgctgatagctgtgtg
tgttagcttgtatatatatttttaaaaatctacctgttcctgacttaaaa
caaaaggaaagaaactacctttttataatgcacaactgttgatggtaggc
tgtatagtttttagtctgtgtagttaatttaatttgcagtttgtgcggca
gattgctgtgccaagatacttgaacactgtgttttattgtggtaattatg
ttttgtgattcaaacttctgtgtactgggtgatgcacccattgtgattgt
ggaagatagaattcaatttgaactcaggttgtttatgaggggaaaaaaac
agttgcatagagtatagctctgtagtggaatatgtcttctgtataactag
gctgttaacctatgattgtaaagtagctgtaagaatttcccagtgaaata
aaaaaaaattttaagtgttctcggggatgcatagattcatcattttctcc
accttaaaaatgcgggcatttaagtctgtccattatctatatagtcctgt
cttgtctattgtatatataatctatatgattaaagaaaatatgcataatc
agacaagcttgaatattgtttttgcaccagacgaacagtgaggaaattcg
gagctatacatatgtgcagaaggttactacctagggtttatgcttaattt
taatcggaggaaatgaatgctgattgtaacggagttaattttattgataa
taaattatacactatgaaaccgccattgggctactgtagatttgtatcct
tgatgaatctggggtttccatcagactgaacttacactgtatattttgca
atagttacctcaaggcctactgaccaaattgttgtgttgagatgatattt
aactttttgccaaataaaatatattgattcttttctaaaaaaaaaaaaaa
aaaaaa.

In another embodiment, the HOXA9 protein is encoded by a nucleic acid molecule having a sequence set forth in GenBank Accession No. BC006537. In another embodiment, the HOXA9 protein is encoded by a nucleic acid molecule having a sequence selected from those set forth in GenBank Accession No. BC010023, NM_{—}152739, U41813, NM_{—}002142, U82759, and BT006990. In another embodiment, the HOXA9 protein has an AA sequence set forth in one of the above GenBank entries. In another embodiment, the HOXA9 protein is encoded by any other HOXA9 gene sequence known in the art. In another embodiment, the HOXA9 protein is any other HOXA9 protein known in the art. In another embodiment, the TVM is an isoform of an HOXA9 protein. In another embodiment, the TVM is a homologue of an HOXA9 protein. In another embodiment, the TVM is a variant of an HOXA9 protein. In another embodiment, the TVM is a fragment of an HOXA9 protein. In another embodiment, the TVM is a fragment of an isoform, homologue, or variant of an HOXA9 protein. Each possibility represents another embodiment of the present invention.

In another embodiment, the TVM is an SLPI protein. In another embodiment, the marker is a nucleic acid molecule encoding an SLPI protein. In another embodiment, the SLPI protein is encoded by a nucleic acid molecule having the sequence:

(SEQ ID No: 86)

agagtcactcctgccttcaccatgaagtccagcggcctcttccccttcct
ggtgctgcttgccctgggaactctggcaccttgggctgtggaaggctctg
gaaagtccttcaaagctggagtctgtcctcctaagaaatctgcccagtgc
cttagatacaagaaacctgagtgccagagtgactggcagtgtccagggaa
gaagagatgttgtcctgacacttgtggcatcaaatgcctggatcctgttg
acaccccaaacccaacaaggaggaagcctgggaagtgcccagtgacttat
ggccaatgtttgatgcttaacccccccaatttctgtgagatggatggcca
gtgcaagcgtgacttgaagtgttgcatgggcatgtgtgggaaatcctgcg
tttcccctgtgaaagcttgattcctgccatatggaggaggctctggagtc
ctgctctgtgtggtccaggtcctttccaccctgagacttggctccaccac
tgatatcctcctttggggaaaggcttggcacacagcaggctttcaagaag
tgccagttgatcaatgaataaataaacgagcctatttctctttgcaaaaa
aaaaaaaaaaaaaaaaaaaaaaaaa.

In another embodiment, the SLPI protein is encoded by a nucleic acid molecule having a sequence set forth in GenBank Accession No. BC020708. In another embodiment, the SLPI protein is encoded by a nucleic acid molecule having a sequence selected from those set forth in GenBank Accession No. NM_{—}003064, X04470, X04503, and AF114471. In another embodiment, the SLPI protein has an AA sequence set forth in one of the above GenBank entries. In another embodiment, the SLPI protein is encoded by any other SLPI gene sequence known in the art. In another embodiment, the SLPI protein is any other SLPI protein known in the art. In another embodiment, the TVM is an isoform of an SLPI protein. In another embodiment, the TVM is a homologue of an SLPI protein. In another embodiment, the TVM is a variant of an SLPI protein. In another embodiment, the TVM is a fragment of an SLPI protein. In another embodiment, the TVM is a fragment of an isoform, homologue, or variant of an SLPI protein. Each possibility represents another embodiment of the present invention.

In another embodiment, the TVM is a KIBRA protein. In another embodiment, the marker is a nucleic acid molecule encoding a KIBRA protein. In another embodiment, the KIBRA protein is encoded by a nucleic acid molecule having the sequence:

(SEQ ID No: 87)

caaccttctcagctacaaatacttgaagaaacagagcagggagctcaagc
cagtgggagtcatggcccctgcctcagggcctgccagcacggacgctgtg
tctgctctgttggaacagacagcagtggagctggagaagaggcaggaggg
caggagcagcacacagacactggaagacagctggaggtatgaggagacca
gtgagaatgaggcagtagccgaggaagaggaggaggaggtggaggaggag
gagggagaagaggatgttttcaccgagaaagcctcacctgatatggatgg
gtacccagcattaaaggtggacaaagagaccaacacggagaccccggccc
catcccccacagtggtgcgacctaaggaccggagagtgggcaccccgtcc
caggggccatttcttcgagggagcaccatcatccgctctaagaccttctc
cccaggaccccagagccagtacgtgtgccggctgaatcggagtgatagtg
acagctccactctgtccaaaaagccaccttttgttcgaaactccctggag
cgacgcagcgtccggatgaagcggccttcctcggtcaagtcgctgcgctc
cgagcgtctgatccgtacctcgctggacctggagttagacctgcaggcga
caagaacctggcacagccaattgacccaggagatctcggtgctgaaggag
ctcaaggagcagctggaacaagccaagagccacggggagaaggagctgcc
acagtggttgcgtgaggacgagcgtttccgcctgctgctgaggatgctgg
agaagcggcagatggaccgagcggagcacaagggtgagcttcagacagac
aagatgatgagggcagctgccaaggatgtgcacaggctccgaggccagag
ctgtaaggaacccccagaagttcagtctttcagggaggagatggcatttt
tcacccggcctcggatgaatatcccagctctctctgcagatgacgtctaa
tcgccagaaaagtatttcctttgttccactgacaggctgtgaacattgac
tgtggctaaagttatttatgtggtgttatatgaaggtactgagtcacaag
tcctctagtgctcttgttggtttgaagatgaaccgactttttagtttggt
cctactgttgttattaaaaaaaaaaaaaaaacaaaaaaaaaaaaaaaaaa
aaaaaaaaa.

In another embodiment, the KIBRA protein is encoded by a nucleic acid molecule having a sequence set forth in GenBank Accession No. BC004394. In another embodiment, the KIBRA protein is encoded by a nucleic acid molecule having a sequence selected from those set forth in GenBank Accession No. AK001727, NM_{—}015238, BC017746, AF506799, AY189820, AF530058, AB020676, and BX640827. In another embodiment, the KIBRA protein has an AA sequence set forth in one of the above GenBank entries. In another embodiment, the KIBRA protein is encoded by any other KIBRA gene sequence known in the art. In another embodiment, the KIBRA protein is any other KIBRA protein known in the art. In another embodiment, the TVM is an isoform of a KIBRA protein. In another embodiment, the TVM is a homologue of a KIBRA protein. In another embodiment, the TVM is a variant of a KIBRA protein. In another embodiment, the TVM is a fragment of a KIBRA protein. In another embodiment, the TVM is a fragment of an isoform, homologue, or variant of a KIBRA protein. Each possibility represents another embodiment of the present invention.

In another embodiment, the TVM is an IL10RA protein. In another embodiment, the marker is a nucleic acid molecule encoding an IL10RA protein. In another embodiment, the IL10RA protein is encoded by a nucleic acid molecule having the sequence:

(SEQ ID No: 142)

tggaggcgcgcaggccggctccgctccggccccggacgatgcggcgcgcc
caggatgctgccgtgcctcgtagtgctgctggcggcgctcctcagcctcc
gtcttggctcagacgctcatgggacagagctgcccagccctccgtctgtg
tggtttgaagcagaatttttccaccacatcctccactggacacccatccc
aaatcagtctgaaagtacctgctatgaagtggcactcctgaggtatggaa
tagagtcctggaactccatctccaactgtagccagaccctgtcctatgac
cttaccgcagtgaccttggacctgtaccacagcaatggctaccgggccag
agtgcgggctgtggacggcagccggcactccaactggaccgtcaccaaca
cccgcttctctgtggatgaagtgactctgacagttggcagtgtgaaccta
gagatccacaatggcttcatcctcgggaagattcagctacccaggcccaa
gatggcccccgcaaatgacacatatgaaagcatcttcagtcacttccgag
agtatgagattgccattcgcaaggtgccgggaaacttcacgttcacacac
aagaaagtaaaacatgaaaacttcagcctcctaacctctggagaagtggg
agagttctgtgtccaggtgaaaccatctgtcgcttcccgaagtaacaagg
ggatgtggtctaaagaggagtgcatctccctcaccaggcagtatttcacc
gtgaccaacgtcatcatctcaagaagcccagccccttcatcttcatcagc
cagcgtccctccccagagacccaagacaccatccacccgcttgatgagga
ggcctttttgaaggtgtccccagagctgaagaacttggacctgcacggca
gcacagacagtggctttggcagcaccaagccatccctgcagactgaagag
ccccagttcctcctccctgaccctcacccccaggctgacagaacgctggg
aaacggggagccccctgtgctgggggacagctgcagtagtggcagcagca
atagcacagacagcgggatctgcctgcaggagcccagcctgagccccagc
acagggcccacctgggagcaacaggtggggagcaacagcaggggccagga
tgacagtggcattgacttagttcaaaactctgagggccgggctggggaca
cacagggtggctcggccttgggccaccacagtcccccggagcctgaggtg
cctggggaagaagacccagctgctgtggcattccagggttacctgaggca
gaccagatgtgctgaagagaaggcaaccaagacaggctgcctggaggaag
aatcgcccttgacagatggccttggccccaaattcgggagatgcctggtt
gatgaggcaggcttgcatccaccagccctggccaagggctatttgaaaca
ggatcctctagaaatgactctggcttcctcaggggccccaacgggacagt
ggaaccagcccactgaggaatggtcactcctggccttgagcagctgcagt
gacctgggaatatctgactggagctttgcccatgaccttgcccctctagg
ctgtgtggcagccccaggtggtctcctgggcagctttaactcagacctgg
tcaccctgcccctcatctctagcctgcagtcaagtgagtgactcgggctg
agaggctgcttttgattttagccatgcctgctcctctgcctggaccagga
ggagggcccctggggcagaagttaggcacgaggcagtctgggcacttttc
tgcaagtccactggggctggccccagccaggccctgcagggctggtcagg
gtgtctggggcaggaggaggccaactcactgaactagtgcagggtatgtg
ggtggcactgacctgttctgttgactggggccctgcagactctggcagag
ctgagaagggcagggaccttctccctcctaggaactctttcctgtatcat
aaggattatttgctcaggggaaccatggggctttctggagttgtggtgag
gccaccaggctgaagtcagctcagacccagacctccctgcttaggccact
cgagcatcagagcttccagcaggaggaagggctgtaggaatggaagcttc
agggccttgctgctggggtcatttttaggggaaaaaggaggatatgatgg
tcacatggggaacctcccctcatcgggcctctggggcaggaagcttgtca
ctggaagatcttaaggtatatattttctggacactcaaacacatcataat
ggattcactgaggggagacaaagggagccgagaccctggatggggcttcc
agctcagaacccatccctctggtgggtacctctggcacccatctgcaaat
atctccctctctccaacaaatggagtagcatccccctggggcacttgctg
aggccaagccactcacatcctcactttgctgccccaccatcttgctgaca
acttccagagaagccatggttttttgtattggtcataactcagccctttg
ggcggcctctgggcttgggcaccagctcatgccagccccagagggtcagg
gttggaggcctgtgcttgtgtttgctgctaatgtccagctacagacccag
aggataagccactgggcactgggctggggtccctgccttgttggtgttca
gctgtgtgattttggactagccacttgtcagagggcctcaatctcccatc
tgtgaaataaggactccacctttaggggaccctccatgtttgctgggtat
tagccaagctggtcctgggagaatgcagatactgtccgtggactaccaag
ctggcttgtttcttatgccagaggctaacagatccaatgggagtccatgg
tgtcatgccaagacagtatcagacacagccccagaagggggcattatggg
ccctgcctccccataggccatttggactctgccttcaaacaaaggcagtt
cagtccacaggcatggaagctgtgaggggacaggcctgtgcgtgccatcc
agagtcatctcagccctgcctttctctggagcattctgaaaacagatatt
ctggcccagggaatccagccatgacccccacccctctgccaaagtactct
taggtgccagtctggtaactgaactccctctggaggcaggcttgagggag
gattcctcagggttcccttgaaagctttatttatttattttgttcattta
tttattggagaggcagcattgcacagtgaaagaattctggatatctcagg
agccccgaaattctagctctgactttgctgtttccagtggtatgaccttg
gagaagtcacttatcctcttggagcctcagtttcctcatctgcagaataa
tgactgacttgtctaattcgtagggatgtgaggttctgctgaggaaatgg
gtatgaatgtgccttgaacacaaagctctgtcaataagtgatacatgttt
tttattccaataaattgtcaagaccacaaaaaaaaaaaaaaaaaaaaaaa
aaaaaaaaaaaaaaaaaaaaaaaaaaaaaaaaaaaaaaaa.

In another embodiment, the IL10RA protein is encoded by a nucleic acid molecule having a sequence set forth in GenBank Accession No. BC028082. In another embodiment, the IL10RA protein is encoded by a nucleic acid molecule having a sequence selected from those set forth in GenBank Accession No. NM_{—}001558, AB209626, U00672, and BC028082. In another embodiment, the IL10RA protein has an AA sequence set forth in one of the above GenBank entries. In another embodiment, the IL10RA protein is encoded by any other IL10RA gene sequence known in the art. In another embodiment, the IL10RA protein is any other IL10RA protein known in the art. In another embodiment, the TVM is an isoform of an IL10RA protein. In another embodiment, the TVM is a homologue of an IL10RA protein. In another embodiment, the TVM is a variant of an IL10RA protein. In another embodiment, the TVM is a fragment of an IL10RA protein. In another embodiment, the TVM is a fragment of an isoform, homologue, or variant of an IL10RA protein. Each possibility represents another embodiment of the present invention.

In another embodiment, the TVM is an ADAM12 protein. In another embodiment, the marker is a nucleic acid molecule encoding an ADAM12 protein. In another embodiment, the ADAM12 nucleotide is a long isoform of ADAM12. In another embodiment, the ADAM12 nucleotide is a short isoform of ADAM12. In another embodiment, the ADAM12 protein is encoded by a nucleic acid molecule having the sequence:

(SEQ ID No: 1)

cactaacgctcttcctagtccccgggccaactcggacagtttgctcattt
attgcaacggtcaaggctggcttgtgccagaacggcgcgcgcgcgacgca
cgcacacacacggggggaaacttttttaaaaatgaaaggctagaagagct
cagcggcggcgcgggccgtgcgcgagggctccggagctgactcgccgagg
caggaaatccctccggtcgcgacgcccggccccgctcggcgcccgcgtgg
gatggtgcagcgctcgccgccgggcccgagagctgctgcactgaaggccg
gcgacgatggcagcgcgcccgctgcccgtgtcccccgcccgcgccctcct
gctcgccctggccggtgctctgctcgcgccctgcgaggcccgaggggtga
gcttatggaaccaaggaagagctgatgaagttgtcagtgcctctgttcgg
agtggggacctctggatcccagtgaagagcttcgactccaagaatcatcc
agaagtgctgaatattcgactacaacgggaaagcaaagaactgatcataa
atctggaaagaaatgaaggtctcattgccagcagtttcacggaaacccac
tatctgcaagacggtactgatgtctccctcgctcgaaattacacggtaat
tctgggtcactgttactaccatggacatgtacggggatattctgattcag
cagtcagtctcagcacgtgttctggtctcaggggacttattgtgtttgaa
aatgaaagctatgtcttagaaccaatgaaaagtgcaaccaacagatacaa
actcttcccagcgaagaagctgaaaagcgtccggggatcatgtggatcac
atcacaacacaccaaacctcgctgcaaagaatgtgtttccaccaccctct
cagacatgggcaagaaggcataaaagagagaccctcaaggcaactaagta
tgtggagctggtgatcgtggcagacaaccgagagtttcagaggcaaggaa
aagatctggaaaaagttaagcagcgattaatagagattgctaatcacgtt
gacaagttttacagaccactgaacattcggatcgtgttggtaggcgtgga
agtgtggaatgacatggacaaatgctctgtaagtcaggacccattcacca
gcctccatgaatttctggactggaggaagatgaagcttctacctcgcaaa
tcccatgacaatgcgcagcttgtcagtggggtttatttccaagggaccac
catcggcatggccccaatcatgagcatgtgcacggcagaccagtctgggg
gaattgtcatggaccattcagacaatccccttggtgcagccgtgaccctg
gcacatgagctgggccacaatttcgggatgaatcatgacacactggacag
gggctgtagctgtcaaatggcggttgagaaaggaggctgcatcatgaacg
cttccaccgggtacccatttcccatggtgttcagcagttgcagcaggaag
gacttggagaccagcctggagaaaggaatgggggtgtgcctgtttaacct
gccggaagtcagggagtctttcgggggccagaagtgtgggaacagatttg
tggaagaaggagaggagtgtgactgtggggagccagaggaatgtatgaat
cgctgctgcaatgccaccacctgtaccctgaagccggacgctgtgtgcgc
acatgggctgtgctgtgaagactgccagctgaagcctgcaggaacagcgt
gcagggactccagcaactcctgtgacctcccagagttctgcacaggggcc
agccctcactgcccagccaacgtgtacctgcacgatgggcactcatgtca
ggatgtggacggctactgctacaatggcatctgccagactcacgagcagc
agtgtgtcacactctggggaccaggtgctaaacctgcccctgggatctgc
tttgagagagtcaattctgcaggtgatccttatggcaactgtggcaaagt
ctcgaagagttcctttgccaaatgcgagatgagagatgctaaatgtggaa
aaatccagtgtcaaggaggtgccagccggccagtcattggtaccaatgcc
gtttccatagaaacaaacatccccctgcagcaaggaggccggattctgtg
ccgggggacccacgtgtacttgggcgatgacatgccggacccagggcttg
tgcttgcaggcacaaagtgtgcagatggaaaaatctgcctgaatcgtcaa
tgtcaaaatattagtgtctttggggttcacgagtgtgcaatgcagtgcca
cggcagaggggtgtgcaacaacaggaagaactgccactgcgaggcccact
gggcacctcccttctgtgacaagtttggctttggaggaagcacagacagc
ggccccatccggcaagcagataaccaaggtttaaccataggaattctggt
gaccatcctgtgcttcttgctgccggatttgtggtttatctcaaaaggaa
gaccttgatacgactgctgtttacaaataagaagaccaccattgaaaaac
taaggtgtgtgcgcccttcccggccaccccgtggcttccaaccctgtcag
gctcacctcggccaccttggaaaaggcctgatgaggaagccgccagattc
ctacccaccgaaggacaatcccaggagattgctgcagtgtcagaatgttg
acatcagcagacccctcaacggcctgaatgtccctcagccccagtcaact
cagcgagtgcttcctcccctccaccgggccccacgtgcacctagcgtccc
tgccagacccctgccagccaagcctgcacttaggcaggcccaggggacct
gtaagccaaacccccctcagaagcctctgcctgcagatcctctggccaga
acaactcggctcactcatgccttggccaggaccccaggacaatgggagac
tgggctccgcctggcacccctcagacctgctccacaatatccacaccaag
tgcccagatccacccacaccgcctatattaagtgagaagccgacaccttt
tttcaacagtgaagacagaagtttgcactatctttcagctccagttggag
ttttttgtaccaacttttaggattttttttaatgtttaaaacatcattac
tataagaactttgagctactgccgtcagtgctgtgctgtgctatggtgct
ctgtctacttgcacaggtacttgtaaattattaatttatgcagaatgttg
attacagtgcagtgcgctgtagtaggcatttttaccatcactgagttttc
catggcaggaaggcttgttgtgcttttagtattttagtgaacttgaaata
tcctgcttgatgggattctggacaggatgtgtttgctttctgatcaaggc
cttattggaaagcagtcccccaactacccccagctgtgcttatggtacca
gatgcagctcaagagatcccaagtagaatctcagttgattttctggattc
cccatctcaggccagagccaaggggcttcaggtccaggctgtgtttggct
ttcagggaggccctgtgccccttgacaactggcaggcaggctcccaggga
cacctgggagaaatctggcttctggccaggaagctttggtgagaacctgg
gttgcagacaggaatcttaaggtgtagccacaccaggatagagactggaa
cactagacaagccagaacttgaccctgagctgaccagccgtgagcatgtt
tggaaggggtctgtagtgtcactcaaggcggtgcttgatagaaatgccaa
gcacttctttttctcgctgtcctttctagagcactgccaccagtaggtta
tttagcttgggaaaggtggtgtttctgtaagaaacctactgcccaggcac
tgcaaaccgccacctccctatactgcttggagctgagcaaatcaccacaa
actgtaatacaatgatcctgtattcagacagatgaggactttccatggga
ccacaactattttcagatgtgaaccattaaccagatctagtcaatcaagt
ctgtttactgcaaggttcaacttattaacaattaggcagactctttatgc
ttgcaaaaactacaaccaatggaatgtgatgttcatgggtatagttcatg
tcgctatcattattcgtagatattggacaaagaaccttctctatggggca
tcctctttttccaacttggctgcaggaatctttaaaagatgcttttaaca
gagtctgaacctatttcttaaacacttgcaacctacctgttgagcatcac
agaatgtgataaggaaatcaacttgcttatcaacttcctaaatattatga
gatgtggcttgggcagcatccccttgaactcttcactcttcaaatgcctg
actagggagccatgtttcacaaggtctttaaagtgactaatggcatgaga
aatacaaaaatactcagataaggtaaaatgccatgatgcctctgtcttct
ggactggttttcacattagaagacaattgacaacagttacataattcact
ctgagtgttttatgagaaagccttcttttggggtcaacagttttcctatg
ctttgaaacagaaaaatatgtaccaagaatcttggtttgccttccagaaa
acaaaactgcatttcactttcccggtgttccccactgtatctaggcaaca
tagtattcatgactatggataaactaaacacgtgacacaaacacacacaa
aagggaacccagctctaatacattccaactcgtatagcatgcatctgttt
attctatagttattaagttctttaaaatgtaaagccatgctggaaaataa
tactgctgagatacatacagaattactgtaactgattacacttggtaatt
gtactaaagccaaacatatatatactattaaaaaggtttacagaatttta
tggtgcattacgttgggcattgtctttttagatgcccaaatccttagatc
tggcatgttagcccttcctccaattataagaggatatgaaccaaaaaaaa
aaaaaaaaaaa.

In another embodiment, the ADAM12 protein is a long isoform of ADAM12. In another embodiment, the ADAM12 protein is a short isoform of ADAM12. In another embodiment, the ADAM12 protein is encoded by a nucleic acid molecule having a sequence set forth in GenBank Accession No. AF023476. In another embodiment, the ADAM12 protein is encoded by a nucleic acid molecule having a sequence set forth in GenBank Accession No. AF023477. In another embodiment, the ADAM12 protein is encoded by a nucleic acid molecule having a sequence set forth in GenBank Accession No. NM_{—}003474. In another embodiment, the ADAM12 protein has an AA sequence set forth in 1 of the above GenBank entries. In another embodiment, the ADAM12 protein is encoded by any other ADAM12 gene sequence known in the art. In another embodiment, the ADAM12 protein is any other ADAM12 protein known in the art. In another embodiment, the TVM is an isoform of an ADAM12 protein. In another embodiment, the TVM is a homologue of an ADAM12 protein. In another embodiment, the TVM is a variant of an ADAM12 protein. In another embodiment, the TVM is a fragment of an ADAM12 protein. In another embodiment, the TVM is a fragment of an isoform, homologue, or variant of an ADAM12 protein. Each possibility represents another embodiment of the present invention.

In another embodiment, the TVM is a PCDH17 protein. In another embodiment, the marker is a nucleic acid molecule encoding a PCDH17 protein. In another embodiment, the PCDH17 protein is encoded by a nucleic acid molecule having the sequence set forth in SEQ ID No: 49. In another embodiment, the PCDH17 protein is encoded by a nucleic acid molecule having a sequence set forth in GenBank Accession No. AL137505. In another embodiment, the PCDH17 protein has an AA sequence set forth in GenBank Accession No. AL137505. In another embodiment, the PCDH17 protein is encoded by any other PCDH17 gene sequence known in the art. In another embodiment, the PCDH17 protein is any other PCDH17 protein known in the art. In another embodiment, the TVM is an isoform of a PCDH17 protein. In another embodiment, the TVM is a homologue of a PCDH17 protein. In another embodiment, the TVM is a variant of a PCDH17 protein. In another embodiment, the TVM is a fragment of a PCDH17 protein. In another embodiment, the TVM is a fragment of an isoform, homologue, or variant of a PCDH17 protein. Each possibility represents another embodiment of the present invention.

In another embodiment, the TVM is an AML-1 protein. In another embodiment, the marker is a nucleic acid molecule encoding an AML-1 protein. In another embodiment, the AML-1 protein is encoded by a nucleic acid molecule having the sequence:

(SEQ ID No: 5)

catagagccagcgggcgcgggcgggacgggcgccccgcggccggacccag
ccagggcaccacgctgcccggccctgcgccgccaggcacttctttccggg
gctcctagggacgccagaaggaagtcaacctctgctgcttctccttggcc
tgcgttggaccttcctttttttgttgtttttttttgtttttcccctttct
tccttttgaattaactggcttcttggctggatgttttcaacttctttcct
ggctgcgaacttttccccaattgttttccttttacaacagggggagaaag
tgctctgtggtccgaggcgagccgtgaagttgcgtgtgcgtggcagtgtg
cgtggcaggatgtgcgtgcgtgtgtaacccgagccgcccgatctgtttcg
atctgcgccgcggagccctccctcaaggcccgctccacctgctgcggtta
cgcggcgctcgtgggtgttcgtgcctcggagcagctaaccggcgggtgct
gggcgacggtggaggagtatcgtctcgctgctgcccgagtcagggctgag
tcacccagctgatgtagacagtggctgccttccgaagagtgcgtgtttgc
atgtgtgtgactctgcggctgctcaactcccaacaaaccagaggaccagc
cacaaacttaaccaacatccccaaacccgagttcacagatgtgggagagc
tgtagaaccctgagtgtcatcgactgggccttcttatgattgttgtttta
agattagctgaagatctctgaaacgctgaattttctgcactgagcgtttt
gacagaattcattgagagaacagagaacatgacaagtacttctagctcag
cactgctccaactactgaagctgattttcaaggctacttaaaaaaatctg
cagcgtacattaatggatttctgttgtgtttaaattctccacagattgta
ttgtaaatattttatgaagtagagcatatgtatatatttatatatacgtg
cacatacattagtagcactacctttggaagtctcagctcttgcttttcgg
gactgaagccagttttgcatgataaaagtggccttgttacgggagataat
tgtgttctgttgggactttagacaaaactcacctgcaaaaaactgacagg
cattaactactggaacttccaaataatgtgtttgctgatcgttttactct
tcgcataaatattttaggaagtgtatgagaattttgccttcaggaacttt
tctaacagccaaagacagaacttaacctctgcaagcaagattcgtggaag
atagtctccactttttaatgcactaagcaatcggtitgctaggagcccat
cctgggtcagaggccgatccgcagaaccagaacgttttcccctcctggac
tgttagtaacttagtctccctcctcccctaaccacccccgccccccccca
ccccccgcagtaataaaggcccctgaacgtgtatgttggtctcccgggag
ctgcttgctgaagatccgcgcccctgtcgccgtctggtaggagctgtttg
cagggtcctaactcaatcggcttgttgtgatgcgtatccccgtagatgcc
agcacgagccgccgcttcacgccgccttccaccgcgctgagcccaggcaa
gatgagcgaggcgttgccgctgggcgccccggacgccggcgctgccctgg
ccggcaagctgaggagcggcgaccgcagcatggtggaggtgctggccgac
cacccgggcgagctggtgcgcaccgacagccccaacttcctctgctccgt
gctgcctacgcactggcgctgcaacaagaccctgcccatcgctttcaagg
tggtggccctaggggatgttccagatggcactctggtcactgtgatggct
ggcaatgatgaaaactactcggctgagctgagaaatgctaccgcagccat
gaagaaccaggttgcaagatttaatgacctcaggtttgtcggtcgaagtg
gaagagggaaaagcttcactctgaccatcactgtcttcacaaacccaccg
caagtcgccacctaccacagagccatcaaaatcacagtggatgggccccg
agaacctcgaagacatcggcagaaactagatgatcagaccaagcccggga
gcttgtccttttccgagcggctcagtgaactggagcagctgcggcgcaca
gccatgagggtcagcccacaccacccagcccccacgcccaaccctcgtgc
ctccctgaaccactccactgcctttaaccctcagcctcagagtcagatgc
aggatacaaggcagatccaaccatccccaccgtggtcctacgatcagtcc
taccaatacctgggatccattgcctctccttctgtgcacccagcaacgcc
catttcacctggacgtgccagcggcatgacaaccctctctgcagaacttt
ccagtcgactctcaacggcacccgacctgacagcgttcagcgacccgcgc
cagttccccgcgctgccctccatctccgacccccgcatgcactatccagg
cgccttcacctactccccgacgccggtcacctcgggcatcggcatcggca
tgtcggccatgggctcggccacgcgctaccacacctacctgccgccgccc
taccccggctcgtcgcaagcgcagggaggcccgttccaagccagctcgcc
ctcctaccacctgtactacggcgcctcggccggctcctaccagttctcca
tggtgggcggcgagcgctcgccgccgcgcatcctgccgccctgcaccaac
gcctccaccggctccgcgctgctcaaccccagccrcccgaaccagagcga
cgtggtggaggccgagggcagccacagcaactcccccaccaacatggcgc
cctccgcgcgcctggaggaggccgtgtggaggccctactgaggcgccagg
cctggcccggctgggccccgcgggccgccgccttcgcctccgggcgcgcg
ggcctcctgttcgcgacaagcccgccgggatcccgggccctgggcccggc
caccgtcctggggccgagggcgcccgacggccaggatctcgctgtaggtc
aggcccgcgcagcctcctgcgcccagaagcccacgccgccgccgtctgct
ggcgccccggccctcgcggaggtgtccgaggcgacgcacctcgagggtgt
ccgccggccccagcacccaggggacgcgctggaaagcaaacaggaagatt
cccggagggaaactgtgaatgcttctgatttagcaatgctgtgaataaaa
agaaagattttatacccttgacttaactttttaaccaagttgtttattcc
aaagagtgtggaattttggttggggtggggggagaggagggatgcaactc
gccctgtttggcatctaattcttatttttaatttttccgcaccttatcaa
ttgcaaaatgcgtatttgcatttgggtggtttttatttttatatacgttt
atataaatatatataaattgagcttgcttctttcttgctttgaccatgga
aagaaatatgattcccttttctttaagttttatttaacttttcttttgga
cttttgggtagttgtttttttttgttttgttttgtttttttgagaaacag
ctacagctttgggtcatttttaactactgtattccacaaggaatccccag
atatttatgtatcttgatgttcagacatttatgtgttgataattttttaa
ttatttaaatgtacttatattaagaaaaatatcaagtactacattttctt
ttgttcttgatagtagccaaagttaaatgtatcacattgaagaaggctag
aaaaaaagaatgagtaatgtgatcgcttggttatccagaagtattgttta
cattaaactccctttcatgttaatcaaacaagtgagtagctcacgcagca
acgtttttaataggatttttagacactgagggtcactccaaggatcagaa
gtatggaattttctgccaggctcaacaagggtctcatatctaacttcctc
cttaaaacagagaaggtcaatctagttccagagggttgaggcaggtgcca
ataattacatctttggagaggatttgatttctgcccagggatttgctcac
cccaaggtcatctgataatttcacagatgctgtgtaacagaacacagcca
aagtaaactgtgtaggggagccacatttacataggaaccaaatcaatgaa
tttaggggttacgattatagcaatttaagggccaccagaagcaggcctcg
aggagtcaatttgcctctgtgtgcctcagtggagacaagtgggaaaacat
ggtcccacctgtgcgagaccccctgtcctgtgctgctcactcaacaacat
ctttgtgttgctttcaccaggctgagaccctaccctatggggtatatggg
cttttacctgtgcaccagtgtgacaggaaagattcatgtcactactgtcc
gtggctacaattcaaaggtatccaatgtcgctgtaaattttatggcacta
tttttattggaggatttggtcagaatgcagttgttgtacaactcataaat
actaactgctgattttgacacatgtgtgctccaaatgatctggtggttat
ttaacgtacctcttaaaattcgttgaaacgatttcaggtcaactctgaag
agtatttgaaagcaggacttcagaacagtgtttgatttttattttataaa
tttaagcattcaaattaggcaaatctttggctgcaggcagcaaaaacagc
tggacttatttaaaacaacttgtttttgagttttcttatatatatattga
ttatttgttttacacacatgcagtagcactttggtaagagttaaagagta
aagcagcttatgttgtcaggtcgttcttatctagagaagagctatagcag
atctcggacaaactcagaatatattcactttcatttttgacaggattccc
tccacaactcagtttcatatattattccgtattacatttttgcagctaaa
ttaccataaaatgtcagcaaatgtaaaaatttaatttctgaaaagcacca
ttagcccatttcccccaaattaaacgtaaatgttttttttcagcacatgt
taccatgtctgacctgcaaaaatgctggagaaaaatgaaggaaaaattat
gtttttcagtttaattctgttaactgaagatattccaactcaaaaccagc
ctcatgctctgattagataatcttttacattgaacctttactctcaaagc
catgtgtggagggggcttgtcactattgtaggctcactggattggtcatt
tagagtttcacagactcttaccagcatatatagtatttaattgtttcaaa
aaaaatcaaactgtagttgttttggcgataggtctcacgcaacacatttt
tgtatgtgtgtgtgtgtgcgtgtgtgtgtgtgtgtgtaaaaattgcattc
attgacttcaggtagattaaggtatctttttattcattgccctcaggaaa
gttaaggtatcaatgagacccttaagccaatcatgtaataactgcatgtg
tctggtccaggagaagtattgaataagccatttctactgcttactcatgt
ccctatttatgatttcaacatggatacatatttcagttcttctttttctc
actatctgaaaatacatttccctccctctcttccccccaatatctccctt
tttttctctcttcctctatcttccaaaccccactttctccctcctccttt
tcctgtgttctcttaagcagatagcacatacccccacccagtaccaaatt
tcagaacacaagaaggtccagttcttcccccttcacataaaggaacatgg
tttgtcagcctttctcctgtttatgggtttcttccagcagaacagagaca
ttgccaaccatattggatctgcttgctgtccaaaccagcaaactttcctg
ggcaaatcacaatcagtgagtaaatagacagcctttctgctgccttgggt
ttctgtgcagataaacagaaatgctctgattagaaaggaaatgaatggtc
cactcaaatgtcctgcaatttaggattgcagatttctgccttgaaatacc
tgtttctttgggacattccgtcctgatgatttttatttttgttggttttt
atttttggggggaatgacatgtttgggtcttttatacatgaaaatttgtt
tgacaataatctcacaaaacatattttacatctgaacaaaatgccttttt
gtttaccgtagcgtatacatttgttttgggatttttgtgtgtttgttggg
aattttgtttttagccaggtcagtattgatgaggctgatcatttggctct
ttttttccttccagaagagttgcatcaacaaagttaattgtatttatgta
tgtaaatagattttaagcttcattataaaatattgttaatgcctataact
ttttttcaatttttttgtgtgtgtttctaaggactttttcttaggtttgc
taaatactgtagggaaaaaaatgcttctttctactttgtttattttagac
tttaaaatgagctacttcttattcacttttgtaaacagctaatagcatgg
ttccaattttttttaagttcactttttttgttctaggggaaatgaatgtg
caaaaaaagaaaaagaactgttggttatttgtgttattctggatgtataa
aaatcaatggaaaaaaataaactttcaaattgaaatgacggtataacaca
tctactgaaaaagcaacgggaaatgtggtcctatttaagccagcccccac
ctagggtctatttgtgtggcagttattgggtttggtcacaaaacatcctg
aaaattcgtgcgtgggcttctttctccctggtacaaacgtatggaatgct
tcttaaaggggaactgtcaagctggtgtcttcagccagatgacatgagag
aatatcccagaaccctctctccaaggtgtttctagatagcacaggagagc
aggcactgcactgtccacagtccacggtacacagtcgggtgggccgcctc
ccctctcctgggagcattcgtcgtgcccagcctgagcagggcagctggac
tgctgctgttcaggagccaccagagccttcctctctttgtaccacagttt
cttctgtaaatccagtgttacaatcagtgtgaatggcaaataaacagttt
gacaagtacatacaccataaaaaaaaaaaaaaaa.

In another embodiment, the AML-1 protein is encoded by a nucleic acid molecule having a sequence set forth in GenBank Accession No. NM_{—}001001890. In another embodiment, the AML-1 protein is encoded by a nucleic acid molecule having a sequence set forth in GenBank Accession No. NM_{—}001754. In another embodiment, the AML-1 protein has an AA sequence set forth in 1 of the above GenBank entries. In another embodiment, the AML-1 protein is encoded by any other AML-1 gene sequence known in the art. In another embodiment, the AML-1 protein is any other AML-1 protein known in the art. In another embodiment, the TVM is an isoform of an AML-1 protein. In another embodiment, the TVM is a homologue of an AML-1 protein. In another embodiment, the TVM is a variant of an AML-1 protein. In another embodiment, the TVM is a fragment of an AML-1 protein. In another embodiment, the TVM is a fragment of an isoform, homologue, or variant of an AML-1 protein. Each possibility represents another embodiment of the present invention.

In another embodiment, the TVM is a SLIT2 protein. In another embodiment, the marker is a nucleic acid molecule encoding a SLIT2 protein. In another embodiment, the SLIT2 protein is encoded by a nucleic acid molecule having the sequence:

(SEQ ID No: 59)

cagagcagggtggagagggcggtgggaggcgtgtgcctgagtgggctcta
ctgccttgttccatattattttgtgcacattttccctggcactctgggtt
gctagccccgccgggcactgggcctcagacactgcgcggttccctcggag
cagcaagctaaagaaagcccccagtgccggcgaggaaggaggcggcgggg
aaagatgcgcggcgttggctggcagatgctgtccctgtcgctggggttag
tgctggcgatcctgaacaaggtggcaccgcaggcgtgcccggcgcagtgc
tcttgctcgggcagcacagtggactgtcacgggctggcgctgcgcagcgt
gcccaggaatatcccccgcaacaccgagagactggatttaaatggaaata
acatcacaagaattacgaagacagattttgctggtcttagacatctaaga
gttcttcagcttatggagaataagattagcaccattgaaagaggagcatt
ccaggatcttaaagaactagagagactgcgtttaaacagaaatcaccttc
agctgtttcctgagttgctgtttcttgggactgcgaagctatacaggctt
gatctcagtgaaaaccaaattcaggcaatcccaaggaaagctttccgtgg
ggcagttgacataaaaaatttgcaactggattacaaccagatcagctgta
ttgaagatggggcattcagggctctccgggacctggaagtgctcactctc
aacaataacaacattactagactttctgtggcaagtttcaaccatatgcc
taaacttaggacttttcgactgcattcaaacaacctgtattgtgactgcc
acctggcctggctctccgactggcttcgccaaaggcctcgggttggtctg
tacactcagtgtatgggcccctcccacctgagaggccataatgtagccga
ggttcaaaaacgagaatttgtctgcagtggtcaccagtcatttatggctc
cttcttgtagtgttttgcactgccctgccgcctgtacctgtagcaacaat
atcgtagactgtcgtgggaaaggtctcactgagatccccacaaatcttcc
agagaccatcacagaaatacgtttggaacagaacacaatcaaagtcatcc
ctcctggagctttctcaccatataaaaagcttagacgaattgacctgagc
aataatcagatctctgaacttgcaccagatgctttccaaggactacgctc
tctgaattcacttgtcctctatggaaataaaatcacagaactccccaaaa
gtttatttgaaggactgttttccttacagctcctattattgaatgccaac
aagataaactgccttcgggtagatgcttttcaggatctccacaacttgaa
ccttctctccctatatgacaacaagcttcagaccatcgccaaggggacct
tttcacctcttcgggccattcaaactatgcatttggcccagaaccccttt
atttgtgactgccatctcaagtggctagcggattatctccataccaaccc
gattgagaccagtggtgcccgttgcaccagcccccgccgcctggcaaaca
aaagaattggacagatcaaaagcaagaaattccgttgttcagctaaagaa
cagtatttcattccaggtacagaagattatcgatcaaaattaagtggaga
ctgctttgcggatctggcttgccctgaaaagtgtcgctgtgaaggaacca
cagtagattgctctaatcaaaagctcaacaaaatcccggagcacattccc
cagtacactgcagagttgcgtctcaataataatgaatttaccgtgttgga
agccacaggaatctttaagaaacttcctcaattacgtaaaataaacttta
gcaacaataagatcacagatattgaggagggagcatttgaaggagcatct
ggtgtaaatgaaatacttcttacgagtaatcgtttggaaaatgtgcagca
taagatgttcaagggattggaaagcctcaaaactttgatgttgagaagca
atcgaataacctgtgtggggaatgacagtttcataggactcagttctgtg
cgtttgctttctttgtatgataatcaaattactacagttgcaccaggggc
atttgatactctccattctttatctactctaaacctcttggccaatcctt
ttaactgtaactgctacctggcttggttgggagagtggctgagaaagaag
agaattgtcacgggaaatcctagatgtcaaaaaccatacttcctgaaaga
aatacccatccaggatgtggccattcaggacttcacttgtgatgacggaa
atgatgacaatagttgctccccactttctcgctgtcctactgaatgtact
tgcttggatacagtcgtccgatgtagcaacaagggtttgaaggtcttgcc
gaaaggtattccaagagatgtcacagagttgtatctggatggaaaccaat
ttacactggttcccaaggaactctccaactacaaacatttaacacttata
gacttaagtaacaacagaataagcacgctttctaatcagagcttcagcaa
catgacccagctcctcaccttaattcttagttacaaccgtctgagatgta
ttcctcctcgcacctttgatggattaaagtctcttcgattactttctcta
catggaaatgacatttctgttgtgcctgaaggtgctttcaatgatctttc
tgcattatcacatctagcaattggagccaaccctctttactgtgattgta
acatgcagtggttatccgactgggtgaagtcggaatataaggagcctgga
attgctcgttgtgctggtcctggagaaatggcagataaacttttactcac
aactccctccaaaaaatttacctgtcaaggtcctgtggatgtcaatattc
tagctaagtgtaacccctgcctatcaaatccgtgtaaaaatgatggcaca
tgtaatagtgatccagttgacttttaccgatgcacctgtccatatggttt
caaggggcaggactgtgatgtcccaattcatgcctgcatcagtaacccat
gtaaacatggaggaacttgccacttaaaggaaggagaagaagatggattc
tggtgtatttgtgctgatggatttgaaggagaaaattgtgaagtcaacgt
tgatgattgtgaagataatgactgtgaaaataatctacatgtgtcgatgg
cattaataactacacatgcctttgcccacctgagtatacaggtgagttgt
gtgaggagaagctggacttctgtgcccaggacctgaacccctgccagcac
gattcaaagtgcatcctaactccaaagggattcaaatgtgactgcacacc
agggtacgtaggtgaacactgcgacatcgattttgacgactgccaagaca
acaagtgtaaaaacggagcccactgcacagatgcagtgaacggctatacg
tgcatatgccccgaaggttacagtggcttgttctgtgagttttctccacc
catggtcctccctcgtaccagcccctgtgataattttgattgtcagaatg
gagctcagtgtatcgtcagaataaatgagccaatatgtcagtgtttgcct
ggctatcagggagaaaagtgtgaaaaattggttagtgtgaattttataaa
caaagagtcttatcttcagattccttcagccaaggttcggcctcagacga
acataacacttcagattgccacagatgaagacagcggaatcctcctgtat
aagggtgacaaagaccatatcgcggtagaactctatcgggggcgtgttcg
tgccagctatgacaccggctctcatccagcttctgccatttacagtgtgg
agacaatcaatgatggaaacttccacattgtggaactacttgccttggat
cagagtctctctttgtccgtggatggtgggaaccccaaaatcatcactaa
cttgtcaaagcagtccactctgaattttgactctccactctatgtaggag
gcatgccagggaagagtaacgtggcatctctgcgccaggcccctgggcag
aacggaaccagcttccacggctgcatccggaacctttacatcaacagtga
gctgcaggacttccagaaggtgccgatgcaaacaggcattttgcctggct
gtgagccatgccacaagaaggtgtgtgcccatggcacatgccagcccagc
agccaggcaggcttcacctgcgagtgccaggaaggatggatggggcccct
ctgtgaccaacggaccaatgacccttgccttggaaataaatgcgtacatg
gcacctgcttgcccatcaatgcgttctcctacagctgtaagtgcttggag
ggccatggaggtgtcctctgtgatgaagaggaggatctgtttaacccatg
ccaggcgatcaagtgcaagcacgggaagtgcaggctttcaggtctggggc
agccctactgtgaatgcagcagtggatacacgggggacagctgtgatcga
gaaatctcttgtcgaggggaaaggataagagattattaccaaaagcagca
gggctatgctgcttgccaaacaaccaagaaggtgtcccgattagagtgca
gaggtgggtgtgcaggagggcagtgctgtggaccgctgaggagcaagcgg
cggaaatactctttcgaatgcactgacggctcctcctttgtggacgaggt
tgagaaagtggtgaagtgcggctgtacgaggtgtgtgtcctaaacacact
cccggcagctctgtctttggaaaaggttgtatacttctgaccatgtggga
ctaatgaatgcttcatagtggaaatatttgaaatatattgtaaaatacag
aacagacttatttttattatgagaataaagactttttttctgcatttg.

In another embodiment, the SLIT2 protein is encoded by a nucleic acid molecule having a sequence set forth in GenBank Accession No. NM_{—}004787. In another embodiment, the SLIT2 protein is encoded by a nucleic acid molecule having a sequence selected from those set forth in GenBank Accession No. AB017168 and AK027326. In another embodiment, the SLIT2 protein has an AA sequence set forth in 1 of the above GenBank entries. In another embodiment, the SLIT2 protein is encoded by any other SLIT2 gene sequence known in the art. In another embodiment, the SLIT2 protein is any other SLIT2 protein known in the art. In another embodiment, the TVM is an isoform of a SLIT2 protein. In another embodiment, the TVM is a homologue of a SLIT2 protein. In another embodiment, the TVM is a variant of a SLIT2 protein. In another embodiment, the TVM is a fragment of a SLIT2 protein. In another embodiment, the TVM is a fragment of an isoform, homologue, or variant of a SLIT2 protein. Each possibility represents another embodiment of the present invention.

In another embodiment, the TVM is SLC11A1 (Solute carrier family 11; proton-coupled divalent metal ion transporters, member 1; NRAMP). In another embodiment, the TVM is a nucleotide molecule encoding SLCA1. In another embodiment, the TVM is an isoform of a SLC11A1 protein. In another embodiment, the TVM is a homologue of a SLC11A1 protein. In another embodiment, the TVM is a variant of a SLC11A1 protein. In another embodiment, the TVM is a fragment of a SLC11A1 protein. In another embodiment, the TVM is a fragment of an isoform, homologue, or variant of a SLC11A1 protein. Each possibility represents another embodiment of the present invention.

In another embodiment, the TVM is SEC23B. In another embodiment, the TVM is a nucleotide molecule encoding SEC23B. In another embodiment, the TVM is an isoform of a SEC23B protein. In another embodiment, the TVM is a homologue of a SEC23B protein. In another embodiment, the TVM is a variant of a SEC23B protein. In another embodiment, the TVM is a fragment of a SEC23B protein. In another embodiment, the TVM is a fragment of an isoform, homologue, or variant of a SEC23B protein. Each possibility represents another embodiment of the present invention.

In another embodiment, the TVM is DKFZp762E1312. In another embodiment, the TVM is a nucleotide molecule encoding DKFZp762E1312. In another embodiment, the TVM is an isoform of a DKFZp762E1312 protein. In another embodiment, the TVM is a homologue of a DKFZp762E1312 protein. In another embodiment, the TVM is a variant of a DKFZp762E1312 protein. In another embodiment, the TVM is a fragment of a DKFZp762E1312 protein. In another embodiment, the TVM is a fragment of an isoform, homologue, or variant of a DKFZp762E1312 protein. Each possibility represents another embodiment of the present invention.

In another embodiment, the TVM is KIAA1892. In another embodiment, the TVM is a nucleotide molecule encoding KIAA1892. In another embodiment, the TVM is a protein encoded by KIAA1892. In another embodiment, the TVM is an isoform of a KIAA1892 protein. In another embodiment, the TVM is a homologue of a KIAA1892 protein. In another embodiment, the TVM is a variant of a KIAA1892 protein. In another embodiment, the TVM is a fragment of a KIAA1892 protein. In another embodiment, the TVM is a fragment of an isoform, homologue, or variant of a KIAA1892 protein. Each possibility represents another embodiment of the present invention.

In another embodiment, the TVM is MS4A6A (Membrane-spanning 4-domains, subfamily A, member 6A). In another embodiment, the TVM is a nucleotide molecule encoding MS4A6A. In another embodiment, the TVM is an isoform of a MS4A6A protein. In another embodiment, the TVM is a homologue of a MS4A6A protein. In another embodiment, the TVM is a variant of a MS4A6A protein. In another embodiment, the TVM is a fragment of a MS4A6A protein. In another embodiment, the TVM is a fragment of an isoform, homologue, or variant of a MS4A6A protein. Each possibility represents another embodiment of the present invention.

In another embodiment, the TVM is KCNE3 (Potassium voltage-gated channel, Isk-related family, member 3). In another embodiment, the TVM is a nucleotide molecule encoding KCNE3. In another embodiment, the TVM is an isoform of a KCNE3 protein. In another embodiment, the TVM is a homologue of a KCNE3 protein. In another embodiment, the TVM is a variant of a KCNE3 protein. In another embodiment, the TVM is a fragment of a KCNE3 protein. In another embodiment, the TVM is a fragment of an isoform, homologue, or variant of a KCNE3 protein. Each possibility represents another embodiment of the present invention.

In another embodiment, the TVM is KCNE4 (Potassium voltage-gated channel, Isk-related family, member 4). In another embodiment, the TVM is a nucleotide molecule encoding KCNE4. In another embodiment, the TVM is an isoform of a KCNE4 protein. In another embodiment, the TVM is a homologue of a KCNE4 protein. In another embodiment, the TVM is a variant of a KCNE4 protein. In another embodiment, the TVM is a fragment of a KCNE4 protein. In another embodiment, the TVM is a fragment of an isoform, homologue, or variant of a KCNE4 protein. Each possibility represents another embodiment of the present invention.

In another embodiment, the TVM is SDC1 (Syndecan 1). In another embodiment, the TVM is a nucleotide molecule encoding SDC1. In another embodiment, the TVM is an isoform of a SDC1 protein. In another embodiment, the TVM is a homologue of a SDC1 protein. In another embodiment, the TVM is a variant of a SDC1 protein. In another embodiment, the TVM is a fragment of a SDC1 protein. In another embodiment, the TVM is a fragment of an isoform, homologue, or variant of a SDC1 protein. Each possibility represents another embodiment of the present invention.

In another embodiment, the TVM is ST14 (Suppression of tumorigenicity 14 (colon carcinoma)). In another embodiment, the TVM is a nucleotide molecule encoding ST14. In another embodiment, the TVM is an isoform of a ST14 protein. In another embodiment, the TVM is a homologue of a ST14 protein. In another embodiment, the TVM is a variant of a ST14 protein. In another embodiment, the TVM is a fragment of a ST14 protein. In another embodiment, the TVM is a fragment of an isoform, homologue, or variant of a ST14 protein. Each possibility represents another embodiment of the present invention.

In another embodiment, the TVM is CDCP1 (CUB domain containing protein 1). In another embodiment, the TVM is a nucleotide molecule encoding CDCP1. In another embodiment, the CDCP1 nucleotide is a short isoform of CDCP1. In another embodiment, the CDCP1 nucleotide is a long isoform of CDCP1. In another embodiment, the CDCP1 protein is encoded by a nucleic acid molecule having the sequence:

(SEQ ID No: 119)

gggcggggctcgggccggtccgcccgcgcgcaggtgagtgagccagggcg
gagcgcagctgcgccgggcttgggcgcctggggccgccgctccccaccgt
cgttttccccaccgaggccgaggcgtcccggagtcatggccggcctgaac
tgcggggtctctatcgcactgctaggggttctgctgctgggtgcggcgcg
cctgccgcgcggggcagaagcttttgagattgctctgccacgagaaagca
acattacagttctcataaagctggggaccccgactctgctggcaaaaccc
tgttacatcgtcatttctaaaagacatataaccatgttgtccatcaagtc
tggagaaagaatagtctttacctttagctgccagagtcctgagaatcact
ttgtcatagagatccagaaaaatattgactgtatgtcaggcccatgtcct
tttggggaggttcagcttcagccctcgacatcgttgttgcctaccctcaa
cagaactttcatctgggatgtcaaagctcataagagcatcggtttagagc
tgcagttttccatccctcgcctgaggcagatcggtccgggtgagagctgc
ccagacggagtcactcactccatcagcggccgaatcgatgccaccgtggt
caggatcggaaccttctgcagcaatggcactgtgtcccggatcaagatgc
aagaaggagtgaaaatggccttacacctcccatggttccaccccagaaat
gtctccggcttcagcattgcaaaccgctcatctataaaacgtctgtgcat
catcgagtctgtgtttgagggtgaaggctcagcaaccctgatgtctgcca
actacccagaaggcttccctgaggatgagctcatgacgtggcagtttgtc
gttcctgcacacctgcgggccagcgtctccttcctcaacttcaacctctc
caactgtgagaggaaggaggagcgggttgaatactacatcccgggctcca
ccaccaaccccgaggtgttcaagctggaggacaagcagcctgggaacatg
gcggggaacttcaacctctctctgcaaggctgtgaccaagatgcccaaag
tccagggatcctccggctgcagttccaagttttggtccaacatccacaaa
atgaaagcagtgagtgagccccactttcctttttcttcctcctccagcac
cttcgttgtttcctgggtagtctgcctgggtgaggctcccttcctgtttc
tcatctgtggcttctgaaacacttagactctggacccagcaagagtttca
ggaagtgggttgctaggcagttagacaggcttgttggtgaacacccggta
tgtagttccatttcagcacaataaaaagaaatcttgcattcaaaaaaaaa
aaaaaaaaaa.

In another embodiment, the CDCP1 protein is encoded by a nucleic acid molecule with a sequence set forth in SEQ ID No: 268. In another embodiment, the CDCP1 protein is a short isoform of CDCP1. In another embodiment, the CDCP1 protein is a long isoform of CDCP1. In another embodiment, the CDCP1 protein is encoded by a nucleic acid molecule having a sequence set forth in GenBank Accession No. AK026329. In another embodiment, the sequence of the CDCP1-encoding nucleotide is set forth in GenBank Accession No. NM_{—}178181. In another embodiment, the sequence of the CDCP1-encoding nucleotide is set forth in GenBank Accession No. BC021099. In another embodiment, the sequence of the CDCP1-encoding nucleotide is set forth in GenBank Accession No. BC069254. In another embodiment, the sequence of the CDCP1-encoding nucleotide is set forth in Genbank Accession No. AY026461. In another embodiment, the sequence of the CDCP1-encoding nucleotide is set forth in GenBank Accession No. AF468010. In another embodiment, the sequence of the CDCP1-encoding nucleotide is set forth in GenBank Accession No. AY167484. In another embodiment, the CDCP1 protein has an AA sequence set forth in 1 of the above GenBank entries. In another embodiment, the CDCP1 protein is encoded by any other CDCP1 gene sequence known in the art. In another embodiment, the CDCP1 protein is any other CDCP1 protein known in the art. In another embodiment, the TVM is an isoform of a CDCP1 protein. In another embodiment, the TVM is a homologue of a CDCP1 protein. In another embodiment, the TVM is a variant of a CDCP1 protein. In another embodiment, the TVM is a fragment of a CDCP1 protein. In another embodiment, the TVM is a fragment of an isoform, homologue, or variant of a CDCP1 protein. Each possibility represents another embodiment of the present invention.

In another embodiment, the TVM is a homologue of a CDCP1 protein. In another embodiment, the TVM is a variant of a CDCP1 protein. In another embodiment, the TVM is a fragment of a CDCP1 protein. In another embodiment, the TVM is a fragment of an isoform, homologue, or variant of a CDCP1 protein. Each possibility represents another embodiment of the present invention.

In another embodiment, the TVM is MOBK1B (C2orf6; MOB1, Mps One Binder kinase activator-like 1B). In another embodiment, the TVM is an isoform of a MOBK1B protein. In another embodiment, the TVM is a homologue of a MOBK1B protein. In another embodiment, the TVM is a variant of a MOBK1B protein. In another embodiment, the TVM is a fragment of a MOBK1B protein. In another embodiment, the TVM is a fragment of an isoform, homologue, or variant of a MOBK1B protein. Each possibility represents another embodiment of the present invention.

In another embodiment, the TVM is a protein encoded by C14orf28. In another embodiment, the TVM is C14orf28. In another embodiment, the TVM is a nucleotide molecule encoding a protein encoded by C14orf28. In another embodiment, the TVM is an isoform of a C14orf28 protein. In another embodiment, the TVM is a homologue of a C14orf28 protein. In another embodiment, the TVM is a variant of a C14orf28 protein. In another embodiment, the TVM is a fragment of a C14orf28 protein. In another embodiment, the TVM is a fragment of an isoform, homologue, or variant of a C14orf28 protein. Each possibility represents another embodiment of the present invention.

In another embodiment, the TVM is PCDHB2 (Protocadherin beta 2). In another embodiment, the TVM is a nucleotide molecule encoding PCDHB2. In another embodiment, the TVM is an isoform of a PCDHB2 protein. In another embodiment, the TVM is a homologue of a PCDHB2 protein. In another embodiment, the TVM is a variant of a PCDHB2 protein. In another embodiment, the TVM is a fragment of a PCDHB2 protein. In another embodiment, the TVM is a fragment of an isoform, homologue, or variant of a PCDHB2 protein. Each possibility represents another embodiment of the present invention.

In another embodiment, the TVM is GPR105 (Purinergic receptor P2Y, G-protein coupled, 14). In another embodiment, the TVM is a nucleotide molecule encoding GPR105. In another embodiment, the TVM is an isoform of a GPR105 protein. In another embodiment, the TVM is a homologue of a GPR105 protein. In another embodiment, the TVM is a variant of a GPR105 protein. In another embodiment, the TVM is a fragment of a GPR105 protein. In another embodiment, the TVM is a fragment of an isoform, homologue, or variant of a GPR105 protein. Each possibility represents another embodiment of the present invention.

In another embodiment, the TVM is CSPG2 (chondroitin sulfate proteoglycan 2). In another embodiment, the TVM is a nucleotide molecule encoding CSPG2. In another embodiment, the TVM is an isoform of a CSPG2 protein. In another embodiment, the TVM is a homologue of a CSPG2 protein. In another embodiment, the TVM is a variant of a CSPG2 protein. In another embodiment, the TVM is a fragment of a CSPG2 protein. In another embodiment, the TVM is a fragment of an isoform, homologue, or variant of a CSPG2 protein. Each possibility represents another embodiment of the present invention.

In another embodiment, the TVM is ESM1 (Endothelial cell-specific molecule 1). In another embodiment, the TVM is a nucleotide molecule encoding ESM1. In another embodiment, the TVM is an isoform of a ESM1 protein. In another embodiment, the TVM is a homologue of a ESM1 protein. In another embodiment, the TVM is a variant of a ESM1 protein. In another embodiment, the TVM is a fragment of a ESM1 protein. In another embodiment, the TVM is a fragment of an isoform, homologue, or variant of a ESM1 protein. Each possibility represents another embodiment of the present invention.

In another embodiment, the TVM is WFDC2 (WAP four-disulfide core domain 2). In another embodiment, the TVM is a nucleotide molecule encoding WFDC2. In another embodiment, the TVM is an isoform of a WFDC2 protein. In another embodiment, the TVM is a homologue of a WFDC2 protein. In another embodiment, the TVM is a variant of a WFDC2 protein. In another embodiment, the TVM is a fragment of a WFDC2 protein. In another embodiment, the TVM is a fragment of an isoform, homologue, or variant of a WFDC2 protein. Each possibility represents another embodiment of the present invention.

In another embodiment, the TVM is SPP1 (Secreted phosphoprotein 1 (osteopontin, bone sialoprotein I, early T-lymphocyte activation 1)). In another embodiment, the TVM is a nucleotide molecule encoding SPP1. In another embodiment, the TVM is an isoform of a SPP1 protein. In another embodiment, the TVM is a homologue of a SPP1 protein. In another embodiment, the TVM is a variant of a SPP1 protein. In another embodiment, the TVM is a fragment of a SPP1 protein. In another embodiment, the TVM is a fragment of an isoform, homologue, or variant of a SPP1 protein. Each possibility represents another embodiment of the present invention.

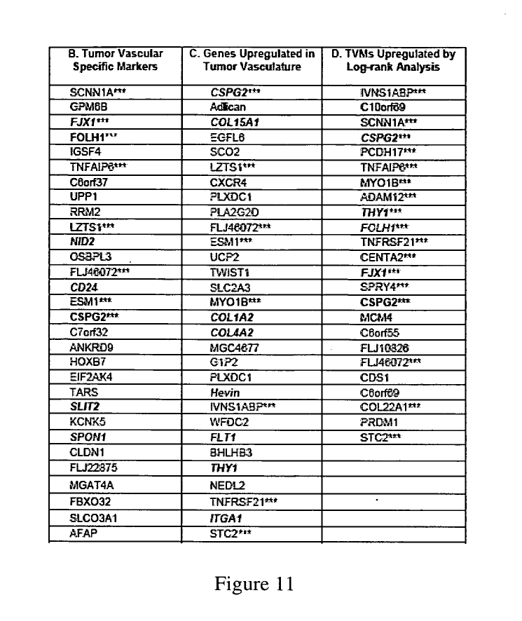

In another embodiment, the TVM is a TM protein listed in FIG. 11. In another embodiment, the TVM is MGAT4A. In another embodiment, the TVM is a nucleotide molecule encoding MGAT4A. In another embodiment, the TVM is AFAP. In another embodiment, the TVM is a nucleotide molecule encoding AFAP. In another embodiment, the TVM is CXCR4. In another embodiment, the TVM is a nucleotide molecule encoding CXCR4. In another embodiment, the TVM is UCP2. In another embodiment, the TVM is a nucleotide molecule encoding UCP2. In another embodiment, the TVM is TWIST. In another embodiment, the TVM is a nucleotide molecule encoding TWIST. In another embodiment, the TVM is SLC2A3. In another embodiment, the TVM is a nucleotide molecule encoding SLC2A3. In another embodiment, the TVM is MYO1B. In another embodiment, the TVM is a nucleotide molecule encoding MYO1B. In another embodiment, the TVM is COL4A2. In another embodiment, the TVM is a nucleotide molecule encoding COL4A2. In another embodiment, the TVM is MGC4677. In another embodiment, the TVM is a nucleotide molecule encoding MGC4677. In another embodiment, the TVM is G1P2. In another embodiment, the TVM is a nucleotide molecule encoding G1P2. In another embodiment, the TVM is BHLHB3. In another embodiment, the TVM is a nucleotide molecule encoding BHLHB3. In another embodiment, the TVM is NEDL2. In another embodiment, the TVM is a nucleotide molecule encoding NEDL2. In another embodiment, the TVM is ITGA1. In another embodiment, the TVM is a nucleotide molecule encoding ITGA1. Each possibility represents a separate embodiment of the present invention.

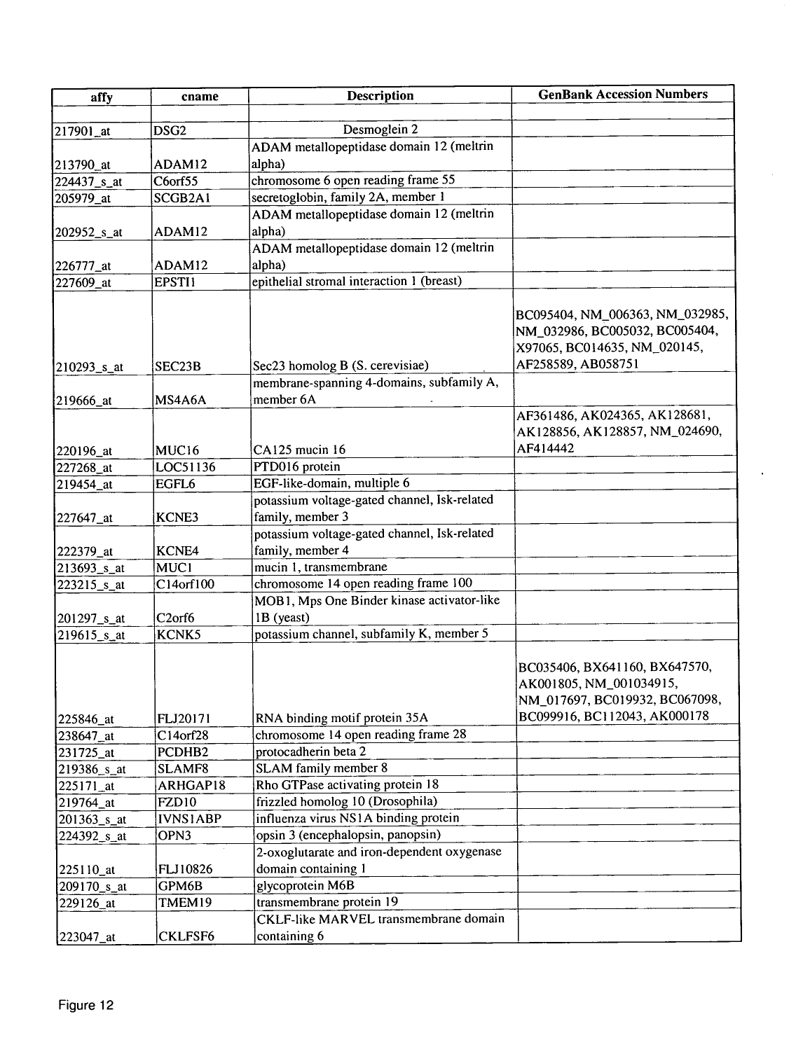

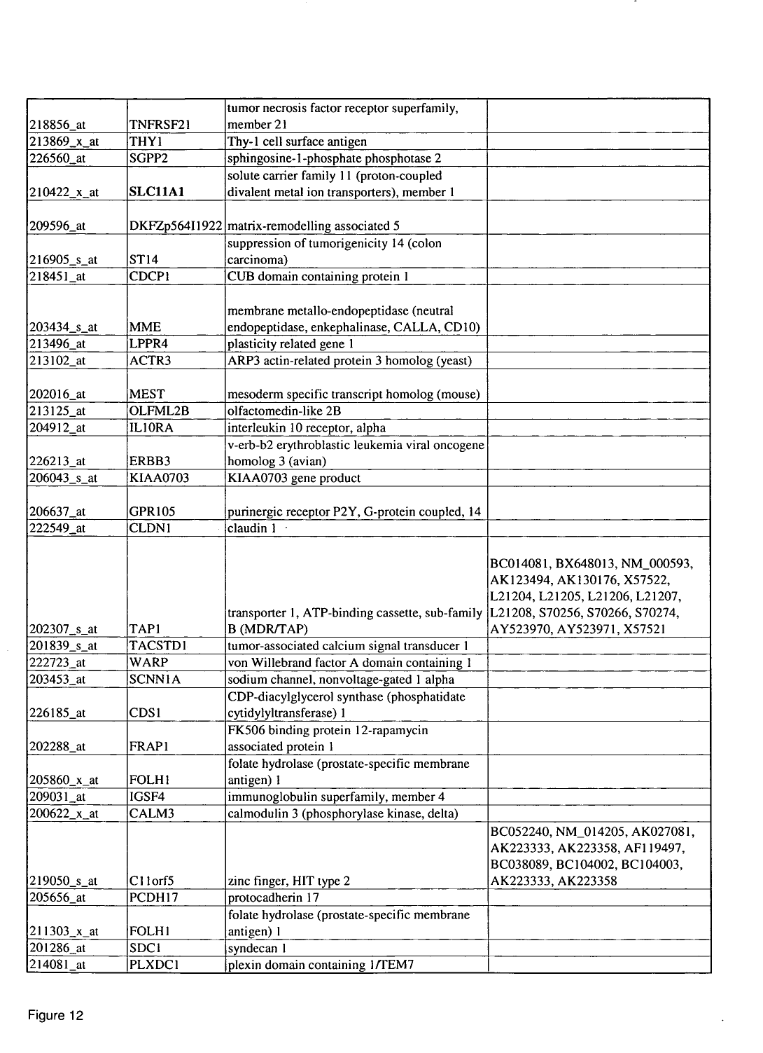

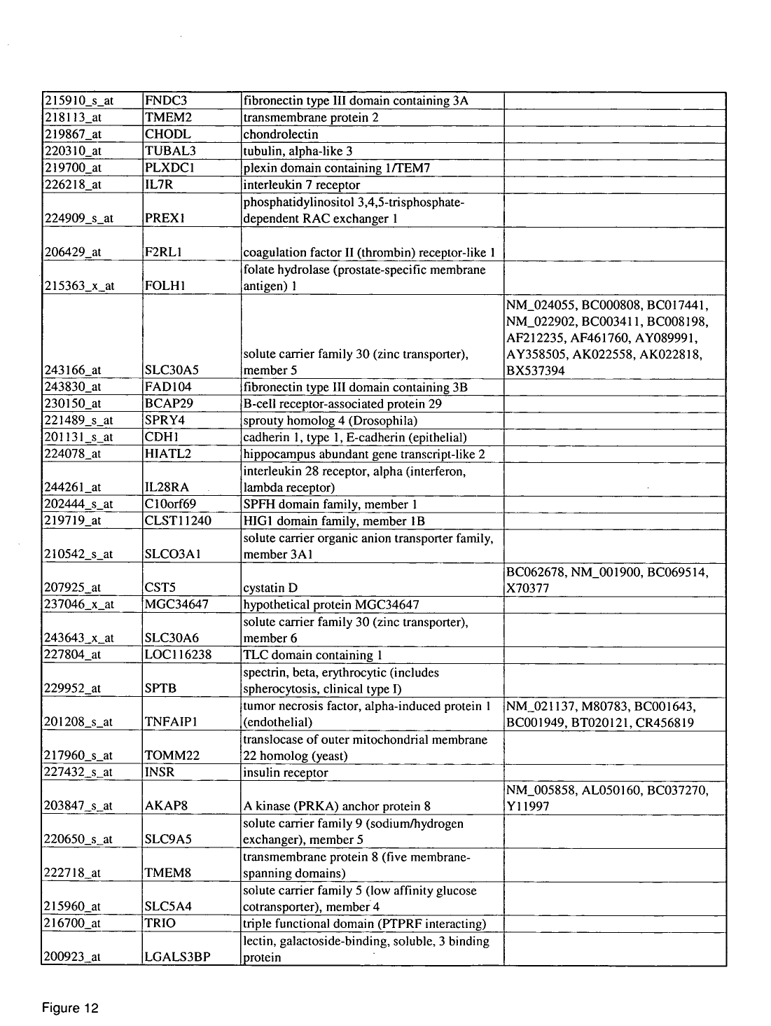

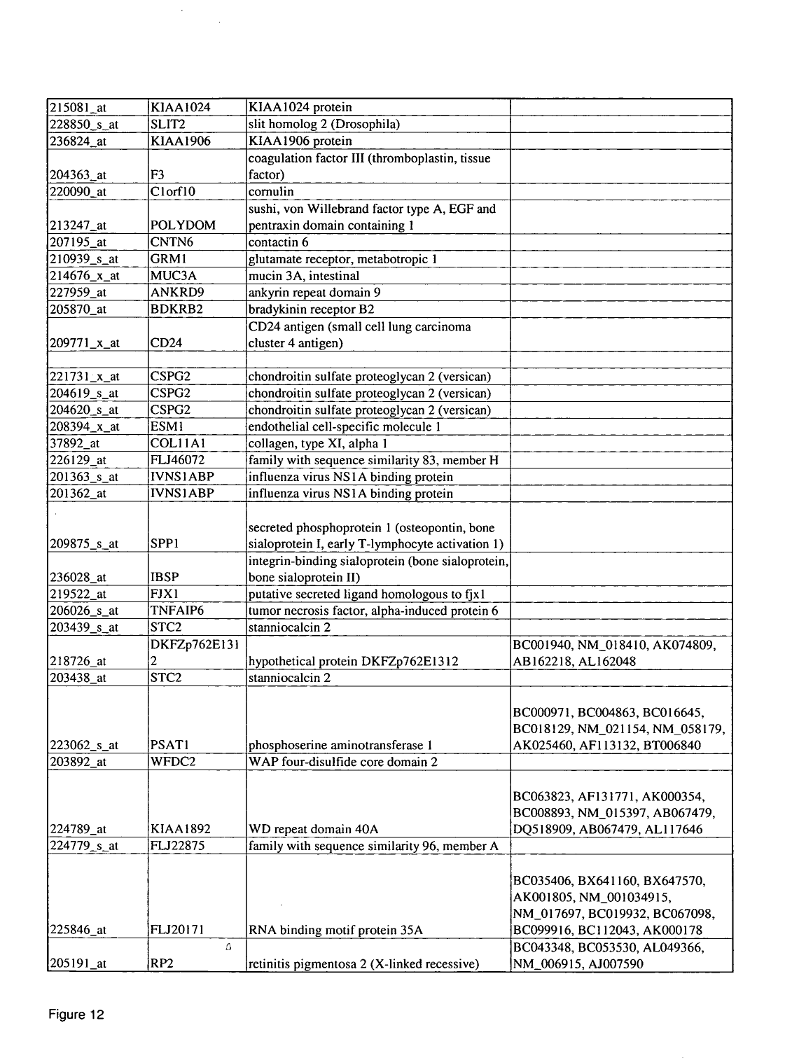

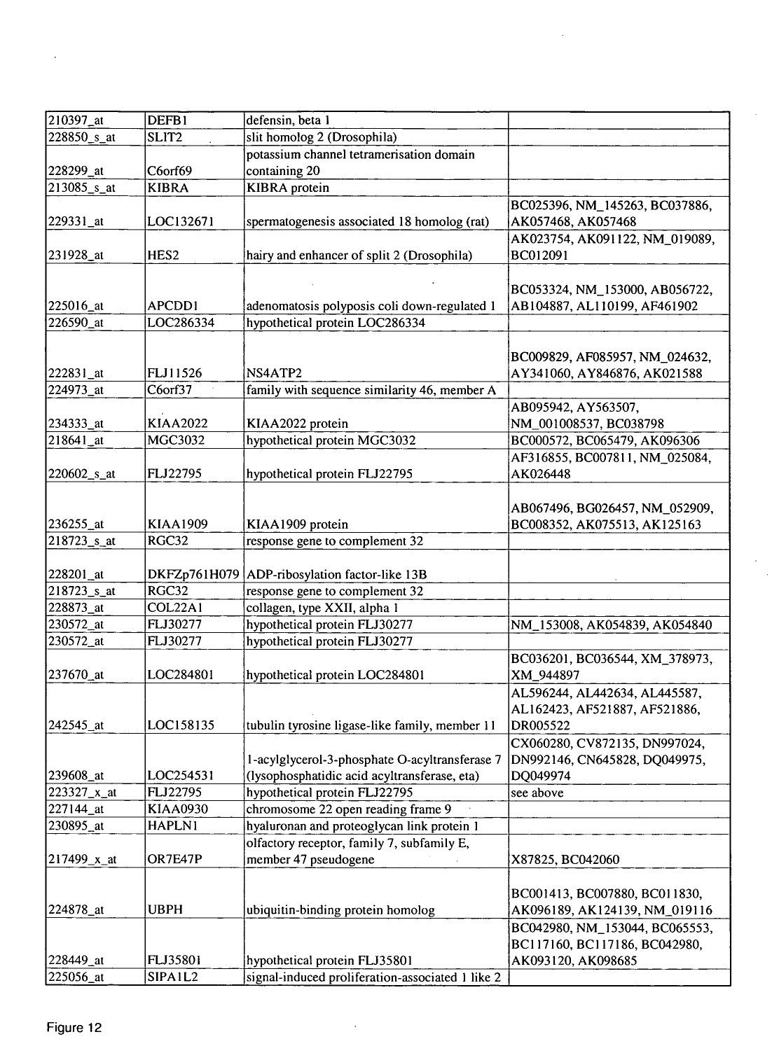

In another embodiment, the TVM is a TM protein listed in FIG. 12. In another embodiment, the TVM is MUC16. In another embodiment, the TVM is a nucleotide molecule encoding MUC16. In another embodiment, the TVM is FLJ20171. In another embodiment, the TVM is a nucleotide molecule encoding FLJ20171. In another embodiment, the TVM is TAP1. In another embodiment, the TVM is a nucleotide molecule encoding TAP1. In another embodiment, the TVM is C11orf5. In another embodiment, the TVM is a nucleotide molecule encoding C11orf5. In another embodiment, the TVM is SLC30A5. In another embodiment, the TVM is a nucleotide molecule encoding SLC30A5. In another embodiment, the TVM is CST5. In another embodiment, the TVM is a nucleotide molecule encoding CST5. In another embodiment, the TVM is TNFAIP1. In another embodiment, the TVM is a nucleotide molecule encoding TNFAIP1. In another embodiment, the TVM is AKAP8. In another embodiment, the TVM is a nucleotide molecule encoding AKAP8. In another embodiment, the TVM is PSAT1. In another embodiment, the TVM is a nucleotide molecule encoding PSAT1. In another embodiment, the TVM is FLJ20171. In another embodiment, the TVM is a nucleotide molecule encoding FLJ20171. In another embodiment, the TVM is RP2. In another embodiment, the TVM is a nucleotide molecule encoding RP2. In another embodiment, the TVM is LOC132671. In another embodiment, the TVM is a nucleotide molecule encoding LOC132671. In another embodiment, the TVM is HES2. In another embodiment, the TVM is a nucleotide molecule encoding HES2. In another embodiment, the TVM is APCDD1. In another embodiment, the TVM is a nucleotide molecule encoding APCDD1. In another embodiment, the TVM is LOC286334. In another embodiment, the TVM is a nucleotide molecule encoding LOC286334. In another embodiment, the TVM is FLJ11526. In another embodiment, the TVM is a nucleotide molecule encoding FLJ11526. In another embodiment, the TVM is KIAA2022. In another embodiment, the TVM is a nucleotide molecule encoding KIAA2022. In another embodiment, the TVM is MGC3032. In another embodiment, the TVM is a nucleotide molecule encoding MGC3032. In another embodiment, the TVM is FLJ22795. In another embodiment, the TVM is a nucleotide molecule encoding FLJ22795. In another embodiment, the TVM is KIAA1909. In another embodiment, the TVM is a nucleotide molecule encoding KIAA1909. In another embodiment, the TVM is FLJ30277. In another embodiment, the TVM is a nucleotide molecule encoding FLJ30277. In another embodiment, the TVM is LOC284801. In another embodiment, the TVM is a nucleotide molecule encoding LOC284801. In another embodiment, the TVM is LOC158135. In another embodiment, the TVM is a nucleotide molecule encoding LOC158135. In another embodiment, the TVM is LOC254531. In another embodiment, the TVM is a nucleotide molecule encoding LOC254531. In another embodiment, the TVM is OR7E47P. In another embodiment, the TVM is a nucleotide molecule encoding OR7E47P. In another embodiment, the TVM is UBPH. In another embodiment, the TVM is a nucleotide molecule encoding UBPH. In another embodiment, the TVM is FLJ35801. In another embodiment, the TVM is a nucleotide molecule encoding FLJ35801. In another embodiment, the TVM is LOC150271. In another embodiment, the TVM is a nucleotide molecule encoding LOC150271. In another embodiment, the TVM is SIPA1L3. In another embodiment, the TVM is a nucleotide molecule encoding SIPA1L3. In another embodiment, the TVM is LOC158563. In another embodiment, the TVM is a nucleotide molecule encoding LOC158563. In another embodiment, the TVM is NAV1. In another embodiment, the TVM is a nucleotide molecule encoding NAV1. In another embodiment, the TVM is LOC401022. In another embodiment, the TVM is a nucleotide molecule encoding LOC401022. In another embodiment, the TVM is C9orf113. In another embodiment, the TVM is a nucleotide molecule encoding C9orf113. In another embodiment, the TVM is GPT2. In another embodiment, the TVM is a nucleotide molecule encoding GPT2. In another embodiment, the TVM is PHLDB1. In another embodiment, the TVM is a nucleotide molecule encoding PHLDB1. In another embodiment, the TVM is FLJ12748. In another embodiment, the TVM is a nucleotide molecule encoding FLJ12748. In another embodiment, the TVM is LOC130355. In another embodiment, the TVM is a nucleotide molecule encoding LOC130355. In another embodiment, the TVM is BECN1. In another embodiment, the TVM is a nucleotide molecule encoding BECN1. In another embodiment, the TVM is LOC283713. In another embodiment, the TVM is a nucleotide molecule encoding LOC283713. Each possibility represents a separate embodiment of the present invention.

In another embodiment, the TVM is a TM protein listed in Table 6. In another embodiment, the TVM is a TM protein listed in Table 7. In another embodiment, the TVM is a plasma-membrane-associated (PM) protein listed in Table 6. In another embodiment, the TVM is a PM protein listed in Table 7. In another embodiment, a PM protein of the present invention is a TM protein. In another embodiment, the PM protein is associated with the intracellular face of the PM. In another embodiment, the PM protein is associated with the extracellular face of the PM. Each possibility represents a separate embodiment of the present invention.

In another embodiment, the TVM is FAD104 (FNDC3B; Fibronectin type III domain containing 3B). In another embodiment, the TVM is a nucleotide molecule encoding FAD104. In another embodiment, the TVM is WARP (Von Willebrand factor A domain containing 1). In another embodiment, the TVM is a nucleotide molecule encoding WARP. In another embodiment, the TVM is B-cell receptor-associated protein 29 (BCAP29). In another embodiment, the TVM is a nucleotide molecule encoding BCAP29. In another embodiment, the TVM is CDH1 (Cadherin 1, type 1, E-cadherin (epithelial). In another embodiment, the TVM is a nucleotide molecule encoding CDH1. In another embodiment, the TVM is FLJ10826 (OGFOD1; 2-oxoglutarate and iron-dependent oxygenase domain containing 1). In another embodiment, the TVM is a nucleotide molecule encoding FLJ10826. In another embodiment, the TVM is OPN3 (Opsin 3; encephalopsin, panopsin). In another embodiment, the TVM is a nucleotide molecule encoding OPN3. In another embodiment, the TVM is HIATL2 (Hippocampus abundant gene transcript-like 2). In another embodiment, the TVM is a nucleotide molecule encoding HIATL2. In another embodiment, the TVM is IL28RA (Interleukin 28 receptor, alpha; interferon, lambda receptor). In another embodiment, the TVM is a nucleotide molecule encoding IL28RA. In another embodiment, the TVM is TMEM19 (Transmembrane protein 19). In another embodiment, the TVM is a nucleotide molecule encoding TMEM19. In another embodiment, the TVM is C10orf69 (SPFH domain family, member 1). In another embodiment, the TVM is a nucleotide molecule encoding C10orf69. In another embodiment, the TVM is FRAP1 (FK506 binding protein 12-rapamycin associated protein 1). In another embodiment, the TVM is a nucleotide molecule encoding FRAP1. In another embodiment, the TVM is CKLFSF6 (CKLF-like MARVEL transmembrane domain containing 6). In another embodiment, the TVM is a nucleotide molecule encoding CKLFSF6. In another embodiment, the TVM is MPHOSPH9 (M-phase phosphoprotein 9). In another embodiment, the TVM is a nucleotide molecule encoding MPOHSPH9. In another embodiment, the TVM is CLST11240 (HIGD1B; HIG1 domain family, member 1B). In another embodiment, the TVM is a nucleotide molecule encoding CLST11240. In another embodiment, the TVM is SGPP2 (Sphingosine-1-phosphate phosphotase 2). In another embodiment, the TVM is a nucleotide molecule encoding SGPP2. In another embodiment, the TVM is SLC03A1 (Solute carrier organic anion transporter family, member 3A 1). In another embodiment, the TVM is a nucleotide molecule encoding SLC03A1. In another embodiment, the TVM is LOC51136 (PTD016 protein). In another embodiment, the TVM is a nucleotide molecule encoding LOC51136. In another embodiment, the TVM is DKFZp56411922 (MXRA5 (Matrix-remodelling associated 5). In another embodiment, the TVM is a nucleotide molecule encoding DKFZp564I1922. In another embodiment, the TVM is CALM3 (Calmodulin 3; phosphorylase kinase, delta). In another embodiment, the TVM is a nucleotide molecule encoding CALM3. In another embodiment, the TVM is MGC34647. In another embodiment, the TVM is a nucleotide molecule encoding MGC34647. In another embodiment, the TVM is MUC1 (Mucin 1, transmembrane). In another embodiment, the TVM is a nucleotide molecule encoding MUC1. In another embodiment, the TVM is SLC30A6 (Solute carrier family 30 (zinc transporter), member 6). In another embodiment, the TVM is a nucleotide molecule encoding SLC30A6. In another embodiment, the TVM is TLCD1 (LOC116238). In another embodiment, the TVM is a nucleotide molecule encoding TLCD1. In another embodiment, the TVM is SPTB (Spectrin, beta, erythrocytic (includes spherocytosis, clinical type I)). In another embodiment, the TVM is a nucleotide molecule encoding SPTB. In another embodiment, the TVM is FNDC3 (Fibronectin type III domain containing 3A). In another embodiment, the TVM is a nucleotide molecule encoding FNDC3. In another embodiment, the TVM is SPRY1 (Sprouty homolog 1, antagonist of FGF signaling (Drosophila). In another embodiment, the TVM is a nucleotide molecule encoding SPRY1. In another embodiment, the TVM is MME (Membrane metallo-endopeptidase; neutral endopeptidase, enkephalinase, CALLA, CD10). In another embodiment, the TVM is a nucleotide molecule encoding MME. In another embodiment, the TVM is INSR (Insulin receptor). In another embodiment, the TVM is a nucleotide molecule encoding INSR. In another embodiment, the TVM is LPPR4 (Plasticity related gene 1). In another embodiment, the TVM is a nucleotide molecule encoding LPPR1. In another embodiment, the TVM is a C14orf100-encoded protein. In another embodiment, the TVM is a nucleotide molecule encoding a C14orf100-encoded protein. In another embodiment, the TVM is a C14orf100 nucleotide molecule. In another embodiment, the TVM is SLC9A5 (Solute carrier family 9 (sodium/hydrogen exchanger), member 5). In another embodiment, the TVM is a nucleotide molecule encoding SLC9A5. In another embodiment, the TVM is SCGB2A1 (Secretoglobin, family 2A, member 1). In another embodiment, the TVM is a nucleotide molecule encoding SCGB2A1. In another embodiment, the TVM is FLT1 (Fms-related tyrosine kinase 1 (vascular endothelial growth factor/vascular permeability factor receptor). In another embodiment, the TVM is a nucleotide molecule encoding FLT1. In another embodiment, the TVM is a nucleotide molecule encoding MOBK1B. In another embodiment, the TVM is TMEM2 (Transmembrane protein 2). In another embodiment, the TVM is a nucleotide molecule encoding TMEM2. In another embodiment, the TVM is TMEM8 (Transmembrane protein 8; five membrane-spanning domains). In another embodiment, the TVM is a nucleotide molecule encoding TMEM8. In another embodiment, the TVM is SLC5A4 (Solute carrier family 5 (low affinity glucose cotransporter), member 4). In another embodiment, the TVM is a nucleotide molecule encoding SLC5A4. In another embodiment, the TVM is MEST (Mesoderm specific transcript homolog (mouse). In another embodiment, the TVM is a nucleotide molecule encoding MEST. In another embodiment, the TVM is CHODL (Chondrolectin). In another embodiment, the TVM is a nucleotide molecule encoding CHODL. In another embodiment, the TVM is TRIO (Triple functional domain (PTPRF interacting)). In another embodiment, the TVM is a nucleotide molecule encoding TRIO. In another embodiment, the TVM is IL10RA (Interleukin 10 receptor, alpha). In another embodiment, the TVM is a nucleotide molecule encoding IL10RA. In another embodiment, the TVM is LGALS3BP (Lectin, galactoside-binding, soluble, 3 binding protein). In another embodiment, the TVM is a nucleotide molecule encoding LGALS3BP. In another embodiment, the TVM is STK4 (Serine/threonine kinase 4). In another embodiment, the TVM is a nucleotide molecule encoding STK4. In another embodiment, the TVM is ERBB3 (V-erb-b2 erythroblastic leukemia viral oncogene homolog 3 (avian). In another embodiment, the TVM is a nucleotide molecule encoding ERBB3. In another embodiment, the TVM is KIAA1024. In another embodiment, the TVM is a nucleotide molecule encoding KIAA1024. In another embodiment, the TVM is KIAA1906. In another embodiment, the TVM is a nucleotide molecule encoding KIAA1906. In another embodiment, the TVM is F3 (Coagulation factor III (thromboplastin, tissue factor)). In another embodiment, the TVM is a nucleotide molecule encoding F3. In another embodiment, the TVM is KIAA0703. In another embodiment, the TVM is a nucleotide molecule encoding KIAA0703. In another embodiment, the TVM is C1orf10 (CRNN; Cornulin). In another embodiment, the TVM is a nucleotide molecule encoding C1orf10. In another embodiment, the TVM is POLYDOM (SVEP1 (Sushi, von Willebrand factor type A, EGF and pentraxin domain containing 1). In another embodiment, the TVM is a nucleotide molecule encoding POLYDOM. In another embodiment, the TVM is TUBAL3 (Tubulin, alpha-like 3). In another embodiment, the TVM is a nucleotide molecule encoding TUBAL3. In another embodiment, the TVM is IL7R (Interleukin 7 receptor). In another embodiment, the TVM is a nucleotide molecule encoding IL7R. In another embodiment, the TVM is ARHGAP18 (Rho GTPase activating protein 18). In another embodiment, the TVM is a nucleotide molecule encoding ARHGAP18. In another embodiment, the TVM is GRM1 (Glutamate receptor, metabotropic 1). In another embodiment, the TVM is a nucleotide molecule encoding GRM1. In another embodiment, the TVM is PREX1 (Phosphatidyl-inositol 3,4,5-trisphosphate-dependent RAC exchanger 1). In another embodiment, the TVM is a nucleotide molecule encoding PREX1. In another embodiment, the TVM is MUC3A (Mucin 3A, intestinal). In another embodiment, the TVM is a nucleotide molecule encoding MUC3A. In another embodiment, the TVM is EPSTI1 (Epithelial stromal interaction 1 (breast)). In another embodiment, the TVM is a nucleotide molecule encoding EPSTI1. In another embodiment, the TVM is UBE2J1 (Ubiquitin-conjugating enzyme E2, J1 (UBC6 homolog, yeast). In another embodiment, the TVM is a nucleotide molecule encoding UBE2J1. Each possibility represents a separate embodiment of the present invention.

As provided herein, the long isoform of ADAM12 was particularly efficacious, under the conditions utilized, in distinguishing between tumor vasculature and healthy tissue (Example 20). In another embodiment, the ADAM12 nucleotide of methods and compositions of the present invention is a long isoform thereof. In another embodiment, the ADAM12 nucleotide is a short isoform. In another embodiment, the ADAM12 nucleotide is any other ADAM12 nucleotide known in the art. Each possibility represents a separate embodiment of the present invention.

An ADAM12 protein of methods and compositions of the present invention is, in another embodiment, a long isoform thereof. In another embodiment, the ADAM12 protein is a short isoform. In another embodiment, the ADAM12 protein is any other ADAM12 protein known in the art. Each possibility represents a separate embodiment of the present invention.

As provided herein, the short isoform of CDCP1-CUB was particularly efficacious, under the conditions utilized, in distinguishing between tumor vasculature and healthy tissue (Example 20). In another embodiment, the CDCP1-CUB nucleotide of methods and compositions of the present invention is a short isoform thereof. In another embodiment, the CDCP1-CUB nucleotide is a long isoform. In another embodiment, the CDCP1-CUB nucleotide is any other CDCP1-CUB nucleotide known in the art. Each possibility represents a separate embodiment of the present invention.

A CDCP1-CUB protein of methods and compositions of the present invention is, in another embodiment, a short isoform thereof. In another embodiment, the CDCP1-CUB protein is a long isoform. In another embodiment, the CDCP1-CUB protein is any other CDCP1-CUB protein known in the art. Each possibility represents a separate embodiment of the present invention.

The cancer treated by a method of present invention is, in another embodiment, a cervical cancer tumor. In another embodiment, the cancer is a head and neck cancer tumor. In another embodiment, the cancer is a breast cancer tumor. In another embodiment, the cancer is an ano-genital cancer tumor. In another embodiment, the cancer is a melanoma. In another embodiment, the cancer is a sarcoma. In another embodiment, the cancer is a carcinoma. In another embodiment, the cancer is a lymphoma. In another embodiment, the cancer is a leukemia. In another embodiment, the cancer is mesothelioma. In another embodiment, the cancer is a glioma. In another embodiment, the cancer is a germ cell tumor. In another embodiment, the cancer is a choriocarcinoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment, the cancer is an acute myelogenous leukemia (AML). In another embodiment, the cancer is a myelodysplastic syndrome (MDS). In another embodiment, the cancer is a non-small cell lung cancer (NSCLC). In another embodiment, the cancer is a Wilms' tumor. In another embodiment, the cancer is a leukemia. In another embodiment, the cancer is a lymphoma. In another embodiment, the cancer is a desmoplastic small round cell tumor. In another embodiment, the cancer is a mesothelioma (e.g. malignant mesothelioma). In another embodiment, the cancer is a gastric cancer. In another embodiment, the cancer is a colon cancer. In another embodiment, the cancer is a lung cancer. In another embodiment, the cancer is a breast cancer. In another embodiment, the cancer is a germ cell tumor. In another embodiment, the cancer is an ovarian cancer. In another embodiment, the cancer is a uterine cancer. In another embodiment, the cancer is a thyroid cancer. In another embodiment, the cancer is a hepatocellular carcinoma. In another embodiment, the cancer is a thyroid cancer. In another embodiment, the cancer is a liver cancer. In another embodiment, the cancer is a renal cancer. In another embodiment, the cancer is a kaposis. In another embodiment, the cancer is a sarcoma. In another embodiment, the cancer is another carcinoma or sarcoma. Each possibility represents a separate embodiment of the present invention.

In another embodiment of methods and compositions of the present invention, a subject is contacted with a nucleotide molecule, antibody, ligand, or composition utilized in a method of the present invention. In another embodiment, a solid tumor is contacted with the nucleotide molecule, antibody, ligand, or composition. In another embodiment, an ovarian tumor is contacted with the nucleotide molecule, antibody, ligand, or composition. In another embodiment, a breast cancer tumor is contacted with the nucleotide molecule, antibody, ligand, or composition. In another embodiment, a renal tumor is contacted with the nucleotide molecule, antibody, ligand, or composition. In another embodiment, any other type of solid tumor known in the art is contacted with the nucleotide molecule, antibody, ligand, or composition. Each possibility represents a separate embodiment of the present invention.

In another embodiment, a transmembrane (TM) protein of the present invention is accessible to antibodies and/or non-cell membrane-permeable agents and ligands. In another embodiment, a plasma membrane-associated protein of the present invention is accessible to antibodies and/or non-cell membrane-permeable agents and ligands. In another embodiment, a plasma membrane-associated protein of the present invention is a TM protein. In another embodiment, the plasma membrane-associated protein is an extracellular peripheral membrane protein. In another embodiment, the plasma membrane-associated protein is an intracellular peripheral membrane protein. Each possibility represents a separate embodiment of the present invention.

In another embodiment, a TVM of the present invention is specific for vasculogenesis. In another embodiment, a TVM is associated with vasculogenesis. “Vasculogenesis” refers, in another embodiment, to recruitment of endothelial progenitors of hematopoietic origin.” In another embodiment, the term refers to de novo formation of tumor vasculature. In another embodiment, a method of present invention is capable to detecting or localizing vasculogenesis. In another embodiment, a method of present invention is capable to inhibiting vasculogenesis. Each possibility represents a separate embodiment of the present invention.

In another embodiment, the TVM is a secreted protein. In another embodiment, the TVM is an extracellular matrix (ECM) protein. In another embodiment, the TVM is a protein associated with the plasma membrane of the TVC, on the extracellular side. In another embodiment, the TVM is capable of shedding from the shed into a bodily fluid. In another embodiment, the TVM can be detected in a bodily fluid. In another embodiment, the bodily fluid is blood. In another embodiment, the bodily fluid is lymph. In another embodiment, the bodily fluid is saliva. In another embodiment, the bodily fluid is sperm. In another embodiment, the bodily fluid is cerebro-spinal fluid. In another embodiment, the bodily fluid is cervico-vaginal fluid. In another embodiment, the bodily fluid is any other bodily fluid known in the art. Each possibility represents a separate embodiment of the present invention.

In another embodiment, the TVM is IBSP (Integrin-binding sialoprotein). In another embodiment, the TVM is a nucleotide molecule encoding IBSP. In another embodiment, the TVM is CKLFSF6 (CKLF-like MARVEL transmembrane domain containing 6). In another embodiment, the TVM is a nucleotide molecule encoding CKLFSF6. In another embodiment, the TVM is HAPLN1 (Hyaluronan and proteoglycan link protein 1). In another embodiment, the TVM is a nucleotide molecule encoding HAPLN1. In another embodiment, the TVM is FLT1 (Fms-related tyrosine kinase 1 (vascular endothelial growth factor/vascular permeability factor receptor). In another embodiment, the TVM is a nucleotide molecule encoding FLT1. In another embodiment, the TVM is LGALS3BP (Lectin, galactoside-binding, soluble, 3 binding protein). In another embodiment, the TVM is a nucleotide molecule encoding LGALS3BP. In another embodiment, the TVM is CCL15 (chemokine (C-C motif) ligand 15). In another embodiment, the TVM is a nucleotide molecule encoding CCL15. In another embodiment, the TVM is PLA2G2D (Phospholipase A2, group IID). In another embodiment, the TVM is a nucleotide molecule encoding PLA2G2D. In another embodiment, the TVM is MUC3A (Mucin 3A, intestinal). In another embodiment, the TVM is a nucleotide molecule encoding MUC3A. In another embodiment, the TVM is LTBP2 (Latent transforming growth factor beta binding protein 2). In another embodiment, the TVM is a nucleotide molecule encoding LTBP2. In another embodiment, the TVM is CELSR2 (Cadherin, EGF LAG seven-pass G-type receptor 2). In another embodiment, the TVM is a nucleotide molecule encoding CELSR2. Each possibility represents a separate embodiment of the present invention.

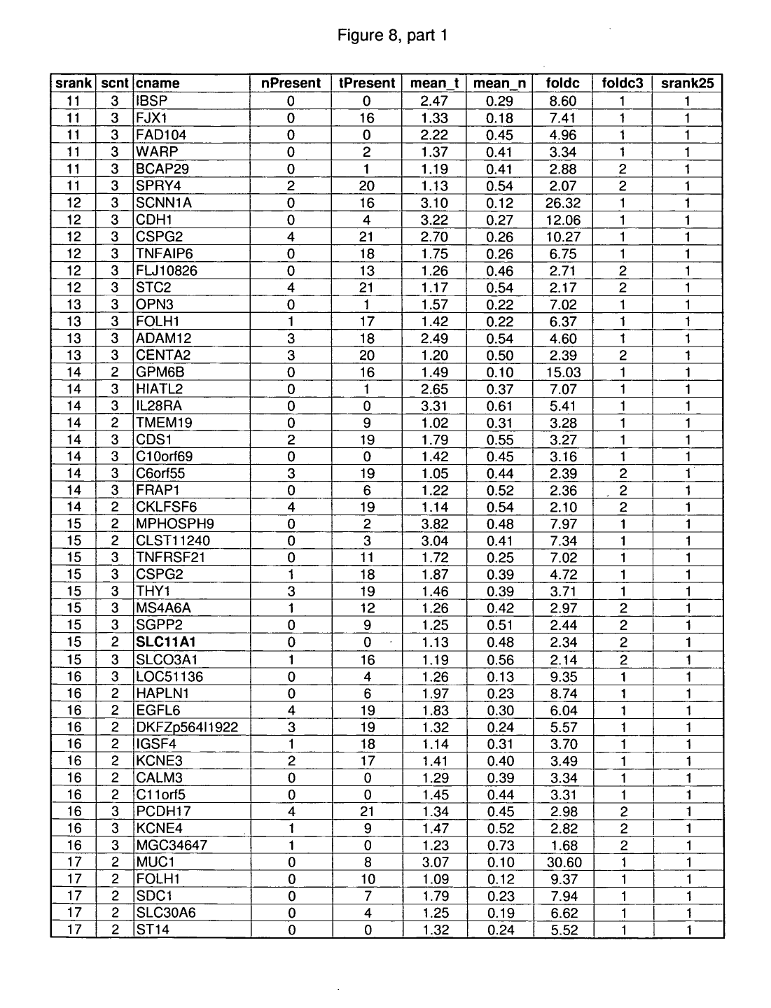

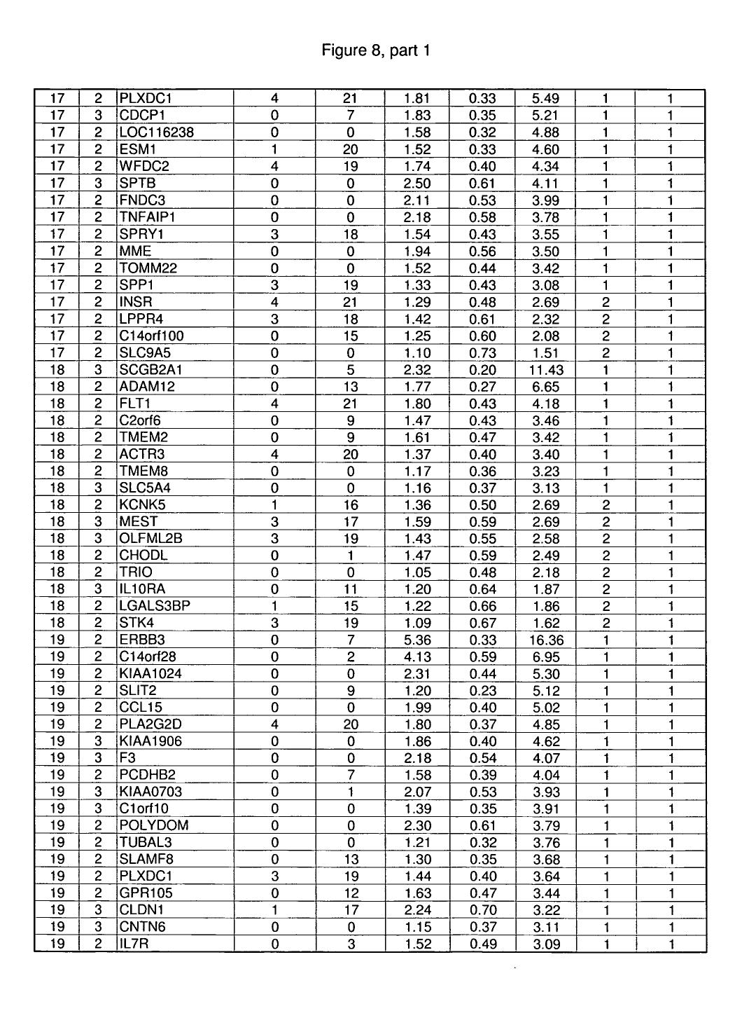

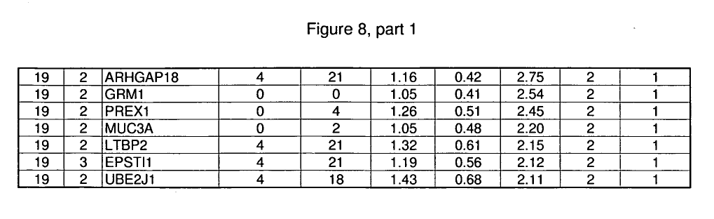

In another embodiment, the TVM is another nucleotide molecule listed in FIG. 8. In another embodiment, the TVM is a protein encoded by a nucleotide molecule listed in FIG. 8. Each possibility represents a separate embodiment of the present invention.

In another embodiment, the TVM is a solute carrier (SLC) family protein. As provided herein, several SLC proteins (SLC9A5, SLC30A6, SLC11A1) were identified as TVM, showing that proteins belonging to this family are efficacious TVM.

In another embodiment, the TVM is a TMEM protein. In another embodiment, the TVM is a protein containing a TMEM region of homology. In another embodiment, the TVM is a protein containing a TMEM domain. As provided herein, several TMEM proteins (TMEM8, TMEM2, TMEM19) were identified as TVM, showing that proteins belonging to this family are efficacious TVM.

In another embodiment, the TVM is a KCN family protein. As provided herein, several KCN proteins (KCNE3, KCNE4) were identified as TVM, showing that proteins belonging to this family are efficacious TVM.

In another embodiment, the TVM is a CD74 protein. As provided herein, CD74 is a marker of tumor vasculature.

In another embodiment, the TVM is an SYCP1 (Synaptonemal complex protein 1).

In another embodiment, the TVM is a CTD (carboxy-terminal domain, RNA polymerase II, polypeptide A) small phosphatase 1.

Each of TVM disclosed herein, refers, in another embodiment, to a human TVM. In another embodiment, certain TVM are homologues of proteins known by a different name in another species, as indicated herein.

Each TVM, nucleic acid molecule, and protein represents a separate embodiment of the present invention.

In another embodiment, the TVM of the present invention exhibit the advantage over tumor cell markers that TVC are genetically stable, relative to tumor cells; thus, TVC are much less likely to switch their expression of the TVM, thus evading localization, detection and therapeutic methods of the present invention. In another embodiment, the TVM of the present invention exhibit the advantage that tumor vasculature is significantly different than physiologic vasculature (for example, as demonstrated herein in Example 3). In another embodiment, the TVM of the present invention exhibit the advantage over tumor cell markers that TVC are more accessible via the bloodstream, relative to tumor cells; thus, TVC are more accessible for localization, detection and anti-tumor therapy by methods of the present invention. In another embodiment, a ligand that binds a TVM of the present invention is administered to a subject via the bloodstream. In another embodiment, the TVM of the present invention exhibit the advantage over tumor cell markers that the TVM are expressed on early—as well as late stage tumors. Each possibility represents a separate embodiment of the present invention.

In another embodiment, the therapeutic methods of the present invention exhibit the advantage that they can target any tumors above 2-3 mm in size, as tumors require vascularization to grow beyond this size. In another embodiment, the diagnostic methods of the present invention exhibit the advantage that they can target any tumors above 2-3 mm in size, as tumors require vascularization to grow beyond this size; thus, these methods enable early detection of solid tumors. In another embodiment, the localization methods of the present invention exhibit the advantage that they can target any tumors above 2-3 mm in size, as tumors require vascularization to grow beyond this size; thus, these methods enable localization of small solid tumors. Each possibility represents a separate embodiment of the present invention.

In another embodiment, a nucleic acid molecule or peptide of the present invention is homologous to a nucleic acid molecule or peptide disclosed herein. The terms “homology,” “homologous,” etc, when in reference to any protein or peptide, refer, in one embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

Homology is, in another embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from SEQ ID No: 1-16, 18-23, 25-26, 28-32, 34-46, 48-58, 60-66, 68-70, and 85-211 of greater than 70%. In another embodiment, “homology” refers to identity to a sequence selected from SEQ ID No: 1-16, 18-23, 25-26, 28-32, 34-46, 48-58, 60-66, 68-70, and 85-211 of greater than 72%. In another embodiment, “homology” refers to identity to one of SEQ ID No: 1-16, 18-23, 25-26, 28-32, 34-46, 48-58, 60-66, 68-70, and 85-211 of greater than 75%. In another embodiment, “homology” refers to identity to a sequence selected from SEQ ID No: 1-16, 18-23, 25-26, 28-32, 34-46, 48-58, 60-66, 68-70, and 85-211 of greater than 78%. In another embodiment, “homology” refers to identity to one of SEQ ID No: 1-16, 18-23, 25-26, 28-32, 34-46, 48-58, 60-66, 68-70, and 85-211 of greater than 80%. In another embodiment, “homology” refers to identity to one of SEQ ID No: 1-16, 18-23, 25-26, 28-32, 34-46, 48-58, 60-66, 68-70, and 85-211 of greater than 82%. In another embodiment, “homology” refers to identity to a sequence selected from SEQ ID No: 1-16, 18-23, 25-26, 28-32, 34-46, 48-58, 60-66, 68-70, and 85-211 of greater than 83%. In another embodiment, “homology” refers to identity to one of SEQ ID No: 1-16, 18-23, 25-26, 28-32, 34-46, 48-58, 60-66, 68-70, and 85-211 greater than 85%. In another embodiment, “homology” refers to identity to one of SEQ ID No: 1-16, 18-23, 25-26, 28-32, 34-46, 48-58, 60-66, 68-70, and 85-211 of greater than 87%. In another embodiment, “homology” refers to identity to a sequence selected from SEQ ID No: 1-16, 18-23, 25-26, 28-32, 34-46, 48-58, 60-66, 68-70, and 85-211 of greater than 88%. In another embodiment, “homology” refers to identity to one of SEQ ID No: 1-20 of greater than 90%. In another embodiment, “homology” refers to identity to one of SEQ ID No: 1-16, 18-23, 25-26, 28-32, 34-46, 48-58, 60-66, 68-70, and 85-211 of greater than 92%. In another embodiment, “homology” refers to identity to a sequence selected from SEQ ID No: 1-16, 18-23, 25-26, 28-32, 34-46, 48-58, 60-66, 68-70, and 85-211 of greater than 93%. In another embodiment, “homology” refers to identity to one of SEQ ID No: 1-16, 18-23, 25-26, 28-32, 34-46, 48-58, 60-66, 68-70, and 85-211 of greater than 95%. In another embodiment, “homology” refers to identity to a sequence selected from SEQ ID No: 1-16, 18-23, 25-26, 28-32, 34-46, 48-58, 60-66, 68-70, and 85-211 of greater than 96%. In another embodiment, “homology” refers to identity to one of SEQ ID No: 1-16, 18-23, 25-26, 28-32, 34-46, 48-58, 60-66, 68-70, and 85-211 of greater than 97%. In another embodiment, “homology” refers to identity to one of SEQ ID No: 1-16, 18-23, 25-26, 28-32, 34-46, 48-58, 60-66, 68-70, and 85-211 of greater than 98%. In another embodiment, “homology” refers to identity to one of SEQ ID No: 1-16, 18-23, 25-26, 28-32, 34-46, 48-58, 60-66, 68-70, and 85-211 of greater than 99%. In another embodiment, “homology” refers to identity to one of SEQ ID No: 1-16, 18-23, 25-26, 28-32, 34-46, 48-58, 60-66, 68-70, and 85-211 of 100%. Each possibility represents a separate embodiment of the present invention.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

Protein and/or peptide homology for any amino acid sequence listed herein is determined, in another embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

“Contacting,” in another embodiment, refers to directly contacting the target cell with a composition of the present invention. In another embodiment, “contacting” refers to indirectly contacting the target cell with a composition of the present invention. Each possibility represents a separate embodiment of the present invention. In another embodiment, the composition of the present invention is carried in the subjects' bloodstream to the target cell. In another embodiment, the composition is carried by diffusion to the target cell. In another embodiment, the composition is carried by active transport to the target cell. In another embodiment, the composition is administered to the subject in such a way that it directly contacts the target cell. Each possibility represents a separate embodiment of the present invention.

### Pharmaceutical Compositions and Methods of Administration

Pharmaceutical compositions containing compositions of the present invention can be, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment of methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, the pharmaceutical compositions are administered topically to body surfaces and are thus formulated in a form suitable for topical administration. Suitable topical formulations include gels, ointments, creams, lotions, drops and the like. In another embodiment, for topical administration, the compositions are prepared and applied as solutions, suspensions, or emulsions in a physiologically acceptable diluent with or without a pharmaceutical carrier.

In another embodiment, the active compound is delivered in a vesicle, e.g. a liposome.

In other embodiments, carriers or diluents used in methods of the present invention include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In another embodiment, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In other embodiments, the compositions further comprises binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants. Each of the above excipients represents a separate embodiment of the present invention.

The compositions also include, in another embodiment, incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions influence, in another embodiment, the physical state, solubility, stability, rate of in vivo release, and rate of in vivo clearance.

The preparation of pharmaceutical compositions that contain an active component, for example by mixing, granulating, or tablet-forming processes, is well understood in the art. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the active agents are converted into a solution, suspension, or emulsion, if desired with the substances customary and suitable for this purpose, for example, solubilizers or other substances.

Each of the above additives, excipients, formulations and methods of administration represents a separate embodiment of the present invention.

In one embodiment, the term “administering” refers to bringing a subject in contact with an active compound of the present invention. In another embodiment, administration is accomplished in vitro, i.e. in a test tube. In another embodiment, administration is accomplished in vivo, i.e. in cells or tissues of a living organism. Each possibility represents a separate embodiment of the present invention.

In one embodiment, the methods of the present invention comprise administering an active composition or compound of the present invention as the sole active ingredient. However, also encompassed within the scope of the present invention are methods for chemotherapy that comprise administering the active composition or compound in combination with one or more therapeutic agents (e.g. anti-tumor agents or cancer chemotherapy agents).

### EXPERIMENTAL DETAILS SECTION

### Example 1

### CD31-Based Immuno-LCM Successfully Captures Tumor Vascular Cells, Including Myeloid Cells

### Materials and Experimental Methods

### Tissues

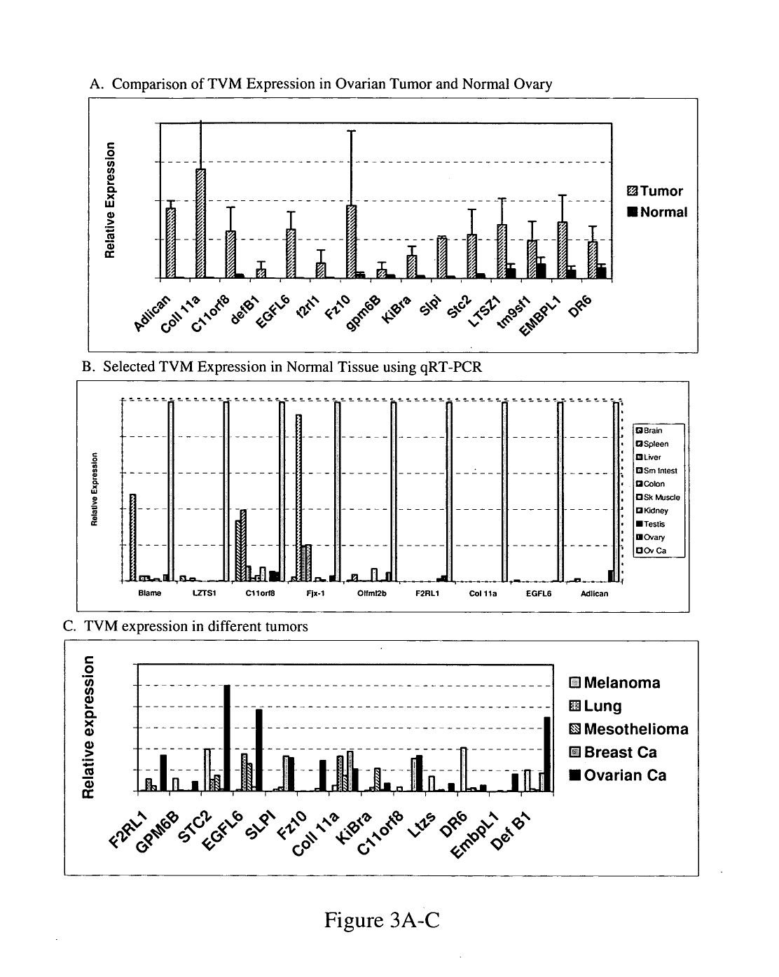

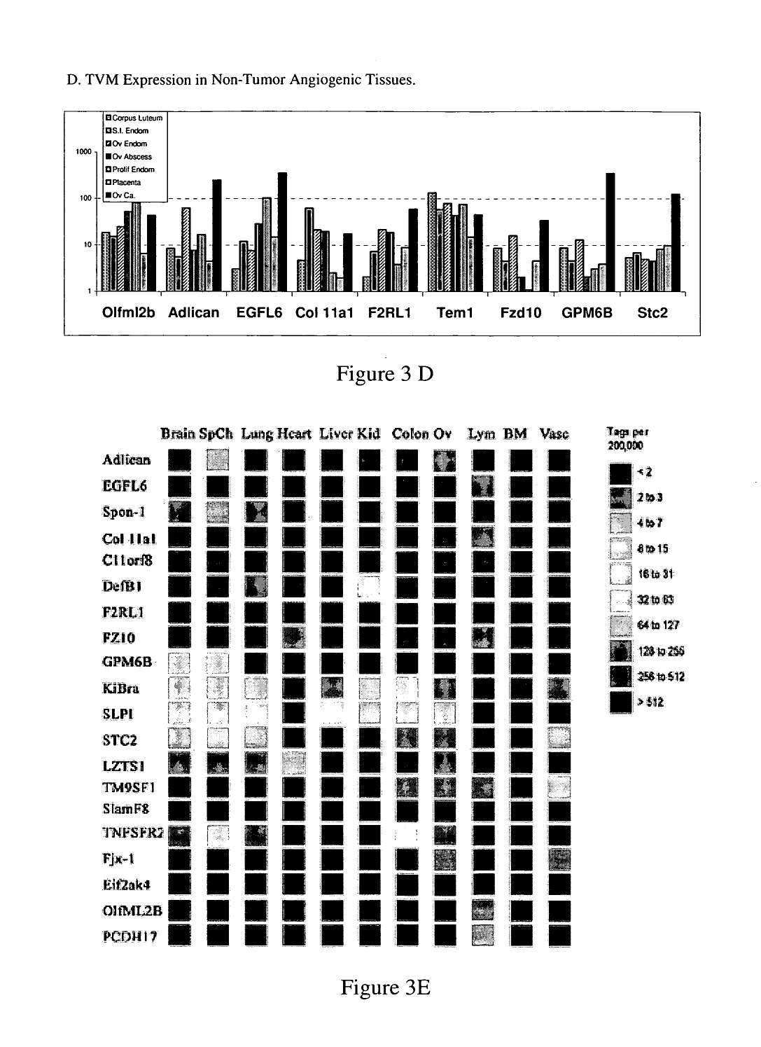

Stage-III epithelial ovarian cancer and ductal breast cancer specimens were collected at the University of Turin, Italy, following informed consent, from previously untreated patients. Additional ovarian cancer specimens, and normal ovaries were collected at the University of Pennsylvania Medical Center after obtaining written informed consent under Institutional Review Board (IRB)-approved protocols. Malignant mesothelioma (n=3), non-small cell lung carcinoma (n=3) (provided by Dr. Steven M. Albelda) and malignant melanoma (n=3) (provided by Dr. David Elder) were collected after obtaining written informed consent under IRB-approved protocols. A panel of normal human tissues (FIG. 3B) was provided by the Cooperative Human Tissue Network. All specimens were processed in compliance with HIPAA requirements.

### Reagents

Antibody against human CD31 (BD Pharmingen) followed by secondary antibodies (Vector, Burlingame, Calif.) were diluted (1:10) in PBS containing RNA Protector (1:10, Roche, Basel, Switzerland). Streptavidin conjugate and AEC chromagen (Dako, Carpenteria, Calif.) were diluted in PBS containing RNA Protector. Laser Capture Microdissection (LCM) was performed using Microcut (MMI, Glattbrugg, Switzerland), employing less than three hours per slide.

### RNA Isolation

In order to increase RNA yield, dissected samples were treated with pre-digested proteinase-K. RNA was isolated using TRIzol reagent microprotocol (Gibco, Carlsbad, Calif.). Glycogen carrier (20 μg) was utilized to increase RNA yield in all protocols. RNA integrity and quantity were assayed using the Bioanalyzer (Agilent, Foster City, Calif.).

### RNA Amplification

RNA was amplified using Messageamp® (Ambion, Austin, Tex.), with the following modifications: First-strand synthesis was performed at 42° C. (2 hours), then 48° C. (10 min). After second-strand synthesis, RNA was transcribed at 37° C. (12 hours); T7-polymerase and RNAse inhibitor were added and transcription was continued for 12 more hours. After 2 rounds of amplification, cRNA was biotin-labeled (12-24 hours, ENZO RNA labeling kit, Farmingdale, N.Y.) and purified using RNA cleanup (Qiagen, Valencia, Calif.).

### Array Analysis

Biotin-labeled cRNA was fragmented and hybridized to U133 (HG-U133) Set arrays (Affymetrix, Santa Clara, Calif.) representing more than 39,000 transcripts were derived from approximately 33,000 well-substantiated human genes, and scanned.

qRT-PCR

qRT-PCR was performed using primers to the 3′ end of transcripts spanning intron-exon boundaries whenever possible (Table 3) for 35 cycles using Sybergreen® (ABI, Foster City, Calif.), with primers at 150 nM concentrations. Primers were 18-24 nucleotides and were designed to have a TM of 59-61° C. All transcripts were confirmed using 3% agarose gel electrophoresis. Gene expression was normalized against β-actin in all studies unless stated otherwise.

### Immunostaining

For validation studies, immunohistochemistry (IHC) was performed using the VECTASTAIN ABC kit (Vector, Burlingame, Calif.). All primary antibodies were incubated for one hour. Immuno-reaction was visualized with 3,3′-diaminobenzidine (Vector). All staining steps were performed at room temperature.

### In Situ Hybridization (ISH)

DIG-labeled antisense RNA probes were generated by PCR amplification of 500-600-bp products incorporating T7 promoters into the antisense primers, followed by in vitro transcription with DIG RNA labeling reagents and T7 RNA polymerase (Roche, Indianapolis, Ind.). For immunodetection, an HRP-rabbit anti-DIG antibody (GenPoint kit; DAKO) was used and biotinyl-tyramide was included to catalyze biotin deposition (Perkin Elmer, Boston, Mass.). A second round of amplification utilized anti-biotin-HRP linked antibody (DAKO) followed by biotinyl-tyramide. Biotin was detected using a streptavidin-Cy3 conjugate (Arcturus) at 1:200 for 5 minutes. Slides were then washed and mounted with fluorescent mounting media containing DAPI (Vector).

### Cells

Human PBMCs were obtained through leukapheresis from healthy donors and were cultured for 7 days in DC media supplemented with 800 IU/ml human GM-CSF and 400 IU of IL-4. Cells were collected on day 7, labeled with CFSE, and incubated in media enriched with recombinant human (rh)VEGF₁₆₅ (60 ng/ml, Peprotech, Rocky Hill, N.J.) with or without VEGF-A neutralizing antibody (R&D Systems, Minneapolis Minn.) for seven days. Human peripheral blood monocytes were collected through elutriation. This population is highly enriched (greater than 95%) for CD14⁺ monocytes. Cells were processed exactly as above, treating them with GM-CSF and IL-4 for 7 days and with rhVEGF-A with or without VEGF-A neutralizing antibody for 7 additional days.

Endothelial progenitor assays were performed using the Endocult® assay (Stem Cell Technologies, Vancouver, Canada).

Human CD45⁺ VE-cadherin⁺ vascular leukocytes and CD45⁻ VE-cadherin⁺ tumor endothelial cells were sorted from mechanically dispersed ovarian cancers by FACS performed with MoFlo Cell Sorter (Cytomation, Fort Collins, Colo.) using a FITC-labeled rabbit anti-human VE-cadherin antibody (Medsystems Diagnostics GmbH, Vienna, Austria) and APC-anti-CD45 (HI30; BD Pharmingen, San Diego, Calif.). PBS containing 10% normal murine serum (Sigma, St. Louis, Mo.) and 25 mg/ml anti-mouse Fc receptor (2.4G2 BD Pharmingen) was added prior to incubation to avoid nonspecific binding.

### Mice and Xenograft Tumors

NOD/LtSz-SCID/β2-microglobulin knockout (NOD/SCID/β2M^{-/-}) mice were bred and maintained under conditions approved by the Animal Care Committee of the University of Pennsylvania. These mice lack adaptive immunity and exhibit a severe defect in NK cell function, permitting superior engraftment of human leukocytes. Female mice were transplanted subcutaneously with 0.3 ml cold Matrigel® (BD Biosciences, Bedford, Mass.) containing 1×10⁶ 2008 cells alone or enriched with 1×10⁵ DC, VLC or TEC into bilateral axillae. Tumors were resected at 3 weeks, RNA was extracted by the TRIzol method and analyzed for tumor vascular markers by qRT-PCR.

### Flow Cytometry

DCs generated in vitro from PBMC or monocytes, and CFSE-labeled DC treated in vitro with VEGF-A were subjected to flow cytometry, using FACSCanto® flow cytometer and CellQuest 3.2.1fl software (both Becton Dickinson). These studies used biotin anti-VE-cadherin antibody (Medsystems Diagnostics GmbH, Vienna, Austria) coupled with streptavidin-PE-Cy7, APC anti human CD45 antibody and IgG control (BD Pharmingen).

### Bioinformatic and Statistical Analyses

Statistical significance for mRNA expression differences between tissue types was determined using a two-tailed Student's t-Test. Pearson's correlation was used to determine linearity of arrays performed with one versus two rounds of amplification or before and after immuno-LCM. Analyses of expression profiles were performed using Genespring software (Agilent); all samples were normalized with the median defined as 1.0. A heat map condition tree was developed using a hierarchical clustering algorithm (Genespring®) excluding all genes where the difference between the means of the tumor and normal vascular samples was less than its standard error. Descriptive statistics were performed with the SPSS® statistics software package (SPSS, IL, USA). The algorithm for the nonparametric method based on the ranks of the expression level for tumor and normal samples was developed in SAS 9.1.

### Optimization of Immunostaining

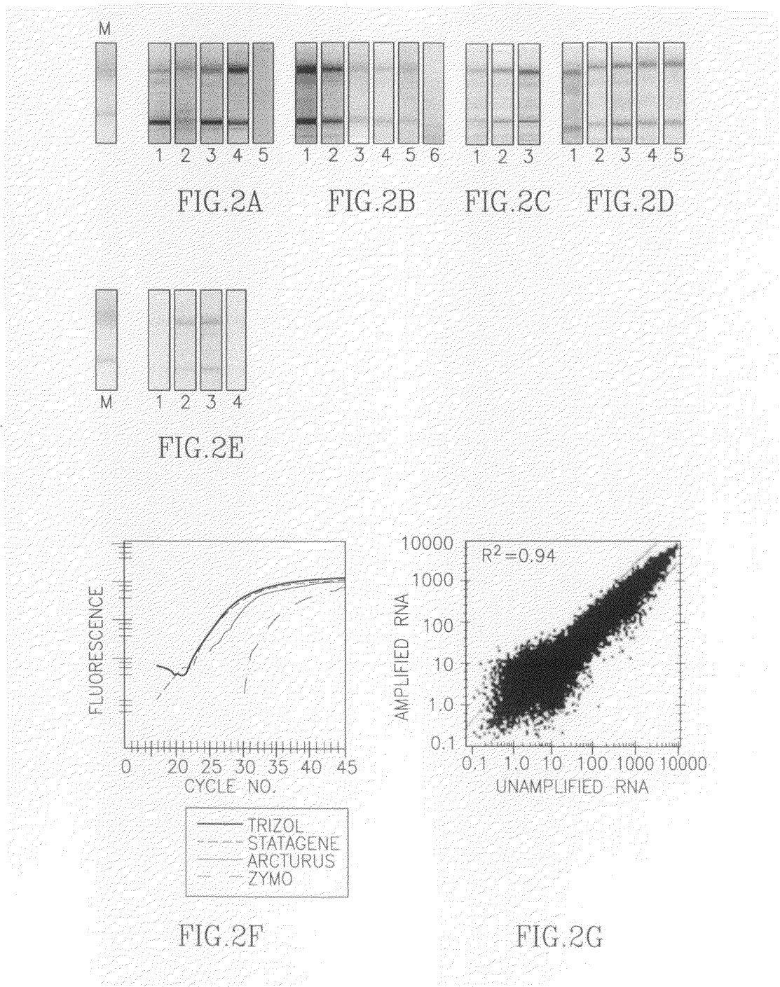

To procure highly purified tumor vasculature, a rapid and reliable immuno-LCM protocol was established for microarray applications. Different fixation conditions were tested, including −20° C. acetone; 70% ethanol:10% acetic acid (1:1, vol:vol); methanol; or 4% paraformaldehyde. Fixation with acetone or ethanol-based fixatives resulted in the greatest RNA yield (FIG. 2A). Acetic acid:ethanol fixation did not enable optimal IHC visualization of select target proteins. Acetone fixation was chosen for all further experiments.

Next, immunostaining was optimized. RNA isolated from tissue sections after standard IHC using LSAB (Dako) or Vectastain (Vector) showed near-complete degradation (FIG. 2B-6). We thus developed an ultra-fast IHC protocol with increased concentrations of reagents. High concentrations of RNAse inhibitor were added to all aqueous solutions. The choice of RNAse inhibitor was critical for RNA integrity. RNA Protector® (Roche) was found to be superior to placental RNAse inhibitor (Stratagene) or SuperRNAsin® (Ambion), leading to two-fold increase in RNA yield and integrity (FIG. 2B). Combining RNAse inhibitors reduced the efficacy of RNA Protector. Addition of RNA Protector to IHC allowed for 90% preservation of RNA integrity, based upon comparison of ribosomal RNA ratios determined using the Agilent Bioanalyzer.

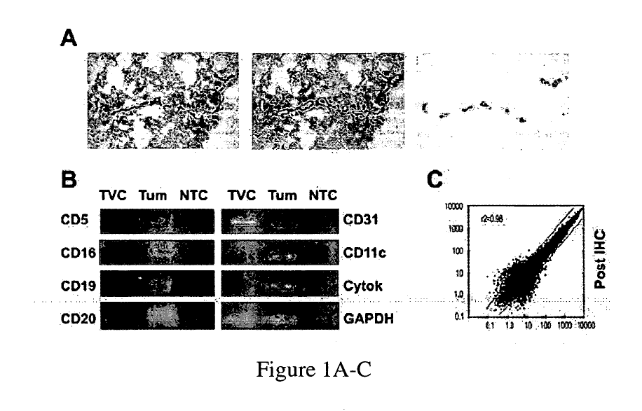

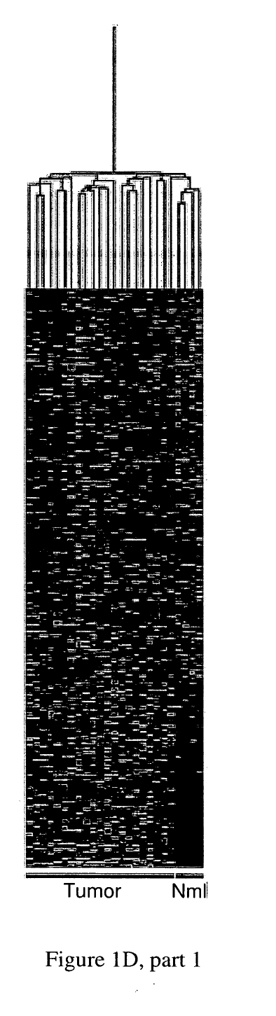

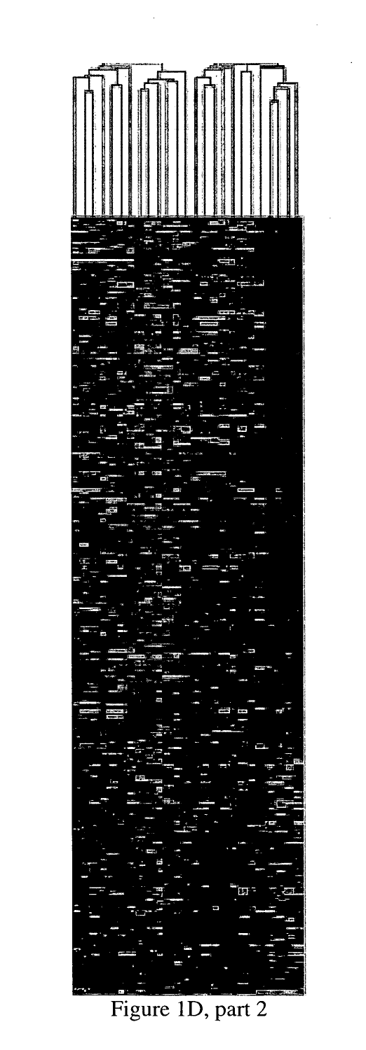

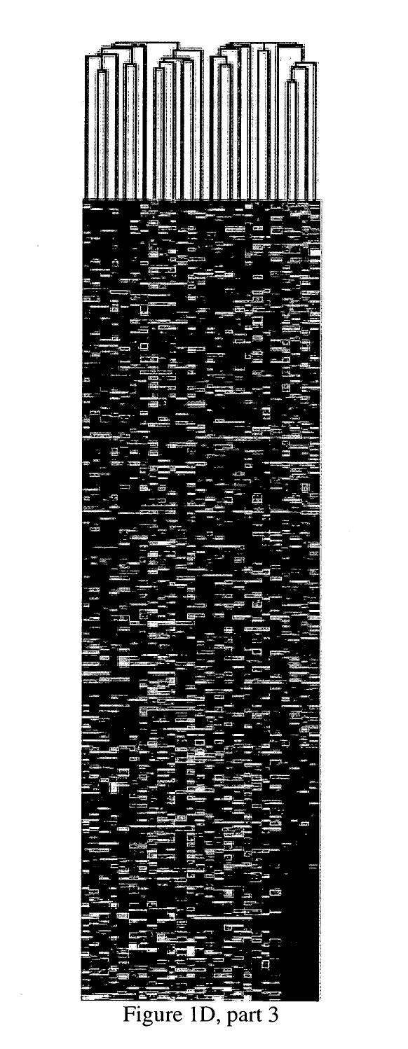

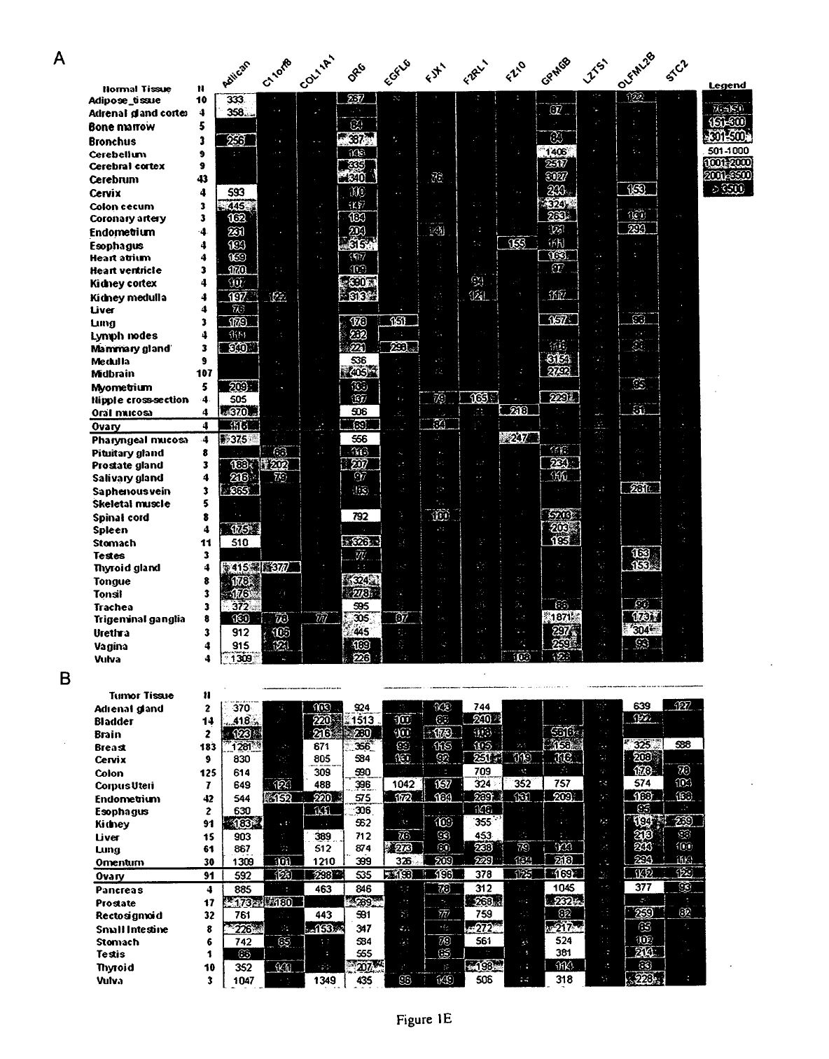

Next detection systems were compared. AEC chromagen resulted in 40% greater RNA yield than DAB. Immunofluorescence resulted in 100% increased RNA yield compared to AEC (FIG. 1C), but contaminating cells were poorly identifiable without counterstaining, as assessed by qRT-PCR detection of the T-cell marker CD3-ε. Furthermore, fluorescence quenching limited LCM time. Thus, AEC IHC was used for subsequent experiments.

In addition, the effect of LCM time on RNA yield and integrity was examined. Leaving immunostained tissue sections at room temperature for up to three hours before RNA was isolated had no significant impact on the quality or quantity of RNA isolated (FIG. 1D).

### Optimization of RNA Purification.

RNA amplification methods (Arcturus Picopure kit, Stratagene microRNA isolation kit, Zymo mini RNA isolation kit and the modified TRIzol method for less than 10⁵ cells) were compared for RNA yield and quality after immuno-LCM. Arcturus Picopure gave the highest RNA yield for tissues stained with hematoxylin alone, but not following IHC (FIG. 2E). The protocol from ZYMO also resulted in low RNA yield. Conversely, the Stratagene micro RNA isolation kit and the modified TRIzol method gave significantly better and similar yields by quantification with the Agilent Bioanalyzer.

RNA quality was tested by qRT-PCR of GADPH and β-actin transcripts in total RNA procured from 1×10⁶ cells microdissected and processed as in Table 2. GADPH or β-actin transcripts were detected at similar levels in RNA isolated with the modified TRIzol method using phase-lock tubes (Eppendorf, Hamburg, Germany) or with the Stratagene micro RNA isolation kit. Arcturus picopure and ZYMO RNA isolation kits were 10-fold and 256-fold less sensitive, respectively (FIG. 2F).

The resulting protocol, requiring approximately 25 minutes for IHC, proved successful for numerous antibodies (Table 1). While some antibodies required longer incubation times (up to 15 minutes), there was no loss of RNA yield or integrity. Staining was quite specific, even with high concentrations of antibody. The protocol was reproducibly able to capture 500,000 μm² of tumor vascular cells in 3 hours of microdissection and recover ˜20 ng total RNA. RNA was reproducibly amplified to 15 μg of biotin-labeled cRNA.

### Optimization of RNA Amplification

The linearity of amplifications using Ambion MessageAmp was tested by comparing the gene expression profile of 10 μg unamplified whole tumor RNA to amplified 6, 24 or 60 ng of the same RNA. Transcriptional profiling was performed using Affymetrix U133 chips. Correlation between unamplified RNA and 24 or 60 ng input RNA was high (r²=0.93 and 0.94, respectively) (FIG. 2G). Correlation was lower with 6 ng input RNA (r²=0.87). High correlation was found between gene expression profiles from amplifications of input. RNA procured from the same tumor performed within the same experiment (intra-assay, r²=0.99) or in different experiments (inter-assay, r²=0.97). Immuno-LCM had no impact on expression profile (FIG. 1C).

TABLE 1
List of antibodies tested, company and clone used in the study
as indicated. Success with staining using the rapid IHC protocol
for LCM following fixation in acetone or acetic acid/ethanol
(AA/EtOH) is reported on the side for tested antibodies.

| Antibody | Company | Clone | Acetone | AA/EtOH |
|---|---|---|---|---|
| Biot hCD45 | BD Pharm | H130 | *** | −− |
| Biot hCD31 | Ancell | 158-2B3 | *** | ** |
| Biot hCD31 | Caltag | MBC 78.2 | ** | ** |
| hCD31 | Dako | JC70A | ** | ** |
| Biot CD146 | Chemicon | MAB16985B | *** | *** |
| CytoKeratin | Dako | AO575 | * | −− |
| Biot hCD3 | BD Pharm | UCHT1 | *** | −− |
| Fitc hCD31 | BD Pharm | WM59 | *** | *** |
| SMA-α-Cy3 | Sigma | 1A4 | ND | ND |
| FOLH1 | Zymed | ZMD.80 | ND | ND |
| STC2 | Genway | A22017 | ND | ND |
| Biot CD74 | BD Pharm | Mb741 | ND | ND |
| AML-1 | Active Motif | Polyclonal | ND | ND |
| hCD34-PE | BD Pharm | 581 | ND | ND |
| F-Spon | Abcam | Ab14271-50 | ND | ND |
| Lrp4 | Abcam | Ab13388-25 | ND | ND |
| Endothelial Lipase | Cayman | Polyclonal | ND | ND |
| | Chemical | | | |

(−−), poor stain,
(*) fair stain,
(**) good stain,
(***) excellent stain.
ND, not determined.

The optimized Immuno-LCM protocol is summarized in Table 2.

TABLE 2
Summary of Immuno-LCM Protocol

| | |
|---|---|
| Tumor | Freshly cut 8 μm sections of snap frozen tumor |
| IHC** | Fix in −20° C. Acetone - 4 min |
| | Incubate with primary Ab 1:10 - 5 min |
| | Incubate with 3x biotinylated anti-mouse Ab |
| | (Vector) - 5 min. |
| | Brief wash in PBS |
| | 2.5X Streptavidin-biotin amplification (DAKO) - |
| | 5 min |
| | Brief wash in PBS |
| | AEC (DAKO) stain - 3 to 5 min |
| | Brief wash in PBS |
| | Stain with dilute hematoxylin |
| | Rinse |
| | (** All steps with 1:10 RNAse Protector) |
| LCM | Dry tissue sections with hair dryer - 1 min |
| | Microdissect cells - up to 3 hours |
| RNA isolation | Treat with Proteinase K (10 μg/ml) - 8 min |
| | Extract RNA with TRIzol in phase lock gel - 1 hour |
| RNA Amplification | Use Ambion MessageAmp |

During the optimization of RNA isolation and amplification methodology, it was found that the immuno-LCM procedure had minimal impact on RNA integrity (FIG. 2A-F) or gene expression profiling (FIGS. 1C and 2G).

The PCR primers used for qRT-PCR and in situ hybridization are set forth in Table 3 below. A: qRT-PCR; B: T7 polymerase. (This table also includes primers used to amplify controls [those from CD3eps to GAPDH] and additional markers that do not appear in Table 5; selection of these additional markers is described below in Example 11).

TABLE 3A

| | | |
|---|---|---|
| GENE | FORWARD PRIMER; SEQ ID | REVERSE PRIMER; SEQ ID |

| | | |
|---|---|---|
| Adlican 5′ | cattgctagacacgtggaaaga; 212 | tctcattgccgtgaatcataag; 213 |
| Adlican 3′ | aggaaccctcaagctggact; 214 | cattggcaaacaccttgattc; 215 |
| C11ORF8 | cttcaaagagtgggctgtgtg; 216 | gaggcattgatgtacgttgtg; 217 |
| CD31 | tttggctagtccttgttctttgg; 218 | cggtgtcttcaggttggtattc; 219 |
| Coll11α1 | agggtgaagatggagatcctg; 220 | tttcaccttgtcttccctctg; 221 |
| Cytokeratin 18 | gcgtacagagatggagaacgaat; 222 | cgagactccagctctaccttgtt; 223 |
| EGFL6 | agaagaccacgagtgaggatg; 224 | ccacgttctgcttcaaaaatg; 225 |
| F2RL1 | tgctagcagcctctctctcc; 226 | tttccagtgacgtgggatg; 227 |
| FJX1 | actacctgacggccaacttc; 228 | cgcctcattgtccagaaag; 229 |
| FZ10 | cagactaaaacgctggactgc; 230 | tcttggaggtccaaatccac; 231 |
| GAPDH | cctgcaccaccaactgctta; 232 | catgagtccttccacgatacca; 233 |
| GPM6B | tcctatcacctgttcattgtgg; 234 | gcagcaatcttcccgactc; 235 |
| LZTS1 | agggagaagaccagcttcg; 236 | aggatctcgctagccttgg; 237 |
| OLFML2B | ccaaaggatgagcggatttac; 238 | gctgtacgggagcttgtagg; 239 |
| α-SMA | actgagcgtggctattccttc; 240 | agtggccatctcattttcaaag; 241 |
| SLIT2 | gaaatctcttgtcgaggggaaag; 242 | acacccacctctgcactctaatc; 243 |
| SPON-1 | agtgagcagctgaaggaagagt; 244 | catgtaacgttcctgaattccac; 245 |
| STC2 | tacctcaagcacgacctgtg; 246 | gtcagcagcaagttcacgag; 247 |
| TNFαIP6 | cgcaacttacaagcagctagag; 248 | taatgccagtttttccaaatcc; 249 |
| TNFRSF21 | ggcttcttcgtggatgagtc; 250 | ccgcaacactgtgtccttc; 251 |
| (DR6) | | |

TABLE 3B

| | | |
|---|---|---|
| Gene | FORWARD, ISH; SEQ ID | REVERSE, T7; SEQ ID |

| | | |
|---|---|---|
| Adlican | caagatcgaggccagtatatgtg; | gcaaacaccttgattctgctatc; |
| | 252 | 253 |
| CD31 | tgaggttctgagggtgaagg; | ggatcctaatacgactcactatagggagattgggatctga |
| | 254 | cattttctcg; 255 |
| EGFL6 | agaagaccacgagtgaggatg; | ggatcctaatacgactcactatagggagatgaggaggg |
| | 256 | gagataaactgac; 257 |
| FJX1 | actacctgacggccaacttc; | ggatcctaatacgactcactatagggagattcctcccggt |
| | 258 | gacactaag; 259 |
| FZ10 | cagactaaaacgctggactgc; | ggatcctaatacgactcactatagggagatcctttcagtgt |
| | 260 | atcacagcatc; 261 |
| GPM6B | tcctatcacctgttcattgtgg; | ggatcctaatacgactcactatagggagatgaaactggtt |
| | 262 | atcaggcaatc; 263 |
| TNFαIP6 | acggtctggcaaatacaagc; | ggatcctaatacgactcactatagggagaactgatgatgt |
| | 264 | catctggaagc; 265 |
| TNFRSF21 | ggcttcttcgtggatgagtc; | ggatcctaatacgactcactatagggagacacacacaca |
| | 266 | caccccaaac; 267 |

### Results

A rapid protocol was developed and optimized for immuno-LCM, followed by extraction and amplification of RNA for array analysis of tumor vascular cells (TVC). 4×10³ CD31⁺ cells with vascular morphology were isolated (FIG. 1A). RT-PCR analysis of RNA from purified TVC confirmed the absence of lineage-specific markers CD3-ε and CD5 (T-cells); CD16 (NK cells); CD19 and CD20 (B-cells); and cytokeratin-18 (tumor cells) (FIG. 1B). In addition, expression of the myeloid markers CD45, CD11c and CD14 was detected, confirming that TVC had been isolated. These findings were further confirmed by quantitative real-time polymerase chain reaction (qRT-PCR), which revealed a 15-fold enrichment of CD31 mRNA, similar expression of monocyte markers, and absence of cytokeratins and CD3-ε in micro-dissected TVC specimens relative to whole tumor. Smooth muscle α-actin was detectable in only 50% of samples and at 10-fold reduced levels relative to whole tumor, showing that few, in any perivascular cells were present.

Thus, the method successfully captured vascular cells, including vascular cells of monocytic lineage.

### Example 2

### mRNA Profile of Immuno-LCM Procured Tumor Vascular Cells

The mRNA profiles of micro-dissected TVC from 2 tumor samples were analyzed in parallel to 3 micro-dissected normal ovary vascular cells and to cultured human umbilical vein endothelial cells (HUVEC) using Affymetrix-U133 arrays. 13/13 known pan-endothelial markers in were detected in the TVC arrays. Similarly, 14/15 known tumor endothelial-specific markers (those listed below, Tem-4, and Tem-9) were exclusively expressed or markedly overexpressed (p<0.001) in TVC arrays (Table 4). These findings indicate that the protocol exhibits a sensitivity of greater than 90%.

TABLE 4
Expression of pan-endothelial and tumor endothelial markers in
HUVEC; endothelial cells from normal ovary isolated through
immuno-LCM (Normal); and tumor endothelial cells from ovarian
cancer specimens isolated through immuno-LCM (Tumor1 and
Tumor2).

| | HUVEC | Normal | Tumor 1 | Tumor 2 |
|---|---|---|---|---|
| Pan Endothelial Markers | | | | |
| Angiomodulin | + | + | + | + |
| Hevin | + | + | + | + |
| Connective tissue growth | + | + | + | + |
| factor | | | | |
| Collagen, type VI, alpha 1 | + | + | + | + |
| Interferon induced | + | + | + | + |
| transmembrane protein 1 | | | | |
| Guanine nucleotide binding | + | + | + | + |
| protein 11 | | | | |
| Von Willebrand factor | + | + | + | + |
| SmLIM | + | + | + | + |
| Collagen, type XVIII, | + | + | + | + |
| alpha 1 | | | | |
| Insulin-like growth | + | + | −− | + |
| factor-binding protein 4 | | | | |
| P1H12 (CD146) | +/− | +/− | +/− | +/− |
| Osteonectin | + | + | + | + |
| Matrix Gla protein | + | + | + | + |
| Tumor Endothelial Markers | | | | |
| TEM-1 | −− | + | + | + |
| TEM-3/TEM-7 | −− | + | + | + |
| TEM-5 | +/− | +/− | +/− | + |
| TEM-6 | + | + | + | + |
| TEM-8 | −− | −− | −− | −− |
| MMP-11 | −− | −− | +/− | + |
| Gelatinase A | +/− | +/− | + | + |
| Nidogen | + | −− | +/− | + |
| Collagen, type VI, alpha 3 | −− | + | + | + |
| Thy-1 cell surface antigen | −− | −− | + | + |
| Collagen, type III, alpha 1 | + | + | + | + |
| Bone morphogenetic | −− | −− | +/− | + |
| protein 1 | | | | |
| Cystatin S | + | −− | −− | −− |
| Macrophage mannose | +/− | +/− | +/− | +/− |
| receptor family | | | | |

(+) indicates detection of the gene with all associated probe sets, when more than one probe set exists;
(+/−): detection with one but not all probe sets associated with the gene;
(−−): lack of detection with any associated probe sets for the indicated gene.

Next, vascular cells from 21 stage-III ovarian tumors and 4 normal ovaries were microdissected, profiled and analyzed. In order to determine if there was a distinct signature that could distinguish tumor from normal vasculature, unsupervised hierarchical clustering was performed using 17,920 genes (after elimination of all genes wherein the difference between tumor and normal means was less than the standard error of the difference in the means). Hierarchical clustering of these samples accurately classified tumor from normal vascular cells, demonstrating a clear difference in the molecular profile between tumor and normal vasculature and further confirming the validity and accuracy of the method used (FIG. 1D).

To identify robust, tumor vasculature-specific genes, the 17,920 genes were further characterized using (1) the fold difference comparing tumor to normal vascular samples; (2) the number of tumor and normal vascular samples where a gene was classified as “present” or “absent” by MAS5.0 Suite (Affymetrix); and (3) the gene-specific ranks of the normalized gene expression values for all samples. 70 genes were selected as tumor vascular markers (TVM) that fulfilled at least 1 of the following 3 criteria: (1) present in more than 15/21 TVC samples and absent in more than 3/4 normal vascular samples; (2) present in more than 18/21 tumor vascular samples and at least 3-fold up-regulated on average in tumor vascular samples; or (3) at least 3/4 normal samples had 1 of the 5 lowest ranked expression values (therefore at least 20/21 tumor vascular samples had higher expression values relative to normal samples) (Table 5). By this method, a number of transcripts previously not known to be TVM were identified, e.g. adlican, GPM6B, TNFaIP6, LZTS1 and numerous expressed sequence tags (EST). The identified markers also included several known TVM (FolH1 (PSMA), Thy-1, TEM-7, SLIT2, and chondroitin-sulfate proteoglycan-2 (versican)), further validating the methods used to identify TVM.

TABLE 5

| | Fold | | | |
|---|---|---|---|---|
| | Change | | | |
| Common Gene | Tumor | No. Present in | | Rank of |
| Name or | vs. | Tumor | Normal | Normal |
| Genebank No. | Normal | [21) | (4] | Specimens |

part 1. List of 70 TVM identified by three independent analyses.
Genes were included in the list if they fulfilled tumor vascular
marker criteria in at least one of the analyses. The table describes
results from all analyses for each gene. First column lists genes
reported by common gene name and ESTs reported by Genebank
admission; second column describes expression fold change in
tumor relative to normal vascular cells; third column describes the
number of tumor (total of 21) and normal (total of four) vascular cell
specimens for which the gene was determined to be expressed;
fourth column describes the expression rank of the four normal
vascular cell samples (with 1 being the lowest
expression and 25 the highest).

| | | | | |
|---|---|---|---|---|
| ADAM12 | 5.1 | 13 | 0 | 1, 2, 4, 6 |
| Adlican | 5.4 | 19 | 2 | 1, 2, 5, 8 |
| AI798118 | 7.7 | 15 | 0 | 1, 2, 3, 5 |
| AK000168 | 3.0 | 17 | 1 | 4, 6, 7, 15 |
| AMI-1 | 30 | 12 | 0 | 1, 2, 3, 5 |
| ANKRD9 | 3.2 | 16 | 1 | 1, 4, 7, 16 |
| Av758342 | 4.0 | 17 | 2 | 1, 2, 9, 10 |
| AW242409 | 4.9 | 17 | 2 | 1, 3, 5, 9 |
| Aw510657 | 3.9 | 18 | 4 | 4, 5, 6, 7 |
| AW517716 | 7.4 | 12 | 0 | 1, 2, 3, 13 |
| BDKRB2 | 3.7 | 15 | 1 | 2, 3, 5, 11 |
| C6orf37 | 5.2 | 15 | 1 | 2, 3, 5, 7 |
| C6orf55 | 2.4 | 18 | 1 | 1, 2, 3, 8 |
| C6orf69 | 2.0 | 19 | 3 | 1, 2, 4, 7 |
| C7orf32 | 3.4 | 16 | 1 | 1, 2, 4, 7 |
| C11orf8 | 4.08 | 13 | 1 | 1, 2, 5, 17 |
| CD24 | 3.7 | 16 | 1 | 2, 6, 7, 8 |
| CD51 | 3.7 | 15 | 2 | 1, 2, 3, 8 |
| CENTA2 | 2.1 | 16 | 3 | 1, 3, 4, 5 |
| CLDN1 | 2.4 | 16 | 1 | 1, 2, 3, 13 |
| COL11A1 | 10.1 | 15 | 0 | 1, 2, 7, 8 |
| COL15A1 | 4.8 | 21 | 4 | 1, 3, 8, 12 |
| COL22A1 | 3.5 | 11 | 0 | 1, 2, 3, 5 |
| CSPG2 | 5.8 | 21 | 4 | 1, 2, 3, 6 |
| DEFB1 | 2.4 | 11 | 0 | 1, 2, 4, 13 |
| DSG2 | 4.2 | 11 | 1 | 2, 3, 15, 17 |
| EGFI6 | 4.6 | 19 | 4 | 1, 2, 6, 7 |
| EIF2AK4 | 2.8 | 18 | 1 | 1, 2, 4, 9 |
| EBP41L3 | 4.0 | 16 | 2 | 1, 4, 5, 13 |
| ESM1 | 3.7 | 20 | 1 | 1, 4, 5, 7 |
| FBXO32 | 2.2 | 16 | 1 | 1, 2, 5, 8 |
| FJX1 | 11.3 | 16 | 0 | 1, 2, 3, 5 |
| FOLH1 | 8.8 | 13 | 1 | 1, 2, 4, 6 |
| FLJ22875 | 2.3 | 16 | 0 | 2, 3, 6, 7 |
| FLJ46072 | 3.3 | 20 | 1 | 1, 2, 3, 5 |
| Fz2RL1 | 3.6 | 13 | 0 | 1, 7, 10, 12 |
| FZ010 | 2.0 | 15 | 0 | 2, 3, 6, 10 |
| GPM68 | 14.63 | 16 | 0 | 1, 2, 5, 6 |

part 2.

| | | | | |
|---|---|---|---|---|
| GPR105 | 3.3 | 11 | 0 | 1, 2, 6, 10 |
| HOXB7 | 2.8 | 16 | 1 | 1, 2, 7, 14 |
| IGSF4 | 5.0 | 18 | 1 | 1, 4, 5, 6 |
| IVNS1ABP | 3.3 | 21 | 4 | 1, 2, 3, 5 |
| KCNK5 | 2.5 | 16 | 1 | 1, 3, 5, 9 |
| LZTS1 | 4.3 | 19 | 1 | 1, 2, 6, 7 |
| MCM4 | 2.4 | 13 | 0 | 1, 2, 4, 6 |
| NID2 | 4.1 | 16 | 1 | 1, 4, 8, 15 |
| OlfML28 | 2.4 | 18 | 1 | 1, 2, 4, 11 |
| O88PL3 | 4.1 | 17 | 1 | 1, 3, 4, 20 |
| PCDH17 | 2.9 | 21 | 4 | 1, 3, 4, 7 |
| PLCE1 | 3.8 | 13 | 1 | 1, 3, 4, 16 |
| PLXDC1 | 4.0 | 21 | 3 | 1, 3, 5, 8 |
| PRDM1 | 2.4 | 15 | 0 | 1, 3, 4, 5 |
| RGC32 | 9.0 | 16 | 2 | 1, 3, 6, 8 |
| RGS1 | 8.0 | 12 | 1 | 2, 3, 5, 16 |
| RRM2 | 4.7 | 16 | 1 | 2, 4, 5, 21 |
| SCNN1A | 15.8 | 16 | 0 | 1, 2, 3, 6 |
| SCD2 | 4.42 | 20 | 2 | 1, 2, 6, 10 |
| SlamF6 (BLAME) | 3.5 | 13 | 0 | 2, 3, 6, 8 |
| SLIT2 | 2.0 | 16 | 1 | 3, 4, 6, 9 |
| SPON1 | 2.5 | 16 | 1 | 1, 6, 7, 17 |
| SPRY4 | 2.8 | 20 | 2 | 1, 2, 3, 5 |
| STC2 | 9.0 | 11 | 0 | 1, 2, 4, 5 |
| STCH | 4.0 | 15 | 2 | 1, 6, 7, 9 |
| TACSTD1 | 11.6 | 16 | 2 | 1, 2, 7, 10 |
| TARS | 2.8 | 16 | 1 | 2, 5, 9, 11 |
| TBX2 | 2.2 | 14 | 1 | 1, 5, 6, 24 |
| THY1 | 3.1 | 19 | 3 | 1, 2, 4, 8 |
| TNFAIP8 | 5.6 | 17 | 0 | 1, 2, 3, 6 |
| TNFSFR21 (DRS) | 8.2 | 11 | 0 | 1, 3, 4, 7 |
| UPP1 | 4.97 | 15 | 1 | 1, 3, 5, 20 |

TABLE 6

Sequences of the TVM and/or exemplary GenBank Accession No(s). Any GenBank
Accession No. that appears in this table or in any table or part of this specification
is merely exemplary, and is not intended to limit the sequence of the present invention
to a particular isoform, a particular species, or any other type of limitation. Each
GenBank Accession No. represents a separate embodiment of the present invention. Further,
sequences presented herein as cDNA sequences (i.e. containing “t,” signifying thymidine)
will be understood to also represent the corresponding RNA sequence (i.e. containing “u”
for uridine). Further, some of the sequences presented herein contain non-coding sequences
5′ and/or 3′ to the coding sequence of the mRNA. In these cases, the isolated coding
sequence of the mRNA and methods comprising same represent another embodiment of the
present invention.

| | | |
|---|---|---|
| Gene name | | |
| and/or | | |
| Exemplary | | |
| GenBank | SEQ ID | Sequence and/or exemplary GenBank Accession |
| Accession No(s) | No. | Numbers |

| | | |
|---|---|---|
| meltrin-L | 1 | AF023476 |
| precursor | | NM_003474 |
| (ADAM12) | | |
| Adlican | 2 | AF245505; Sequence below. |

atgcccaagcgcgcgcactggggggccctctccgtggtgctgatcctgctttggggccatccgcgagtggcgctggcctgcccgcatccttgt
gcctgctacgtccccagcgaggtccactgcacgttccgatccctggcttccgtgcccgctggcattgctagacacgtggaaagaatcaatttgg
ggtttaatagcatacaggccctgtcagaaacctcatttgcaggactgaccaagttggagctacttatgattcacggcaatgagatcccaagcatc
cccgatggagctttaagagacctcagctctcttcaggttttcaagttcagctacaacaagctgagagtgatcacaggacagaccctccagggtct
ctctaacttaatgaggctgcacattgaccacaacaagatcgagtttatccaccctcaagctttcaacggcttaacgtctctgaggctactccatttg
gaaggaaatctcctccaccagctgcaccccagcaccttctccacgttcacatttttggattatttcagactctccaccataaggcacctctacttag
cagagaacatggttagaactcttcctgccagcatgcttcggaacatgccgcttctggagaatctttacttgcagggaaatccgtggacctgcgat
tgtgagatgagatggtttttggaatgggatgcaaaatccagaggaattctgaagtgtaaaaaggacaaagcttatgaaggcggtcagttgtgtgc
aatgtgcttcagtccaaagaagttgtacaaacatgagatacacaagctgaaggacatgacttgtctgaagccttcaatagagtcccctctgagac
agaacaggagcaggagtattgaggaggagcaagaacaggaagaggatggtggcagccagctcatcctggagaaattccaactgccccagt
ggagcatctctttgaatatgaccgacgagcacgggaacatggtgaacttggtctgtgacatcaagaaaccaatggatgtgtacaagattcacttg
aaccaaacggatcctccagatattgacataaatgcaacagttgccttggactttgagtgtccaatgacccgagaaaactatgaaaagctatggaa
attgatagcatactacagtgaagttcccgtgaagctacacagagagctcatgctcagcaaagaccccagagtcagctaccagtacaggcagg
atgctgatgaggaagctctttactacacaggtgtgagagcccagattcttgcagaaccagaatgggtcatgcagccatccatagatatccagct
gaaccgacgtcagagtacggccaagaaggtgctactttcctactacacccagtattctcaaacaatatccaccaaagatacaaggcaggctcg
gggcagaagctgggtaatgattgagcctagtggagctgtgcaaagagatcagactgtcctggaagggggtccatgccagttgagctgcaacg
tgaaagcttctgagagtccatctatcttctgggtgcttccagatggctccatcctgaaagcgcccatggatgacccagacagcaagttctccattc
tcagcagtggctggctgaggatcaagtccatggagccatctgactcaggcttgtaccagtgcattgctcaagtgagggatgaaatggaccgca
tggtatatagggtacttgtgcagtctccctccactcagccagccgagaaagacacagtgacaattggcaagaacccaggggagtcggtgacat
tgccttgcaatgctttagcaatacccgaagcccaccttagctggattcttccaaacagaaggataattaatgatttggctaacacatcacatgtata
catgttgccaaatggaactctttccatcccaaaggtccaagtcagtgatagtggttactacagatgtgtggctgtcaaccagcaaggggcagac
cattttacggtgggaatcacagtgaccaagaaagggtctggcttgccatccaaaagaggcagacgcccaggtgcaaaggctctttccagagtc
agagaagacatcgtggaggatgaagggggctcgggcatgggagatgaagagaacacttcaaggagacttctgcatccaaaggaccaagag
gtgttcctcaaaacaaaggatgatgccatcaatggagacaagaaagccaagaaagggagaagaaagctgaaactctggaagcattcggaaa
aagaaccagagaccaatgttgcagaaggtcgcagagtgtttgaatctagacgaaggataaacatggcaaacaaacagattaatccggagcgc
tgggctgatattttagccaaagtccgtgggaaaaatctccctaagggcacagaagtacccccgttgattaaaaccacaagtcctccatccttgag
cctagaagtcacaccaccttttcctgctgtttctcccccctcagcatctcctgtgcagacagtaaccagtgctgaagaatcctcagcagatgtacct
ctacttggtgaagaagagcacgttttgggtaccatttcctcagccagcatggggctagaacacaaccacaatggagttattcttgttgaacctgaa
gtaacaagcacacctctggaggaagttgttgatgacctttctgagaagactgaggagataacttccactgaaggagacctgaaggggacagca
gcccctacacttatatctgagccttatgaaccatctcctactctgcacacattagacacagtctatgaaaagcccacccatgaagagacggcaac
agagggttggtctgcagcagatgttggatcgtcaccagagcccacatccagtgagtatgagcctccattggatgctgtctccttggctgagtctg
agcccatgcaatactttgacccagatttggagactaagtcacaaccagatgaggataagatgaaagaagacacctttgcacaccttactccaac
ccccaccatctgggttaatgactccagtacatcacagttatttgaggattctactataggggaaccaggtgtcccaggccaatcacatctacaag
gactgacagacaacatccaccttgtgaaaagtagtctaagcactcaagacaccttactgattaaaaagggtatgaaagagatgtctcagacact
acagggaggaaatatgctagagggagaccccacacactccagaagttctgagagtgagggccaagagagcaaatccatcactttgcctgact
ccacactgggtataatgagcagtatgtctccagttaagaagcctgcggaaaccacagttggtaccctcctagacaaagacaccacaacagtaa
caacaacaccaaggcaaaaagttgctccgtcatccaccatgagcactcacccttctcgaaggagacccaacgggagaaggagattacgccc
caacaaattccgccaccggcacaagcaaaccccacccacaacttttgccccatcagagactttttctactcaaccaactcaagcacctgacatta
agatttcaagtcaagtggagagttctctggttcctacagcttgggtggataacacagttaatacccccaaacagttggaaatggagaagaatgca
gaacccacatccaagggaacaccacggagaaaacacgggaagaggccaaacaaacatcgatataccccttctacagtgagctcaagagcg
tccggatccaagcccagcccttctccagaaaataaacatagaaacattgttactcccagttcagaaactatacttttgcctagaactgtttctctgaa
aactgagggcccttatgattccttagattacatgacaaccaccagaaaaatatattcatcttaccctaaagtccaagagacacttccagtcacatat
aaacccacatcagatggaaaagaaattaaggatgatgttgccacaaatgttgacaaacataaaagtgacattttagtcactggtgaatcaattact
aatgccataccaacttctcgctccttggtctccactatgggagaatttaaggaagaatcctctcctgtaggctttccaggaactccaacctggaatc
cctcaaggacggcccagcctgggaggctacagacagacatacctgttaccacttctggggaaaatcttacagaccctccccttcttaaagagct
tgaggatgtggatttcacttccgagtttttgtcctctttgacagtctccacaccatttcaccaggaagaagctggttcttccacaactctctcaagcat
aaaagtggaggtggcttcaagtcaggcagaaaccaccacccttgatcaagatcatcttgaaaccactgtggctattctcctttctgaaactagac
cacagaatcacacccctactgctgcccggatgaaggagccagcatcctcgtccccatccacaattctcatgtctttgggacaaaccaccaccac
taagccagcacttcccagtccaagaatatctcaagcatctagagattccaaggaaaatgttttcttgaattatgtggggaatccagaaacagaag
caaccccagtcaacaatgaaggaacacagcatatgtcagggccaaatgaattatcaacaccctcttccgaccgggatgcatttaacttgtctaca
aagctggaattggaaaagcaagtatttggtagtaggagtctaccacgtggcccagatagccaacgccaggatggaagagttcatgcttctcatc
aactaaccagagtccctgccaaacccatcctaccaacagcaacagtgaggctacctgaaatgtccacacaaagcgcttccagatactttgtaac
ttcccagtcacctcgtcactggaccaacaaaccggaaataactacatatccttctggggctttgccagagaacaaacagtttacaactccaagatt
atcaagtacaacaattcctctcccattgcacatgtccaaacccagcattcctagtaagtttactgaccgaagaactgaccaattcaatggttactcc
aaagtgtttggaaataacaacatccctgaggcaagaaacccagttggaaagcctcccagtccaagaattcctcattattccaatggaagactccc
tttctttaccaacaagactctttcttttccacagttgggagtcacccggagaccccagatacccacttctcctgccccagtaatgagagagagaaa
agttattccaggttcctacaacaggatacattcccatagcaccttccatctggactttggccctccggcacctccgttgttgcacactccgcagac
cacgggatcaccctcaactaacttacagaatatccctatggtctcttccacccagagttctatctcctttataacatcttctgtccagtcctcaggaa
gcttccaccagagcagctcaaagttctttgcaggaggacctcctgcatccaaattctggtctcttggggaaaagccccaaatcctcaccaagtcc
ccacagactgtgtccgtcaccgctgagacagacactgtgttcccctgtgaggcaacaggaaaaccaaagcctttcgttacttggacaaaggttt
ccacaggagctcttatgactccgaataccaggatacaacggtttgaggttctcaagaacggtaccttagtgatacggaaggttcaagtacaagat
cgaggccagtatatgtgcaccgccagcaacctgcacggcctggacaggatggtggtcttgctttcggtcaccgtgcagcaacctcaaatccta
gcctcccactaccaggacgtcactgtctacctgggagacaccattgcaatggagtgtctggccaaagggaccccagccccccaaatttcctgg
atcttccctgacaggagggtgtggcaaactgtgtcccccgtggagagccgcatcaccctgcacgaaaaccggaccctttccatcaaggaggc
gtccttctcagacagaggcgtctataagtgcgtggccagcaatgcagccggggcggacagcctggccatccgcctgcacgtggcggcactg
ccccccgttatccaccaggagaagctggagaacatctcgctgcccccggggctcagcattcacattcactgcactgccaaggctgcgcccct
gcccagcgtgcgctgggtgctcggggacggtacccagatccgcccctcgcagttcctccacgggaacttgtttgttttccccaacgggacgct
ctacatccgcaacctcgcgcccaaggacagcgggcgctatgagtgcgtggccgccaacctggtaggctccgcgcgcaggacggtgcagct
gaacgtgcagcgtgcagcagccaacgcgcgcatcacgggcacctccccgcggaggacggacgtcaggtacggaggaaccctcaagctg
gactgcagcgcctcgggggacccctggccgcgcatcctctggaggctgccgtccaagaggatgatcgacgcgctcttcagttttgatagcag
aatcaaggtgtttgccaatgggaccctggtggtgaaatcagtgacggacaaagatgccggagattacctgtgcgtagctcgaaataaggttggt
gatgactacgtggtgctcaaagtggatgtggtgatgaaaccggccaagattgaacacaaggaggagaacgaccacaaagtcttctacgggg
gtgacctgaaagtggactgtgtggccaccgggcttcccaatcccgagatctcctggagcctcccagacgggagtctggtgaactccttcatgc
agtcggatgacagcggtggacgcaccaagcgctatgtcgtcttcaacaatgggacactctactttaacgaagtggggatgagggaggaagga
gactacacctgctttgctgaaaatcaggtcgggaaggacgagatgagagtcagagtcaaggtggtgacagcgcccgccaccatccggaaca
agacttacttggcggttcaggtgccctatggagacgtggtcactgtagcctgtgaggccaaaggagaacccatgcccaaggtgacttggttgtc
cccaaccaacaaggtgatccccacctcctctgagaagtatcagatataccaagatggcactctccttattcagaaagcccagcgttctgacagc
ggcaactacacctgcctggtcaggaacagcgcgggagaggataggaagacggtgtggattcacgtcaacgtccagccacccaagatcaac
ggtaaccccaaccccatcaccaccgtgcgggagatagcagccgggggcagtcggaaactgattgactgcaaagctgaaggcatccccacc
ccgagggtgttatgggcttttcccgagggtgtggttctgccagctccatactatggaaaccggatcactgtccatggcaacggttccctggacat
caggagtttgaggaagagcgactccgtccagctggtatgcatggcacgcaacgagggaggggaggcgaggttgatcgtgcagctcactgtc
ctggagcccatggagaaacccatcttccacgacccgatcagcgagaagatcacggccatggcgggccacaccatcagcctcaactgctctg
ccgcggggaccccgacacccagcctggtgtgggtccttcccaatggcaccgatctgcagagtggacagcagctgcagcgcttctaccacaa
ggctgacggcatgctacacattagcggtctctcctcggtggacgctggggcctaccgctgcgtggcccgcaatgccgctggccacacggag
aggctggtctccctgaaggtgggactgaagccagaagcaaacaagcagtatcataacctggtcagcatcatcaatggtgagaccctgaagct
cccctgcacccctcccggggctgggcagggacgtttctcctggacgctccccaatggcatgcatctggagggcccccaaaccctgggacgc
gtttctcttctggacaatggcaccctcacggttcgtgaggcctcggtgtttgacaggggtacctatgtatgcaggatggagacggagtacggcc
cttcggtcaccagcatccccgtgattgtgatcgcctatcctccccggatcaccagcgagcccaccccggtcatctacacccggcccgggaaca
ccgtgaaactgaactgcatggctatggggattcccaaagctgacatcacgtgggagttaccggataagtcgcatctgaaggcaggggttcag
gctcgtctgtatggaaacagatttcttcacccccagggatcactgaccatccagcatgccacacagagagatgccggcttctacaagtgcatgg
caaaaaacattctcggcagtgactccaaaacaacttacatccacgtcttctgaaatgtggattccagaatgattgcttaggaactgacaacaaag
cggggtttgtaagggaagccaggttggggaataggagctcttaaataatgtgtcacagtgcatggtggcctctggtgggtttcaagttgaggttg
atcttgatctacaattgttgggaaaaggaagcaatgcagacacgagaaggagggctcagccttgctgagacactttcttttgtgtttacatcatgc
caggggcttcattcagggtgtctgtgctctgactgcaatttttcttcttttgcaaatgccactcgactgccttcataagcgtccataggatatctgagg
aacattcatcaaaaataagccatagacatgaacaacacctcactaccccattgaagacgcatcacctagttaacctgctgcagtttttacatgata
gactttgttccagattgacaagtcatctttcagttatttcctctgtcacttcaaaactccagcttgcccaataaggatttagaaccagagtgactgata
tatatatatatattttaattcagagttacatacatacagctaccattttatatgaaaaaagaaaaacatttcttcctggaactcacttttatataatgtttta
tatatatattttttcctttcaaatcagacgatgagactagaaggagaaatactttctgtcttattaaaattaataaattattggtctttacaagacttggat
acattacagcagacatggaaatataattttaaaaaatttctctccaacctccttcaaattcagtcaccactgttatattaccttctccaggaaccctcc
agtggggaaggctgcgatattagatttccttgtatgcaaagtttttgttgaaagctgtgctcagaggaggtgagaggagaggaaggagaaaact
gcatcataactttacagaattgaatctagagtcttccccgaaaagcccagaaacttctctgcagtatctggcttgtccatctggtctaaggtggctg
cttcttccccagccatgagtcagtttgtgcccatgaataatacacgacctgttatttccatgactgctttactgtatttttaaggtcaatatactgtacat
ttgataataaaataatattctcccaaaaaaaaaa

| | | |
|---|---|---|
| AI798118 | 3 | tttcaaatgttgccaaagtcatgggaactctagttaggaagatttcccctacttaga |
| | | gcatgtcactctggcgtggaccttcattcatctctttccccggcctggggttgatcc |
| | | attctcccttcttcccccttctcctgccctgcccccaccttctcagtcacaatctttgt |
| | | cttcccagtttggcaggacatcccaggggcccctgggctttccttcctgaggtta |
| | | gaagatggtgctttgaaagaacaattgctcctaccacccatcaaatcaaactttta |
| | | gaagctgataaaaaccttaaaaatcattcaatctcctcttctttctccttctgtagatg |
| | | aggaatctgagggtctaagaggagacatgagctgctgagattgactgagctag |
| | | gcagggccagacatagactccacgggcaggcccgcagtctttccaccaggtc |
| | | aggccactccctcgcccagggctcaagattccttttagagttcaacagagcagct |
| | | cccgagaacagaccagccctcaagactcaaatttcag |
| AK000168 | 4 | Sequence below. |

gttttctcatttgcaaccacccatgctggcatgaaagaaagtgaagctgctgggttgaactccatcttctgggggacatttgcagcctgcagggg
cctggcaatcttttttgctacctgtttacagcctggaaccatgattgtgttgagcaacattggcagcctgacttcatctttatttctggtgctttttgaca
agaacccaatttgtctctggatagcaacttcagtgtatggggcttcaatggcaaccacatttccgagtggtgtttcttggattgagcagtacacgac
catccatgggaaatctgcagcattttttgtaattggtgcttccctgggagaaatggctattcctgcagtcattggaattcttcaaggaaaataccctg
atttgcctgtagttctgtatacctctttgggagcatcaatagctactggtattttatttcctgtgctatataaattagccacttcacctcttgatcgccagc
gaaaagaagacagaaagagtgaggaccagaaagctctgctctctagctccgggctaaatgaatatgaggaagagaatgaagaggaggatg
cagaaaaatggaatgaaatggattttgaaatgattgaaacgaatgatacaatgaggcattctataatagagacatctagaagtagtctgacggag
cccacagctgaagtctataatcaatacccatcaaatgcactggtgtttgagtcttctccttttaatactggcagtgcccatgtgaagcacttgccag
aaaccaggacaaaagggactaacgtttagagaagatggattactcactgacatctttgaataactgccacttctaaggacgccattcagagcag
agcattagcagaatttcaccatgctttggggattaaaatttttaggtccatattatagcaaatgctaaaaatttttgaaatgttctgtaattcccagagt
cttccataaataaccaaatggtctcatacacgtacaataggatcttgttataacgctatcatttgacatgtgactgattaggaaatattttatggtctcc
acccctcagagcacccaaagaaggcattgttttitgagaaggggggtattctcagaatgagaccatgtaggagttgaattgattggttgaacaaat
tatcaggtatcaatatttttccaggaaagttgaggagaatagtttcataaaagagaaatgggccgggattacaggcttgagcccccgcgcccag
ccatcaaaatgctttttatttctgcatatgttgaatactttttacaatttaaaaaaatgatctgttttgaaggcaaaattgcaaatcttgaaattaagaagg
caaaaatgtaaaggagtcaaaactataaatcaagtatttgggaagtgaagactggaagctaatttgcattaaattcacaaacttttatactctttctgt
atatacattttttttctttaaaaaacaactatggatcagaatagccacatttagaacactttttgttatcagtcaatatttttagatagttagaacctggtcc
taagcctaaaagtgggcttgattctgcagtaaatcttttacaactgcctcgacacacataaacctttttaaaaatagacactccccgaagtcttttgtt
cgcatggtcacacactgatgcttagatgttccagtaatctaatatggccacagtagtcttgatgaccaaagtcctttttttccatctttagaaaactac
atgggaacaaacagatcgaacagttttgaagctactgtgtgtgtgaatgaacactcttgctttattccagaatgctgtacatctattttggattgtatat
tgtgtttgtgtatttacgctttgattcatagtaacttcttatggaattgatttgcattgaacacaaactgtaaataaaaagaaatggctgaaagaaaaaa
aaaaaaaaaaaaaaaaaaaaaaaaaa

| | | |
|---|---|---|
| acute myeloid | 5 | NM_001001890 |
| leukemia 1 | | NM_001754 |
| (AML-1) | | |
| (TEL/AML1; or | | |
| ETV6/RUNX1) | | |
| ankyrin repeat | 6 | BC015422 |
| domain 9 | | BC027479 |
| (ANKRD9) | | NM_152326 |
| AV758342 | 7 | atttganccccttnccttgaanccccatcgtatcgggacgcgcgccattgtgttggta |
| | | cccgggaattcggccattatggccgggagaaacgagatgggggtgaagctggag |
| | | atatttcggatgataatctacctcactttccctgtggctatgttctgggtttccaatcagg |
| | | ccgagtggtttgaggacgatgtcatacagcgcaagagggagctgtggccacctga |
| | | gaagcttcaagagatagaggaattcaaagagaggttacggaagcggcgggagga |
| | | gaagctccttcgcgacgcccagcagaactcctgaggcctccaagtgggagtccta |
| | | gcccctcccctgatgaaatatacatatactcagttccttgttaagaaaaaaaaannaa |
| | | aaaaaaaaaaaaaaaaacatgtcggccgcctcggcccagtcgactctagactcga |
| | | gcaagcttatgcatgcggccgcaattcgagctcacttggccaattcgccctatagtg |
| | | agtcgtatcacaattcactggcccgcgttttacaacgtcgtgacttggaaaaactcttg |
| | | ccgttacccaacctaaattgcctttgaacaaaaatcccctttgcgcaagttggctaata |
| | | tccaagagagcccgaaccaatcggcctttccaacagttgcccagcttgatgccaat |
| | | gaaattgtgagccttaaattttggttaaattcgggtaaatttttggttaatcagctcaatt |
| | | ttaacccaaaggccatggcccttt |
| AW242409 | 8 | atttcaaacaaatccaaaacttatttttgtttatagaaacaagacaacacacagtaaaa |
| | | tgaggaaatacaatagttttttcaatatagtaccacaagccattttttctgtaaaggacc |
| | | aaatattagatactatcacaaaacattttatcactcattcaaaacaagaatcattcagtt |
| | | gccccaggaaaaaacacactgaaattattttagatagcataataagctagaggatat |
| | | agaacatttgaaaaacagttccaaaaaatggaatactaaagatgcaacattaattctt |
| | | agtacatttgttatggtaatgaattaaagcattatagaatatattcaagtcagaaatcct |
| | | gtagtatgaagacataccttcatatgtgaaaatattattttatattaataataattattctg |
| | | gtatttatatcagatttatcatcagattttctttcctgaatttattaataatttgaccatggaa |
| | | cacaaaaatatttcattcattttataatactttg |
| AW510657 | 9 | tttgatatcattaaagtttattcaagaattgtttgacagacaacccttttaggtaagga |
| | | aaaaagtaactactgaagaacaaagaaagttccaagagggaattaactctagttc |
| | | tacaaattcaggactatacagtgacgattaggagctgagttcatgccttttagaact |
| | | aattacaattgttcgtaagtacgagtttaacataaatctacagctcttttctagagtac |
| | | aatagtaactaaaatagctggttttacatgacaggaaacacaatgtcctttgtaaca |
| | | ggtaaatactaaaaaagtacaattttttcctttttttccctcatataaatacataatgta |
| | | gggggataaataatacaaaaaagaaaatattttaacaggctataaccaataaatat |
| | | atatgaaaatgttcactagaacacacgcttttttgagcaatgctggacttctgcagg |
| | | cccagacatctctcacagttgtttttacatccattaaaatgtaaattctaagtgcaac |
| | | aaaagtgtcctgtcag |
| AW517716 | 10 | ttacgttttatttttatttatttactttttttagagatggagtctcactctgttgccaggctgg |
| | | agtgcagcggtgtgatcttggctcactgcaacttccgccccctgggttcaagtgatt |
| | | tcctgcctcagccttccaagtagctgggattacaggcatgcaccaccacacctggc |
| | | aatttttgtatctttagtagagacggggtttcaccatgttggccaggatggtcttgatct |
| | | cttgacctggtgatctgcctgcctcagcctcccaaagtgctgggatacaggtgtgag |
| | | ccaccgtgcctggcctatgtatttttttttttaaacacacacataaatctaaagcaaagg |
| | | gtatgccaagattcagctggggccaggtctgacccccactctgagcatctcattagc |
| | | ttcccatcagtggaatacaatggagacatattccacttggtctggcaatgtcctcc |
| B2-bradykinin | 11 | X86163; Sequence below. |
| receptor | | |
| (BDKRB2) | | |

cagattcacaaactgcaggactgggcagggagcagacagtgagcaaacgccagcagggctgctgtgaatttgtgtaaggattgagggacag
ttgcttttcagcatgggcccaggaatgccaaggagacatctatgcacgaccttgggaaatgagttgatgtctccggtaaaacaccggagactaa
ttcctgccctgcccaattttgcagggagcatggctgtgaggatggggtgaactcacgcacagccaaggactccaaaatcacaacagcattact
gttcttatttgctgccacacctgagccagcctgctccttcccaggagtggaggaggcctggggggagggagaggagtgactgagcttccctcc
cgtgtgttctccgtccctgccccagcaagacaacttagatctccaggagaactgccatccagctttggtgcaatggctgagtgcacaagtgagtt
gttgccctgggtttctttaatctattcagctagaactttgaaggacaatttcttgcattaataaaggttaagccctgaggggtccctgataacaacct
ggagaccaggattttatggctcccctcactgatggacaaggaggtctgtgccaaagaagaatccaataagcacatattgagcacttgctgtatat
gcagtattgagcactgtaggcaagacccaagaaagagaaggagccatctccatcttgaaggaactcaaagactcaagtgggaacgactggg
cactgccaccaccagaaagctgttcgacgagacggtcgagcagggtgctgtgggtgatatggacagcagaagggggagaccaaggttcca
gctcaaccaataactattgcacaaccacctgtccctgcctcagttcccttttatgtaacatgaagtcgttgtgagggttaaaggcagtaacaggtat
aaagtacttagaaaagcaaagggtgctacgtacatgtgaggcatcattacgcagacgtaactgggatatgtttactataaggaaaagacactga
ggtctagaaatagctccgtggagcagaatcagtattgggagccggtggcggtgtgaagcaccagtgtctggcacacagtaggtgctcattgg
ctcccttccacctgtcattcccaccaccctgaggccccaaccgccacacacacaggagcatttggagagaaggccatgtcttcaaagtctgattt
gtgatgaggcagaggaagatatttctaatcggtcttgcccagaggatcacagtgctgagaccccccaccaccagccggtacctgggaagggg
gagagtgcaggcctgctcagggactgttcctgtctcagcaaccaagggattgttcctgtcaatcaatggtttattggaaggtggcccagtatgag
ccctagaagagtgtgaaaaggaatggcaatggtgttcaccatcggcagtgccagggcagcactcattcacttgataaatgaatatttattagctg
gttggagagctagaacctggagagctagaacctggagaactagaacctggagggctagaacctggagaggctagaaccaagaagggctag
aacctggaggggctagaacctagagaagctaaaacctgagctagaagctggaggactagaacctggagggctggaatctgaagggctaga
acctggagggctggaatctggagagctagaacctggagggctagaacctggagggctagaacctagaagggctagaacctggagggctg
gaatctggagagctagaacctggagggctagaacctggagggctagaacctagaagggctagaacctggagggctagaacctggcaggtt
agaacctagaagggctagaacctggagagccagaacctggagggctagaacctggaagggctagaacctgtagagctagaacatggaga
gctagaacccggcaggctagaacctggcaagctagaacctggagggaatgaacctggagggctagaacctggagaatgagaaaaatttaca
tggcaaagagcccataaatcctgaccaatccaactctgaattttaaagcaaaagcgtgaaaaaaaagattccctccttacccccaacccactcttt
tttcccaccacccactctcctctgcctcagtaagtatctggaggaagaaaacaggtgaaagaagaagtaaaaaccatttagtattagtattagaat
gaagtcaaactgtgccacacatggtgaatgaaaaaaaaaaaaaagaggctgtgttttgtcacacagggcagtcattcagcaccagagcacgtg
atggtctgagactctcttaggagcagagctctgccgcaatggccatgtggggatccacacctggtctgaggggcaactgagtctgcgggaga
agagcggccctatgcatggtgtagatgccctgataaagaacatctgtcctgtgaaagactcaatgagctgttatgttgtaaacaggaagcatttc
acatccaaacgagaaaatcatgtaaacatgtgtcttttctgtagagcataataaatggatgaggtttttgcaaaaaaaaaaaaaaaaa

| | | |
|---|---|---|
| C6orf37 | 12 | AF350451; Sequence below |

attttccccgcacaactgctaaagctccagagacacgagcgtgtgtggcagcaagagccgccagttcgggaccaccgcagctggggtggca
gcggcgcaggaggggtcgcggggagggagtggtgagcgcaggcggcaggggtctgggaaagacgaagtcgctatttgctgtctgagcg
cgctcgcagctcctggaagtgttgccgcctctcggtttgtctcgcctgctgcgctcctagaagggcggccgcctccaggactgaccagggcca
agtggcgctcggcgggcactacatggcggagggtgaagggtacttcgccatgtctgaggacgagctggcctgcagcccctacatcccccta
ggcggcgacttcggcggcggcgacttcggcggcggcgacttcggcggcggcggcacgttcggtgggcattgcttggactattgcgaaagc
cctacggcgcactgcaatgtgctgaactgggagcaagtgcagcggctggacggcatcctgagcgagaccattccgattcacgggcgcggca
acttccccacgctcgagctgcagccgagcctgatcgtgaaggtggtgcggcggcgcctggccgagaagcgcattggcgtccgcgacgtgc
gcctcaacggctcggcagccagccatgtcctgcaccaggacagcggcctgggctacaaggacctggacctcatcttctcggccgacctgcg
cggggaaggggagtttcagactgtgaaggacgtcgtgctggactgcctgttggacttcttacccgagggggtgaacaaagagaagatcacac
cactcacgctcaaggaagcttatgtgcagaaaatggttaaagtgtgcaatgactctgaccgatggagtcttatatccctgtcaaacaacagtggc
aaaaatgtggaactgaaatttgtggattccctccggaggcagtttgaattcagtgtagattcttttcaaatcaaattagactctcttctgctcttttatga
atgttcagagaacccaatgactgagacatttcaccccacaataatcggggagagcgtctatggcgatttccaggaagcctttgatcacctttgtaa
caagatcattgccaccaggaacccagaggaaatccgagggggaggcctgcttaagtactgcaacctcttggtgaggggctttaggcccgcct
ctgatgaaatcaagacccttcaaaggtatatgtgttccaggtttttcatcgacttctcagacattggagagcagcagagaaaactggagtcctattt
gcagaaccactttgtgggattggaagaccgcaagtatgagtatctcatgacccttcatggagtggtaaatgagagcacagtgtgcctgatggga
catgaaagaagacagactttaaaccttatcaccatgctggctatccgggtgttagctgaccaaaatgtcattcctaatgtggctaatgtcacttgct
attaccagccagccccctatgtagcagatgccaactttagcaattactacattgcacaggttcagccagtattcacgtgccagcaacagacctac
tccacttggctaccctgcaattaagaatcatttaaaaatgtcctgtggggaagccatttcagacaagacaggagagaaaaaaaaaaaaaagaaa
aaaaaaagagtgatccagcccttattagggatgtgttttgtgcaatgatgatatgctcctggttttaagtttggcaaagcttatgtatcttttaatagat
gtgggagcatgatctcgaaaggatccttttcccttctcttattctcctacccaattggattctatcctgcaaaaaaagagagacctgtcattagaagc
aaccaggttctcctgatacaagagaagaaatgtgtgatgacaatatgggtttgctgtatctgctcccatagctttgccataggaaaaaaaaaagtg
gaaagtttcttttaagatggaattcataaaagggaaaatacggaggaaaaaaggtctcactccaacttgtgaatcagtttaggagttcagatattaa
tagtaacaatacaggaaaaaggggaactccaacgttgggattactgtctgaggcttgtagcaagtgctttctgtggaatgatcttgttttgctaaca
aacggcttgctccaaatgaacagtagtaggttggtgcagttctcgtaacaatcagcagaacttatgatgacacaatccattaattccagctgcgtg
catagatcacatttttaaaatgtaaaaatgcaagcaaaaacagctgtaacaaagaaagtgtgctcaaggaccaaagatttaacagataaaaatac
ccaattagaagagatatagtagactatatgaagagagattatatttgttacacaccaatatacatcaaagtgcctgttgccttctgaaaatttgaagt
ggcaaaattattttatggtttaatgattattttattttatcagggactgcctcaagaagaaaataacataagcttgtgaatggtggagaaaatgcccta
ttttttcttgcaaatacttgtataaagttaacatttgttgatctgatattatcataggtacatgtgtatgtgtgtataaattatatgtgtgtgtgtatatata
cattttatatatacattttatatgtatatatacacagtagattgactatgatctagaataatgtctcaaataggaaatgtttaaatactgtgtgtttttatgt
tttcaacaggataacatgagacgtgggcatattgcaatgatgaattaaatccacatctaaaaaaattaaatgaaggagggaaccaagtaatatatttcat
aggaagagcagaaattatactgttttagtgggatttttttttctttttttttttttctttggtgagccataaaattccacaaatgggagaatatttgtttggc
agagcactcttttttatattgaactgccattttgacagttggaacccatttattaaaaaaaaaattgcattcctctatgatgtttaatctagtggatcatgg
atcagtaataggctacttaaatccctgactgctaaaaaggatttccggtgatctaaacactacttgctaatgtttaaatgaattttaatgaatgcattct
gcatttctggaccactagaatttagtaatgtgaaatgaccctttttacagaatatttgcacaattgcttaaaatttatatatgagatatatattatatataa
cattttataaatcatgtcaatatgaaacatctttgatctggttgtcacactgcatttaaatatttagtactgtactttaaatcgctttccattaaatcaaatc
caactttattttctttcttacaaaaataccagttatacctttgtgaaatgaactggcattactatttcagttcaataacagctaatcctaaaaccacccttt
ctcctagccagtagttcctctagatactggtctctgaaaatgcatttgttaaaaacaaaacaaaactaacacataagaaccttccctttgtgttgtga
aacaaccacataatctccacaaccttagtggatgactgcttgctatgataattcctcgaagacccaattagaagattttcatcatcagttaaagaga
gaccacgggagaaaaaaatatcctcctgttggcagtataatttgtttgtttgtttatctagggatcctcagatgcttagtgctaggttaatccaggtta
atccgtctggactaccttttgtgcatctttctttgaagccttaatgggaacctgatgggtttgctgtagcagcttccttgtgaattctgtcagagctgca
acagccgctgcactgccactcagttttctaaggaactcctcctactaccatcttggctcagtctccctcacttaagccctgggtttgaaaaattaatt
gcaacttcccaggaaacattgttcagtttgcagattaagcctggcactcacctatcagaaaccagagctccgcctgcttagttgtttcaaagttttct
gaaagaaaactaggggagcacttgtgaacacaggagcagctggtgatctgctttcttaccctaactcttgacaaatgagtcgtctactattttaaa
gagtctggaggtctctgactctgccataacaataacctgctgttaatttataacacagatttttgtttggaagagccttatttgaaatacactttgattta
ttttcttaaatatttatattcttttcttgcttacttcagggttggtagcttagttggaagtgccagcacctggcacctattcatatagaacaggctgtactc
aagacaacttctagcatttactttaagacttatataatttatttctattttgtgtgtactatagtcttgtgcatatgtagttgaacacacagtgaaatatatg
tctctctttgtggatgtgcggcctaaaaatttgaatgtctggtgagagagagccatgtgtataggtcagagaaaagaacagctcccgactccctat
tagcgcctgtgatttgtttccttttgtgtttatctggcctagtgtgctgtttctttaaaccaggaagaagttttgtcttttggaggctcttctcacctgtcca
gcctggcatgtcagagaacacatagcctgtgacaatgccgtttttaaaggtttacttaatttgcagtaaatccagctgcctcaagaactcctacacc
aagatggacatttcctttccagaaatgggatcaagtatctgctcactttggtattggatggactaataatgtagctccaaaaatgcaaggatggaa
gaatatgtgtaatccaaaccaaggaaggaaatgaaaagtgaacgtactgtttttaccacccctttctgtttgcttattgttggttgcttcactgtgcat
aaagtgttttcaatgcaacgcttgttaaataaatattgtgaactattttgtaaatgaaatgtattatgttgaaagctgtcagttcaaaaataagctttttt
gttgttgttgaagatgaagtgtgttaggtgaaaccaaaaagccaaaaaaagtaatttcatatatagcatctatttgaatataatctttctttaaaatttct
tttagcatagcattttcagtgctaagaaagaatctctatgttatattttgttaaaataatggctttctaacaaagcaaatggtaaagtacaaagttggaa
gatgtcaagttaacgagacttgctgcaaagccttgcagaacggaggaggctcgcctgctggctgtctctccctccaacctctctacaatcatgcc
tgctttgaggtgttctgttgcagcaagctgcaccttgggtcactcttttggaatattttgactataggctgcgtcacaggcagaaaaggagttgatg
gaaaatggactaaaaaactgacatgtttgaatcagtgctagagggaacagattgtgaattttgtttacagcatccaatatttggatttttttgtaaata
aaaaagttatttttttctattg

| | | |
|---|---|---|
| C6orf55 | 13 | BC022536 |
| | | NM_016485 |
| | | AF271994 |
| C6orf69 | 14 | BC023525 |
| | | BC035879 |
| C7orf32 | 15 | BC015899, AF452639, NM_145230, AK057700, |
| | | AK094602, AK098362, AK172725, BX640846 |
| C11orf8 | 16 | NM_001584, U57911 |
| CD24 antigen | 17 | BC064619, AK026603, BC007674 NM_013230, |
| | | AK125531, D87667, L33930 |
| CDP- | 18 | NM_001263 |
| diacylglycerol | | U60808 |
| synthase | | U65887 |
| (CDS1) | | |
| centaurin, | 19 | NM_018404 |
| alpha 2 | | AJ272195 |
| (CENTA2) | | |
| claudin 1 | 20 | NM_021101, AF086514, AF101051 |
| (CLDN1) | | |
| collagen, type | 21 | NM_001854 |
| XI, alpha 1 | | NM_080629 |
| (COL11A1) | | NM_080630 |
| | | AB208844 |
| collagen, type | 22 | NM_001855; Sequence below |
| XV, alpha 1 | | |
| (COL15A1) | | |

ttctgcccgccgccgccgctgccgagcgccgcctttgttccctgcaggaagggcgagcgcgcgggccagcgctcagccagcgcctcacga
cccttcgtcctccgctaagctccaacgctctgctcgactagccgcgcgccttccggggctccgcagacccgcgagatggcaccaaggagga
acaacgggcagtgctggtgtctgctgatgctgctctcggtctccacgcccctccctgctgtcacccagacccgcggtgcgacagagactgctt
cccagggtcacctggacctcacgcagctcatcggtgtcccgctgccctcgtccgtatcctttgtcacaggctatggtggcttcccggcctacagt
ttcgggcctggtgccaatgttggccgcccagccaggactctcatcccatccaccttcttcagggacttcgccatcagggtcgtggtgaagccca
gcagcacccgtggtggcgtgctcttcgccatcactgacgccttccagaaggtcatctacctgggcctgcggctctcaggtgtggaggacggc
caccagcggatcatcctctactacacggagccaggctcccatgtgtcccaagaagctgctgccttctcggtgcctgtgatgacccacaggtgg
aaccgcttcgccatgattgtccagggtgaggaagtgaccctcctcgtgaactgtgaggagcacagccgcatccccttccagcggtcctcccag
gctttggcttttgagtccagcgctggaatcttcatgggcaatgcaggagctacagggctcgagagattcactggctccctccagcagctcaccg
tgcaccccgaccccaggactcccgaggagctgtgtgaccctgaagagtcctcggcatctggagagaccagtgggctgcaggaggcagacg
gagtagctgagatcttagaagccgtcacctacactcaagcctcgcccaaagaagcaaaagttgaacccataaacacacctccaactccatcct
ccccctttgaagacatggaactttctggtgaacctgtacccgaggggaccctggaaaccaccaacatgagcatcatccagcacagcagcccc
aaacaagggtctggtgagatcctgaatgacacactggagggggttcattctgtggatggtgaccccattactgacagcggctcaggggctgg
ggccttccttgacattgctgaagaaaagaatttagcagcaacagcagcggggctggccgaggtgcccatcagcactgctggagaagcagag
gccagcagtgtgcccaccgggggaccaaccctctctatgtccacggagaacccagaggaaggggtcactccaggtccagataatgaagag
cgtttacgagcaacagcagcaggggaggccgaggcactcgccagcatgcctggggaagtggaggccagtggtgtggcccccggggagct
ggacctctccatgtccgcccagagcctcggggaagaggccactgtgggtccaagcagtgaagacagtttaacaacagctgcagctgcaacc
gaagtgtccctcagtacttttgaggatgaggaagccagtggggtccccacagatggcctggctcccctcacagccaccatggcccctgagcg
ggcagtcacttctggtcctggtgatgaagaagacttggcagcagccacaacagaggagcccctcatcacagctgggggtgaagagtccggc
agccctccccctgatgggccaccgctgcccctgcccacagtggctcctgaaagatggatcactccagctcaaagagaacatgtgggaatgaa
aggacaggctgggcccaaaggagaaaagggtgatgctggggaggagcttcctggccctcctgaaccttctgggcctgttggacccacggca
ggagcagaagcagagggctctggcctaggctggggctcggacgtcggctctggctctggtgacctggtgggcagtgagcagctgctgaga
ggtcctccaggacccccagggccacctggcttacctgggattccaggaaaaccaggaactgatgttttcatgggaccccctggatctcctgga
gaggatggacctgctggtgaacctgggcccccgggccctgagggacagcctggagttgatggagccaccggccttcccgggatgaaagg
ggagaagggagcaagagggcctaatggctcagttggtgaaaagggtgaccctggcaacagaggcttacctggacccccggggaaaaagg
gacaagctggccctcctggggtcatgggacccccagggcctcctggaccccctgggcccccaggccctggatgcacaatgggacttggatt
cgaggataccgaaggctctggaagcacccagctattgaatgaacccaaactctccagaccaacggctgcaattggtctcaaaggagagaaag
gagaccggggacccaagggagaaagggggatggatggagccagtattgtgggaccccctgggccgagagggccacctgggcacatcaa
ggtcttgtctaattccttgatcaatatcacccatggattcatgaatttctcggacattcctgagctggtggggcctccggggccggacgggttgcct
gggctgccaggatttccaggtcctagaggaccaaaaggtgacactggtttacctggctttccaggactaaaaggagaacagggcgagaagg
gagagccgggtgccatcctgacagaggacattcctctggaaaggctgatggggaaaaagggtgaacctggaatgcatggagccccaggac
caatggggcccaaaggaccaccaggacataaaggagaatttggccttcccgggcgacctggtcgcccaggactgaatggcctcaagggtac
caaaggagatccaggggtcattatgcagggcccacctggcttacctggccctccaggcccccctgggccacctggagctgtgattaacatca
aaggagccattttcccaatacccgtccgaccacactgcaaaatgccagttgatactgctcatcctgggagtccagagctcatcacttttcacggt
gttaaaggagagaaaggatcctggggtcttcctggctcaaagggagaaaaaggcgaccagggagcccagggaccaccaggtcctccactt
gatctagcttacctgagacactttctgaacaacttgaagggggagaatggagacaaggggttcaaaggtgaaaaaggagaaaaaggagacat
taatggcagcttccttatgtctgggcctccaggcctgcccggaaatccaggcccggctggccaaaaaggggagacagtcgttgggccccaag
gacccccaggtgctcctggtctgcctgggccacctggctttggaagacctggtgatcctgggccaccggggcccccggggccaccaggacc
tccagctatcctgggagcagctgtggcccttccaggtccccctggccctccaggacagccagggcttcccggatccagaaacctggtcacag
cattcagcaacatggatgacatgctgcagaaagcgcatttggttatagaaggaacattcatctacctgagggacagcactgagtttttcattcgtg
ttagagatggctggaaaaaattacagctgggagaactgatccccattcctgccgacagccctccaccccctgcgctttccagcaacccacatca
gcttctgcctccaccaaaccctatttcaagtgccaattatgagaagcctgctctgcatttggctgctctgaacatgccattttctggggacattcga
gctgattttcagtgcttcaagcaggccagagctgcaggactgttgtccacctaccgagcattcttatcttcccatttgcaagatctgtccaccattgt
gaggaaagcagagagatacagccttcccatagtgaacctcaagggccaagtactttttaataattgggactcaattttttctggccacggaggtc
agttcaatatgcatattccaatatactcctttgatggtcgagacataatgacagatccttcttggccccagaaagtcatttggcatggctccagccc
ccatggcgtccgccttgtggataactactgtgaagcatggcgaaccgcggacacagcggtcacgggacttgcctccccgctgagcacgggg
aagattctggaccagaaagcatacagctgtgctaatcggctaattgtcctatgtatcgaaaacagtttcatgacagacgctaggaagtaatggcc
ttctgatgattcttaaagagttttcaattttttcttatgtgaagagttgacactgaaatctaaaatgtttaattgttgtaaatattacagttttttttttttt
actacatattctttacaacagcaaccaaagaaaacatacctcaatacactcaaaactgaagacatagaggactcagatcaaagacaaaatctgatccat
atattggtgctagattctgcaggaaaccccagcagtgtgaacgcatcccaacataggttaagagcaagttgaaaacaaaggccagattctgcca
ctgcatccttcagacagttatatcctccttttaaaccattgttgttgagtgtaagatgtccttcatgttttcttataaagtcagtgtttagaaatgttaccct
ttctaagttatatacagatcaaatgcttttttctttcacgtacatccatcatttgcaactgctgttcgtacacagaaacaggactgctcaaatgatcctat
ttgtattttctgatgctatcagactctaatgtttttttccctaaaatattattgccatcatgctttaggaatttttatatttttacacaatcatattttagta
tggtgtctgtttatgtaactctgacttgctggaaaagttgaaactccaaataatctgaaactagaaaagaaatagcacataattactaccttccccttggcg
gctctcctcccccaacccccaccccacaattttatgacttccatttggcaattgttgaattataactgcgactgaaacaaacaggttcatagagatg
aattttctgagaaacatatatctacatgttgtataattggattttttttccatgtaagtgaacataaaaacatcttttccggg

| | | |
|---|---|---|
| collagen, type | 23 | NM_152888 |
| XXII, alpha 1 | | AF406780 |
| (COL22A1) | | BC042075 |
| chondroitin | 24 | NM_004385; Sequence below |
| sulfate | | |
| proteoglycan 2 | | |
| (CSPG2) | | |

gctgccccgagcctttctggggaagaactccaggcgtgcggacgcaacagccgagaacattaggtgttgtggacaggagctgggaccaag
atcttcggccagccccgcatcctcccgcatcttccagcaccgtcccgcaccctccgcatccttccccgggccaccacgcttcctatgtgacccg
cctgggcaacgccgaacccagtcgcgcagcgctgcagtgaattttccccccaaactgcaataagccgccttccaaggccaagatgttcataaa
tataaagagcatcttatggatgtgttcaaccttaatagtaacccatgcgctacataaagtcaaagtgggaaaaagcccaccggtgaggggctcc
ctctctggaaaagtcagcctaccttgtcatttttcaacgatgcctactttgccacccagttacaacaccagtgaatttctccgcatcaaatggtctaa
gattgaagtggacaaaaatggaaaagatttgaaagagactactgtccttgtggcccaaaatggaaatatcaagattggtcaggactacaaaggg
agagtgtctgtgcccacacatcccgaggctgtgggcgatgcctccctcactgtggtcaagctgctggcaagtgatgcgggtctttaccgctgtg
acgtcatgtacgggattgaagacacacaagacacggtgtcactgactgtggatggggttgtgtttcactacagggcggcaaccagcaggtaca
cactgaattttgaggctgctcagaaggcttgtttggacgttggggcagtcatagcaactccagagcagctctttgctgcctatgaagatggatttg
agcagtgtgacgcaggctggctggctgatcagactgtcagatatcccatccgggctcccagagtaggctgttatggagataagatgggaaag
gcaggagtcaggacttatggattccgttctccccaggaaacttacgatgtgtattgttatgtggatcatctggatggtgatgtgttccacctcactgt
ccccagtaaattcaccttcgaggaggctgcaaaagagtgtgaaaaccaggatgccaggctggcaacagtgggggaactccaggcggcatg
gaggaacggctttgaccagtgcgattacgggtggctgtcggatgccagcgtgcgccaccctgtgactgtggccagggcccagtgtggaggt
ggtctacttggggtgagaaccctgtatcgttttgagaaccagacaggcttccctccccctgatagcagatttgatgcctactgctttaaacctaaa
gaggctacaaccatcgatttgagtatcctcgcagaaactgcatcacccagtttatccaaagaaccacaaatggtttctgatagaactacaccaatc
atccctttagttgatgaattacctgtcattccaacagagttccctcccgtgggaaatattgtcagttttgaacagaaagccacagtccaacctcagg
ctatcacagatagtttagccaccaaattacccacacctactggcagtaccaagaagccctgggatatggatgactactcaccttctgcttcagga
cctcttggaaagctagacatatcagaaattaaggaagaagtgctccagagtacaactggcgtctctcattatgctacggattcatgggatggtgt
cgtggaagataaacaaacacaagaatcggttacacagattgaacaaatagaagtgggtcctttggtaacatctatggaaatcttaaagcacattc
cttccaaggaattccctgtaactgaaacaccattggtaactgcaagaatgatcctggaatccaaaactgaaaagaaaatggtaagcactgtttctg
aattggtaaccacaggtcactatggattcaccttgggagaagaggatgatgaagacagaacacttacagttggatctgatgagagcaccttgat
ctttgaccaaattcctgaagtcattacggtgtcaaagacttcagaagacaccatccacactcatttagaagacttggagtcagtctcagcatccac
aactgtttcccctttaattatgcctgataataatggatcatccatggatgactgggaagagagacaaactagtggtaggataacggaagagtttctt
ggcaaatatctgtctactacaccttttccatcacagcatcgtacagaaatagaattgtttccttattctggtgataaaatattagtagagggaatttcc
acagttatttatccttctctacaaacagaaatgacacatagaagagaaagaacagaaacactaataccagagatgagaacagatacttatacaga
tgaaatacaagaagagatcactaaaagtccatttatgggaaaaacagaagaagaagtcttctctgggatgaaactctctacatctctctcagagc
caattcatgttacagagtcttctgtggaaatgaccaagtcttttgatttcccaacattgataacaaagttaagtgcagagccaacagaagtaagaga
tatggaggaagactttacagcaactccaggtactacaaaatatgatgaaaatattacaacagtgcttttggcccatggtactttaagtgttgaagca
gccactgtatcaaaatggtcatgggatgaagataatacaacatccaagcctttagagtctacagaaccttcagcctcttcaaaattgccccctgcc
ttactcacaactgtggggatgaatggaaaggataaagacatcccaagtttcactgaagatggagcagatgaatttactcttattccagatagtact
caaaagcagttagaggaggttactgatgaagacatagcagcccatggaaaattcacaattagatttcagccaactacatcaactggtattgcaga
aaagtcaactttgagagattctacaactgaagaaaaagttccacctatcacaagcactgaaggccaagtttatgcaaccatggaaggaagtgctt
tgggtgaagtagaagatgtggacctctctaagccagtatctactgttccccaatttgcacacacttcagaggtggaaggattagcatttgttagtta
tagtagcacccaagagcctactacttatgtagactcttcccataccattcctctttctgtaattcccaagacagactggggagtgttagtaccttctgt
tccatcagaagatgaagttctaggtgaaccctctcaagacatacttgtcattgatcagactcgccttgaagcgactatttctccagaaactatgaga
acaacaaaaatcacagagggaacaactcaggaagaattcccttggaaagaacagactgcagagaaaccagttcctgctctcagttctacagct
tggactcccaaggaggcagtaacaccactggatgaacaagagggcgatggatcagcatatacagtctctgaagatgaattgttgacaggttct
gagagggtcccagttttagaaacaactccagttggaaaaattgatcacagtgtgtcttatccaccaggtgctgtaactgagcacaaagtgaaaac
agatgaagtggtaacactaacaccacgcattgggccaaaagtatctttaagtccagggcctgaacaaaaatatgaaacagaaggtagtagtac
aacaggatttacatcatctttgagtccttttagtacccacattacccagcttatggaagaaaccactactgagaaaacatccctagaggatattgatt
taggctcaggattatttgaaaagcccaaagccacagaactcatagaattttcaacaatcaaagtcacagttccaagtgatattaccactgccttca
gttcagtagacagacttcacacaacttcagcattcaagccatcttccgcgatcactaagaaaccacctctcatcgacagggaacctggtgaaga
aacaaccagtgacatggtaatcattggagaatcaacatctcatgttcctcccactacccttgaagatattgtagccaaggaaacagaaaccgata
ttgatagagagtatttcacgacttcaagtcctcctgctacacagccaacaagaccacccactgtggaagacaaagaggcctttggacctcaggc
gctttctacgccacagcccccagcaagcacaaaatttcaccctgacattaatgtttatattattgaggtcagagaaaataagacaggtcgaatgag
tgatttgagtgtaattggtcatccaatagattcagaatctaaagaagatgaaccttgtagtgaagaaacagatccagtgcatgatctaatggctga
aattttacctgaattccctgacataattgaaatagacctataccacagtgaagaaaatgaagaagaagaagaagagtgtgcaaatgctactgatg
tgacaaccaccccatctgtgcagtacataaatgggaagcatctcgttaccactgtgcccaaggacccagaagctgcagaagctaggcgtggc
cagtttgaaagtgttgcaccttctcagaatttctcggacagctctgaaagtgatactcatccatttgtaatagccaaaacggaattgtctactgctgt
gcaacctaatgaatctacagaaacaactgagtctcttgaagttacatggaagcctgagacttaccctgaaacatcagaacatttttcaggtggtga
gcctgatgttttccccacagtcccattccatgaggaatttgaaagtggaacagccaaaaaaggggcagaatcagtcacagagagagatactga
agttggtcatcaggcacatgaacatactgaacctgtatctctgtttcctgaagagtcttcaggagagattgccattgaccaagaatctcagaaaat
agcctttgcaagggctacagaagtaacatttggtgaagaggtagaaaaaagtacttctgtcacatacactcccactatagttccaagttctgcatc
agcatatgtttcagaggaagaagcagttaccctaataggaaatccttggccagatgacctgttgtctaccaaagaaagctgggtagaagcaact
cctagacaagttgtagagctctcagggagttcttcgattccaattacagaaggctctggagaagcagaagaagatgaagatacaatgttcaccat
ggtaactgatttatcacagagaaatactactgatacactcattactttagacactagcaggataatcacagaaagcttttttgaggttcctgcaacca
ccatttatccagtttctgaacaaccttctgcaaaagtggtgcctaccaagtttgtaagtgaaacagacacttctgagtggatttccagtaccactgtt
gaggaaaagaaaaggaaggaggaggagggaactacaggtacggcttctacatttgaggtatattcatctacacagagatcggatcaattaattt
taccctttgaattagaaagtccaaatgtagctacatctagtgattcaggtaccaggaaaagttttatgtccttgacaacaccaacacagtctgaaag
ggaaatgacagattctactcctgtctttacagaaacaaatacattagaaaatttgggggcacagaccactgagcacagcagtatccatcaacctg
gggttcaggaagggctgaccactctcccacgtagtcctgcctctgtctttatggagcagggctctggagaagctgctgccgacccagaaacca
ccactgtttcttcattttcattaaacgtagagtatgcaattcaagccgaaaaggaagtagctggcactttgtctccgcatgtggaaactacattctcc
actgagccaacaggactggttttgagtacagtaatggacagagtagttgctgaaaatataacccaaacatccagggaaatagtgatttcagagc
gattaggagaaccaaattatggggcagaaataaggggcttttccacaggttttcctttggaggaagatttcagtggtgactttagagaatactcaa
cagtgtctcatcccatagcaaaagaagaaacggtaatgatggaaggctctggagatgcagcatttagggacacccagacttcaccatctacagt
acctacttcagttcacatcagtcacatatctgactcagaaggacccagtagcaccatggtcagcacttcagccttcccctgggaagagtttacatc
ctcagctgagggctcaggtgagcaactggtcacagtcagcagctctgttgttccagtgcttcccagtgctgtgcaaaagttttctggtacagcttc
ctccattatcgacgaaggattgggagaagtgggtactgtcaatgaaattgatagaagatccaccattttaccaacagcagaagtggaaggtacg
aaagctccagtagagaaggaggaagtaaaggtcagtggcacagtttcaacaaactttccccaaactatagagccagccaaattatggtctagg
caagaagtcaaccctgtaagacaagaaattgaaagtgaaacaacatcagaggaacaaattcaagaagaaaagtcatttgaatcccctcaaaac
tctcctgcaacagaacaaacaatctttgattcacagacatttactgaaactgaactcaaaaccacagattattctgtactaacaacaaagaaaactt
acagtgatgataaagaaatgaaggaggaagacacttctttagttaacatgtctactccagatccagatgcaaatggcttggaatcttacacaactc
tccctgaagctactgaaaagtcacattttttcttagctactgcattagtaactgaatctataccagctgaacatgtagtcacagattcaccaatcaaa
aaggaagaaagtacaaaacattttccgaaaggcatgagaccaacaattcaagagtcagatactgagctcttattctctggactgggatcaggag
aagaagttttacctactctaccaacagagtcagtgaattttactgaagtggaacaaatcaataacacattatatccccacacttctcaagtggaaag
tacctcaagtgacaaaattgaagactttaacagaatggaaaatgtggcaaaagaagttggaccactcgtatctcaaacagacatctttgaaggta
gtgggtcagtaaccagcacaacattaatagaaattttaagtgacactggagcagaaggacccacggtggcacctctccctttctccacggacat
cggacatcctcaaaatcagactgtcaggtgggcagaagaaatccagactagtagaccacaaaccataactgaacaagactctaacaagaattc
ttcaacagcagaaattaacgaaacaacaacctcatctactgattttctggctagagcttatggttttgaaatggccaaagaatttgttacatcagcac
caaaaccatctgacttgtattatgaaccttctggagaaggatctggagaagtggatattgttgattcatttcacacttctgcaactactcaggcaacc
agacaagaaagcagcaccacatttgtttctgatgggtccctggaaaaacatcctgaggtgccaagcgctaaagctgttactgctgatggattccc
aacagtttcagtgatgctgcctcttcattcagagcagaacaaaagctcccctgatccaactagcacactgtcaaatacagtgtcatatgagaggtc
cacagacggtagtttccaagaccgtttcagggaattcgaggattccaccttaaaacctaacagaaaaaaacccactgaaaatattatcatagacc
tggacaaagaggacaaggatttaatattgacaattacagagagtaccatccttgaaattctacctgagctgacatcggataaaaatactatcatag
atattgatcatactaaacctgtgtatgaagacattcttggaatgcaaacagatatagatacagaggtaccatcagaaccacatgacagtaatgatg
aaagtaatgatgacagcactcaagttcaagagatctatgaggcagctgtcaacctttctttaactgaggaaacatttgagggctctgctgatgttct
ggctagctacactcaggcaacacatgatgaatcaatgacttatgaagatagaagccaactagatcacatgggctttcacttcacaactgggatcc
ctgctcctagcacagaaacagaattagacgttttacttcccacggcaacatccctgccaattcctcgtaagtctgccacagttattccagagattga
aggaataaaagctgaagcaaaagccctggatgacatgtttgaatcaagcactttgtctgatggtcaagctattgcagaccaaagtgaaataatac
caacattgggccaatttgaaaggactcaggaggagtatgaagacaaaaaacatgctggtccttcttttcagccagaattctcttcaggagctgag
gaggcattagtagaccatactccctatctaagtattgctactacccaccttatggatcagagtgtaacagaggtgcctgatgtgatggaaggatcc
aatcccccatattacactgatacaacattagcagtttcaacatttgcgaagttgtcttctcagacaccatcatctcccctcactatctactcaggcagt
gaagcctctggacacacagagatcccccagcccagtgctctgccaggaatagacgtcggctcatctgtaatgtccccacaggattcttttaagg
aaattcatgtaaatattgaagcaactttcaaaccatcaagtgaggaataccttcacataactgagcctccctctttatctcctgacacaaaattagaa
ccttcagaagatgatggtaaacctgagttattagaagaaatggaagcttctcccacagaacttattgctgtggaaggaactgagattctccaagat
ttccaaaacaaaaccgatggtcaagtttctggagaagcaatcaagatgtttcccaccattaaaacacctgaggctggaactgttattacaactgcc
gatgaaattgaattagaaggtgctacacagtggccacactctacttctgcttctgccacctatggggtcgaggcaggtgtggtgccttggctaag
tccacagacttctgagaggcccacgctttcttcttctccagaaataaaccctgaaactcaagcagctttaatcagagggcaggattccacgatag
cagcatcagaacagcaagtggcagcgagaattcttgattccaatgatcaggcaacagtaaaccctgtggaatttaatactgaggagcaacacc
accattttcccttctggagacttctaatgaaacagatttcctgattggcattaatgaagagtcagtggaaggcacggcaatctatttaccaggacct
gatcgctgcaaaatgaacccgtgccttaacggaggcacctgttatcctactgaaacttcctacgtatgcacctgtgtgccaggatacagcggag
aacagtgtgaacttgattttgatgaatgtcactctaatccctgtcgtaatggagccacttgtgttgatggttttaacacattcaggtgcctctgccttcc
aagttatgttggtgcactttgtgagcaagataccgagacatgtgactatggctggcacaaattccaagggcagtgctacaaatactttgcccatc
gacgcacatgggatgcagctgaacgggaatgccgtctgcagggtgcccatctcacaagcatcctgtctcacgaagaacaaatgtttgttaatcg
tgtgggccatgattatcagtggataggcctcaatgacaagatgtttgagcatgacttccgttggactgatggcagcacactgcaatacgagaatt
ggagacccaaccagccagacagcttcttttctgctggagaagactgtgttgtaatcatttggcatgagaatggccagtggaatgatgttccctgc
aattaccatctcacctatacgtgcaagaaaggaacagttgcttgcggccagccccctgttgtagaaaatgccaagacctttggaaagatgaaac
ctcgttatgaaatcaactccctgattagataccactgcaaagatggtttcattcaacgtcaccttccaactatccggtgcttaggaaatggaagatg
ggctatacctaaaattacctgcatgaacccatctgcataccaaaggacttattctatgaaatactttaaaaattcctcatcagcaaaggacaattcaa
taaatacatccaaacatgatcatcgttggagccggaggtggcaggagtcgaggcgctgatccctaaaatggcgaacatgtgttttcatcatttca
gccaaagtcctaacttcctgtgcctttcctatcacctcgagaagtaattatcagttggtttggatttttggaccaccgttcagtcattttgggttgccgt
gctcccaaaacattttaaatgaaagtattggcattcaaaaagacagcagacaaaatgaaagaaaatgagagcagaaagtaagcatttccagcct
atctaatttctttagttttctatttgcctccagtgcagtccatttcctaatgtataccagcctactgtactatttaaaatgctcaatttcagcaccgatggc
catgtaaataagatgatttaatgttgattttaatcctgtatataaaataaaaagtcacaatgagtttgggcatatttaatgatgattatggagccttaga
ggtctttaatcattggttcggctgcttttatgtagtttaggctggaaatggtttcacttgctctttgactgtcagcaagactgaagatggcttttcctgg
acagctagaaaacacaaaatcttgtaggtcattgcacctatctcagccataggtgcagtttgcttctacatgatgctaaaggctgcgaatgggatc
ctgatggaactaaggactccaatgtcgaactcttctttgctgcattcctttttcttcacttacaagaaaggcctgaatggaggacttttctgtaaccagg

| | | |
|---|---|---|
| Defensin, | 25 | BC033298, BC047677, NM_005218, X92744 |
| beta 1 | | ctccaaaggagccagcgtctccccagttcctgaaatcctgggtgttgcctgccagtc |
| (DEFB1) | | gccatgagaacttcctaccttctgctgtttactctctgcttacttttgtctgagatggcct |
| | | caggtggtaactttctcacaggccttggccacagatctgatcattacaattgcgtcag |
| | | cagtggagggcaatgtctctattctgcctgcccgatctttaccaaaattcaaggcacc |
| | | tgttacagagggaaggccaagtgctgcaagtgagctgggagtgaccagaagaaa |
| | | gacgcagaagtgaaatgaactttttataagcattcttttaataaaggaaaattgcttttg |
| | | aagtataaaaaaaaaaaaaaaaaaaaaaaaaaaaaaaaaa |
| Desmoglein 2 | 26 | NM_001943, BC099656, BC099657, BC099655, Z26317 |
| (DSG2) | | BC014581, BC014584, BC017410, BC022413 |
| | | sequence below |

ggcgcggagcggtgcggcggcgggaggcggaggcgagggtgcgatggcgcggagcccgggacgcgcgtacgccctgctgcttctcct
gatctgctttaacgttggaagtggacttcacttacaggtcttaagcacaagaaatgaaaataagctgcttcctaaacatcctcatttagtgcggcaa
aagcgcgcctggatcaccgcccccgtggctcttcgggagggagaggatctgtccaagaagaatccaattgccaagatacattctgatcttgca
gaagaaagaggactcaaaattacttacaaatacactggaaaagggattacagagccaccttttggtatatttgtctttaacaaagatactggagaa
ctgaatgttaccagcattcttgatcgagaagaaacaccattttttctgctaacaggttacgctttggatgcaagaggaaacaatgtagagaaaccc
ttagagctacgcattaaggttcttgatatcaatgacaacgaaccagtgttcacacaggatgtctttgttgggtctgttgaagagttgagtgcagcac
atactcttgtgatgaaaatcaatgcaacagatgcagatgagcccaataccctgaattcgaaaatttcctatagaatcgtatctctggagcctgcttat
cctccagtgttctacctaaataaagatacaggagagatttatacaaccagtgttaccttggacagagaggaacacagcagctacactttgacagt
agaagcaagagatggcaatggagaagttacagacaaacctgtaaaacaagctcaagttcagattcgtattttggatgtcaatgacaatatacctg
tagtagaaaataaagtgcttgaagggatggttgaagaaaatcaagtcaacgtagaagttacgcgcataaaagtgttcgatgcagatgaaatagg
ttctgataattggctggcaaattttacatttgcatcaggaaatgaaggaggttatttccacatagaaacagatgctcaaactaacgaaggaattgtg
acccttattaaggaagtagattatgaagaaatgaagaatcttgacttcagtgttattgtcgctaataaagcagcttttcacaagtcgattaggagtaa
atacaagcctacacccattcccatcaaggtcaaagtgaaaaatgtgaaagaaggcattcattttaaaagcagcgtcatctcaatttatgttagcga
gagcatggatagatcaagcaaaggccaaataattggaaattttcaagcttttgatgaggacactggactaccagcccatgcaagatatgtaaaat
tagaagatagagataattggatctctgtggattctgtcacatctgaaattaaacttgcaaaacttcctgattttgaatctagatatgttcaaaatggca
catacactgtaaagattgtggccatatcagaagattatcctagaaaaaccatcactggcacagtccttatcaatgttgaagacatcaacgacaact
gtcccacactgatagagcctgtgcagacaatctgtcacgatgcagagtatgtgaatgttactgcagaggacctggatggacacccaaacagtg
gccctttcagtttctccgtcattgacaaaccacctggcatggcagaaaaatggaaaatagcacgccaagaaagtaccagtgtgctgctgcaaca
aagtgagaaaaagcttgggagaagtgaaattcagttcctgatttcagacaatcagggttttagttgtcctgaaaagcaggtccttacactcacagt
ttgtgagtgtctgcatggcagcggctgcagggaagcacagcatgactcctatgtgggcctgggacccgcagcaattgcgctcatgattttggc
ctttctgctcctgctattggtaccacttttactgctgatgtgccattgcggaaagggcgccaaaggctttacccccatacctggcaccatagagat
gctgcatccttggaataatgaaggagcaccacctgaagacaaggtggtgccatcatttctgccagtggatcaagggggcagtctagtaggaag
aaatggagtaggaggtatggccaaggaagccacgatgaaaggaagtagctctgcttccattgtcaaagggcaacatgagatgtccgagatgg
atggaaggtgggaagaacacagaagcctgctttctggtagagctacccagtttacaggggccacaggcgctatcatgaccactgaaaccacg
aagaccgcaagggccacaggggcttccagagacatggccggagctcaggcagctgctgttgcactgaacgaagaattcttaagaaattatttc
actgataaagcggcctcttacactgaggaagatgaaaatcacacagccaaagattgccttctggtttattctcaggaagaaactgaatcgctgaa
tgcttctattggttgttgcagttttattgaaggagagctagatgaccgcttcttagatgatttgggacttaaattcaagacactagctgaagtttgcct
gggtcaaaaaatagatataaataaggaaattgagcagagacaaaaacctgccacagaaacaagtatgaacacagcttcacattcactctgtga
gcaaactatggttaattcagagaatacctactcctctggcagtagcttcccagttccaaaatctttgcaagaagccaatgcagagaaagtaactca
ggaaatagtcactgaaagatctgtgtcttctaggcaggcgcaaaaggtagctacacctcttcctgacccaatggcttctagaaatgtgatagcaa
cagaaacttcctatgtcacagggtccactatgccaccaaccactgtgatcctgggtcctagccagccacagagccttattgtgacagagagggt
gtatgctccagcttctaccttggtagatcagccttatgctaatgaaggtacagttgtggtcactgaaagagtaatacagcctcatgggggtggatc
gaatcctctggaaggcactcagcatcttcaagatgtaccttacgtcatggtgagggaaagagagagcttccttgcccccagctcaggtgtgcag
cctactctggccatgcctaatatagcagtaggacagaatgtgacagtgacagaaagagttctagcacctgcttccactctgcaatccagttacca
gattcccactgaaaattctatgacggctaggaacaccacggtgtctggagctggagtccctggccctctgccagattttggtttagaggaatctg
gtcattctaattctaccataaccacatcttccaccagagttaccaagcatagcactgtacagcattcttactcctaaacagcagtcagccacaaact
gacccagagtttaattagcagtgactaatt

| | | |
|---|---|---|
| EGF-like- | 27 | NM_015507, BC038587, AY358333, AF186084, |
| domain, | | DQ031339 |
| multiple 6 | | NM_001408, AF234887, AK124727, D87469 |
| (EGFL6) | | Sequence below |

ccgcagaggagcctcggccaggctagccagggcgcccccagcccctccccaggccgcgagcgcccctgccgcggtgcctggcctcccct
cccagactgcagggacagcacccggtaactgcgagtggagcggaggacccgagcggctgaggagagaggaggcggcggcttagctgct
acggggtccggccggcgccctcccgaggggggctcaggaggaggaaggaggacccgtgcgagaatgcctctgccctggagccttgcgct
cccgctgctgctctcctgggtggcaggtggtttcgggaacgcggccagtgcaaggcatcacgggttgttagcatcggcacgtcagcctgggg
tctgtcactatggaactaaactggcctgctgctacggctggagaagaaacagcaagggagtctgtgaagctacatgcgaacctggatgtaagtt
tggtgagtgcgtgggaccaaacaaatgcagatgctttccaggatacaccgggaaaacctgcagtcaagatgtgaatgagtgtggaatgaaac
cccggccatgccaacacagatgtgtgaatacacacggaagctacaagtgcttttgcctcagtggccacatgctcatgccagatgctacgtgtgt
gaactctaggacatgtgccatgataaactgtcagtacagctgtgaagacacagaagaagggccacagtgcctgtgtccatcctcaggactccg
cctggccccaaatggaagagactgtctagatattgatgaatgtgcctctggtaaagtcatctgtccctacaatcgaagatgtgtgaacacatttgg
aagctactactgcaaatgtcacattggtttcgaactgcaatatatcagtggacgatatgactgtatagatataaatgaatgtactatggatagccata
cgtgcagccaccatgccaattgcttcaatacccaagggtccttcaagtgtaaatgcaagcagggatataaaggcaatggacttcggtgttctgct
atccctgaaaattctgtgaaggaagtcctcagagcacctggtaccatcaaagacagaatcaagaagttgcttgctcacaaaaacagcatgaaaa
agaaggcaaaaattaaaaatgttaccccagaacccaccaggactcctacccctaaggtgaacttgcagcccttcaactatgaagagatagtttc
cagaggcgggaactctcatggaggtaaaaaagggaatgaagagaaaatgaaagaggggcttgaggatgagaaaagagaagagaaagccc
tgaagaatgacatagaggagcgaagcctgcgaggagatgtgtttttccctaaggtgaatgaagcaggtgaattcggcctgattctggtccaaag
gaaagcgctaacttccaaactggaacataaagatttaaatatctcggttgactgcagcttcaatcatgggatctgtgactggaaacaggatagag
aagatgattttgactggaatcctgctgatcgagataatgctattggcttctatatggcagttccggccttggcaggtcacaagaaagacattggcc
gattgaaacttctcctacctgacctgcaaccccaaagcaacttctgtttgctctttgattaccggctggccggagacaaagtcgggaaacttcgag
tgtttgtgaaaaacagtaacaatgccctggcatgggagaagaccacgagtgaggatgaaaagtggaagacagggaaaattcagttgtatcaa
ggaactgatgctaccaaaagcatcatttttgaagcagaacgtggcaagggcaaaaccggcgaaatcgcagtggatggcgtcttgcttgtttcag
gcttatgtccagatagccttttatctgtggatgactgaatgttactatctttatatttgactttgtatgtcagttccctggtttttttgatattgcatcatag
gacctctggcattttagaattactagctgaaaaattgtaatgtaccaacagaaatattattgtaagatgcctttcttgtataagatatgccaatatttgctt
taaatatcatatcactgtatcttctcagtcatttctgaatctttccacattatattataaaatatggaaatgtcagtttatctcccctcctcagtatatctga
tttgtataagtaagttgatgagcttctctctacaacatttctagaaaatagaaaaaaaagcacagagaaatgtttaactgtttgactcttatgatacttct
tggaaactatgacatcaaagatagacttttgcctaagtggcttagctgggtctttcatagccaaacttgtatatttaaattctttgtaataataatatcca
aatcatcaaaaaaaaaaaaaaaaa

| | | |
|---|---|---|
| Eukaryotic | 28 | AK027011, BC078179, AK074361, AL137627, |
| translation | | AL137676, AL157497, BC009350, NM_001013703, |
| initiation | | AB037759, AJ243428 |
| factor 2 alpha | | |
| kinase 4 | | |
| (EIF2AK4) | | |
| EPB41L3 | 29 | AF515797-Sequence below |
| (Erythrocyte | | AL832598 NM_012307, AB023204, AK090875, |
| membrane | | AK094952 |
| protein band | | |
| 4.1-like 3) | | |

atttacaataaatgaagattaccctcaaatgctagaagctgtctaggtccgtccggtgtgtcagattttcctcagattagatgtgccaataaccaagt
ttattcagtaaacaacttgtacttgtttcatctggttttattactctcacccataaacaggaatgactctttgaccctctggaaatatgtaatgcttccaat
cttgctttgtgtatctcatttaatttgttataaggtagtactgattttagcatattaatgcgatttcttccttgttgtttgctttggtctgtgttcaatccag
agagcttaaattgtcattattttgggaagaaaacctgtatttttgttagtttacaatattatgaaatttcacttcaggagaaactgctgggcttcctgtggct
ttgttttcttagttactttttccgtgccgtgtattttttaattgatttttcttcttttacttgaaaagaaagtgttttattttcaaatctggtccatatttac
attctagttcagagccaagccttaaactgtacagaatttccactgtaattaaaactatttagtgttagttataaatagccttcaaaaagagagattctccatt
cacgatcacctgcatcacagcccatggtgaatgtatgtttctgcatagcgaaataaaaatggcaaatgcaaaaaaaaaaaaaaaaaaaaaaaaaaaa

| | | |
|---|---|---|
| Endothelial | 30 | gctgcttcccaccagcaaagaccacgactggagagccgagccggaggcagctg |
| cell-specific | | ggaaacatgaagagcgtcttgctgctgaccacgctcctcgtgcctgcacacctggt |
| molecule 1 | | ggccgcctggagcaataattatgcggtggactgccctcaacactgtgacagcagtg |
| (ESM1; | | agtgcaaaagcagcccgcgctgcgagaggacagtgctcgacgactgtggctgct |
| endocan) | | gccgagtgtgcgctgcagggcggggagaaacttgctaccgcacagtctcaggca |
| BCO11989 | | ggatggcatgaagtgtggcccggggctgaggtgtcagccttctaatggggaggat |
| | | ccttttggtgaagagtttggtatctgcaaagactgtccctacggcaccttcgggatgg |
| | | attgcagagagacctgcaactgccagtcaggcatctgtgacagggggacgggaa |
| | | aatgcctgaaattccccttcttccaatattcagtaaccaagtcttccaacagatttgtttc |
| | | tctcacggagcatgacatggcatctggagatggcaatattgtgagagaagaagttg |
| | | gaaagagaatgctgccgggtctcccgtaatgaggaaatggttaaatccacgctgat |
| | | cccggctgtgatttctgagagaaggctctattttcgtgattgttcaacacacagccaa |
| | | cattttaggaactttctagattatagcataaggacatgtaatttttgaagaccaaatgtg |
| | | atgcatggtggatccagaaaacaaaaagtaggatacttacaatccataacatccata |
| | | tgactgaacacttgtatgtgtttgttaaatattcgaatgcatgtagatttgttaaatgtgtg |
| | | tgtatagtaacactgaagaactaaaaatgcaatttaggtaatcttacatggagacagg |
| | | tcaaccaaagagggagctaggcaaagctgaagaccgcagtgagtcaaattagttc |
| | | ttgactttgatgtacattaatgttgggatatggaatgaagacttaagagcaggagaag |
| | | atggggagggggtgggagtgggaaataaaatatttagcccttccttggtaggtagc |
| | | tctctagaatttaattgtgctttttttttttggctttgggaaaagtcaaaataaaacaa |
| | | ccagaaaacccctgaaggaagtaagatgtttgaagcttatggaaatttgagtaacaaaca |
| | | gctttgaactgagagcaatttcaaaaggctgctgatgtagttcccgggttacctgtatt |
| | | gaaggacggttctggggcataggaaacacatacacttccataaatagctttaacgtatgc |
| | | cacctcagagataaatctaagaagtattttacccactggtggtttgtgtgtgtatgaagg |
| | | taaatatttatatatttttataaataaatgtgttagtgcaagtcatcttccctacccata |
| | | tttatcatcctcttgaggaaagaaatctagtattatttgttgaaaatggttagaataaaa |
| | | ctatgactctataaggttttcaaacatctgaggcatgataaatttattatccataattat |
| | | agtaataataaccttaataagcataagaaaaacagagtcactctggatttcaaaaatgtc |
| | | aaaaaaaaaaaaaa |
| F-box protein | 31 | NM_058229, NM_148177, BC024030, AJ420108, |
| 32 (FBXO32) | | AK056986 |
| FJX1 (Four | 32 | NM_014344, AJ245599, AK026069, BC049171, |
| jointed box 1) | | BC071898 |
| FOLH1 (Folate | 33 | BC025672 |
| hydrolase | | NM_004476 |
| (prostate- | | AF176574 |
| specific | | M99487 |
| membrane | | NM_001014986 |
| antigen) 1 | | |
| Family with | 34 | BC008865 |
| sequence | | NM_032231 |
| similarity 96, | | NM_001014812 |
| member A | | |
| (FLJ22875) | | |
| FLJ46072 | 35 | BC033256, NM_198488, AK127960 |
| Coagulation | 36 | BC012453 |
| factor II | | BC018130 |
| (thrombin) | | NM_005242 |
| receptor-like 1 | | |
| (F2RL1; | | |
| GPR11) | | |
| Frizzled 10 | 37 | AB027464, BC070037, NM_007197 |
| (FZD10) | | Sequence below |

acacgtccaacgccagcatgcagcgcccgggcccccgcctgtggctggtcctgcaggtgatgggctcgtgcgccgccatcagctccatgga
catggagcgcccgggcgacggcaaatgccagcccatcgagatcccgatgtgcaaggacatcggctacaacatgactcgtatgcccaacctg
atgggccacgagaaccagcgcgaggcagccatccagttgcacgagttcgcgccgctggtggagtacggctgccacggccacctccgcttct
tcctgtgctcgctgtacgcgccgatgtgcaccgagcaggtctctacccccatccccgcctgccgggtcatgtgcgagcaggcccggctcaagt
gctccccgattatggagcagttcaacttcaagtggcccgactccctggactgccggaaactccccaacaagaacgaccccaactacctgtgca
tggaggcgcccaacaacggctcggacgagcccacccggggctcgggcctgttcccgccgctgttccggccgcagcggccccacagcgcg
caggagcacccgctgaaggacgggggccccgggcgcggcggctgcgacaacccgggcaagttccaccacgtggagaagagcgcgtcg
tgcgcgccgctctgcacgcccggcgtggacgtgtactggagccgcgaggacaagcgcttcgcagtggtctggctggccatctgggcggtgc
tgtgcttcttctccagcgccttcaccgtgctcaccttcctcatcgacccggcccgcttccgctaccccgagcgccccatcatcttcctctccatgtg
ctactgcgtctactccgtgggctacctcatccgcctcttcgccggcgccgagagcatcgcctgcgaccgggacagcggccagctctatgtcat
ccaggagggactggagagcaccggctgcacgctggtcttcctggtcctctactacttcggcatggccagctcgctgtggtgggtggtcctcac
gctcacctggttcctggccgccggcaagaagtggggccacgaggccatcgaagccaacagcagctacttccacctggcagcctgggccatc
ccggcggtgaagaccatcctgatcctggtcatgcgcagggtggcgggggacgagctcaccggggtctgctacgtgggcagcatggacgtc
aacgcgctcaccggcttcgtgctcattcccctggcctgctacctggtcatcggcacgtccttcatcctctcgggcttcgtggccctgttccacatc
cggagggtgatgaagacgggcggcgagaacacggacaagctggagaagctcatggtgcgtatcgggctcttctctgtgctgtacaccgtgc
cggccacctgtgtgatcgcctgctacttttacgaacgcctcaacatggattactggaagatcctggcggcgcagcacaagtgcaaaatgaaca
accagactaaaacgctggactgcctgatggccgcctccatccccgccgtggagatcttcatggtgaagatctttatgctgctggtggtggggat
caccagcgggatgtggatttggacctccaagactctgcagtcctggcagcaggtgtgcagccgtaggttaaagaagaagagccggagaaaa
ccggccagcgtgatcaccagcggtgggatttacaaaaaagcccagcatccccagaaaactcaccacgggaaatatgagatccctgcccagt
cgcccacctgcgtgtgaacagggctggagggaagggcacaggggcgcccggagctaagatgtggtgcttttcttggttgtgtttttctttcttctt
cttcttttttttttttttataaaagcaaaagagaaatacataaaaaagtgtttaccctgaaattcaggatgctgtgatacactgaaaggaaaaatgtact
taaagggttttgttttgttttggttttccagcgaagggaagctcctccagtgaagtagcctcttgtgtaactaatttgtggtaaagtagttgattcagcc
ctcagaagaaaacttttgtttagagccctccgtaaatatacatctgtgtatttgagttggctttgctacccatttacaaataagaggacagataactg
ctttgcaaattcaagagcctcccctgggttaacaaatgagccatccccagggcccacccccaggaaggccacagtgctgggcggcatccctg
cagaggaaagacaggacccggggcccgcctcacaccccagtggatttggagttgcttaaaatagactctggccttcaccaatagtctctctgc
aagacagaaacctccatcaaacctcacatttgtgaactcaaacgatgtgcaatacatttttttctctttccttgaaaataaaaagagaaacaagtattt
tgctatatataaagacaacaaaagaaatctcctaacaaaagaactaagaggcccagccctcagaaacccttcagtgctacattttgtggcttttta
atggaaaccaagccaatgttatagacgtttggactgatttgtggaaaggaggggggaagagggagaaggatcattcaaaagttacccaaagg
gcttattgactctttctattgttaaacaaatgatttccacaaacagatcaggaagcactaggttggcagagacactttgtctagtgtattctcttcaca
gtgccaggaaagagtggtttctgcgtgtgtatatttgtaatatatgatatttttcatgctccactattttattaaaaataaaatatgttctttaaaaaaa

| | | |
|---|---|---|
| Glycoprotein | 38 | BC008151, BC047295, NM_005278, NM_001001994, |
| M6B (GPM6B) | | NM_001001996, U45955 |
| P2RY14 | 39 | BC034989 |
| (Purinergic | | NM_014879 |
| receptor P2Y, | | D13626 |
| G-protein | | |
| coupled, 14 | | |
| (GPR105) | | |
| Homeo box B7 | 40 | cgtaaaaccgacactaaaacgtccctgcctacaaatcatccggccaaattatga |
| HOXB7) | | gttcattgtattatgcgaatgctttattttctaaatatccagcctcaagttcggttttcg |
| BC015345 | | ctaccg |
| | | gagccttcccagaacaaacttcttgtgcgtttgcttccaacccccagcgcccggg |
| | | ctatggagcgggttcgggcgcttccttcgccgcctcgatgcagggcttgtaccc |
| | | cggcggggggggcatggcgggccagagcgcggccggcgtctacgcggcc |
| | | ggctatgggctcgagccgagttccttcaacatgcactgcgcgccctttgagcag |
| | | aacctctccggggtgtgtcccggcgactccgccaaggcggcgggcgccaag |
| | | gagcagagggactcggacttggcggccgagagtaacttccggatctacccctg |
| | | gatgcgaagctcaggaactgaccgcaaacgaggccgccagacctacacccg |
| | | ctaccagaccctggagctggagaaagaatttcactacaatcgctacctgacgcg |
| | | gcggcggcgcatcgagatcgcgcacacgctctgcctcacggaaagacagatc |
| | | aagatttggtttcagaaccggcgcatgaagtggaaaaaggagaacaagaccgc |
| | | gggcccggggaccaccggccaagacagggctgaagcagaggaggaagag |
| | | gaagagtgagggatggagaaagggcagaggaagagacatgagaaagggag |
| | | aggaagagaagcccagctctgggaactgaatcaggaaactcaaatcgaatag |
| | | ggaagtaaaaaaacaaaacaaaaaacaaaaaaaaaaaaaa |
| Immunoglobulin | 41 | NM_014333, BC047021, NM_145296, NM_021189, |
| superfamily, | | BC125103, AB017563, AK172730 AL080181 |
| member 4 | | |
| (IGSF4) | | |
| FLJ35593 fis | 42 | NM_006469, NM_016389, DQ050632, AK001273, |
| (IVNS1ABP) | | AK023020, AK092912, AK093912, AJ012449 |
| Potassium | 43 | BC060793 |
| channel, | | NM_003740 |
| subfamily K, | | AF084830 |
| member 5 | | AK001897 |
| (KCNK5) | | |
| Leucine zipper, | 44 | NM_021020 |
| putative tumor | | AF123659 |
| suppressor 1 | | |
| (LZTS1) | | |
| clone | 45 | BC038772 |
| IMAGE:5270727 | | Sequence below. |
| (MCM4) | | |

gctccgagtggcggttgtttcaagatggcggacgtggcgggcccctcccgccccagtgccgcggcgttctggagccgggacttttctgatga
agaacaatcagtagtatacgttccaggaatttctgctgaaggaaatgtcagatcaagacacaagctgatgagtccaaaagctgatgttaaactta
agacttccagggtgactgatgcttcaatctccatggagtccttaaaaggcacaggagattcagtagatgaacagaattcctgcaggggagaaat
aaagagtgcatcattgaaggatttatgtcttgaagacaaaagacgcattgcaaacttaattaaagaactggccagagtaagtgaggaaaaggaa
gtgacagaggaaagactgaaagctgagcaggagtcatttgagaagaagatcaggcagttggaagaacagaatgaactgatcatcaaagaaa
gggaagatatccttttgaaagttctcttttttaacgttcaagaagtttgactatttctttttttttttgagacgaaatctcgctcttgtcgcctaggctgcag
tgcaatggtgcgatttcagttcaccgcaacctccacctcccggttcaagcgattctcctaccccagctactcgggaggttgaggcaggagaatc
gcttaaacccaggcggcagaggttgcagtgagccaagatcgcgccactgcactccagcctcagcaatagagtgagactgtctcaaaaaaaaa
aaaaaaaaaaacctgccaattttcaaacataccgtagagattattttcaggtgccattttatagtatagcagcagggcttttactctgtgtatgcaca
gatgcagtctggggcatggtttgtgtgctggactttctcatggccatcatcagtatgcttatggatttgatgacaggcatagcctgggcatatcacc
tcattggtaaagggcatagagcctttcttttttatggcacttctttttttgagatagggtcttactctgtcaccctggctagagtacactggtacaatcac
ggctcaatgtaggcttaacctcctgggctcaggtgtatgtcactatgcccggctactttttgtattttttggtagagacggcttcgccacgttgccca
ggctgcaagcgatatgcctaggctcaagcgatctgcccacctcaacttccggaagtgctgagattacaggtgtgagccactgcacccagccttt
gttttattttttattttttgagaggtatgattctttctagagattttttctcatggctactattagatcaggaatgggtgattggagattattagattctagg
ttaacttctaccactttaccctaatacataaaactttttcctaaataaatgatggaaggaataatacttggttacctggcattatttttcagtaagaaaaaa
gctttactaaccactacatttatggaaatttgtaggggtaagtattttataggtcataaaaaacaccataatataacgaatctcattttctttaaatgtga
attaaatcctaacagtcatctttataaaatgaccataggctaaaatcttacgtgtaagtactactacaataaataatttctgaaacctttaaaaaaaaa
aaaaaaaaaaaaagaaaaaaaaaaaaaaaaa

| | | |
|---|---|---|
| Nidogen 2 | 46 | BC035608, NM_007361, AJ223500, D86425 |
| (osteonidogen) | | |
| (NID2) | | |
| Olfactomedin- | 47 | BC067274 |
| like 2B | | NM_015441 |
| (OlfM12B) | | BC047472 |
| Oxysterol | 48 | BC017731 |
| binding protein- | | AL832293 |
| like 3 | | |
| (OSBPL3) | | |
| Protocadherin | 49 | AL137505 |
| 17 (PCDH17) | | Sequence below. |

cggacgcgtgggcatgtatgtaccagcagtggttacttgcattgtgtagtgtttttcaagaggtctgggtcttaacaaaatgttttcctttatctcagt
gctcttctgcctctttttgttggtgtcctttgagaacaatacaccttctattccttcatttggttacacctttccttgtgacatttagcgagtttcaaactta
cttccatatgaggctaagaaacctcaaatttcaggaattgggaaaaataaaattagcacttgcagaagtagcagcagatgggaaaatgccttgatt
gacattttctttcagcatttaaaatttttggcattttacagcttcatgacaaacagttttgtgcccataccttagaaaatgtggtgctgagttaaataaag
gctgtttgagcactggagcagaaaaatgcattatttgcaaactggtggataattttgtgccttctcttctggccaccaagccagtgtagaaacagc
aaaaatgtcataaaaattcttatatttaaaacaaaaacaaaagcaaaaacaaacattgaattaaattaagttttgtaattttaaactttaaaaacttctac
tgaaaatatttccgccaaatgccatcaatattttagactgtacctcgtttgcaaaactgctttgagagggaagagtggacaactcccatcagcctta
ttctcttgagaactatattttggttcctagtaacagcctttccaaagctctactcttggtttttattactcataaatgtttaaattagaaaagaagggacct
tgtacatgtgaaacctaattgactctctatattttggacaatttatgtatctgaaatgtgttgtctctgttatatgatgttatttttgccaggagactacag
gttgatttagcttgatagctgaaatttgatggaaaactgatttccatttagtcttaccaagtgttgcttctctcttactagacagatatccacttagtaaa
atctaaagcagtatgtaaatgaaaccagcaaagagagtagggtttattttataaacattcttaatgctaagtaaccagttgttcaatttattatatgtgt
ctgaggacattaaaacaccataagattgtaataattggttgtgccaatgtgtgagggatttacctttaggctctctgtcaccagtgatttactagtgtt
agctgtttaacacattatctgtatttagtagtgattatttatttacaagttggtggtaattcagcagtcaggactctaagcttttatagttgaattgagga
aatctcgcttttattcatttagctggcaactgcctttattgcagacctctggtgcttggctttcaaggaagcctatgagatgccaaaatcacaccttta
gagagcaccttgctctaataggtgatgcatgagcaaacagtgagatttgaaggggttttaacataatttagaatgtgaaaaaaatatcaattcatat
ctttcaagtactaacccctcaaaaaagcccacacatacaaaatatgtgatgtgataccactttgtcttttaggtctttaagtaactgaagttaagcac
agaaaaaaaaatcacttcatggaaatttcagtaagaaacccaaacttctaaaaattgcttgcagatgagctagaaaaaaaaaaaaaaaaaaa

| | | |
|---|---|---|
| Phospholipase | 50 | NM_016341, AF117948, AF190642, AL050031, |
| C, epsilon 1 | | AB040949, AK022543 |
| (PLCE1) | | |
| Plexin domain | 51 | BC036059, AF279144, AY704670, AY704671, |
| containing 1 | | AY704672, AK093589, AK127539 |
| (PLXDC1) | | |
| PR domain | 52 | NM_001198 |
| containing 1, | | NM_182907 |
| with ZNF | | AY198414 |
| domain | | AY198415 |
| (PRDM1) | | |
| Response gene | 53 | BC066334 NM_014059, AF036549, AK091099, |
| to complement | | AK095079 |
| 32 (RGC32) | | |
| Regulator of | 54 | BC015510 |
| G-protein | | NM_002922 |
| signalling 1 | | |
| (RGS1) | | |
| Ribonucleotide | 55 | NM_001034 |
| reductase M2 | | X59618 |
| polypeptide | | |
| (RRM2) | | |
| Sodium | 56 | BC006526 |
| channel, | | Sequence below. |
| nonvoltage- | | |
| gated 1 alpha | | |
| (SCNN1A) | | |

gggcactcagggcccagagctcagccttgaccctgacccttgctctccccaatccactccggggctcatgaaggggaacaagctggaggag
caggaccctagacctctgcagcccataccaggtctcatggaggggaacaagctggaggagcaggactctagccctccacagtccactccag
ggctcatgaaggggaacaagcgtgaggagcaggggctgggccccgaacctgcggcgccccagcagcccacggcggaggaggaggcc
ctgatcgagttccaccgctcctaccgagagctcttcgagttcttctgcaacaacaccaccatccacggcgccatccgcctggtgtgctcccagc
acaaccgcatgaagacggccttctgggcagtgctgtggctctgcacctttggcatgatgtactggcaattcggcctgcttttcggagagtacttc
agctaccccgtcagcctcaacatcaacctcaactcggacaagctcgtcttccccgcagtgaccatctgcaccctcaatccctacaggtacccgg
aaattaaagaggagctggaggagctggaccgcatcacagagcagacgctctttgacctgtacaaatacagctccttcaccactctcgtggccg
gctcccgcagccgtcgcgacctgcgggggactctgccgcaccccttgcagcgcctgagggtcccgcccccgcctcacggggcccgtcgag
cccgtagcgtggcctccagcttgcgggacaacaacccccaggtggactggaaggactggaagatcggcttccagctgtgcaaccagaacaa
atcggactgcttctaccagacatactcatcaggggtggatgcggtgagggagtggtaccgcttccactacatcaacatcctgtcgaggctgcca
gagactctgccatccctggaggaggacacgctgggcaacttcatcttcgcctgccgcttcaaccaggtctcctgcaaccaggcgaattactctc
acttccaccacccgatgtatggaaactgctatactttcaatgacaagaacaactccaacctctggatgtcttccatgcctggaatcaacaacggtc
tgtccctgatgctgcgcgcagagcagaatgacttcattcccctgctgtccacagtgactggggcccgggtaatggtgcacgggcaggatgaa
cctgcctttatggatgatggtggctttaacttgcggcctggcgtggagacctccatcagcatgaggaaggaaaccctggacagacttgggggc
gattatggcgactgcaccaagaatggcagtgatgttcctgttgagaacctttacccttcaaagtacacacagcaggtgtgtattcactcctgcttcc
aggagagcatgatcaaggagtgtggctgtgcctacatcttctatccgcggccccagaacgtggagtactgtgactacagaaagcacagttcct
gggggtactgctactataagctccaggttgacttctcctcagaccacctgggctgtttcaccaagtgccggaagccatgcagcgtgaccagcta
ccagctctctgctggttactcacgatggccctcggtgacatcccaggaatgggtcttccagatgctatcgcgacagaacaattacaccgtcaac
aacaagagaaatggagtggccaaagtcaacatcttcttcaaggagctgaactacaaaaccaattctgagtctccctctgtcacgatggtcaccct
cctgtccaacctgggcagccagtggagcctgtggttcggctcctcggtgttgtctgtggtggagatggctgagctcgtctttgacctgctggtca
tcatgttcctcatgctgctccgaaggttccgaagccgatactggtctccaggccgagggggcaggggtgctcaggaggtagcctccaccctg
gcatcctcccctccttcccacttctgcccccaccccatgtctctgtccttgtcccagccaggccctgctccctctccagccttgacagcccctccc
cctgcctatgccaccctgggcccccgcccatctccagggggctctgcaggggccagttcctccacctgtcctctgggggggccctgagagg
gaaggagaggtttctcacaccaaggcagatgctcctctggtgggagggtgctggccctggcaagattgaaggatgtgcagggcttcctctca
gagccgcccaaactgccgttgatgtgtggaggggaagcaagatgggtaagggctcaggaagttgctccaagaacagtagctgatgaagctg
cccagaagtgccttggctccagccctgtaccccttggtactgcctctgaacactctggtttccccacccaactgcggctaagtctctttttcccttg
gatcagccaagcgaaacttggagctttgacaaggaactttcctaagaaaccgctgataaccaggacaaaacacaaccaagggtacacgcag
gcatgcacgggtttcctgcccagcgacggcttaagccagcccccgactggcctggccacactgctctccagtagcacagatgtctgctcctcc
tcttgaacttgggtgggaaaccccacccaaaagccccctttgttacttaggcaattccccttccctgactcccgagggctagggctagagcaga
cccgggtaagtaaaggcagacccagggctcctctagcctcatacccgtgccctcacagagccatgccccggcacctctgccctgtgtctttcat
acctctacatgtctgcttgagatatttcctcagcctgaaagtttccccaaccatctgccagagaactcctatgcatcccttagaaccctgctcagac
accattacttttgtgaacgcttctgccacatcttgtcttccccaaaattgatcactccgccttctcctgggctcccgtagcacactataacatctgctg
gagtgttgctgttgcaccatactttcttgtacatttgtgtctcccttcccaactagactgtaagtgccttgcggtcagggactgaatcttgcccgtttat
gtatgctccatgtctagcccatcatcctgcttggagcaagtaggcaggagctcaataaatgtttgttgcatgaagaaaaaaaaaaaaaaaaaaaa
aaaaaaaaaaaaaaaaaaaaaaaaa

| | | |
|---|---|---|
| cytochrome | 57 | AL021683 |
| oxidase | | NM_005138 |
| deficient | | |
| homolog 2 | | |
| (SCO2) | | |
| SlamF8 | 58 | AK074669 |
| (BLAME) | | Sequence below. |

acttttgggacatcctgttctgagtcaagattcctccttctgaacatgggactttccagaaggaccacagctcctcccgtgcatccactcggcctg
ggaggttctggattttggctgtcgagggagtttgcctgcctctccagagaaagatggtcatgaggcccctgtggagtctgcttctctgggaagcc
ctacttcccattacagttactggtgcccaagtgctgagcaaagtcgggggctcggtgctgctggtggcagcgcgtccccctggcttccaagtcc
gtgaggctatctggcgatctctctggccttcagaagagctcctggccacgtttttccgaggctccctagagactctgtaccattcccgcttcctgg
gccgagcccagctacacagcaacctcagcctggagctcgggccgctggagtctggagacagcggcaacttctccgtgttgatggtggacac
aaggggccagccctggacccagaccctccagctcaaggtgtacgatgcagtgcccaggcccgtggtacaagtgttcattgctgtagaaaggg
atgctcagccctccaagacctgccaggttttcttgtcctgttgggcccccaacatcagcgaaataacctatagctggcgacgggagacaaccat
ggactttggtatggaaccacacagcctcttcacagacggacaggtgctgagcatttccctgggaccaggagacagagatgtggcctattcctg
cattgtctccaaccctgtcagctgggacttggccacagtcacgccctgggatagctgtcatcatgaggcagcaccagggaaggcctcctacaa
agatgtgctgctggtggtggtgcctgtctcgctgctcctgatgctggttactctcttctctgcctggcactggtgcccctgctcagggaaaaagaa
aaaggatgtccatgctgacagagtgggtccagagacagagaacccccttgtgcaggatctgccataaaggacaatatgaactgatgcctgga
ctatcagtaaccccactgcacaggcacacgatgctctgggacataactggtgcctggaaatcaccatggtcctcatatctcccatgggaatcctg
tcctgcctcgaaggagcagcctgggcagccatcacaccacgaggacaggaagcaccagcacgtttcacacctcccccttccctctcccatctt
ctcatatcctggctcttctctgggcaagatgagccaagcagaacattccatccaggacactggaagttctccaggatccagatccatggggaca
ttaatagtccaaggcattccctcccccaccactattcataaagtattaaccaactggcaccaaggaattgcctccagcctgagtcctaggctctaa
aagatattacatatttgaactaatagaggaactctgagtcacccatgccagcatcagcttcagccccagaccctgcagtttgagatctgatgcttc
ctgagggccaaggcattgctgtaagaaaaggtctagaaataggtgaaagtgagaggtgggggacaggggtttctctttctggcctaaggacttt
caggtaatcagagttcatgggccctcaaaggtagattgcagttgtagacaccgaggatggttgacaacccatggttgagatgggcaccgttttg
caggaaacaccatattaatagacatcctcaccatctccatccgctctcacgcctcctgcaggatctgggagtgagggtggagagtctttcctcac
gctccagcacagtggccaggaaaagaaatactgaatttgccccagccaacaggacgttcttgcacaacttcaagaaaagcagctcagctcag
gatgagtcttcctgcctgaaactgagagagtgaagaaccataaaacgctatgcagaaggaacattatggagagaaagggtactgaggcactct
agaatctgccacattcattttcaaatgcaaatgcagaagacttaccttagttcaaggggaggggacaaagaccccacagcccaacagcaggac
tgtagaggtcactctgactccatcaaactttttattgtggccatcttaggaaaatacattctgcccctgaatgattctgtctagaaaagctctggagta
ttgatcactactggaaaaacacttaaggagctaaacttaccttcggggattattagctgataaggttcacagtttctctcacccaggtgtaactgga
ttttttctggggcctcaatccagtcttgataacagcgaggaaagaggtattgaagaaacaggggtgggtttgaagtactattttcccagggtggct
tcaatctccccacctaggatgtcagccctgtccaaggaccttccctcttctccccagttcctgggcaatcacttcaccttggacaaaggatcagca
cagctggcctccagatccacatcaccactcttccactcgattgttcccagatcctccctgcctggcctgctcagaggttccctgttggtaacctgg
ctttatcaaattctcatccctttcccacacccacttctctcctatcaccttcccccaagattacctgaacagggtccatggccactcaacctgtcagct
tgcaccatccccacctgccacctacagtcaggccacatgcctggtcactgaatcatgcaaaactggcctcagtccctaaaaatgatgtggaaag
gaaagcccaggatctgacaatgagccctggtggatttgtggggaaaaaatacacagcactccccacctttctttcgttcatctccagggcccca
cctcagatcaaagcagctctggatgagatgggacctgcagctctccctccacaaggtgactcttagcaacctcatttcgacagtggtttgtagcg
tggtgcaccagggccttgttgaacagatccacactgctctaataaagttcccatccttaatgaag

| | | |
|---|---|---|
| SLIT2 | 59 | NM_004787, AB017168, AK027326 |
| Spondin 1, | 60 | NM_006108 |
| extracellular | | AB018305 |
| matrix protein | | AK074803 |
| (SPON1) | | BC041974 |
| sprouty | 61 | Sequence below. |
| homolog 4 | | |
| (SPRY4) | | |

gcgagctgagctgacagcgcggagctggcgctgtggagcgcagggagccttgccggttcctccgaccggcgtctgcgagtacagcggcg
gctaacctgccccggcttcaggatttacacagacgtggggcgatgcttgtgaccctgcagctcctcaaaccagcctgtattgagcggtttgcag
cctgatgctcagccccctccccacagggcccctagaagcctgtttctccgtacagtccaggacctccagccccatggagcccccgatcccaca
gagcgcccccttgactcccaactcagtcatggtccagccccttcttgacagccggatgtcccacagccggctccagcacccactcaccatcct
acccattgaccaggtgaagaccagccatgtggagaatgactacatagacaaccctagcctggccctgaccaccggcccaaagcggacccg
gggcggggccccagagctggccccgacgcccgcccgctgtgaccaggatgtcacccaccattggatctccttcagcgggcgccccagctct
gtgagcagcagcagcagcacatcctctgaccaacggctcttagaccacatggcaccaccacccgtggctgaccaggcctcaccaagggctg
tgcgcatccagcccaaggtggtccactgccagccgctggacctcaagggcccggcggtcccacccgagctggacaagcacttcttgctgtg
cgaggcctgtgggaagtgtaaatgcaaggagtgtgcatccccccggacgttgccttcctgctgggtctgcaaccaggagtgcctgtgctcagc
ccagactctggtcaactatggcacgtgcatgtgtttggtgcagggcatcttctaccactgcacgaatgaggacgatgagggctcctgcgctgac
cacccctgctcctgctcccgctccaactgctgcgcccgctggtccttcatgggtgctctctccgtggtgctgccctgcctgctctgctacctgcct
gccaccggctgcgtgaagctggcccagcgtggctacgaccgtctgcgccgccctggttgccgctgcaagcacacgaacagcgtcatctgca
aagcagccagcggggatgccaagaccagcaggcccgacaagcctttctgacagtttgtgtcgaagccccagtgctctgcctggaaacctggt
tctcttctgacatctaagaagactgcagcaaggtcagaggttttagcctcctgaggctgaccttgctagtctgcccactccctacccccagcttcg
gaaaatacagagaccaccaccacgtaccctgtattccccaaggtgatgaagaagcactttggggctttttttcagggtcctgaaactttgtgtcaa
acagacaatgcaggggcagggtgtggtttggggggaaatttttctttttcagaagacagaacacagatgtggacacatatccggaaactgcag
ctgcttgaatgccttcccagcccctccttctccctccctccctccgccccccccttcctcttttccattgtctttggctctcacaggagctagctgcct
gggaggaattgttaactgagtaccagggtacctttaaagaagacccttggagtcttctataccttcttctccttccccatctcactccaccccacttt
gtccctgatgtcttggggaaggtgtagaacaccctagcagttcctattgtatatacttgggagccactgagaacagaggacggccagtgagtcc
aagcctcgttcctccttctgcctccccggagccacaggatggatttaggagccactgctcagtgcacttctcccttccaactgcatcaactaactc
tcgggggtgttctgctcaccacaccgtccttcggttcttactgagtcacagactcgcctgcccactacgtgtcctgggttctctactcagatccctt
ccagaaactttatatgggtagaggaagccagggcggcaaatgcgagaccaaatatcattttgccaatgagtctgaggctgtggtctctggatcc
agtcattatgtttttatagaataattaaaccggatgctaacggtgttttaaaaaataataataaaacaacttgtttccttttggccacccccaggaagg
gctgatttcaaaatctgggggcgagcaacctcaaggaacacaatttccctccctatcaacaagaggattttaacagcaaagaagagaggcagc
acctcccattggcagaatgaccgctgagccaggctgggtttgggtttcttctcttctgattctgctgctcactgtcatagccttttgtgtatagtgatg
tgtctgtatctttaatgtaaatagagagatgatgaaaaaagagtctattttagtgttaggaagccccagcaggggagtcggaagagcttggaaga
gctggggagagggtaggggaaaggtttttccaggggccactgggtttgagccctgcttctgtgcacagccacaccaccctctcccgacagcc
ctcaaagacgtagcaactctttctctcaaggtgctaaaggactcagaaggtgcagcacgtccagtgggtaggtacttgttgcatgcaaaagctgt
agtgtatctggtccttcctccccagcttttgtgtggggttcttgctttgtgtggtattttgttttcccctctaatgagagggcatggcctgagtcagaag
agctaccccaggtgaaactggaagtgcatgaggcagagcgtccgtagcatttccagtttgttctgtataggacagaggtgcctccgggaagga
ggcagcgaggtaggtagctatgataggcacctaatgcttctcaaggacttattttttccttcttgaagactagtagtaacatcttatgatttagagtaa
gttgattgtaaccataggtatttattgattggaggaagggagggtcataattattttcggctttatttatgtaacatttgctagcttataaaaggcgaatgt
gaaatattgcatctgcattttccaaggctgattcgtgtagctacccttgccacagttgtgacggatgtatggatgttcttgaacatttcagaaggagt
ggtagaaaaaaacacacattcagccaaccacttataigaattgaatgtatcagaagtgtactgaagggactggagatggttttcctcagatgagg
gggccccaaaattgatagtgcacatctgcacgctttctgcgaggcctcagaacttttcccagggcccctccctcaaattgtctccatgggaaactt
gacccagtggcaagttgcactttggtgatcttggtggtctacacacccgttctgtggagagtcgatttacataagctgtgtatacacacacacaca
cacacacacacacacacacacacacacccctaccccacactgactgtctaccgacagagaccctatttcctggcaaacggcctcctgaaccct
gactttttgtgtacatacttgtaaacacggatttttctgggttttggtttgctttttccttttttccccctgcccctgttctagcttgttcttcttggtttgc
tttcaacctgcttgatggatgtctgcagagtgctctctaagagtccacctcagtgcctcgtgtgctcagtggtcatgggaaggagcgaaggaaccatc
cttggttctcccagcttggttgtgtagcaatccctcagcattgtttttctcagcttcttggcaaaaattaaaacaacaacaacaacaacaacaacaac
aacaaacagaaggataaactggcttgcctgtggaccctccccggctctggggccagtcgagagccactgagggacccagcactcagagac
acaacacacatgtgtagctgcttctggctgagtgtgtttcctgtcaccaatggcctgtttggctggacgatgcctcggcttgaccttttttgaaaagt
gctggttagttcccgccccctggtaaacctggggtaggtgggggttctgtcttaactcgaggggcacctgggatccaggacgcttctaggggg
ctctggctgcccgtgttaatgaaggacagcgcttccgcgagcaccctgggaactgggtcttgggtagcaaagccctcccagagaaaagatgg
gcacaactaaggctttcctgagcaggaagggggtgaagaccaatcccttcctttggtcctttggtacgcaccccctcagagctgagatggaag
acatggctagttcttttcagccttgtggagcctgtcagtcgccatcatacctcgagtgaggcccagctagataatgacttgtccaagatggcacac
gtggaaagttgatctgcaccagaacccggatgactgtcaccttgaagcgtcctgttctccttctgtgctgtcccaggaagtgtctggcgggcgtg
ggcagcacagctctacactgtacgattcactagggcatcctgcgagcctcactagccttctggttcatgcctttgacaagcatttttgtgccccctc
tgcttactgtgacagtcgatgatgaatcttgcgttgccattttctgctgtgggtaactgcgtgcagtgtcttgccttgctttctcttcttactgtgggaca
gcttggtttcatgttacaaacagaaaagctcgaggctcccaccccgccacatcccaacttcatttccccctcactgtagcccatttccaccccacc
acaaagttgccacaggttttctttgtatagaatatttattttgaagctctattttaatagtatttattttagaaagtctactattgtaagagttcttctgttt
gtgaagaaaaaaacaagttaaaaactgaatgtactgatttagaaaatatatataaatatatattgttaaatataaa

| | | |
|---|---|---|
| Stanniocalcin 2 | 62 | BC000658, BC006352, BC013958, AF055460, AB012664, |
| (STC2) | | AK027390, AK075406, AK095891 |
| Stress 70 | 63 | BC036370 |
| protein | | NM_006948 |
| chaperone, | | BC017580 |
| microsome- | | AK128540 |
| associated, | | |
| 60 kDa (STCH) | | |
| Tumor- | 64 | BC014785, AK026585 NM_002354, M26481, M32306, |
| associated | | M32325, M33011, X14758 |
| calcium signal | | |
| transducer 1 | | |
| (TACSTD1) | | |
| Threonyl-tRNA | 65 | BC088355 |
| synthetase | | Sequence below. |
| (TARS) | | |

cccacgcgtccgccacctcccacccgcctcttggctcctctcctctaggccgtcgctttcgggttctctcatcgcttcgtcgttcgccaatgtttga
ggagaaggccagcagtccttcagggaagatgggaggcgaggagaagccgattggtgctggtgaagagaagcaaaaggaaggaggcaaa
aagaagaacaaagaaggatctggagatggaggtcgagctgagttgaatccttggcctgaatatatttacacacgtcttgagatgtataatatact
aaaagcagaacatgattccattctggcagaaaaggcagaaaaagatagcaagccaattaaagtcactttgcctgatggtaaacaggttgatgcg
gaatcttggaaaactacaccatatcaaattgcctgtggaattagtcaaggcctggccgacaacaccgttattgctaaagtaaataatgttgtgtgg
gacctggaccgccctctggaagaagattgtaccttggagcttctcaagtttgaggatgaggaagctcaggcagtgtattggcactctagtgctca
cataatgggtgaagccatggaaagagtctatggtggatgtttatgctacggtccgccaatagaaaatggattctattatgacatgtacctcgaaga
agggggtgtgtctagcaatgatttctcttctctggaggctttgtgtaagaaaatcattaaagaaaaacaagcttttgaaagactggaagttaagaaa
gaaactttactggcaatgtttaagtacaacaagttcaaatgccggatattgaatgaaaaggtgaatactccaactaccacagtctatagatgtggc
cctttgatagatctctgccggggtcctcatgttagacacacgggcaaaattaaggctttaaaaatacacaaaaattcctccacgtactgggaagg
caaagcagatatggagactctccagagaatttatggcatttcattcccagatcctaaaatgttgaaagagtgggagaagttccaagaggaagcta
aaaaccgagatcataggaaaattggcagggaccaagaactatatttctttcatgaactcagccctggaagttgcttttttctgccaaaaggagcct
acatttataatgcacttattgaattcattaggagcgaatataggaaaagaggattccaggaggtagtcaccccaaacatcttcaacagccgactct
ggatgacctcgggccactggcagcactacagcgagaacatgttctcctttgaggtggagaaggagctgtttgccctgaaacccatgaactgcc
caggacactgccttatgtttgatcatcggccaaggtcctggcgagaactgcctctgcggctagctgattttggggtacttcataggaacgagctg
tctggagcactcacaggactcacccgggtacgaagattccaacaggatgatgctcacatattctgtgccatggagcagattgaagatgaaataa
aaggttgtttggattttctacgtacggtatatagcgtatttggattttcttttaaactaaacctttctactcgcccagaaaaattccttggagatatcgaa
gtatgggatcaagctgagaaacaacttgaaaacagtctgaatgaatttggtgaaaagtgggagttaaactctggagatggagctttctatggccc
aaagattgacatacagattaaagatgcgattgggcggtaccaccagtgtgcaaccatccagctggatttccagttgcccatcagatttaatcttac
ttatgtaagccatgatggtgatgataagaaaaggccagtgattgttcatcgagccatcttgggatcagtggaaagaatgattgctatcctcacaga
aaactatgggggcaaatggcccttttggctgtcccctcgccaggtaatggtagttccagtgggaccaacctgtgatgaatatgcccaaaaggta
cgacaacaattccacgatgccaaattcatggcagacattgatctggatccaggctgtacattgaataaaaagattcgaaatgcacagttagcaca
gtataacttcattttagttgttggtgaaaaaaaaaaaaaaa

| | | |
|---|---|---|
| T-box 2 | 66 | BC052566, NM_005994, BC070054, AB209378, |
| (TBX2) | | AK127429, U28049 |
| Thy-1 cell | 67 | NM_006288, AF261093, BC005175, AK057865, |
| surface antigen | | AK090996, AK094374 |
| (THY1) | | |
| Tumor necrosis | 68 | BC030205 |
| factor, alpha- | | NM_007115 |
| induced protein | | M31165 |
| 6 (TNFAIP6) | | Sequence below. |

cagtcacatttcagccactgctctgagaatttgtgagcagcccctaacaggctgttacttcactacaactgacgatatgatcatcttaatttacttattt
ctcttgctatgggaagacactcaaggatggggattcaaggatggaatttttcataactccatatggcttgaacgagcagccggtgtgtaccacag
agaagcacggtctggcaaatacaagctcacctacgcagaagctaaggcggtgtgtgaatttgaaggcggccatctcgcaacttacaagcagc
tagaggcagccagaaaaattggatttcatgtctgtgctgctggatggatggctaagggcagagttggataccccattgtgaagccagggccca
actgtggatttggaaaaactggcattattgattatggaatccgtctcaataggagtgaaagatgggatgcctattgctacaacccacacgcaaag
gagtgtggtggcgtctttacagatccaaagcaaatttttaaatctccaggcttcccaaatgagtacgaagataaccaaatctgctactggcacatta
gactcaagtatggtcagcgtattcacctgagttttttagattttgaccttgaagatgacccaggttgcttggctgattatgttgaaatatatgacagtta
cgatgatgtccatggctttgtgggaagatactgtggagatgagcttccagatgacatcatcagtacaggaaatgtcatgaccttgaagtttctaag
tgatgcttcagtgacagctggaggtttccaaatcaaatatgttgcaatggatcctgtatccaaatccagtcaaggaaaaaatacaagtactacttct
actggaaataaaaactttttagctggaagatttagccacttataaaaaaaaaaaaaaggatgatcaaaacacacagtgtttatgttggaatcttttgg
aactcctttgatctcactgttattattaacatttatttattatttttctaaatgtgaaagcaatacataatttagggaaaattggaaaatataggaaacttta
aacgagaaaatgaaacctctcataatcccactgcatagaaataacaagcgttaacattttcatatttttttctttcagtcatttttctatttgtggtatatgt
atatatgtacctatatgtatttgcatttgaaattttggaatcctgctctatgtacagttttgtattatacttttaaatcttgaactttataaacattttctga
aatcattgattattctacaaaaacatgattttaaacagctgtaaaatattctatgatatgaatgttttatgcattatttaagcctgtctctattgttggaatt
tcaggtcattttcataaatattgttgcaataaatatccttgaacacaaaaaaaaaaaaaaaaaa

| | | |
|---|---|---|
| Tumor necrosis | 69 | BC010241, BC017730, NM_014452, AY358304, |
| factor receptor | | BC005192, BC015466, AB209394, AJ420531 |
| superfamily, | | Sequence below. |
| member 21 | | |
| (TNFRSF21) | | |
| (DR6) | | |

gccaccttgtctgtgagctccctgtgccccccatacggtgtgtcctgtgggttggggtgtgcggaagaaagggacagagactgaggatgtgcg
gtgtaagcagtgtgctcggggtaccttctcagatgtgccttctagtgtgatgaaatgcaaagcatacacagactgtctgagtcagaacctggtgg
tgatcaagccggggaccaaggagacagacaacgtctgtggcacactcccgtccttctccagctccacctcaccttcccctggcacagccatctt
tccacgccctgagcacatggaaacccatgaagtcccttcctccacttatgttcccaaaggcatgaactcaacagaatccaactcttctgcctctgt
tagaccaaaggtactgagtagcatccaggaagggacagtccctgacaacacaagctcagcaagggggaaggaagacgtgaacaagaccct
cccaaaccttcaggtagtcaaccaccagcaaggcccccaccacagacacatcctgaagctgctgccgtccatggaggccactgggggcga
gaagtccagcacgcccatcaagggccccaagaggggacatcctagacagaacctacacaagcattttgacatcaatgagcatttgccctggat
gattgtgcttttcctgctgctggtgcttgtggtgattgtggtgtgcagtatccggaaaagctcgaggactctgaaaaaggggccccggcaggatc
ccagtgccattgtggaaaaggcagggctgaagaaatccatgactccaacccagaaccgggagaaatggatctactactgcaatggccatggt
atcgatatcctgaagcttgtagcagcccaagtgggaagccagtggaaagatatctatcagtttctttgcaatgccagtgagagggaggttgctgc
tttctccaatgggtacacagccgaccacgagcgggcctacgcagctctgcagcactggaccatccggggccccgaggccagcctcgccca
gctaattagcgccctgcgccagcaccggagaaacgatgttgtggagaagattcgtgggctgatggaagacaccacccagctggaaactgac
aaactagctctcccgatgagccccagcccgcttagcccgagccccatccccagccccaacgcgaaacttgagaattccgctctcctgacggt
ggagccttccccacaggacaagaacaagggcttcttcgtggatgagtcggagccccttctccgctgtgactctacatccagcggctcctccgc
gctgagcaggaacggttcctttattaccaaagaaaagaaggacacagtgttgcggcaggtacgcctggacccctgtgacttgcagcctatcttt
gatgacatgctccactttctaaatcctgaggagctgcgggtgattgaagagattccccaggctgaggacaaactagaccggctattcgaaattat
tggagtcaagagccaggaagccagccagaccctcctggactctgtttatagccatcttcctgacctgctgtagaacatagggatactgcattctg
gaaattactcaatttagtggcagggtggttttttaattttcttctgtttctgatttttgttgtttggggtgtgtgtgtgtgtttgtgtgtgtgtgtgtgtgtg
tgtgtgtgtgtgtttaacagagaatatggccagtgcttgagttctttctccttctctctctctcttttttttttaaataactcttctgggaagttggtttata
agcctttgccaggtgtaactgttgtgaaatacccaccactaaagttttttaagttccatattttctccattttgccttcttatgtattttcaagattattctg
tgcactttaaatttacttaacttaccataaatgcagtgtgacttttcccacacactggattgtgaggctcttaacttcttaaaagtataatggcatcttgtga
atcctataagcagtctttatgtctcttaacattcacacctactttttaaaaacaaatattattactatttttattattgtttgtcctttataaattttcttaa
agattaagaaaatttaagaccccattgagttactgtaatgcaattcaactttgagttatcttttaaatatgtcttgtatagttcatattcatggctgaaactt
gaccacactattgctgattgtatggttttcacctggacaccgtgtagaatgcttgattacttgtactcttcttatgctaatatgctctgggctggagaaatga
aatcctcaagccatcaggatttgctatttaagtggcttgacaactgggccaccaaagaacttgaacttcaccttttaggatttgagctgttctggaac
acattgctgcactttggaaagtcaaaatcaagtgccagtggcgccctttccatagagaatttgcccagctttgctttaaaagatgtcttgttttttatat
acacataatcaataggtccaatctgctctcaaggccttggtcctggtgggattccttcaccaattactttaattaaaaatggctgcaactgtaagaac
ccttgtctgatatatttgcaactatgctcccatttacaaatgtaccttctaatgctcagttgccaggttccaatgcaaaggtggcgtggactccctttg
tgtgggtggggtttgtgggtagtggtgaaggaccgatatcagaaaaatgccttcaagtgtactaatttattaataaacattaggtgtttgttaaaaaa
aaaaaaaaa

| | | |
|---|---|---|
| Uridine | 70 | BC001405, BC047030, BC053592, X90858 |
| phosphorylase 1 | | |
| (UPP1) | | |

### Example 3

### Further Validation of TVM

To test the specificity of identified genes to tumors relative to normal tissues, previously uninvestigated genes were selected from the above candidates for further validation. As positive controls for our assays, known TVM were included. First, 12 selected TVM were tested for enrichment of expression in ovarian tumors versus normal ovarian tissue, by analyzing their whole tissue expression by qRT-PCR in an independent set of stage-III ovarian cancers (n=20) and normal ovaries (n=5). All 12 TVM tested were upregulated in cancer tissue versus normal ovaries (p<0.05 for all). Many TVM were expressed at or below the lowest limits of detection in normal ovaries (FIG. 3A). TVM expression in normal tissues was also examined by analyzing pre-existing SAGE data (Boon K et al, An anatomy of normal and malignant gene expression. Proc Natl Acad Sci USA 2002 Aug. 20; 99(17):11287-92.) (FIG. 3E). Genes exhibiting no or minimal in silico expression in normal tissues were then screened in a panel of normal tissues with qRT-PCR (FIG. 3B). All TVM tested, except for C11orf8 and FJX1, were highly expressed in tumors and were expressed at or below the lowest limits of detection in normal tissues tested. C11orf8 was significantly enriched in TVC relative to all cell types tested, and was expressed at or below the lowest limit of detection in all cell types except brain and spleen. FJX1 was significantly enriched in TVC relative to all cell types tested except for spleen.

Quantitative Real-time Polymerase Chain Reaction (qRT-PCR) was used to evaluate the expression of select TVM in different malignancies, including lung cancer, mesothelioma, breast cancer and melanoma (FIG. 3C). To ensure expression differences were not due to differences in vascular density, the qRT-PCR data were normalized against CD31 expression, a reliable marker of endothelial cells. Collagen 11α1 was expressed at even higher levels in breast and lung relative to ovarian tumors, LZTS1 was relatively specific for melanoma and ovarian cancer, and FZD10 and EMBPL1 was relatively specific for ovarian tumors. Other TVM exhibited quantitative differences in expression among different tumor types, indicating that, under the conditions utilized, TVC signatures differ among tumor types.

In addition, TVM that exhibited minimal expression in normal tissues were evaluated for expression in tissues with physiologic angiogenesis including corpus luteum, proliferative endometrium and placenta. Data were normalized against CD31 expression. Some of the TVM (adlican, collagen 11α1, F2RL1, GPM6B and STC2) were expressed at 40-350 fold higher levels in tumor versus tissues with physiologic angiogenesis (FIG. 3D). However, EGFL6, OLFML2B and previously established TVM such as TEM-1 and TEM-7 were expressed in reproductive tissues with physiologic angiogenesis.

These findings further validated the identified proteins and transcripts as TVM. Expression of the TVM in other tumors besides ovarian cancer show that their utility (e.g. in tumor localization and diagnostic and therapeutic methods) is not limited to ovarian tumors, but rather includes many other solid tumors. In addition, these findings validated the methods used in the present invention for identifying TVM, with regard to both those TVM tested in vivo and those not yet tested in vivo.

### Example 4

### Localization of TVM Expression to Tumor Vasculature

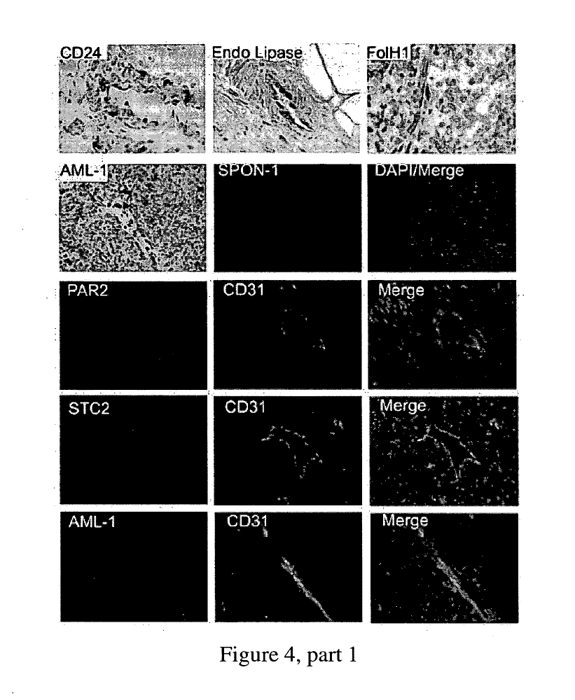

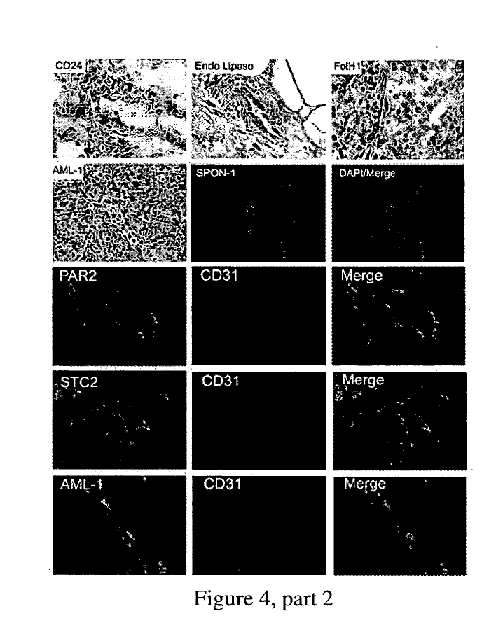

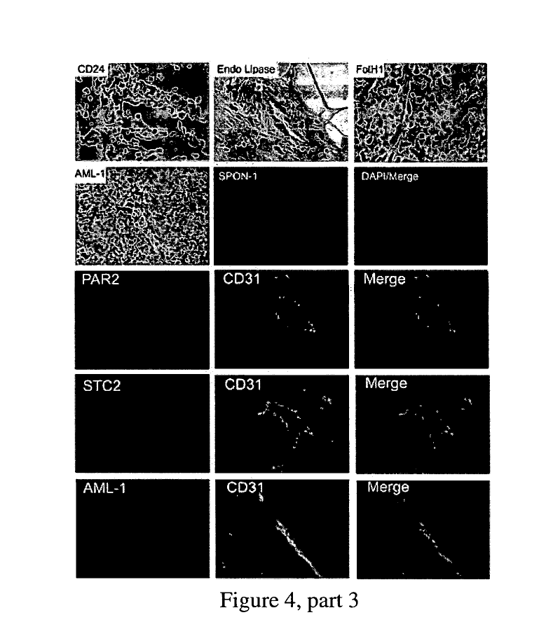

To confirm that genes identified through immuno-LCM and transcriptional profiling were localized to ovarian cancer tumor vasculature, immunohistochemistry (IHC) was performed. Both CD24 (FIG. 4) and CD74, normally expressed by hematopoietic cells of the monocytic/macrophage lineage, but also reported as expressed in endothelium, were clearly expressed in ovarian tumor vessels. Endothelial lipase and FolH1, genes previously identified as expressed in tumor vasculature, were also expressed in ovarian tumor endothelium. SPON-1 was also found to be expressed in a vascular pattern. PAR2 (F2RL1) was localized primarily in peri-endothelial location, while STC2 co-localized with CD31 expression. AML-1 was expressed in stromal cells, but also in vascular structures by IHC (FIG. 4, second row). In addition, AML-1 expression was only detected in small tumor vessels, but was absent in large, smooth muscle actin-positive, mature vessels. Double immunofluorescence indicated that AML-1 co-localized with CD31 expression (FIG. 4, last row); localization in adjacent cells could not be excluded, however.

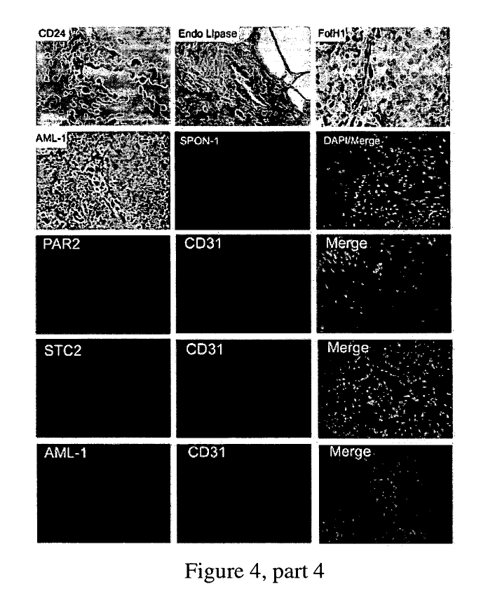

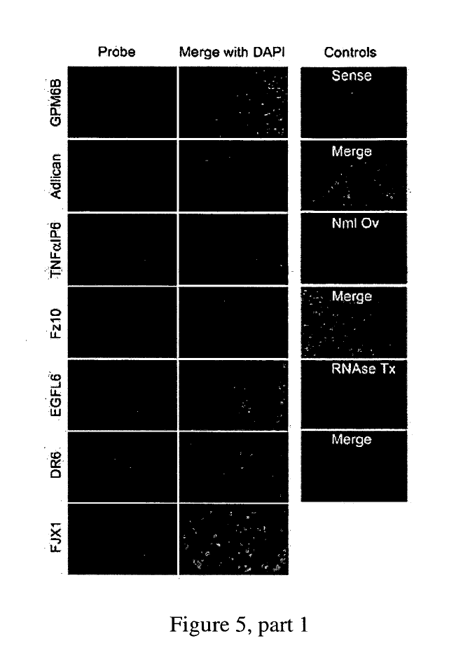

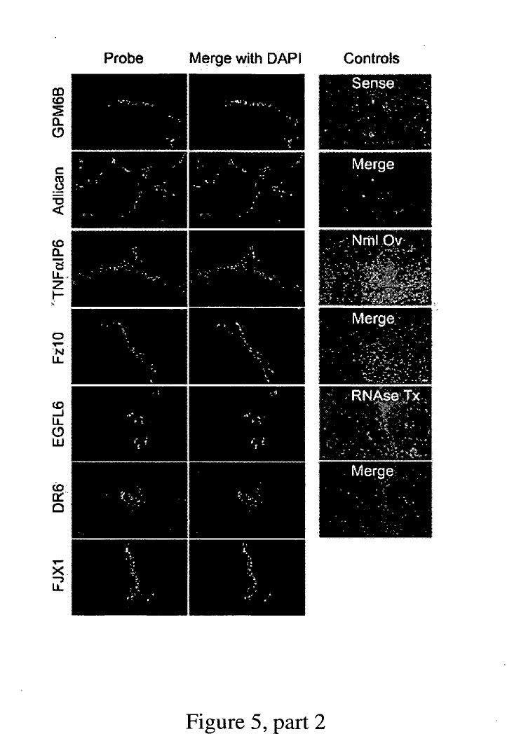

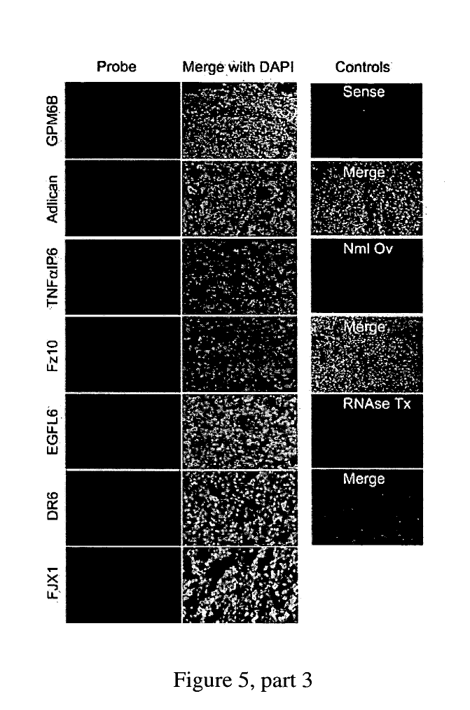

For TVM against which antibodies were unavailable, expression in tumor vasculature was confirmed using in-situ hybridization (ISH). ISH revealed clear expression of GPM6B, adlican, TNFαIP6, FZD10, EGFL6, DR6 and FJX1 in 5 independent ovarian cancer samples (FIG. 5). TVM were detected in vascular structures and were absent in tumor cells. Expression was generally limited to small neo-vascular structures, and with the exception of FZD10, was absent in large, muscular vessels. With the exception of FJX1, which was detected at very low levels in normal ovarian vasculature, TVM were not detected in 3 independent normal ovary specimens. No specific staining was observed with sense strand RNA controls, showing that the signals observed accurately reflected expression of the RNA molecules measured. Pre-treatment of slides with RNAse prior to probe hybridization eliminated all specific staining for all TVM tested, providing an additional control for veracity of the data.

These findings confirm that the identified TVM were expressed in tumor vasculature.

### Example 5

### TVM Expression in Different TVC

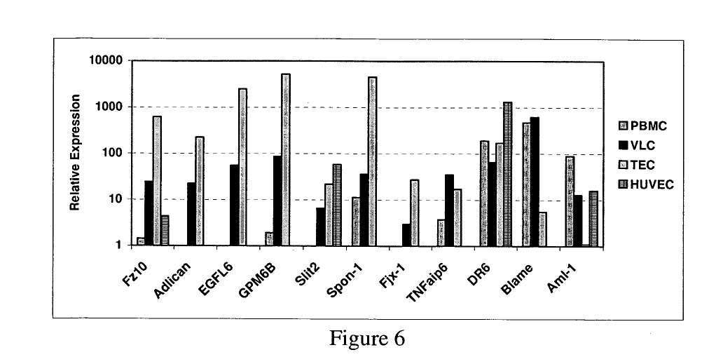

To test which cells in the tumor vasculature express the identified TVM, mRNA expression was evaluated by qRT-PCR in VE-cadherin⁺ CD146⁺ CD45⁻ tumor endothelial cells (TEC) isolated freshly by FACS from ovarian cancer specimens. TVM expression in VE-cadherin⁺ CD146⁺ CD45⁺ vascular leukocytes (VLC); myeloid cells with vasculogenic potential; and control peripheral blood mononuclear cells (PBMC) was also tested. All TVM were expressed by VLC, and all except AML-1 were expressed by purified TEC (FIG. 6). Adlican, EGFL6, FJX1, FZD10, GPM6B and SPON-1 were expressed at approximately 10-fold higher levels in TEC than VLC. TEC expressed these genes at greater than 100-fold higher levels than PBMC. BCM-like membrane protein precursor (BLAME) was higher in VLC (and PBMC) than TEC, and TNFαIP6 was higher in TEC and VLC than PBMC. AML-1 demonstrated highest expression in PBMC and intermediate in VLC, while it was nearly absent in TEC. Thus, genes identified through immuno-LCM and transcriptional profiling are expressed by TVC, at different levels in TEC and VLC.

Thus, expression of most TVM tested in tumor endothelial cells provided still further evidence that the TVM identified were genuine, and that cell contamination did not occur during immuno-LCM.

### Example 6

### Dendritic Cell Precursors Upregulate TVM Under the Influence of VEGF-A

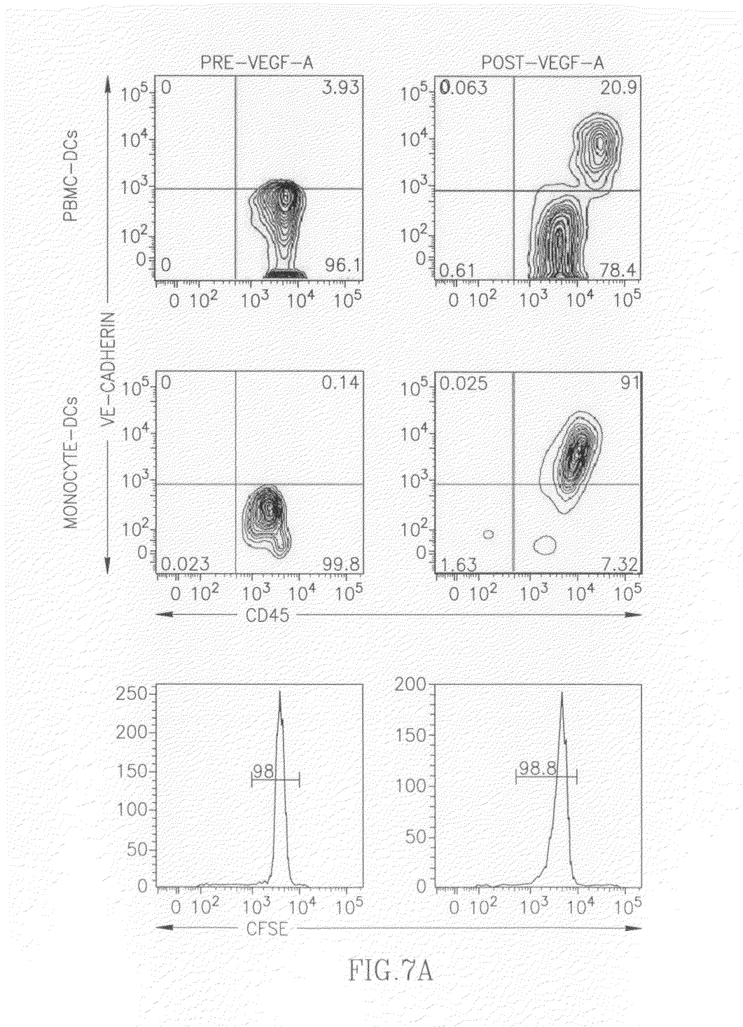

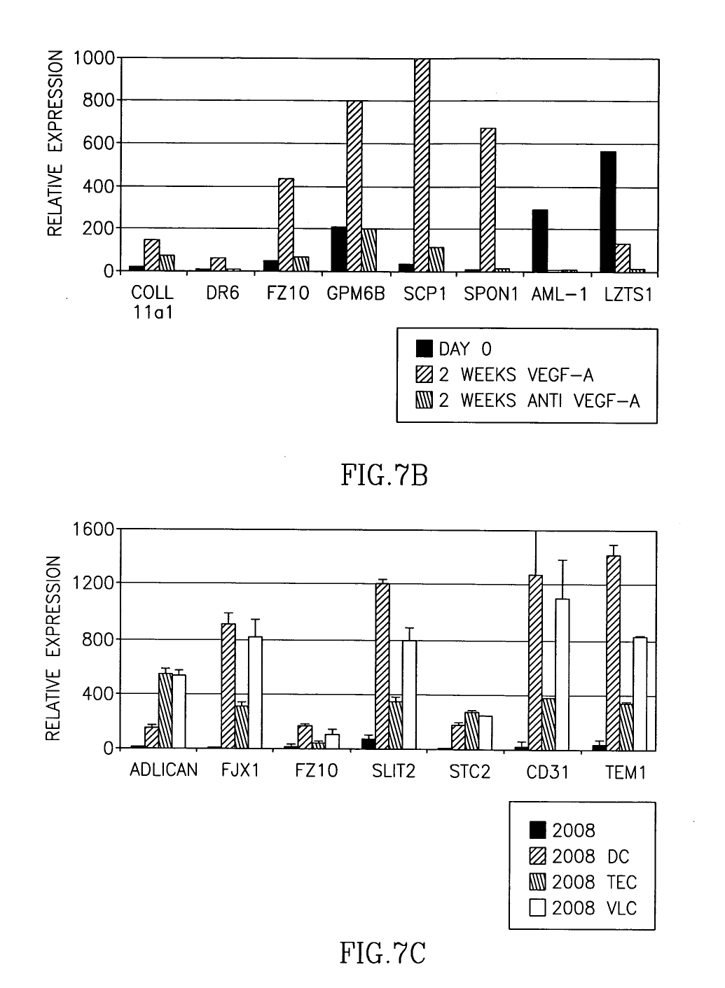

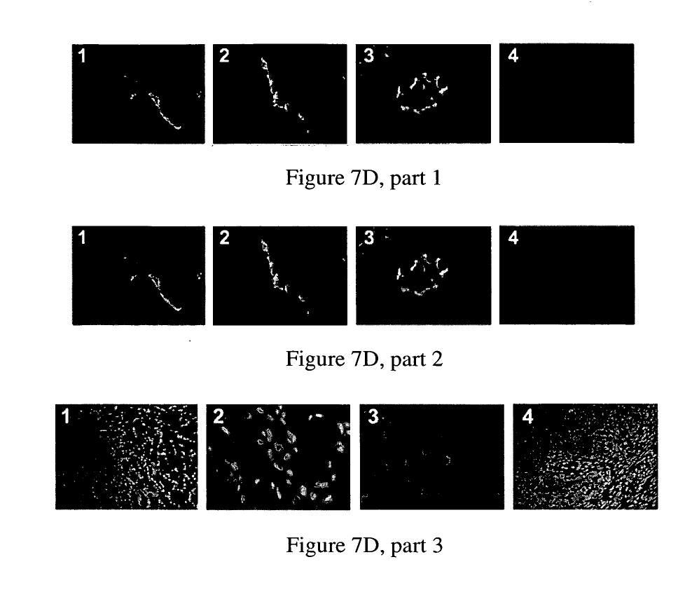

To address the origin of human VLC, DC were generated from human PBMC with GM-CSF and IL-4 (Benencia F et al, HSV oncolytic therapy upregulates interferon-inducible chemokines and recruits immune effector cells in ovarian cancer. Mol. Ther. 2005 November; 12(5):789-802). FACS analysis demonstrated 100% CD45 expression in these cells (FIG. 7A, top left). DC were then labeled with CFSE and treated with recombinant human (rh)VEGF₁₆₅ for 7 days. VEGF₁₆₅ induced significant up-regulation of VE-cadherin in a population of PBMC-derived DC (FIG. 7A, top right). The monocyte fraction of PBMC was primarily responsible for giving rise to the VE-cadherin⁺CD45⁺ population upon treatment with VEGF₁₆₅ (FIG. 7A, middle). Lack of CFSE dye dilution with VEGF-A treatment indicated that the phenotype switch was not due to expansion of endothelial progenitor contaminants (FIG. 7A, bottom). In addition, endothelial progenitor assays performed with these cells demonstrated no proliferation. Thus, under the conditions utilized, VEGF-A induces monocyte-derived DC to acquire VLC features.

To test whether VEGF₁₆₅ treatment induced TVM in human PBMC-derived DC, expression of collagen 11α1, DR6, FZD10, GPM6B, SCP1, SPON-1, and TEM-1 was measured, following VEGF₁₆₅ treatment. All these transcripts were markedly up-regulated, and VEGF-neutralizing antibody abrogated their up-regulation (FIG. 7B). Expression of AML-1 and LZTS1 were down-regulated in DC by VEGF₁₆₅. This down-regulation was abrogated by anti-VEGF-A antibody in 3-day cultures but not after 2 weeks of VEGF₁₆₅ treatment. Other TVM, such as adlican and SLIT2, were not up-regulated in DC by VEGF₁₆₅ treatment, showing that, under the conditions utilized, other mediators besides VEGF-A were necessary to complete the full differentiation of these cells into VLC.

### Example 7

### DC Precursors Contribute to Tumor Vasculature and Express a Larger Repertoire of TVM In Vivo

To determine whether additional TVC could be contributed by DC in vivo, human PBMC-derived DC were treated with rhVEGF₁₆₅ for one week in vitro, admixed with 2008 human ovarian cancer cells (1:10; DC, tumor cell) and transplanted within Matrigel® into NOD/SCID/β2M−/− recipient mice. As controls, 2008 cells were injected alone, or co-injected together with FACS-isolated TEC or VLC in the opposite side. Tumors were assessed for expression of TVM 3 weeks later using qRT-PCR. Three TVM, adlican, FJX1 and STC2, as well as known TVM, SLIT2 and TEM-1, were clearly detected in tumor xenografts enriched with human VEGF-treated DC, VLC or TEC, while they were not detected in tumors generated with tumor cells alone (FIG. 7C). Immunofluorescence analysis to identify human endothelium using antibodies specific to human (h)CD34 and hCD31 identified small clusters of human vascular structures in tumors enriched with rhVEGF-treated DC, VLC or TEC (FIG. 7D). No staining for human endothelium was identified when 2008 cells were injected alone. These cells exhibited markers and in vivo behavior that were similar to tumor-derived VLC, showing that certain tumor VLC derive from myeloid DC.

Induction of TVM by VEGF-A in vitro was not due to expansion of contaminating endothelial precursor cells, as shown by negative CFSE dilution and endothelial progenitor cell assays. When VEGF-treated DC were exposed to the tumor microenvironment in vivo, additional TVM could be induced, showing that, under the conditions utilized, in vivo factors further contribute to acquisition of a VLC phenotype by DC. Importantly, tumors enriched with VEGF-treated DC exhibited vascular structures expressing human CD31 and CD34, while control tumors generated with only tumor cells did not. Thus, tumor cells were not responsible for the expression of TVM in vivo.

Thus, human VEGF-primed monocyte-derived DC can give rise to vascular cells in tumors. These findings demonstrate that many of the novel TVM identified are expressed by vascular cells of myeloid lineage.

### Example 8

### Identification of Additional Transmembrane and Secreted TVM

The tumor vasculature-specific genes identified in Example 2 were further analyzed by t-statistics, ranked and nonparametric analyses, and Genespring®. Over 200 genes were identified that were either exclusively expressed in tumor ECs or significantly overexpressed in tumor ECs and absent in 75% of normal ECs. From among these genes, transcripts were then identified that encoded proteins that were transmembrane or secreted, based on known structure or sequence analysis. The 108 transcripts identified are listed and described in FIG. 8. GenBank Accession Numbers of the new plasma membrane-associated, (“PM”), transmembrane (TM), extracellular matrix (ECM), and secreted TVM (those that do not appear in Table 6) are set forth in Table 7.

TABLE 7
Sequences of the new transmembrane and secreted TVM. Each
GenBank Accession No. represents a separate embodiment
of the present invention.

| | Localization; | |
|---|---|---|
| | SEQ ID | |
| Gene name | Number | Exemplary GenBank Accession Numbers |
| IBSP (Integrin-binding | Secreted; 89 | NM_004967, J05213, NP_004958 |
| sialoprotein) | | |
| FAD104 (FNDC3B; | TM; 90 | NM_022763, BC033635, AK127826, |
| Fibronectin type III domain | | AL157482, BC039297, BC012204, |
| containing 3B) | | AF543840, AY358146, AY358367, |
| | | AK027052, AK075220, AB098597, |
| | | AK223599, AL157482 |
| WARP (Von Willebrand | TM; 91 | AK096773, 30349211, NM_199121, |
| factor A domain containing | | BC059409, NM_022834, AK025868 |
| 1) | | |
| B-cell receptor-associated | ER/TM; 92 | NM_018844, AF126020, |
| protein 29 (BCAP29) | | NM_001008405, NM_001008406, |
| | | NM_001008407, BC008478, |
| | | AF126020, BT006981. |
| CDH1 (Cadherin 1, type 1, | TM; 93 | NM_004360, Z13009, L08599, |
| E-cadherin (epithelial) | | AB025105, AB025106, X12790, |
| | | X52279, Z18923. |
| FLJ10826 (OGFOD1; 2- | TM; 94 | NM_018233, AK024314, BC032919, |
| oxoglutarate and iron- | | NM_001031707, BC032919, |
| dependent oxygenase | | AK001688, AB046832, AK022130, |
| domain containing 1) | | AK022752 |
| OPN3 (Opsin 3; | TM; 95 | AK000933, BC036773, |
| encephalopsin, panopsin) | | NM_001030011, NM_001030012, |
| | | NM_014322, AF140242, BC036773, |
| | | BC062622, BC073872, AF303588 |
| HIATL2 (Hippocampus | TM; 96 | BC005058, NM_032318, XM_939817 |
| abundant gene transcript- | | |
| like 2) | | |
| IL28RA (Interleukin 28 | TM; 97 | NM_170743, NM_173064, |
| receptor, alpha; interferon, | | NM_173065, AF439325, AK160364, |
| lambda receptor) | | BC101408, BC101975, AY129151, |
| | | AY129152, AY129153, AK160364, |
| | | AF439325, AJ534330, AJ534331 |
| TMEM19 (Transmembrane | TM; 98 | BC008596, NM_018279, BC008596, |
| protein 19) | | AK001798, AK223148 |
| C10orf69 (SPFH domain | PM; 99 | BC031791, NM_006459, AK127017, |
| family, member 1) | | AF064093, AK222480 |
| FRAP1 (FK506 binding | PM; 100 | NM_004958, AB209995, L34075, |
| protein 12-rapamycin | | L35478 |
| associated protein 1) | | |
| CKLFSF6 (CKLF-like | TM/ | BC002797, AK000403, NM_017801, |
| MARVEL transmembrane | Secreted; 101 | AF116694, BC027472, AF479261, |
| domain containing 6) | | CR457231 |
| MPHOSPH9 (M-phase | PM/Golgi | X98258, NM_022782, AK023016, |
| phosphoprotein 9) | stacks; 102 | NP_073619 |
| CLST11240 (HIGD1B; | TM; 103 | BC020667, NM_016438, NM_004247, |
| HIG1 domain family, | | BC002360, D21163, AB038021, |
| member 1B) | | AB038022, AJ505017, CR456774 |
| MS4A6A (Membrane- | TM; 104 | BC022854, AF237908, AF253977, |
| spanning 4-domains, | | AF261136, AF286866, AF350502, |
| subfamily A, member 6A) | | AB013104, AF212240, BC108728, |
| | | NM_022349, NM_152851, NM_152852 |
| SGPP2 (Sphingosine-1- | TM; 105 | BC047944, NM_152386, BC106904, |
| phosphate phosphatase 2) | | BC106905, AF542512, AK096323, |
| | | XM_938742 |
| SLC11A1 (Solute carrier | TM; 106 | BC071165, L32185, NM_000578, |
| family 11; proton-coupled | | NM_001032220, BC041787, D38171, |
| divalent metal ion | | D50402, D50403, AB209650 |
| transporters, member 1) | | |
| SLCO3A1 (Solute carrier | TM; 107 | AF205074, NM_013272, AB031050, |
| organic anion transporter | | AK057031, BC000585, AF187816, |
| family, member 3A1) | | BC000585, XM_941061, XM_944958, |
| | | XM_944959 |
| LOC51136 (PTD016 | TM; 108 | BC006971, BC088365, NM_016125, |
| protein) | | AK098649, AF100745, NP_057209 |
| HAPLN1 (Hyaluronan and | ECM; 109 | BC057808, NM_001884, U43328, |
| proteoglycan link protein 1) | | BC057808, X17405 |
| DKFZp564I1922 (MXRA5 | PM; 110 | AK129884, AK130102, NM_000175, |
| (Matrix-remodelling | | NM_015419, K03515, AF245505, |
| associated 5) | | BC004982, BC006342, BC011846, |
| | | BC064986, BC080586, AL049946 |
| KCNE3 (Potassium | TM; 111 | NM_005472, BC113743, BC110612, |
| voltage-gated channel, Isk- | | AF076531, AF302494, DQ192291, |
| related family, member 3) | | DQ192292, CR457209, CR936625 |
| CALM3 (Calmodulin 3; | PM/ | BC005137, BC006182, AL050207, |
| phosphorylase kinase, | cytoplasm; | NM_005184, AK094964, J04046, |
| delta) | 112 | BT006855 |
| KCNE4 (Potassium | TM; 113 | BC014429, NM_080671, AY065987, |
| voltage-gated channel, Isk- | | DQ192293 |
| related family, member 4) | | |
| MGC34647 | TM; 114 | BC029804, NM_152456, AK172744, |
| | | BC029804 |
| MUC1 (Mucin 1, | TM; 115 | NM_001018016, NM_001018017, |
| transmembrane) | | NM_001018021, J05581, NM_002456, |
| | | M34088, M34089, M31823, M32738, |
| | | M32739, M26316, J05582, M21868, |
| | | U60259, U60260, U60261, S81736, |
| | | AY327582-AY327600, AY466157, |
| | | Z17324, Z17325 |
| SDC1 (Syndecan 1) | TM; 116 | NM_001006946, NM_002997, |
| | | AJ551176, BC008765, J05392, |
| | | BC008765, AK222739, X60306 |
| SLC30A6 (Solute carrier | PM; 117 | AL832755, BC032525, BC066903, |
| family 30 (zinc | | NM_017964, BC066903, AK055663 |
| transporter), member 6) | | |
| ST14 (Suppression of | TM; 118 | BC030532, NM_021978, AB030036, |
| tumorigenicity 14 (colon | | AF057145, AF118224, BC005826, |
| carcinoma)) | | BC018146, |
| CDCP1 (CUB domain | TM; 119 | AK026329, NM_022842, NM_178181, |
| containing protein 1) | | BC021099, BC069254, AY026461, |
| | | AF468010, AY167484 |
| LOC116238 (TLCD1) | TM; 120 | BC014072, NM_138463 |
| WFDC2 (WAP four- | Secreted; 121 | BC046106, NM_006103, BC039816, |
| disulfide core domain 2) | | X63187, NM_080733, AF330259, |
| | | NM_080734, NM_080736, AF330260 |
| SPTB; Spectrin, beta, | PM/ | NM_000347, X59511, |
| erythrocytic (includes | cytoskeleton; | NM_001024858, M37885, J05500, |
| spherocytosis, clinical type | 122 | AF465439, X59510, X59511, |
| I) | | BX248270 |
| FNDC3 (Fibronectin type | TM; 123 | NM_014923, BC060816, BC070072, |
| III domain containing 3A) | | BX648706, AK001140, AJ749706, |
| | | AJ749707 |
| SPRY1 (Sprouty homolog | Cytoplasm/ | BC063856, NM_005841, NM_199327, |
| 1, antagonist of FGF | lamelli- | AF041037, AK026960, AY590694 |
| signaling (Drosophila) | podium | |
| | membrane; | |
| | 124 | |
| MME (Membrane metallo- | TM; 125 | AL833459, NM_000902, NM_007287, |
| endopeptidase; neutral | | NM_007288, NM_007289, J03779, |
| endopeptidase, | | BC106070, BC101632, BC101658 |
| enkephalinase, CALLA, | | |
| CD10) | | |
| TOMM22 (Translocase of | mitochondrial | BC009363, NM_020243, AK024731, |
| outer mitochondrial | outer | AB041906 |
| membrane 22 homolog | membrane; | |
| (yeast)) | 126 | |
| SPP1 (Secreted | Secreted; 127 | BC007016, BC017387, BC022844, |
| phosphoprotein 1 | | BC093033, NM_000582, AF052124, |
| (osteopontin, bone | | BX648003, AK057738, AK075463, |
| sialoprotein I, early T- | | J04765, X13694, D28759, D28760, |
| lymphocyte activation 1) | | D28761, M83248 |
| INSR (Insulin receptor) | TM; 128 | BC117172, AL365454, NM_000208, |
| | | M10051, M24555, BC047591, |
| | | AB208861, X02160, NP_000199 |
| LPPR4 (Plasticity related | TM; 129 | NM_014839, AF357013, AF541281, |
| gene 1) | | BC042963, AY304518, AB007924, |
| | | AL834390 |
| C14orf100 | TM; 130 | BC005198, BC010359, NM_016475, |
| | | AF212245, AF529304, AK021683, |
| | | AK056145, AK128628 |
| SLC9A5 (Solute carrier | TM; 131 | NM_004594, AF111173, AL137689, |
| family 9 (sodium/hydrogen | | AB209203, NP_004585 |
| exchanger), member 5 | | |
| SCGB2A1 (Secretoglobin, | TM; 132 | BC062218, AJ224173, NM_002407, |
| family 2A, member 1) | | AF071219, BC062218 |
| FLT1 (Fms-related | Secreted/ | BC039007, NM_002019, S77812, |
| tyrosine kinase 1 (vascular | PM; 133 | S77814, AF063657, U01134, |
| endothelial growth | | BC039007, AB209050 |
| factor/vascular permeability | | |
| factor receptor) | | |
| C2orf6 (MOBK1B; MOB1, | TM; 134 | NM_018221, AK001650, AK023321, |
| Mps One Binder kinase | | BC003398, AK021657, AB016839, |
| activator-like 1B) | | AJ577474 |
| TMEM2 (Transmembrane | TM; 135 | BC044850, AB037833, NM_013390, |
| protein 2) | | AF137030, AK075370 |
| ACTR3 (ARP3 actin- | Arp2/3 | BC044590, NM_005721, AF006083 |
| related protein 3 homolog | protein | |
| (yeast)) | complex*; | |
| | 136 | |
| TMEM8 (Transmembrane | TM; 137 | BC021557, NM_021259, AB045292, |
| protein 8; five membrane- | | AK024725, AK091823, |
| spanning domains) | | |
| SLC5A4 (Solute carrier | TM; 138 | NM_014227, AJ133127, U41897 |
| family 5 (low affinity | | |
| glucose cotransporter), | | |
| member 4) | | |
| MEST (Mesoderm specific | TM; 139 | BC002413, BC011908, BC014564, |
| transcript homolog (mouse) | | BC018695, BC090049, NM_002402, |
| | | NM_177524, NM_177525, D78611, |
| | | D87367, Y11534 |
| CHODL (Chondrolectin) | TM; 140 | BC009418, NM_024944, AF257472, |
| | | AF523313, AF523314, AF523315, |
| | | AY358608 |
| TRIO; Triple functional | TM; 141 | AB115332, AK131423, NM_007118, |
| domain (PTPRF | | U42390, AF091395, BC017268, |
| interacting) | | BC035585, AB209754 |
| IL10RA (Interleukin 10 | TM; 142 | BC028082, NM_001558, AB209626, |
| receptor, alpha) | | U00672, BC028082 |
| LGALS3BP (Lectin, | ECM/PM; | BC002403, BC002998, BC015761, |
| galactoside-binding, | 143 | NM_005567, AK055977, AK057776, |
| soluble, 3 binding protein) | | L13210, X79089 |
| STK4 (Serine/threonine | TM/nucleus; | NM_006282, AK027088, BC042861, |
| kinase 4) | 144 | AB209315, BC093768, U18297, |
| | | U60207, BC005231 |
| ERBB3 (V-erb-b2 | TM; 145 | AK125028, NM_001982, |
| erythroblastic leukemia | | NM_001005915, BC082992, M29366, |
| viral oncogene homolog 3 | | M34309, S61953, U88357, U88358, |
| (avian) | | U88359, U88360, BC002706 |
| C14orf28 | TM; 146 | NM_001017923, BC105030, |
| | | BC105032, BC030258, AF250395 |
| KIAA1024 | TM; 147 | AF035294, AB028947, NM_015206 |
| CCL15 (chemokine (C-C | Secreted; 148 | NM_032965, NM_004167, |
| motif) ligand 15) | | NM_032964, NG_003025, DR005483 |
| PLA2G2D (Phospholipase | Secreted; 149 | AF112982, BC025706, NM_012400, |
| A2, group IID) | | AF188625 |
| KIAA1906 | TM; 150 | BC113366, AB067493, NM_020203, |
| | | AL080061, AB209172, AK090767, |
| | | NM_052907, AK093097 |
| F3; Coagulation factor III | TM; 151 | X07680, NM_001993, AF497569, |
| (thromboplastin, tissue | | AF497570, J02931, M16553, M27436 |
| factor) | | |
| PCDHB2 (Protocadherin | TM; 152 | BC098575, NM_018936, AF217756, |
| beta 2) | | AK027526, BC098347, AF152495, |
| | | BC098347 |
| KIAA0703 | TM; 153 | AK025424, AY791884, AK092737, |
| | | NM_014861, AB014603, CR749829, |
| | | BX648333 |
| C1orf10 (CRNN; Cornulin) | TM; 154 | BC030807, NM_016190, AF077831, |
| | | BC030807 |
| POLYDOM (SVEP1 | TM; 155 | AL079279, AL832416, AJ619977, |
| (Sushi, von Willebrand | | AK075200, AK075234, AY243503, |
| factor type A, EGF and | | AY916667, BX537918 |
| pentraxin domain | | |
| containing 1) | | |
| TUBAL3 (Tubulin, alpha- | TM; 156 | NM_024803, AK025318, BC098247, |
| like 3) | | BC105634, BC099716 |
| GPR105 (Purinergic | TM; 157 | BC034989, NM_014879, D13626 |
| receptor P2Y, G-protein | | |
| coupled, 14) | | |
| CNTN6 (Contactin 6) | PM; 158 | NM_014461, AB003592, BC113118, |
| | | BC113118 |
| IL7R (Interleukin 7 | TM; 159 | AF373204, AL713738, BC067537, |
| receptor) | | BC069999, BC067539, BC067540, |
| | | M29696 |
| ARHGAP18 (Rho GTPase | TM; 160 | BC017223, BC039611, BC052295, |
| activating protein 18) | | BC062417, BC107416, BC101708, |
| | | BC111940 |
| GRM1 (Glutamate | TM; 161 | NM_000838, AB208837, L76627, |
| receptor, metabotropic 1) | | U31215, U31216, L76631 |
| PREX1 (Phosphatidyl- | PM/cytosol; | BC009948, BC053616, NM_020820, |
| inositol 3,4,5-trisphosphate- | 162 | AB037836, AJ320261, AL136579, |
| dependent RAC exchanger | | AL832913 |
| 1) | | |
| MUC3A (Mucin 3A, | ECM/TM; | AW842071, M55405, M55406, |
| intestinal) | 163 | AF007194, AF143373 |
| LTBP2 (Latent | ECM; 164 | BC078659, S82451, Z37976, |
| transforming growth factor | | NM_000428, AF113211, AB209865 |
| beta binding protein 2) | | |
| EPSTI1 (Epithelial stromal | TM; 165 | BC023660, NM_033255, AF396928, |
| interaction 1 (breast)) | | NM_001002264, AL831953 |
| UBE2J1 (Ubiquitin- | TM/ER; 166 | BC013973, U93243, NM_016021, |
| conjugating enzyme E2, J1 | | AF151834, AF161502, AF151039, |
| (UBC6 homolog, yeast) | | AK223464 |

*These complexes are associated with actin filaments, some of which are localized to the plasma membrane.

### Example 9

### Further Testing of Transmembrane and Secreted TVM Materials and Experimental Methods

Cell-free, in vitro Rapid Translation System® (RTS) technology (Roche Applied Science) is used to produce whole proteins or polypeptides derived from extracellular regions.

### Results

The new transmembrane and secreted TVM identified in Example 8 are tested for enrichment of expression in ovarian tumors versus normal ovarian tissue and in different malignancies and tissues with physiologic angiogenesis, as described in Example 3. In other experiments, frozen specimens are used. In other experiments, paraffin-embedded specimens are used. Alternatively, the new TVM are used to localize ovarian cancer tumor vasculature using IHC or ISH, as described in Example 4. In another embodiment, double immuno-staining for CD31 in parallel is used to show co-localization in the endothelium. In another embodiment, antibodies raised against the proteins, or against extracellular fragments thereof, are utilized. In another embodiment, the antibodies are generated using methods described herein. In another embodiment, nucleic acids that hybridize with the identified transcripts are utilized. These experiments confirm that the identified transcripts, and the proteins they encode, are robust TVM.

### Example 10

### Use of Adlican as a Tumor Marker In Vivo

### Materials and Experimental Methods

Anti-Adlican antibody was produced by Prosci Inc., Poway, Calif.

### Results

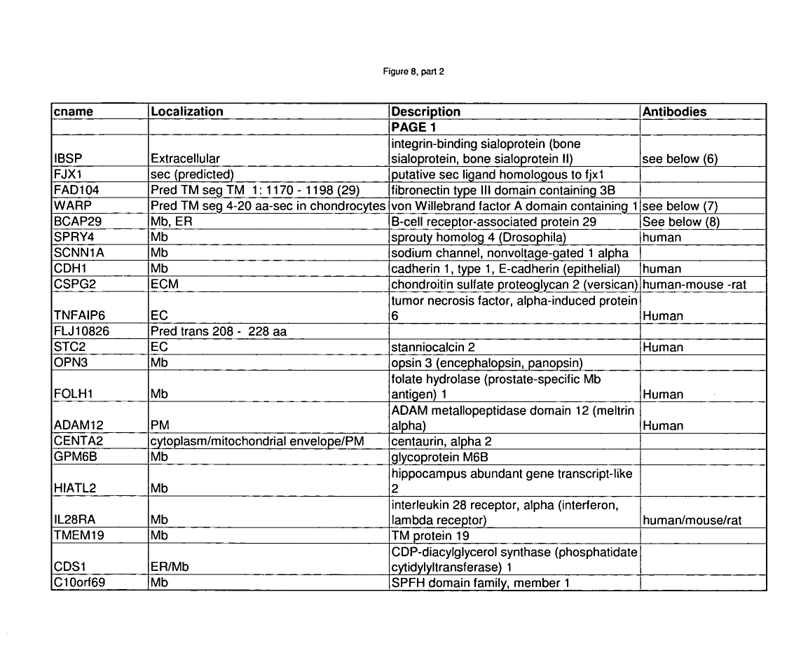

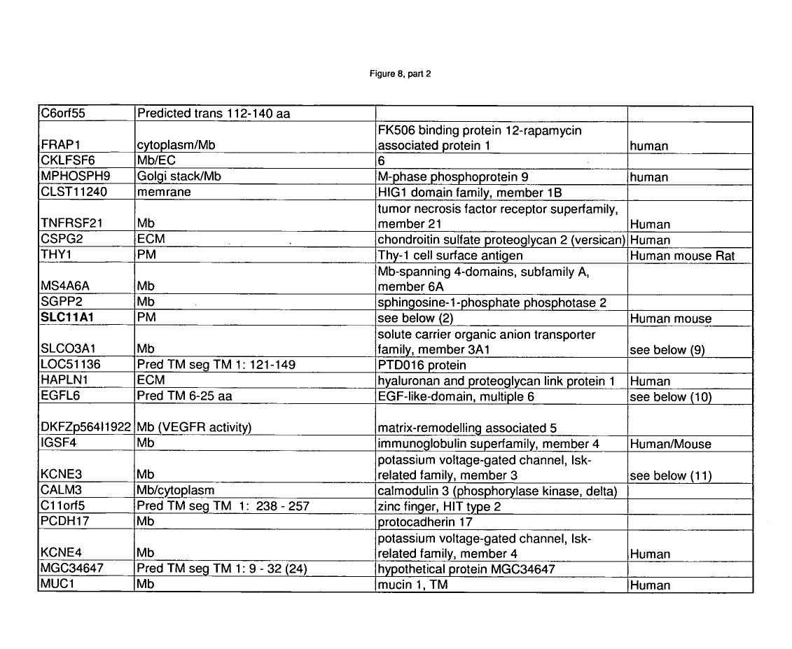

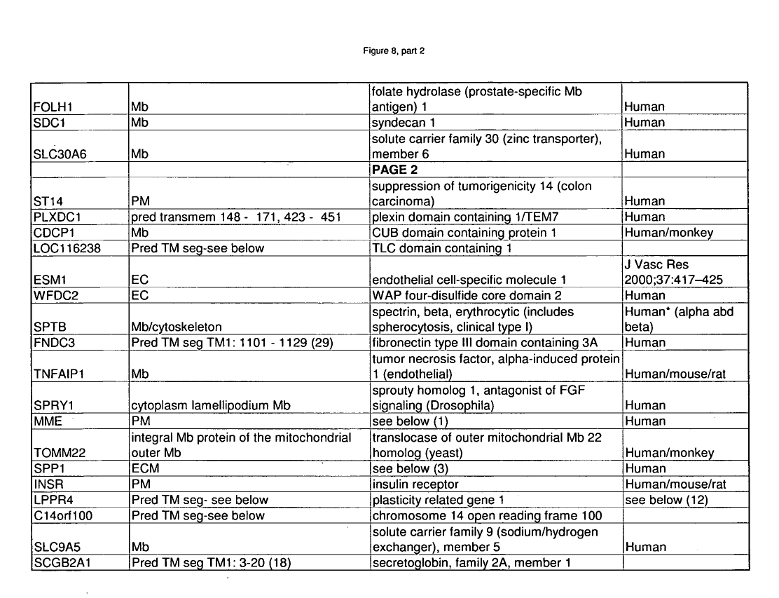

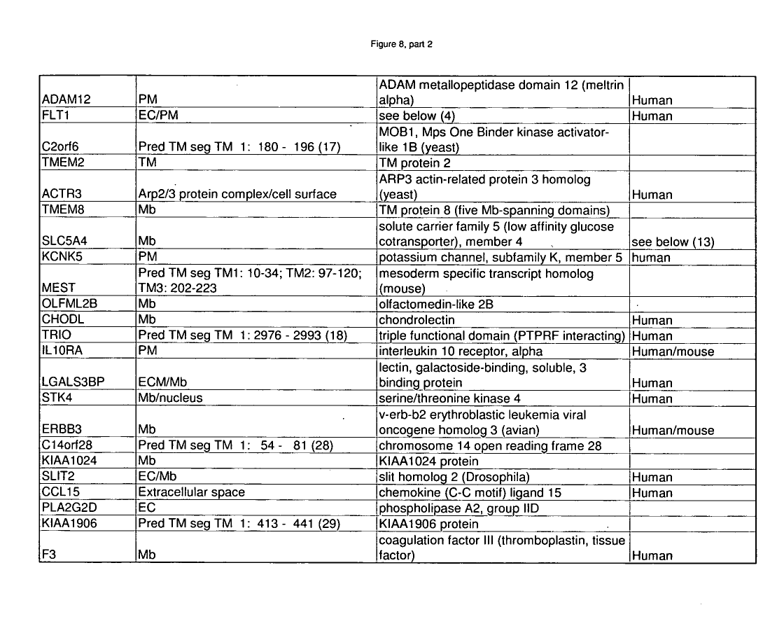

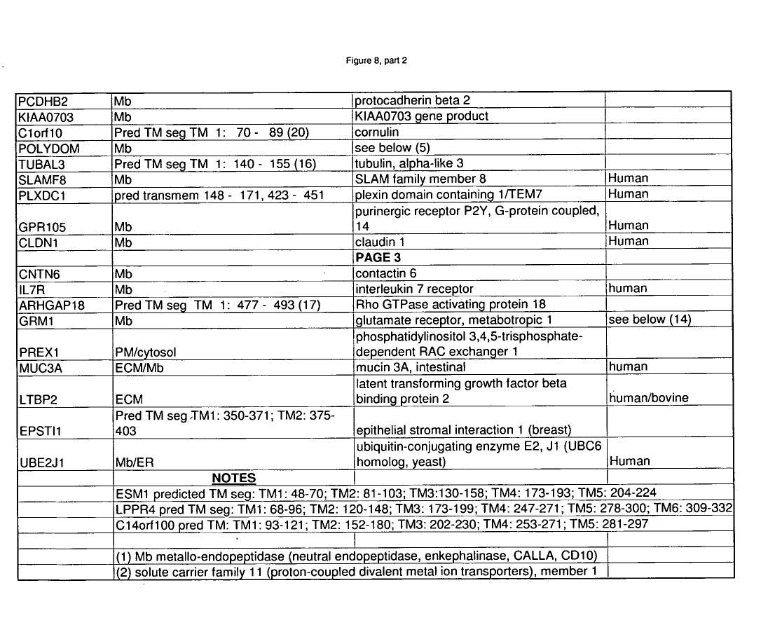

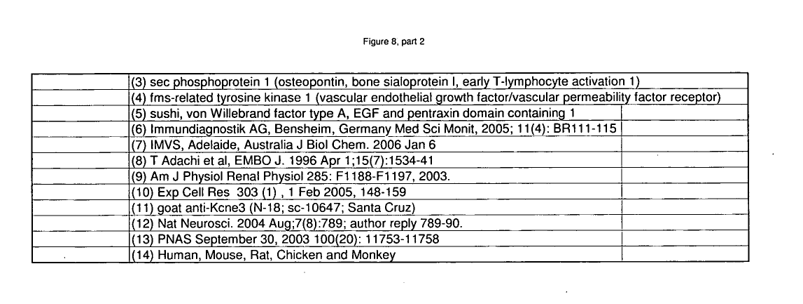

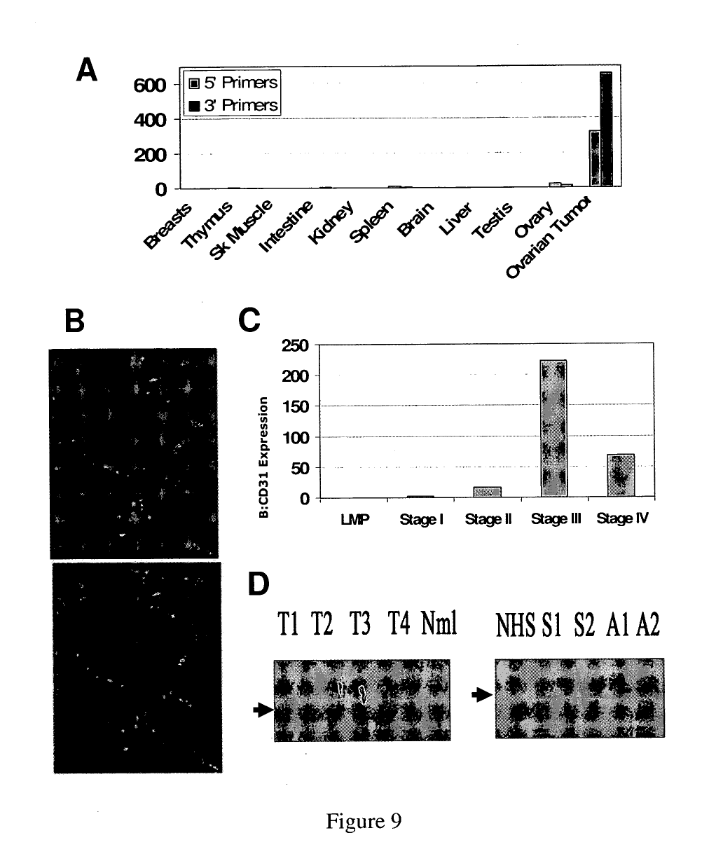

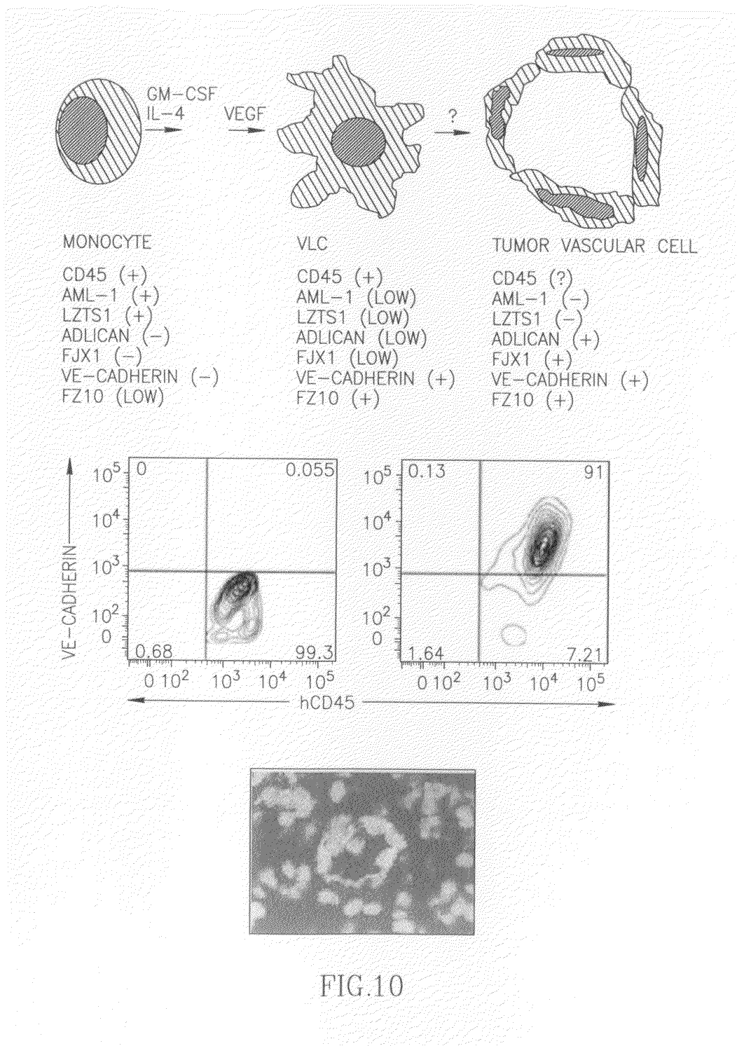

One extracellular matrix marker identified, adlican, was cloned, a fragment of it (CPGAKALSRVREDIVEDE; SEQ ID No: 88) was expressed, and rabbit anti-adlican antiserum was produced and used to screen normal and tumor tissue samples. Adlican was detected by immuno-staining in tumor but not in normal ovary stroma or vasculature. In addition, the antiserum was used to screen serum and ascites of patients with stage III ovarian cancer, and control healthy women. Adlican was detected by Western blot in the tumor samples, but not the control samples (FIG. 9D).

Thus, TMV of the present invention are efficacious in detecting, localizing, and specifically delivering drugs or anti-tumor agents to tumor cells. In addition, these results show that certain secretory TVM of the present invention are shed into the circulation, where they can function as tumor biomarkers. Thus, tests of biological fluids (e.g. blood, lymph, etc) can be successfully used to screen for tumors using TVM of the present invention.

### Example 11

### Identification of Additional TVM

### Materials and Experimental Methods

Additional TVM were identified from the list of genes described in Example 1, according to the following criteria: (1) Genes which were determined to be present in 75% of tumor vasculature specimens and absent in normal vasculature; and (2) genes which were present in 75% of tumor vasculature specimens and greater than 5-fold upregulated versus normal ovarian endothelium.

### Results

Additional TVM were identified; this list overlapped significantly with the list in Table 5. Some of the additional TVM that do not appear in the above tables are set forth in Table 8. Each GenBank Accession No. represents a separate embodiment of the present invention:

| | SEQ ID | Sequence and/or exemplary GenBank |
|---|---|---|
| Gene name: | Number | Accession Numbers |
| HOXA9 | 167 | BC006537, BC010023, NM_152739, |
| (Homeobox A9) | | U41813, NM_002142, U82759, |
| | | BT006990 |
| SLPI (Secretory | 168 | BC020708, NM_003064, X04470, |
| leukocyte peptidase | | X04503, AF114471 |
| inhibitor) | | |
| KIBRA (also | 169 | BC004394, AK001727, NM_015238, |
| known as WWC1; | | BC017746, AF506799, AY189820, |
| WW, C2 and coiled- | | AF530058, AB020676, BX640827 |
| coil domain | | |
| containing 1) | | |

In vivo expression analyses validated the markers in Table 8 and DEFB1, IL10RA, ADAM12, PCDH17, F-spondin, TNFAIP6, BLAME, AML-1, HEVIN, EPB41L3, SLIT2, and LZTS1, which also were identified in this analysis. Measurement of expression in normal ovarian tissue vs. tumor, and an array of normal tissues vs. tumors, performed as described in the above Examples, both validated each of these markers (FIG. 3).

Additional TVM identified in the above analysis (besides those enumerated elsewhere herein) were MGAT4A, AFAP, CXCR4, UCP2, TWIST, SLC2A3, MYO1B, COL4A2, MGC4677, G1P2, BHLHB3, NEDL2, and ITGA1 (FIG. 11).

Thus, as in previous Examples, each of the markers tested in this Example are bona fide TVM.

### Example 12

### Identification of Additional Transmembrane (TM) and Secreted TVM

Additional TVM were identified from the list of genes described in Example 1, according to the following criteria: A. For each gene, the individual rank values of the 5 normal endothelium samples were summed; the sum of ranks had to be less or equal to 25; (2) AND/OR the fold change (mean tumor to mean normal) had to be greater or equal to 3. From these genes, all genes with either TM or extracellular function were selected.

The additional TVM are listed in FIG. 12, together with GenBank Accession Numbers for those markers not listed elsewhere herein (SEQ ID No: 170-211). Each GenBank Accession No. represents a separate embodiment of the present invention.

### Example 13

### Development of New Antibodies Against TVM

### Materials and Experimental Methods

### Peptides

Peptides are synthesized using an Fmoc-based chemical strategy.

### Antibodies

Antibody production is outsourced to specialized biotechnology companies; e.g. Prosci Inc. (Poway, Calif.). To exclude any Fc fragment-mediated effects that may interfere with Ab specificity, Abs are prepared as F(ab′)₂ fragments.

### Results

The TVM identified in the above Examples are used as targets for the development of epitope-specific monoclonal (m)Abs and affinity-purified anti-peptide polyclonal (p)Abs. For target screening, affinity-purified pAbs are initially generated. In other experiments, synthetic peptides spanning epitopic regions of 20-30 extracellular hydrophilic residues from secretory or transmembrane proteins from the above Examples are synthesized. In another embodiment, the epitopic regions of extracellular hydrophilic residues are deduced based on their primary sequence.

Monoclonal antibodies to whole proteins are generated for promising candidates.

The work described herein generates and identifies mAb and pAb for screening and validation studies. Certain of these Abs are also suitable to clinical applications for serum cancer diagnostics, e.g. as described below

### Example 14

### Identification of Additional TVM Using Phage Display Libraries

### Materials and Experimental Methods

### Phage Display Libraries

To derive human Abs, a non-immune library of phage-displayed human single-chain Fv Abs is generated.

### Construction of the VH Library

Total RNA is prepared from ovarian cancer cells and normal ovary samples. cDNA is synthesized from total RNA primed with the HuIgMFOR primer (TGGAAGAGGCACGTTCTTTTCTTT; SEQ ID No: 71). VH gene repertoires are amplified from the cDNA by using Vent DNA polymerase (New England Biolabs) in combination with the HuIgMFOR primer and an equimolar mixture of HuVHBACK primers (CAGGTGCAGCTGGTGCAGTCTGG—SEQ ID No: 72—CAGGTCAACTTAAGGGAGTCTGG—SEQ ID No: 73; GAGGTGCAGCTGGTGGAGTCTGG—SEQ ID No: 74; CAGGTGCAGCTGCAGGAGTCGGG—SEQ ID No: 75; CAGGTACAGCTGCAGCAGTCAGG—SEQ ID No: 76). PCR products are agarose gel-purified and re-amplified to append NcoI and NotI restriction sites by using Tth DNA polymerase (Epicentre Technologies, Madison, Wis.) and an equimolar mixture of the HuVHBACK Sfi primers (GTCCTCGCAACTGCGGCCCAGCCGGCCATGGCCCAGGTCAACTTAAGGGAG TCTGG—SEQ ID No: 77) GTCCTCGCAACTGCGGCCCAGCCGGCCATGGCCGAGGTGCAGCTGGTGGAG TCTGG—SEQ ID No: 78; GTCCTCGCAACTGCGGCCCAGCCGGCCATGGCCCAGGTGCAGCTGCAGGAG TCGGG—SEQ ID No: 79; GTCCTCGCAACTGCGGCCCAGCCGGCCATGGCCCAGGTGCAGCTGTTGCAG TCTGC—SEQ ID No: 80; GTCCTCGCAACTGCGGCCCAGCCGGCCATGGCCCAGGTACAGCTGCAGCAG TCAGG—SEQ ID No: 81) and the HuCMFor Not primer (5′-GAGTCATTCTCGACTTGCGGCCGCTGGAAGAGGCACGTTCTTTTCTTT-3′; SEQ ID No: 82). The PCR products are digested with NcoI and NotI and agarose gel-purified. The resulting DNA fragments are ligated into the plasmid pCITE3A (Novagen; any plasmid that contains the proper restriction sites for cloning and unique sequences for PCR amplification works as well) and cut with restriction enzymes NcoI and NotI, and the ligated DNA is electroporated into the E. coli strain TG1. If desired, cloning efficiency and library diversity ix determined by PCR screening.

Construction of the scFv Library

The VH gene repertoire is PCR-amplified from the pCITE-VH library by using 300 ng of library plasmid DNA as a template, Vent DNA polymerase, the CITE3 primer (GATCTGATCTGGGGCCTCGGTGC—SEQ ID No: 83), and an equimolar mixture of HuJH primers. The VL genes for scFv assembly are obtained from an available scFv phage antibody library. The VL gene repertoire, including DNA encoding the scFv peptide linker (G₄S)₃, is amplified from 300 ng of library plasmid DNA by using Vent DNA polymerase, the Gene3 primer (5′-GCAAGCCCAATAGGAACCCATGTACCG—SEQ ID No: 84), and an equimolar mixture of RHuJH primers. The amplified VH and VL genes are agarose gel-purified and spliced together with overlap extension PCR to create a scFv gene repertoire. To facilitate joining VH and VL gene repertoires with overlap extension PCR, the input DNA fragments have blunt ends. The proofreading DNA polymerase Vent is used to generate the VH and VL DNA fragments for scFv assembly. For all subsequent PCR steps of library construction, Tth DNA polymerase was found to be the optimal enzyme. The VH and VL gene repertoires are spliced together in 100-μl PCR reactions containing 100 ng of the VH and VL DNA fragments and Tth DNA polymerase. The reactions are cycled eight times (95° C. 2 min, 55° C., 1 min, and 72° C. 3 min) to join the fragments. Then the CITE3 and Gene3 primers are added, and the reaction is cycled 30 times (94° C. 1 min, 55° C. 1 min, and 72° C. 3 min) to amplify the assembled scFv genes. The scFv genes are digested with restriction enzymes NcoI and NotI, agarose gel-purified, and ligated into the plasmid pHEN-1 digested with NcoI and NotI. The ligated DNA is electroporated into E. coli TG1 cells

This method enables rapid production of panels (˜7×10⁹) of high-affinity (<1 nM) mAbs, which are desirable for therapeutics and diagnostics.

### Results

Single-chain Fv (scFv) Ab phage display libraries are generated from patients with ovarian cancer and screened to identify antibodies (Abs) recognizing TVM identified in the above Examples. To identify auto-Abs that recognize TVM in ovarian cancer patients, phage display libraries are generated from ovarian cancer tumor samples. Separate IgGκ and IgGλ phage libraries are constructed, using the phagemid vector pComb3X (Scripps Research Institute) from 4×10⁷ peripheral blood or ascites mononuclear cells. For library selection, IgGκ and IgGλ Ab phage display libraries are combined in equal amounts and panned by solid-phase selection on ELISA plates coated with generated peptides or proteins. Binding of individual phage clones isolated from each round of panning can be confirmed by ELISA, if desired. The VH and VL nucleic acid sequences are determined for positive clones to establish a cohort of unique mAbs specific to TVM. scFv monoclonal preparations unlinked to phages are produced using TOP10F′ non-suppressor E. coli. In other experiments, phage display libraries are panned on frozen tissue sections of normal ovaries and then on sections of ovarian cancer. Slides are then subjected to immuno-LCM, as described in previous Examples. With this approach, phages recognizing TVM are removed selectively from the tissue. RNA is isolated and used to infect additional phages. Serial panning between normal and tumor tissue leads to the selection to tumor vascular-specific phages, which are then used to develop Abs as above. With this alternative approach, additional TVM are identified

In other experiments, human scFv Ab libraries from normal donors are screened.

### Example 15

### Use of Transmembrane TVM for Tumor Localization In Vivo

The transmembrane TVM identified in the above Examples are used as targets for localizing ovarian or other solid tumors, as described for Adlican in Example 10. In other embodiments, radio-labeled antibodies are used for imaging. In another embodiment, positron-emission tomography is used.

### Example 16

### Use of Transmembrane TVM for Delivery of Anti-Tumor Agents In Vivo

In another embodiment, an antibody identified in one of the above Examples is used to target an anti-tumor agent to ovarian or other solid tumor in vivo. In some experiments, the anti-tumor agent is a radioactive antibody. In other experiments, the anti-tumor agent is an antibody conjugated to a nanosphere that contains an anti-tumor drug.

### Example 17

### Use of Secreted TVM for Serum Diagnostic Testing of Cancer In Vivo

Existing Abs and novel Abs or scFv identified in the above Examples, recognizing secretory TVM of the present invention, are tested for serum diagnostic applications. ELISA is utilized to test how well each TVM performs as a biomarker for ovarian cancer serum detection. In addition, TVM are tested as biomarkers for other solid tumors.

### Example 18

### Use of Transmembrane TVM for Vasculature-Redirected Lymphocyte Therapy

Chimeric immuno-receptors composed of high-affinity scFv recognizing TVM of the present invention (Example 14) fused to the cytoplasmic signaling modules of T cell receptor, CD28 and 4-1BB are created as described in Ledbetter J A et al (Enhanced transmembrane signaling activity of monoclonal antibody hetero-conjugates suggests molecular interactions between receptors on the T cell surface. Mol. Immunol. 1989 February; 26(2):137-45). The chimeric receptors are then utilized for vasculature-redirected lymphocyte therapy.

### Example 19

### Additional Studies with TNFRSF21 (DR6)

Expression of DR6 mRNA was determined in a variety of normal and tumor tissues by Affymetrix® array. Expression was relatively low in normal tissues (Table 9), while DR6 was heavily expressed in many tumor tissues, particularly bladder (Table 10).

TABLE 9
Affymetrix array scores of DR6 in normal tissues.

| | Affy |
|---|---|
| Tissue | score |
| Adipose tissue | 6 |
| Adrenal gland cortex | 4 |
| Bone marrow | 4 |
| Bronchus | 6 |
| Cerebellum | 5 |
| Cerebral cortex | 6 |
| Cerebrum | 6 |
| Cervix | 5 |
| Coronary artery | 5 |
| Colon cecum | 5 |
| Endometrium | 5 |
| Esophagus | 6 |
| Heart atrium | 5 |
| Heart ventricle | 5 |
| Kidney cortex | 6 |
| Kidney medulla | 6 |
| Liver | 4 |
| Lung | 5 |
| Lymph nodes | 6 |
| Mammary gland | 5 |
| Medulla | 6 |
| Midbrain | 6 |
| Myometrium | 5 |
| Nipple cross section | 5 |
| Oral mucosa | 6 |
| Ovary | 4 |
| Pharyngeal mucosa | 6 |
| Pituitary gland | 5 |
| Prostate gland | 5 |
| Salivary gland | 5 |
| Saphenous vein | 5 |
| Skeletal muscle | 4 |
| Spinal cord | 7 |
| Spleen | 4 |
| Stomach | 6 |
| Testes | 4 |
| Thyroid gland | 4 |
| Tongue | 6 |
| Tonsil | 6 |
| Trachea | 7 |
| Trigeminal ganglia | 6 |
| Urethra | 6 |
| Vagina | 5 |
| Vulva | 5 |

TABLE 10
Affymetrix array scores of DR6 in tumor tissues.

| | Affy |
|---|---|
| Tissue | score |
| Adrenal gland | 7 |
| Bladder | 8 |
| Brain | 6 |
| Breast | 6 |
| Cervix | 7 |
| Colon | 7 |
| Corpus uteri | 6 |
| Endometrium | 7 |
| Esophagus | 6 |
| Kidney | 7 |
| Liver | 7 |
| Lung | 7 |
| Omentum | 6 |
| Ovary | 7 |
| Pancreas | 7 |
| Prostate | 6 |
| Rectosigmoid | 7 |
| Small intestine | 6 |
| Stomach | 7 |
| Testis | 7 |
| Thyroid | 5 |
| Vulva | 6 |

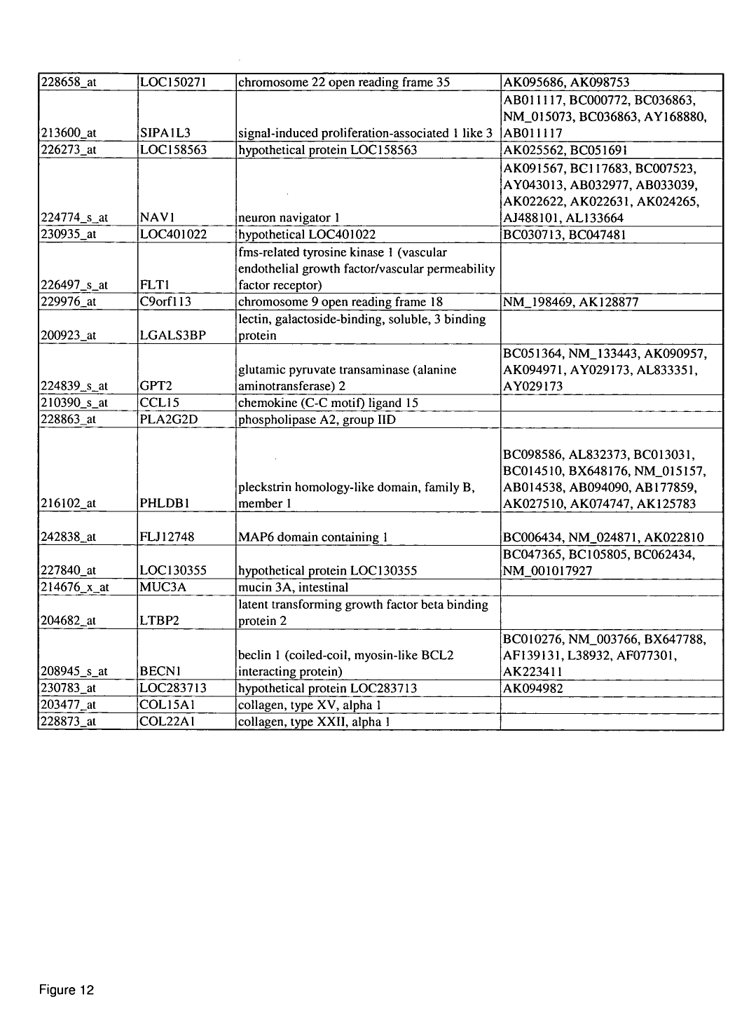

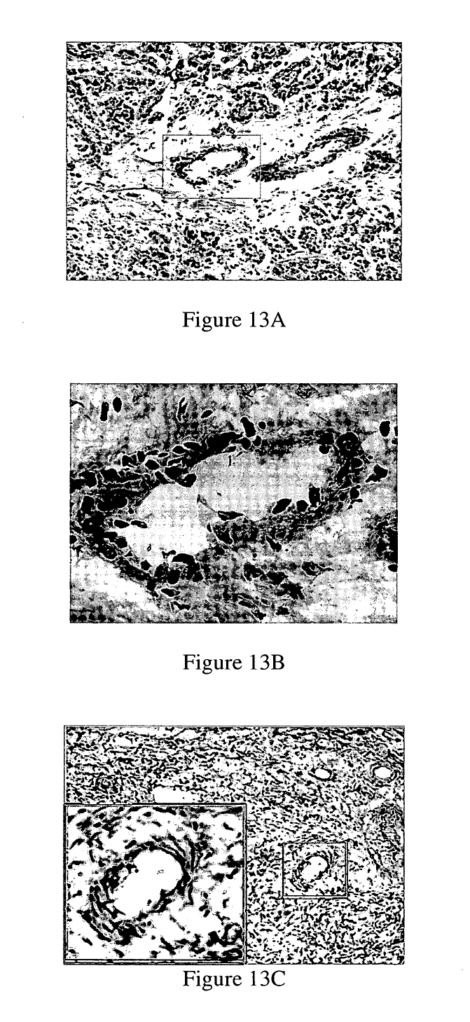

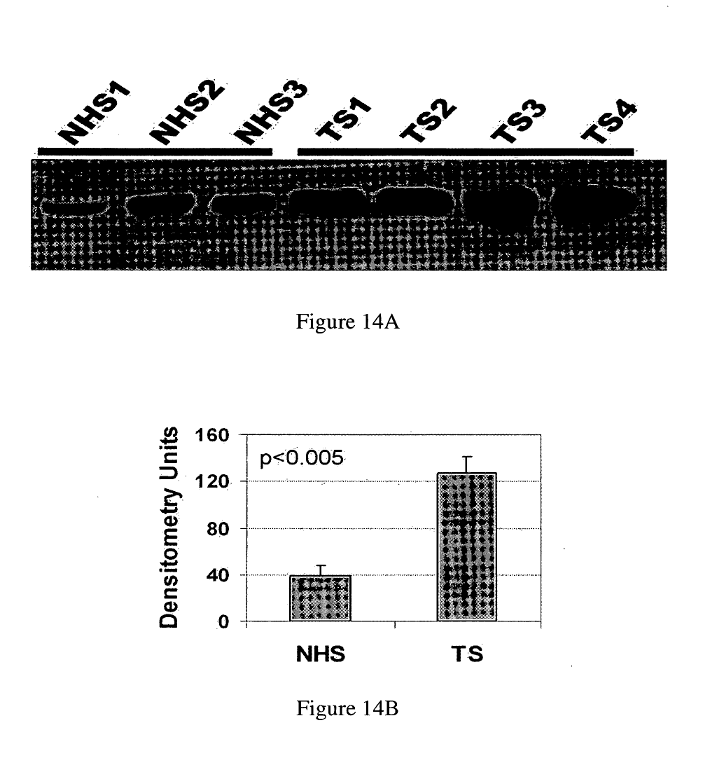

Further, enhanced expression of DR6 in ovarian tumor relative to normal ovary tissue was confirmed by qPCR and IHC (FIG. 13A-B). In addition, increased serum levels of DR6 were found in serum of patients with ovarian tumors compared to healthy individuals (FIG. 14). Thus, DR6 is efficacious as a tumor marker (e.g. for ovarian tumors), both in situ and in tumors.

### Example 20

### Additional Studies with ADAM12

Expression of ADAM12 mRNA was determined in a variety of normal and tumor tissues by Affymetrix® array. Expression was relatively low in normal tissues (Table 11); while DR6 was significantly enriched in several tumor tissues (Table 12).

TABLE 11
Affymetrix array scores of ADAM12 in normal tissues.

| | Affy |
|---|---|
| Tissue | score |
| Adipose tissue | 3 |
| Adrenal gland cortex | 2 |
| Bone marrow | 2 |
| Bronchus | 2 |
| Cerebellum | 2 |
| Cerebral cortex | 2 |
| Cerebrum | 2 |
| Cervix | 2 |
| Colon cecum | 2 |
| Coronary artery | 2 |
| Endometrium | 5 |
| Esophagus | 2 |
| Heart atrium | 2 |
| Heart ventricle | 2 |
| Kidney cortex | 2 |
| Kidney medulla | 2 |
| Liver | 2 |
| Lung | 2 |
| Lymph nodes | 3 |
| Mammary gland | 3 |
| Medulla | 2 |
| Midbrain | 2 |
| Myometrium | 2 |
| Nipple cross section | 2 |
| Oral mucosa | 2 |
| Ovary | 4 |
| Pharyngeal mucosa | 2 |
| Pituitary gland | 2 |
| Prostate gland | 2 |
| Salivary gland | 2 |
| Saphenous vein | 2 |
| Skeletal muscle | 2 |
| Spinal cord | 2 |
| Spleen | 2 |
| Stomach | 2 |
| Testes | 2 |
| Thyroid gland | 2 |
| Tongue | 2 |
| Tonsil | 3 |
| Trachea | 2 |
| Trigeminal ganglia | 2 |
| Urethra | 2 |
| Vagina | 2 |
| Vulva | 3 |

TABLE 12
Affymetrix array scores of ADAM12 in tumor tissues.

| | Affy |
|---|---|
| Tissue | score |
| Adrenal gland | 5 |
| Bladder | 5 |
| Brain | 3 |
| Breast | 6 |
| Cervix | 5 |
| Colon | 5 |
| Corpus uteri | 6 |
| Endometrium | 5 |
| Esophagus | 3 |
| Kidney | 3 |
| Liver | 5 |
| Lung | 5 |
| Omentum | 5 |
| Ovary | 4 |
| Pancreas | 5 |
| Prostate | 3 |
| Rectosigmoid | 5 |
| Small intestine | 3 |
| Stomach | 3 |
| Testis | 3 |
| Thyroid | 3 |
| Vulva | 6 |

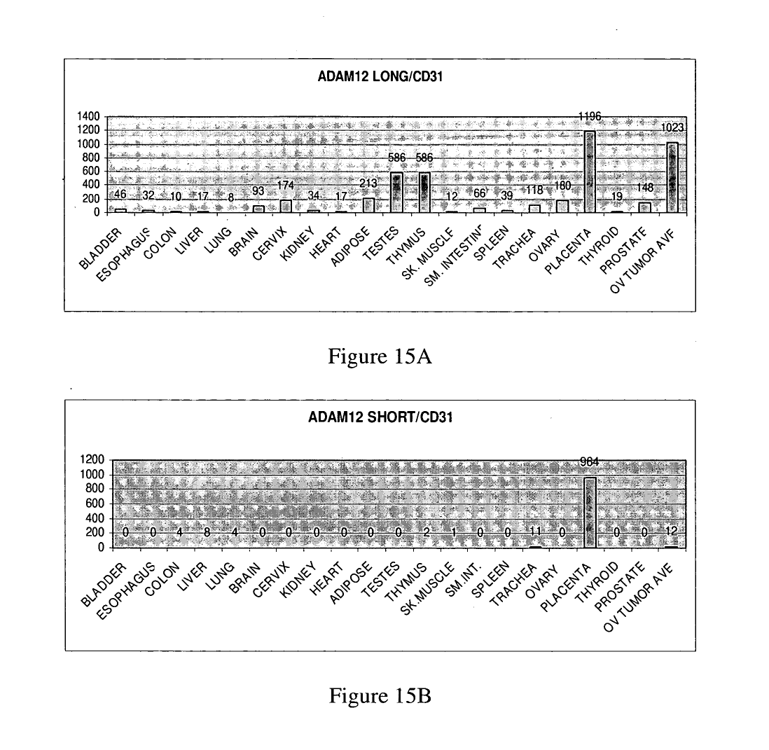

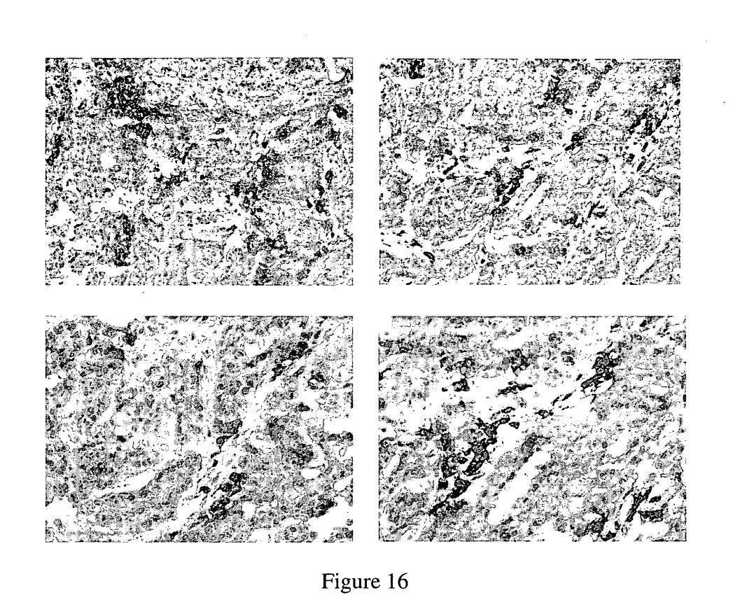

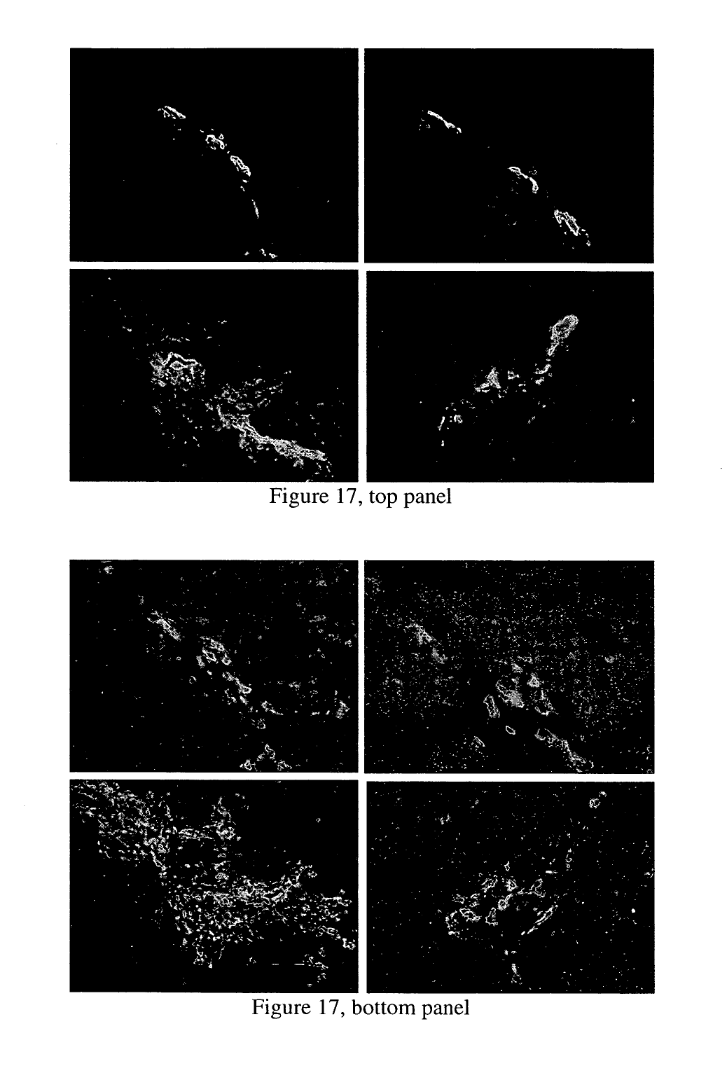

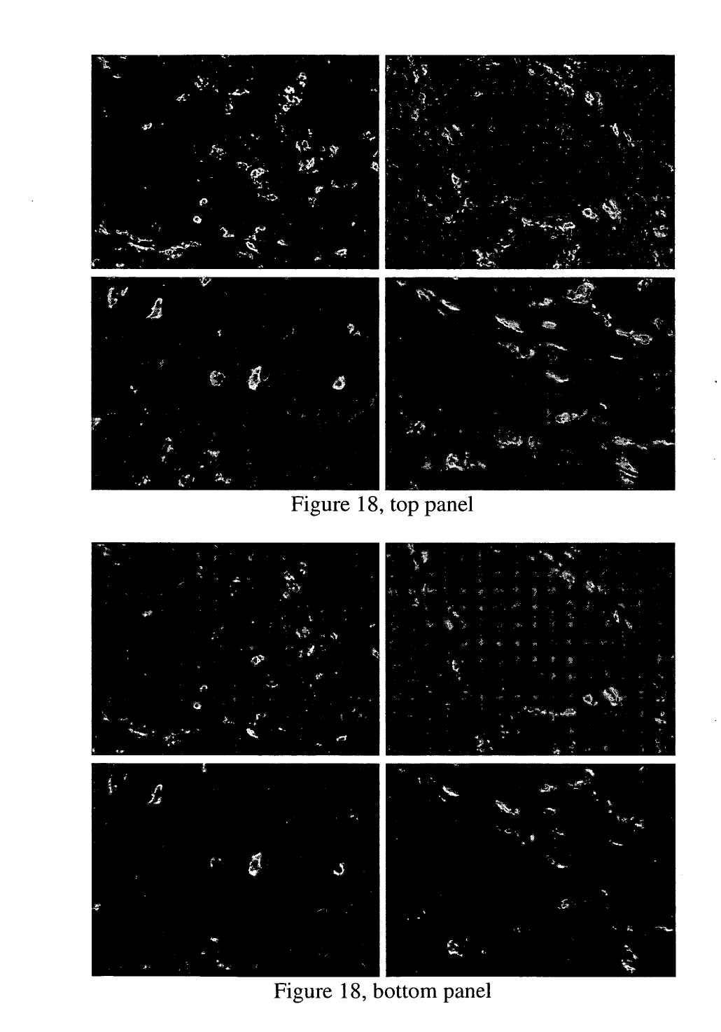

Further, enhanced expression of ADAM12 in ovarian tumor relative to normal tissues was confirmed by qPCR (FIG. 15). In addition, staining of ADAM12 was demonstrated in ovarian tumors (FIG. 16). The relevance of ADAM12 as a TVM was further confirmed by co-staining with CD31 and CD45 (FIGS. 17-18). Thus, ADAM12 is efficacious as a tumor marker (e.g. for ovarian tumors), further validating the results of the present invention.

### Example 21

### Additional Studies with CDCP1

Expression of CDCP1 mRNA was determined in a variety of normal and tumor tissues by Affymetrix® array. Expression was significantly enriched in several tumor tissues compared to most normal tissues (Tables 14 and 13, respectively).

TABLE 13
Affymetrix array scores of CDCP1 in normal tissues.

| | Affy |
|---|---|
| Tissue | score |
| Adipose tissue | 3 |
| Adrenal gland cortex | 3 |
| Bone marrow | 3 |
| Bronchus | 3 |
| Cerebellum | 3 |
| Cerebral cortex | 3 |
| Cerebrum | 3 |
| Cervix | 3 |
| Colon cecum | 3 |
| Coronary artery | 3 |
| Endometrium | 3 |
| Esophagus | 5 |
| Heart atrium | 3 |
| Heart ventricle | 3 |
| Kidney cortex | 3 |
| Kidney medulla | 3 |
| Liver | 3 |
| Lung | 3 |
| Lymph nodes | 3 |
| Mammary gland | 3 |
| Medulla | 3 |
| Midbrain | 3 |
| Myometrium | 3 |
| Nipple cross section | 4 |
| Oral mucosa | 5 |
| Ovary | 3 |
| Pharyngeal mucosa | 5 |
| Pituitary gland | 3 |
| Prostate gland | 4 |
| Salivary gland | 3 |
| Saphenous vein | 3 |
| Skeletal muscle | 3 |
| Spinal cord | 3 |
| Spleen | 3 |
| Stomach | 4 |
| Testes | 3 |
| Thyroid gland | 4 |
| Tongue | 4 |
| Tonsil | 4 |
| Trachea | 4 |
| Trigeminal ganglia | 3 |
| Urethra | 4 |
| Vagina | 4 |
| Vulva | 5 |

TABLE 14
Affymetrix array scores of CDCP1 in tumor tissues.

| | Affy |
|---|---|
| Tissue | score |
| Adrenal gland | 5 |
| Bladder | 5 |
| Brain | 4 |
| Breast | 5 |
| Cervix | 5 |
| Colon | 5 |
| Corpus uteri | 5 |
| Endometrium | 5 |
| Esophagus | 5 |
| Kidney | 4 |
| Liver | 5 |
| Lung | 5 |
| Omentum | 5 |
| Ovary | 5 |
| Pancreas | 5 |
| Prostate | 4 |
| Rectosigmoid | 5 |
| Small intestine | 5 |
| Stomach | 5 |
| Testis | 3 |
| Thyroid | 4 |
| Vulva | 5 |

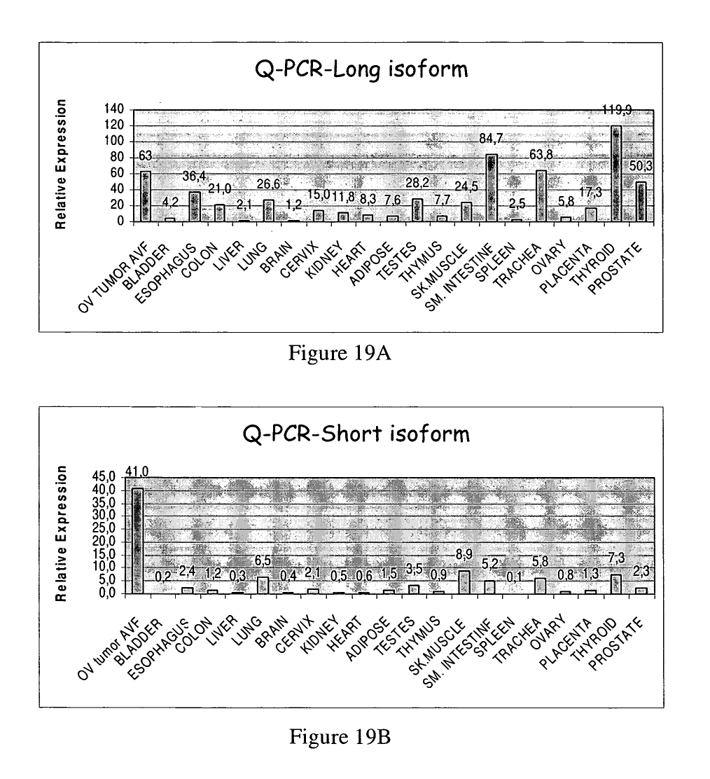

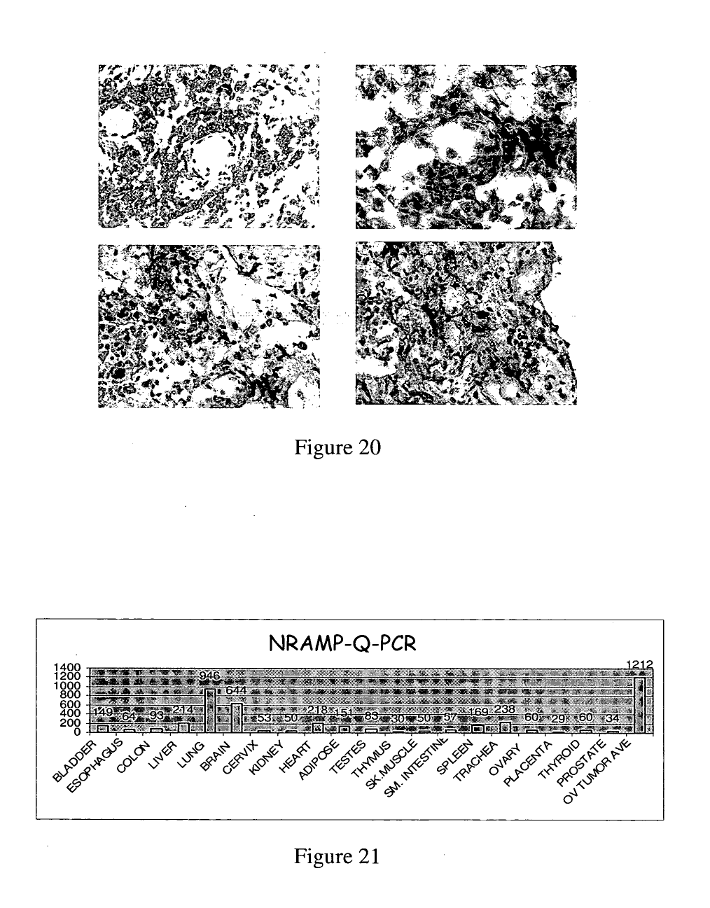

Further, enhanced expression of the short isoform of CDCP1 in ovarian tumor relative to normal tissues was confirmed by qPCR (FIG. 19). In addition, staining of CDCP1 was demonstrated in ovarian tumors (FIG. 20). Thus, CDCP1 is efficacious as a tumor marker (e.g. for ovarian tumors), further validating the results of the present invention.

### Example 22

### Additional Studies with SLC11A1

Expression of SLC11A1 mRNA was determined in a variety of normal and tumor tissues by Affymetrix® array. Expression was significantly enriched in several tumor tissues compared to most normal tissues (Tables 16 and 15, respectively).

TABLE 15
Affymetrix array scores of SLC11A1 in normal tissues.

| | Affy |
|---|---|
| Tissue | score |
| Adipose tissue | 4 |
| Adrenal gland cortex | 4 |
| Bone marrow | 5 |
| Bronchus | 4 |
| Cerebellum | 4 |
| Cerebral cortex | 4 |
| Cerebrum | 4 |
| Cervix | 4 |
| Colon cecum | 4 |
| Coronary artery | 4 |
| Endometrium | 4 |
| Esophagus | 4 |
| Heart atrium | 4 |
| Heart ventricle | 4 |
| Kidney cortex | 4 |
| Kidney medulla | 4 |
| Liver | 4 |
| Lung | 5 |
| Lymph nodes | 4 |
| Mammary gland | 4 |
| Medulla | 4 |
| Midbrain | 4 |
| Myometrium | 4 |
| Nipple cross section | 4 |
| Oral mucosa | 4 |
| Ovary | 4 |
| Pharyngeal mucosa | 4 |
| Pituitary gland | 4 |
| Prostate gland | 4 |
| Salivary gland | 4 |
| Saphenous vein | 4 |
| Skeletal muscle | 5 |
| Spinal cord | 4 |
| Spleen | 4 |
| Stomach | 4 |
| Testes | 4 |
| Thyroid gland | 4 |
| Tongue | 4 |
| Tonsil | 4 |
| Trachea | 4 |
| Trigeminal ganglia | 4 |
| Urethra | 4 |
| Vagina | 4 |
| Vulva | 5 |

TABLE 16
Affymetrix array scores of SLC11A1 in tumor tissues.

| | Affy |
|---|---|
| Tissue | score |
| Adrenal gland | 4 |
| Bladder | 5 |
| Brain | 5 |
| Breast | 5 |
| Cervix | 5 |
| Colon | 5 |
| Corpus uteri | 5 |
| Endometrium | 5 |
| Esophagus | 4 |
| Kidney | 5 |
| Liver | 5 |
| Lung | 5 |
| Omentum | 5 |
| Ovary | 5 |
| Pancreas | 5 |
| Prostate | 5 |
| Rectosigmoid | 5 |
| Small intestine | 5 |
| Stomach | 5 |
| Testis | 5 |
| Thyroid | 5 |
| Vulva | 5 |

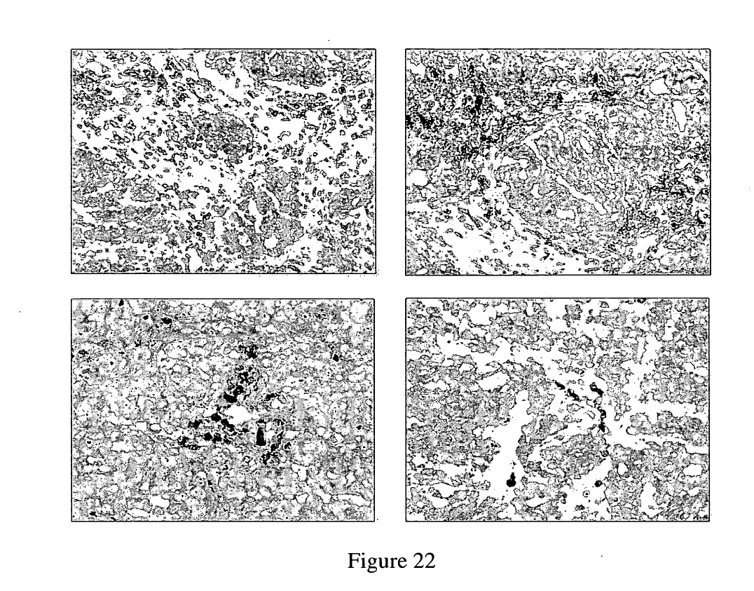

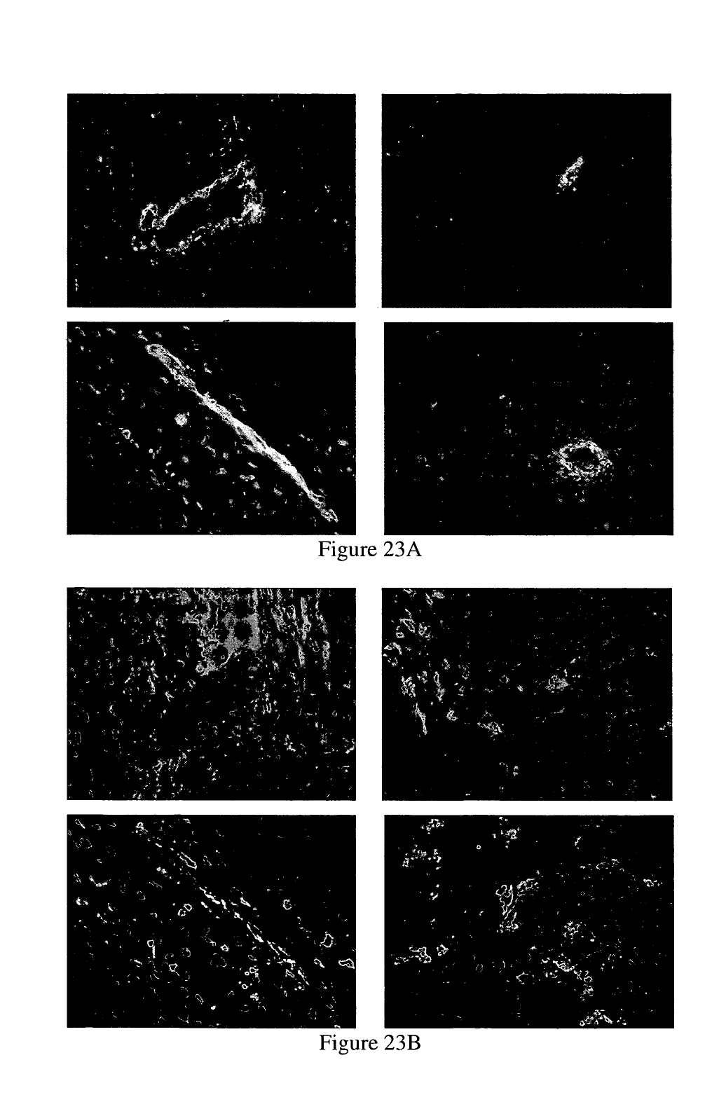

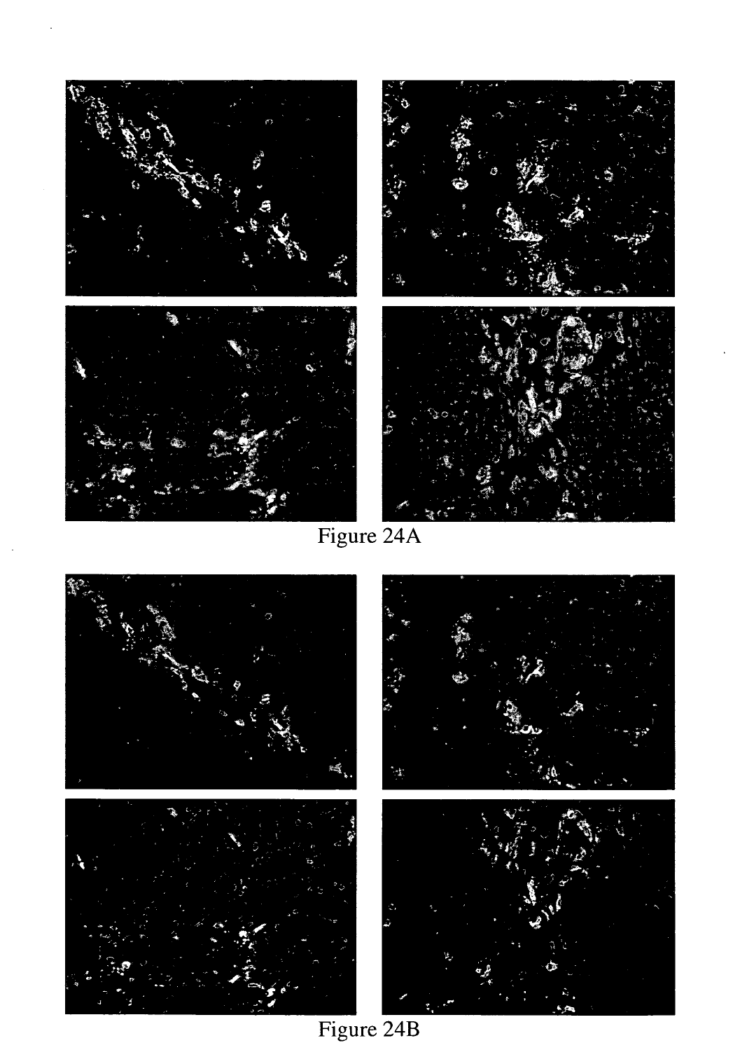

Further, enhanced expression of SLC11A1 in ovarian tumor relative to normal tissues was confirmed by qPCR (FIG. 21). In addition, staining of SLC11A1 was demonstrated in ovarian tumors (FIG. 22). The relevance of SLC11A1 as a TVM was further confirmed by co-staining with CD31 and CD45 (FIGS. 23-24). Thus, SLC11A1 is efficacious as a tumor marker (e.g. for ovarian tumors).

### Example 23

### Additional Studies with Blame

Expression of BLAME mRNA was determined in a variety of normal and tumor tissues by Affymetrix® array. Expression was significantly enriched in most tumor tissues compared to most normal tissues (Tables 18 and 17, respectively).

TABLE 17
Affymetrix array scores of BLAME in normal tissues.

| | Affy |
|---|---|
| Tissue | score |
| Adipose tissue | 3 |
| Adrenal gland cortex | 2 |
| Bone marrow | 3 |
| Bronchus | 3 |
| Cerebellum | 2 |
| Cerebral cortex | 2 |
| Cerebrum | 2 |
| Cervix | 2 |
| Colon cecum | 3 |
| Coronary artery | 3 |
| Endometrium | 2 |
| Esophagus | 2 |
| Heart atrium | 2 |
| Heart ventricle | 2 |
| Kidney cortex | 2 |
| Kidney medulla | 2 |
| Liver | 3 |
| Lung | 4 |
| Lymph nodes | 5 |
| Mammary gland | 3 |
| Medulla | 2 |
| Midbrain | 2 |
| Myometrium | 2 |
| Nipple cross section | 3 |
| Oral mucosa | 3 |
| Ovary | 2 |
| Pharyngeal mucosa | 3 |
| Pituitary gland | 2 |
| Prostate gland | 2 |
| Salivary gland | 2 |
| Saphenous vein | 3 |
| Skeletal muscle | 2 |
| Spinal cord | 2 |
| Spleen | 3 |
| Stomach | 3 |
| Testes | 2 |
| Thyroid gland | 3 |
| Tongue | 3 |
| Tonsil | 4 |
| Trachea | 3 |
| Trigeminal ganglia | 3 |
| Urethra | 3 |
| Vagina | 2 |
| Vulva | 2 |

TABLE 18
Affymetrix array scores of BLAME in tumor tissues.

| | Affy |
|---|---|
| Tissue | score |
| Adrenal gland | 6 |
| Bladder | 5 |
| Brain | 3 |
| Breast | 5 |
| Cervix | 4 |
| Colon | 5 |
| Corpus uteri | 5 |
| Endometrium | 4 |
| Esophagus | 4 |
| Kidney | 5 |
| Liver | 5 |
| Lung | 5 |
| Omentum | 5 |
| Ovary | 4 |
| Pancreas | 5 |
| Prostate | 4 |
| Rectosigmoid | 5 |
| Small intestine | 5 |
| Stomach | 4 |
| Testis | 7 |
| Thyroid | 4 |
| Vulva | 5 |

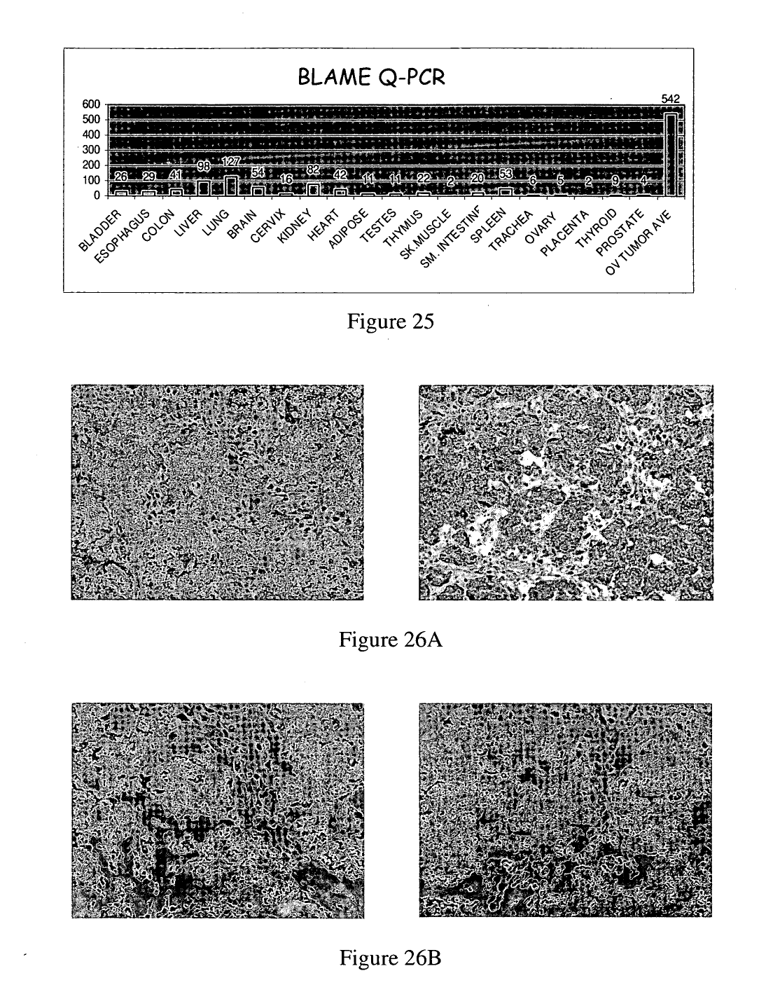

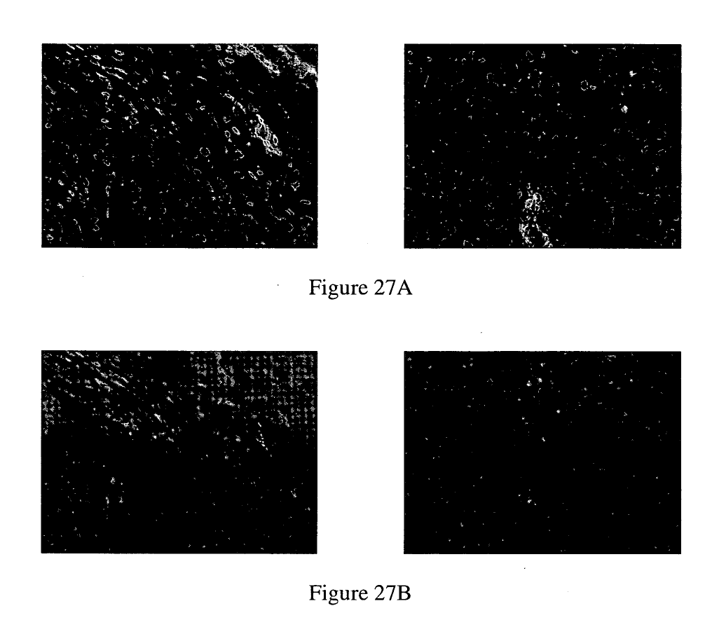

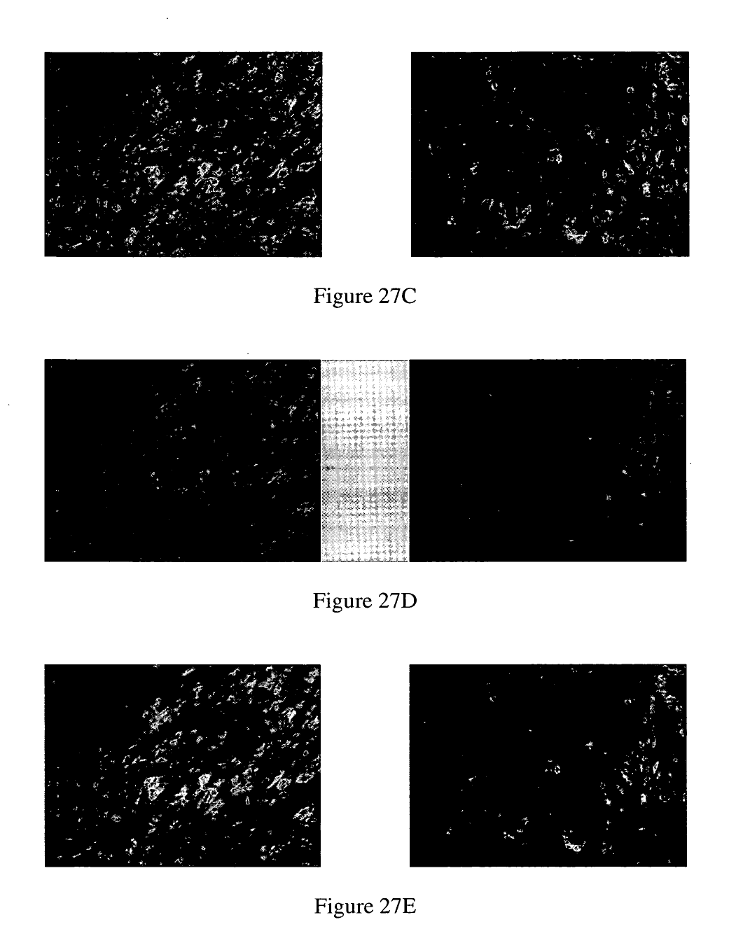

Further, enhanced expression of BLAME in ovarian tumor relative to normal tissues was confirmed by qPCR (FIG. 25). In addition, staining of BLAME was demonstrated in ovarian tumors (FIG. 26). The relevance of BLAME as a TVM was further confirmed by co-staining with CD31 and CD45 (FIG. 27). Thus, BLAME is efficacious as a tumor marker (e.g. for ovarian, adrenal, and testis tumors).

### Example 24

### Additional Studies with ESM1

Expression of ESM1 mRNA was determined in a variety of normal and tumor tissues by Affymetrix® array. Expression was significantly enriched in tumor tissues compared to normal tissues (Tables 20 and 19, respectively).

TABLE 19
Affymetrix array scores of ESM1 in normal tissues.

| | Affy |
|---|---|
| Tissue | score |
| Adipose tissue | 2 |
| Adrenal gland cortex | 2 |
| Bone marrow | 2 |
| Bronchus | 2 |
| Cerebellum | 2 |
| Cerebral cortex | 2 |
| Cerebrum | 2 |
| Cervix | 2 |
| Colon cecum | 2 |
| Coronary artery | 2 |
| Endometrium | 3 |
| Esophagus | 2 |
| Heart atrium | 2 |
| Heart ventricle | 2 |
| Kidney cortex | 3 |
| Kidney medulla | 2 |
| Liver | 2 |
| Lung | 3 |
| Lymph nodes | 2 |
| Mammary gland | 2 |
| Medulla | 2 |
| Midbrain | 2 |
| Myometrium | 2 |
| Nipple cross section | 2 |
| Oral mucosa | 2 |
| Ovary | 2 |
| Pharyngeal mucosa | 2 |
| Pituitary gland | 2 |
| Prostate gland | 2 |
| Salivary gland | 2 |
| Saphenous vein | 2 |
| Skeletal muscle | 2 |
| Spinal cord | 2 |
| Spleen | 2 |
| Stomach | 2 |
| Testes | 2 |
| Thyroid gland | 2 |
| Tongue | 2 |
| Tonsil | 2 |
| Trachea | 2 |
| Trigeminal ganglia | 2 |
| Urethra | 2 |
| Vagina | 2 |
| Vulva | 2 |

TABLE 20
Affymetrix array scores of ESM1 in tumor tissues.

| | Affy |
|---|---|
| Tissue | score |
| Adrenal gland | 6 |
| Bladder | 3 |
| Brain | 3 |
| Breast | 3 |
| Cervix | 3 |
| Colon | 3 |
| Corpus uteri | 4 |
| Endometrium | 3 |
| Esophagus | 3 |
| Kidney | 6 |
| Liver | 3 |
| Lung | 4 |
| Omentum | 4 |
| Ovary | 4 |
| Pancreas | 3 |
| Prostate | 3 |
| Rectosigmoid | 3 |
| Small intestine | 3 |
| Stomach | 3 |
| Testis | 3 |
| Thyroid | 3 |
| Vulva | 3 |

Thus, ESM1 is efficacious as a tumor marker (e.g. for ovarian, adrenal, and kidney tumors).

### Example 25

### Additional Expression Studies

mRNA expression levels of additional markers were determined in a variety of normal and tumor tissues by Affymetrix® array. Results are depicted in Tables 21-28.

TABLE 21
Affymetrix array scores of additional markers in normal tissues.

| | | SCGB- | | | | | |
|---|---|---|---|---|---|---|---|
| Tissue | c6orf55 | 2A1 | DSG2 | EPSTI1 | SEC-23B | MS4A6A | LOC51136 |
| Adipose | 4 | 2 | 4 | 4 | 3 | 5 | 3 |
| tissue | | | | | | | |
| Adrenal | 4 | 2 | 2 | 4 | 2 | 5 | 3 |
| gland | | | | | | | |
| cortex | | | | | | | |
| Bone | 4 | 2 | 2 | 4 | 2 | 5 | 3 |
| marrow | | | | | | | |
| Bronchus | 4 | 3 | 4 | 4 | 5 | 4 | 2 |
| Cerebellum | 4 | 2 | 2 | 3 | 2 | 4 | 2 |
| Cerebral | 4 | 2 | 2 | 3 | 2 | 4 | 2 |
| cortex | | | | | | | |
| Cerebrum | 4 | 2 | 2 | 3 | 2 | 4 | 2 |
| Cervix | 4 | 4 | 3 | 4 | 2 | 4 | 3 |
| Colon | 4 | 2 | 4 | 4 | 2 | 4 | 2 |
| cecum | | | | | | | |
| Coronary | 4 | 2 | 3 | 4 | 4 | 5 | 2 |
| artery | | | | | | | |
| Endo- | 4 | 7 | 4 | 4 | 4 | 4 | 3 |
| metrium | | | | | | | |
| Esophagus | 5 | 2 | 3 | 3 | 2 | 4 | 2 |
| Heart | 4 | 2 | 5 | 4 | 2 | 5 | 3 |
| atrium | | | | | | | |
| Heart | 4 | 2 | 4 | 4 | 2 | 4 | 2 |
| ventricle | | | | | | | |
| Kidney | 4 | 2 | 5 | 4 | 2 | 4 | 2 |
| cortex | | | | | | | |
| Kidney | 4 | 2 | 5 | 4 | 2 | 4 | 2 |
| medulla | | | | | | | |
| Liver | 4 | 2 | 3 | 4 | 2 | 6 | 3 |
| Lung | 4 | 2 | 3 | 5 | 2 | 5 | 3 |
| Lymph | 4 | 2 | 2 | 5 | 2 | 6 | 3 |
| nodes | | | | | | | |
| Mammary | 4 | 4 | 5 | 4 | 3 | 5 | 3 |
| gland | | | | | | | |
| Medulla | 4 | 2 | 2 | 4 | 2 | 4 | 2 |
| Midbrain | 4 | 2 | 2 | 4 | 2 | 4 | 2 |
| Myo- | 4 | 2 | 2 | 3 | 2 | 4 | 2 |
| metrium | | | | | | | |
| Nipple | 4 | 3 | 3 | 3 | 2 | 4 | 2 |
| cross | | | | | | | |
| section | | | | | | | |
| Oral | 5 | 2 | 3 | 4 | 2 | 5 | 3 |
| mucosa | | | | | | | |
| Ovary | 4 | 2 | 3 | 4 | 2 | 4 | 3 |
| Pharyngeal | 5 | 3 | 3 | 3 | 3 | 5 | 3 |
| mucosa | | | | | | | |
| Pituitary | 4 | 3 | 2 | 4 | 2 | 4 | 2 |
| gland | | | | | | | |
| Prostate | 4 | 4 | 5 | 4 | 2 | 4 | 3 |
| gland | | | | | | | |
| Salivary | 4 | 4 | 5 | 4 | 3 | 4 | 3 |
| gland | | | | | | | |
| Saphenous | 4 | 2 | 2 | 4 | 3 | 5 | 2 |
| vein | | | | | | | |
| Skeletal | 5 | 2 | 2 | 4 | 3 | 4 | 2 |
| muscle | | | | | | | |
| Spinal cord | 4 | 2 | 2 | 4 | 2 | 5 | 2 |
| Spleen | 5 | 2 | 2 | 5 | 2 | 5 | 3 |
| Stomach | 4 | 4 | 4 | 4 | 2 | 5 | 3 |
| Testes | 4 | 2 | 2 | 4 | 3 | 4 | 6 |
| Thyroid | 4 | 3 | 5 | 4 | 2 | 5 | 3 |
| gland | | | | | | | |
| Tongue | 5 | 4 | 3 | 4 | 3 | 5 | 3 |
| Tonsil | 4 | 2 | 2 | 5 | 2 | 5 | 3 |
| Trachea | 4 | 4 | 4 | 4 | 6 | 5 | 3 |
| Trigeminal | 4 | 2 | 2 | 4 | 2 | 5 | 2 |
| ganglia | | | | | | | |
| Urethra | 5 | 3 | 4 | 4 | 2 | 5 | 3 |
| Vagina | 5 | 2 | 3 | 4 | 2 | 5 | 3 |
| Vulva | 5 | 2 | 3 | 4 | 2 | 4 | 3 |

TABLE 22
Affymetrix array scores of additional markers in normal tissues.

| Tissue | EGFL6 | KCNE3 | KCNE4 | c14orf-100 | C2orf6 | KCNK5 | C14orf28 |
|---|---|---|---|---|---|---|---|
| Adipose | 4 | 4 | 3 | 5 | 2 | 3 | 2 |
| tissue | | | | | | | |
| Adrenal | 2 | 4 | 2 | 5 | 2 | 3 | 2 |
| gland | | | | | | | |
| cortex | | | | | | | |
| Bone | 3 | 5 | 2 | 5 | 3 | 3 | 2 |
| marrow | | | | | | | |
| Bronchus | 3 | 5 | 2 | 5 | 2 | 3 | 2 |
| Cerebellum | 2 | 3 | 2 | 5 | 2 | 3 | 2 |
| Cerebral | 3 | 3 | 2 | 5 | 2 | 3 | 2 |
| cortex | | | | | | | |
| Cerebrum | 3 | 4 | 2 | 5 | 2 | 3 | 2 |
| Cervix | 3 | 5 | 3 | 5 | 3 | 3 | 2 |
| Colon | 2 | 5 | 2 | 4 | 2 | 3 | 2 |
| cecum | | | | | | | |
| Coronary | 2 | 4 | 3 | 4 | 2 | 3 | 2 |
| artery | | | | | | | |
| Endo- | 4 | 5 | 3 | 5 | 3 | 3 | 2 |
| metrium | | | | | | | |
| Esophagus | 3 | 4 | 2 | 5 | 3 | 3 | 2 |
| Heart | 2 | 4 | 2 | 5 | 2 | 3 | 2 |
| atrium | | | | | | | |
| Heart | 3 | 4 | 2 | 4 | 2 | 3 | 2 |
| ventricle | | | | | | | |
| Kidney | 2 | 5 | 2 | 5 | 3 | 5 | 2 |
| cortex | | | | | | | |
| Kidney | 3 | 5 | 2 | 5 | 2 | 4 | 2 |
| medulla | | | | | | | |
| Liver | 3 | 4 | 2 | 5 | 2 | 4 | 3 |
| Lung | 5 | 5 | 2 | 5 | 2 | 3 | 2 |
| Lymph | 3 | 4 | 3 | 5 | 3 | 3 | 2 |
| nodes | | | | | | | |
| Mammary | 6 | 4 | 2 | 5 | 2 | 3 | 2 |
| gland | | | | | | | |
| Medulla | 3 | 4 | 2 | 5 | 2 | 3 | 2 |
| Midbrain | 3 | 4 | 2 | 5 | 2 | 3 | 2 |
| Myo- | 3 | 4 | 4 | 5 | 2 | 3 | 3 |
| metrium | | | | | | | |
| Nipple | 3 | 4 | 2 | 5 | 2 | 3 | 2 |
| cross | | | | | | | |
| section | | | | | | | |
| Oral | 3 | 3 | 2 | 5 | 3 | 3 | 2 |
| mucosa | | | | | | | |
| Ovary | 3 | 5 | 3 | 5 | 3 | 3 | 3 |
| Pharyngeal | 3 | 4 | 2 | 5 | 3 | 3 | 2 |
| mucosa | | | | | | | |
| Pituitary | 3 | 4 | 2 | 5 | 2 | 3 | 2 |
| gland | | | | | | | |
| Prostate | 3 | 4 | 2 | 6 | 3 | 3 | 3 |
| gland | | | | | | | |
| Salivary | 3 | 4 | 2 | 5 | 2 | 3 | 3 |
| gland | | | | | | | |
| Saphenous | 3 | 4 | 5 | 5 | 2 | 3 | 4 |
| vein | | | | | | | |
| Skeletal | 3 | 3 | 2 | 5 | 2 | 3 | 2 |
| muscle | | | | | | | |
| Spinal cord | 3 | 4 | 2 | 6 | 2 | 3 | 2 |
| Spleen | 2 | 5 | 3 | 5 | 3 | 3 | 2 |
| Stomach | 3 | 6 | 3 | 5 | 3 | 3 | 3 |
| Testes | 2 | 3 | 2 | 5 | 2 | 3 | 4 |
| Thyroid | 2 | 5 | 2 | 5 | 2 | 3 | 3 |
| gland | | | | | | | |
| Tongue | 3 | 4 | 2 | 5 | 3 | 3 | 2 |
| Tonsil | 3 | 4 | 2 | 5 | 3 | 3 | 2 |
| Trachea | 3 | 5 | 2 | 5 | 2 | 3 | 2 |
| Trigeminal | 4 | 4 | 3 | 6 | 2 | 3 | 2 |
| ganglia | | | | | | | |
| Urethra | 3 | 5 | 3 | 6 | 3 | 3 | 3 |
| Vagina | 3 | 5 | 2 | 5 | 3 | 3 | 2 |
| Vulva | 3 | 4 | 2 | 5 | 3 | 3 | 2 |

TABLE 23
Affymetrix array scores of additional markers in normal tissues.

| Tissue | PCDHB2 | FZD10 | ST14 | OLFML2B | GPR105 | SDC1 | FLJ46072 |
|---|---|---|---|---|---|---|---|
| Adipose | 4 | 3 | 3 | 5 | 4 | 4 | 4 |
| tissue | | | | | | | |
| Adrenal | 5 | 2 | 2 | 4 | 2 | 3 | 4 |
| gland | | | | | | | |
| cortex | | | | | | | |
| Bone | 4 | 2 | 2 | 3 | 2 | 3 | 3 |
| marrow | | | | | | | |
| Bronchus | 4 | 3 | 4 | 4 | 2 | 4 | 5 |
| Cerebellum | 5 | 3 | 2 | 3 | 2 | 3 | 3 |
| Cerebral | 6 | 2 | 2 | 3 | 2 | 3 | 4 |
| cortex | | | | | | | |
| Cerebrum | 6 | 2 | 2 | 3 | 2 | 3 | 4 |
| Cervix | 5 | 4 | 3 | 5 | 2 | 5 | 4 |
| Colon | 5 | 3 | 4 | 4 | 5 | 4 | 4 |
| cecum | | | | | | | |
| Coronary | 5 | 2 | 2 | 5 | 3 | 2 | 3 |
| artery | | | | | | | |
| Endo- | 4 | 4 | 3 | 6 | 5 | 3 | 4 |
| metrium | | | | | | | |
| Esophagus | 4 | 5 | 2 | 3 | 3 | 7 | 6 |
| Heart | 4 | 2 | 2 | 4 | 3 | 2 | 4 |
| atrium | | | | | | | |
| Heart | 5 | 2 | 2 | 3 | 3 | 3 | 3 |
| ventricle | | | | | | | |
| Kidney | 5 | 2 | 3 | 3 | 2 | 6 | 5 |
| cortex | | | | | | | |
| Kidney | 4 | 2 | 4 | 3 | 2 | 6 | 5 |
| medulla | | | | | | | |
| Liver | 4 | 2 | 3 | 3 | 2 | 7 | 5 |
| Lung | 6 | | 3 | 5 | 4 | 4 | 4 |
| Lymph | 5 | 3 | 2 | 4 | 3 | 3 | 3 |
| nodes | | | | | | | |
| Mammary | 4 | 3 | 3 | 5 | 3 | 4 | 5 |
| gland | | | | | | | |
| Medulla | 6 | 2 | 2 | 3 | 2 | 2 | 3 |
| Midbrain | 6 | 2 | 2 | 4 | 3 | 3 | 3 |
| Myo- | 5 | 2 | 2 | 4 | 2 | 3 | 3 |
| metrium | | | | | | | |
| Nipple | 4 | 4 | 4 | 4 | 4 | 6 | 6 |
| cross | | | | | | | |
| section | | | | | | | |
| Oral | 4 | 5 | 6 | 4 | 4 | 7 | 7 |
| mucosa | | | | | | | |
| Ovary | 4 | 2 | 2 | 4 | 2 | 3 | 3 |
| Pharyngeal | 4 | 6 | 4 | 4 | 4 | 6 | 6 |
| mucosa | | | | | | | |
| Pituitary | 5 | 3 | 3 | 3 | 4 | 3 | 4 |
| gland | | | | | | | |
| Prostate | 4 | 2 | 4 | 3 | 2 | 5 | 5 |
| gland | | | | | | | |
| Salivary | 4 | 3 | 4 | 4 | 2 | 4 | 5 |
| gland | | | | | | | |
| Saphenous | 5 | 3 | 3 | 6 | 4 | 3 | 3 |
| vein | | | | | | | |
| Skeletal | 4 | 3 | 3 | 4 | 4 | 3 | 3 |
| muscle | | | | | | | |
| Spinal cord | 6 | 3 | 2 | 3 | 2 | 2 | 3 |
| Spleen | 7 | 2 | 3 | 3 | 3 | 3 | 3 |
| Stomach | 5 | 3 | 4 | 4 | 5 | 4 | 5 |
| Testes | 6 | 2 | 2 | 5 | 2 | 4 | 4 |
| Thyroid | 4 | 3 | 4 | 5 | 3 | 4 | 6 |
| gland | | | | | | | |
| Tongue | 4 | 2 | 4 | 4 | 3 | 5 | 5 |
| Tonsil | 4 | 3 | 4 | 4 | 4 | 5 | 5 |
| Trachea | 4 | 3 | 5 | 4 | 3 | 5 | 5 |
| Trigeminal | 5 | 3 | 2 | 5 | 4 | 3 | 4 |
| ganglia | | | | | | | |
| Urethra | 5 | 2 | 4 | 6 | 5 | 6 | 5 |
| Vagina | 4 | 2 | 4 | 4 | 2 | 6 | 5 |
| Vulva | 4 | 5 | 4 | 4 | 3 | 6 | 6 |

TABLE 24
Affymetrix array scores of additional markers in normal tissues.

| Tissue | IVNS1ABP | SPP1 | DKFZp762E1312 | WFDC2 | KIAA1892 | C6orf69 | KIBRA |
|---|---|---|---|---|---|---|---|
| Adipose | 4 | 5 | 3 | 4 | 5 | 6 | 4 |
| tissue | | | | | | | |
| Adrenal | 4 | 3 | 3 | 3 | 7 | 6 | 4 |
| gland | | | | | | | |
| cortex | | | | | | | |
| Bone | 6 | 5 | 5 | 4 | 8 | 5 | 4 |
| marrow | | | | | | | |
| Bronchus | 5 | 5 | 3 | 7 | 5 | 5 | 5 |
| Cerebellum | 4 | 6 | 3 | 4 | 5 | 5 | 5 |
| Cerebral | 5 | 8 | 3 | 4 | 5 | 5 | 5 |
| cortex | | | | | | | |
| Cerebrum | 5 | 8 | 3 | 4 | 5 | 5 | 6 |
| Cervix | 5 | 6 | 3 | 5 | 6 | 6 | 4 |
| Colon | 4 | 5 | 4 | 4 | 5 | 5 | 4 |
| cecum | | | | | | | |
| Coronary | 5 | 5 | 3 | 4 | 5 | 5 | 4 |
| artery | | | | | | | |
| Endo- | 5 | 5 | 4 | 6 | 6 | 5 | 5 |
| metrium | | | | | | | |
| Esophagus | 4 | 3 | 4 | 4 | 6 | 6 | 5 |
| Heart | 4 | 4 | 3 | 3 | 5 | 5 | 4 |
| atrium | | | | | | | |
| Heart | 5 | 3 | 3 | 4 | 5 | 5 | 4 |
| ventricle | | | | | | | |
| Kidney | 6 | 9 | 3 | 5 | 5 | 5 | 6 |
| cortex | | | | | | | |
| Kidney | 6 | 9 | 3 | 5 | 6 | 5 | 6 |
| medulla | | | | | | | |
| Liver | 4 | 5 | 3 | 3 | 5 | 6 | 5 |
| Lung | 5 | 5 | 3 | 5 | 5 | 6 | 5 |
| Lymph | 5 | 4 | 3 | 4 | 6 | 6 | 4 |
| nodes | | | | | | | |
| Mammary | 5 | 6 | 4 | 5 | 5 | 6 | 5 |
| gland | | | | | | | |
| Medulla | 5 | 9 | 3 | 3 | 5 | 5 | 6 |
| Midbrain | 5 | 9 | 3 | 4 | 5 | 5 | 5 |
| Myo- | 4 | 4 | 3 | 4 | 5 | 5 | 3 |
| metrium | | | | | | | |
| Nipple | 4 | 3 | 3 | 5 | 5 | 6 | 5 |
| cross | | | | | | | |
| section | | | | | | | |
| Oral | 4 | 3 | 4 | 4 | 5 | 6 | 6 |
| mucosa | | | | | | | |
| Ovary | 5 | 4 | 4 | 4 | 5 | 6 | 4 |
| Pharyngeal | 5 | 3 | 4 | 5 | 6 | 6 | 5 |
| mucosa | | | | | | | |
| Pituitary | 5 | 4 | 4 | 6 | 5 | 5 | 5 |
| gland | | | | | | | |
| Prostate | 5 | 5 | 3 | 5 | 5 | 5 | 6 |
| gland | | | | | | | |
| Salivary | 5 | 4 | 3 | 7 | 5 | 5 | 4 |
| gland | | | | | | | |
| Saphenous | 4 | 3 | 3 | 5 | 5 | 6 | 4 |
| vein | | | | | | | |
| Skeletal | 4 | 3 | 4 | 4 | 5 | 5 | 4 |
| muscle | | | | | | | |
| Spinal cord | 5 | 9 | 3 | 4 | 5 | 5 | 6 |
| Spleen | 5 | 3 | 3 | 3 | 5 | 6 | 4 |
| Stomach | 5 | 4 | 4 | 4 | 5 | 5 | 5 |
| Testes | 5 | 3 | 5 | 4 | 6 | 4 | 4 |
| Thyroid | 5 | 4 | 3 | 6 | 5 | 5 | 6 |
| gland | | | | | | | |
| Tongue | 5 | 4 | 4 | 6 | 5 | 5 | 5 |
| Tonsil | 5 | 3 | 4 | 4 | 5 | 5 | 4 |
| Trachea | 5 | 5 | 3 | 8 | 5 | 5 | 5 |
| Trigeminal | 5 | 7 | 3 | 6 | 5 | 6 | 4 |
| ganglia | | | | | | | |
| Urethra | 5 | 4 | 3 | 4 | 5 | 6 | 4 |
| Vagina | 5 | 4 | 4 | 4 | 5 | 5 | 4 |
| Vulva | 5 | 3 | 4 | 4 | 5 | 5 | 5 |

TABLE 25
Affymetrix array scores of additional markers in tumor tissues.

| | | SCGB- | | | | | |
|---|---|---|---|---|---|---|---|
| Tissue | c6orf55 | 2A1 | DSG2 | EPSTI1 | SEC-23B | MS4A6A | LOC51136 |
| Adrenal | 6 | 3 | 7 | 7 | 6 | 8 | 5 |
| gland | | | | | | | |
| Bladder | 5 | 3 | 7 | 5 | 5 | 5 | 4 |
| Brain | 5 | 6 | 5 | 5 | 5 | 5 | 3 |
| Breast | 6 | 6 | 7 | 5 | 6 | 6 | 5 |
| Cervix | 5 | 5 | 7 | 5 | 6 | 5 | 4 |
| Colon | 6 | 5 | 8 | 6 | 6 | 6 | 4 |
| Corpus | 6 | 8 | 7 | 5 | 6 | 6 | 4 |
| uteri | | | | | | | |
| Endo- | 5 | 9 | 7 | 5 | 6 | 5 | 4 |
| metrium | | | | | | | |
| Esophagus | 5 | 2 | 5 | 5 | 5 | 5 | 3 |
| Kidney | 5 | 3 | 7 | 5 | 6 | 6 | 4 |
| Liver | 5 | 3 | 7 | 6 | 6 | 6 | 5 |
| Lung | 5 | 5 | 7 | 6 | 6 | 6 | 4 |
| Omentum | 6 | 8 | 7 | 6 | 6 | 6 | 4 |
| Ovary | 6 | 8 | 7 | 6 | 6 | 6 | 4 |
| Pancreas | 5 | 6 | 6 | 6 | 6 | 6 | 3 |
| Prostate | 5 | 4 | 6 | 4 | 6 | 5 | 4 |
| Recto- | 5 | 4 | 8 | 6 | 6 | 6 | 4 |
| sigmoid | | | | | | | |
| Small | 5 | 6 | 6 | 5 | 6 | 5 | 4 |
| intestine | | | | | | | |
| Stomach | 5 | 4 | 7 | 5 | 6 | 5 | 4 |
| Testis | 5 | 2 | 4 | 7 | 6 | 8 | 4 |
| Thyroid | 5 | 4 | 7 | 5 | 5 | 5 | 4 |
| Vulva | 6 | 2 | 6 | 5 | 6 | 5 | 4 |

TABLE 26
Affymetrix array scores of additional markers in tumor tissues.

| Tissue | EGFL6 | KCNE3 | KCNE4 | C14orf-100 | C2orf6 | KCNK5 | C14orf28 |
|---|---|---|---|---|---|---|---|
| Adrenal | 3 | 7 | 5 | 7 | 5 | 5 | 3 |
| gland | | | | | | | |
| Bladder | 5 | 4 | 3 | 5 | 4 | 3 | 3 |
| Brain | 5 | 5 | 3 | 5 | 3 | 3 | 2 |
| Breast | 5 | 5 | 5 | 6 | 4 | 4 | 3 |
| Cervix | 5 | 5 | 4 | 5 | 4 | 3 | 3 |
| Colon | 4 | 6 | 3 | 6 | 4 | 4 | 3 |
| Corpus | 7 | 5 | 4 | 6 | 4 | 3 | 3 |
| uteri | | | | | | | |
| Endo- | 5 | 6 | 4 | 6 | 4 | 4 | 3 |
| metrium | | | | | | | |
| Esophagus | 3 | 6 | 3 | 5 | 3 | 3 | 2 |
| Kidney | 3 | 7 | 4 | 6 | 4 | 4 | 3 |
| Liver | 4 | 6 | 4 | 6 | 4 | 4 | 3 |
| Lung | 6 | 6 | 4 | 6 | 4 | 4 | 3 |
| Omentum | 6 | 5 | 4 | 6 | 4 | 4 | 3 |
| Ovary | 5 | 6 | 4 | 6 | 5 | 4 | 3 |
| Pancreas | 4 | 5 | 5 | 6 | 4 | 4 | 3 |
| Prostate | 3 | 4 | 3 | 6 | 4 | 3 | 3 |
| Recto- | 5 | 7 | 4 | 6 | 4 | 5 | 3 |
| sigmoid | | | | | | | |
| Small | 5 | 5 | 3 | 6 | 4 | 4 | 3 |
| intestine | | | | | | | |
| Stomach | 3 | 7 | 4 | 6 | 4 | 4 | 3 |
| Testis | 3 | 5 | 3 | 6 | 5 | 4 | 3 |
| Thyroid | 5 | 5 | 4 | 6 | 4 | 4 | 3 |
| Vulva | 5 | 5 | 3 | 5 | 4 | 3 | 3 |

TABLE 27
Affymetrix array scores of additional markers in tumor tissues.

| Tissue | PCDHB2 | FZD10 | ST14 | OLFML2B | GPR105 | SDC1 | FLJ46072 |
|---|---|---|---|---|---|---|---|
| Adrenal | 6 | 2 | 6 | 7 | 4 | 7 | 6 |
| gland | | | | | | | |
| Bladder | 5 | 3 | 5 | 5 | 3 | 7 | 6 |
| Brain | 6 | 3 | 4 | 4 | 2 | 5 | 5 |
| Breast | 5 | 3 | 5 | 6 | 4 | 7 | 6 |
| Cervix | 5 | 5 | 5 | 5 | 3 | 7 | 6 |
| Colon | 5 | 4 | 6 | 5 | 4 | 6 | 6 |
| Corpus | 5 | 6 | 5 | 7 | 3 | 5 | 5 |
| uteri | | | | | | | |
| Endo- | 5 | 5 | 5 | 5 | 3 | 6 | 6 |
| metrium | | | | | | | |
| Esophagus | 5 | 2 | 5 | 5 | 3 | 6 | 5 |
| Kidney | 6 | 2 | 4 | 5 | 4 | 6 | 5 |
| Liver | 5 | 3 | 6 | 6 | 3 | 7 | 6 |
| Lung | 6 | 4 | 5 | 6 | 4 | 7 | 6 |
| Omentum | 5 | 5 | 5 | 6 | 3 | 6 | 6 |
| Ovary | 5 | 5 | 5 | 5 | 3 | 6 | 6 |
| Pancreas | 6 | 2 | 5 | 6 | 4 | 6 | 5 |
| Prostate | 4 | 3 | 5 | 4 | 3 | 5 | 5 |
| Recto- | 5 | 4 | 6 | 6 | 5 | 6 | 6 |
| sigmoid | | | | | | | |
| Small | 5 | 4 | 5 | 4 | 3 | 5 | 5 |
| intestine | | | | | | | |
| Stomach | 6 | 3 | 5 | 5 | 6 | 6 | 6 |
| Testis | 4 | 2 | 3 | 6 | 6 | 4 | 4 |
| Thyroid | 5 | 3 | 4 | 4 | 4 | 6 | 5 |
| Vulva | 5 | 4 | 5 | 6 | 3 | 8 | 6 |

TABLE 28
Affymetrix array scores of additional markers in tumor tissues.

| Tissue | IVNS1ABP | SPP1 | DKFZp762E1312 | WFDC2 | KIAA1892 | C6orf69 | KIBRA |
|---|---|---|---|---|---|---|---|
| Adrenal | 7 | 9 | 6 | 6 | 6 | 7 | 5 |
| gland | | | | | | | |
| Bladder | 5 | 7 | 4 | 6 | 5 | 6 | 4 |
| Brain | 5 | 6 | 3 | 6 | 5 | 6 | 5 |
| Breast | 5 | 8 | 4 | 6 | 6 | 6 | 5 |
| Cervix | 6 | 7 | 4 | 6 | 5 | 6 | 4 |
| Colon | 6 | 7 | 4 | 6 | 6 | 6 | 5 |
| Corpus | 6 | 7 | 5 | 7 | 6 | 6 | 5 |
| uteri | | | | | | | |
| Endo- | 6 | 8 | 4 | 8 | 6 | 6 | 5 |
| metrium | | | | | | | |
| Esophagus | 5 | 6 | 4 | 5 | 4 | 5 | 4 |
| Kidney | 7 | 9 | 3 | 6 | 5 | 6 | 6 |
| Liver | 6 | 9 | 4 | 6 | 6 | 6 | 5 |
| Lung | 6 | 8 | 4 | 7 | 6 | 6 | 5 |
| Omentum | 6 | 8 | 5 | 7 | 6 | 6 | 5 |
| Ovary | 6 | 8 | 5 | 8 | 6 | 6 | 5 |
| Pancreas | 5 | 7 | 3 | 5 | 5 | 6 | 5 |
| Prostate | 5 | 5 | 3 | 5 | 5 | 6 | 6 |
| Recto- | 6 | 8 | 4 | 6 | 6 | 6 | 5 |
| sigmoid | | | | | | | |
| Small | 5 | 7 | 4 | 5 | 5 | 6 | 4 |
| intestine | | | | | | | |
| Stomach | 5 | 7 | 4 | 4 | 5 | 6 | 5 |
| Testis | 7 | 6 | 4 | 5 | 7 | 6 | 4 |
| Thyroid | 6 | 7 | 3 | 6 | 5 | 5 | 5 |
| Vulva | 6 | 7 | 4 | 4 | 6 | 6 | 4 |

Thus, TVM of the present invention are enriched in a wide variety of tumor cells. These findings further validate the TVM identified in the present invention, and demonstrate that the TVM are relevant in general to tumor cells. Further, the results show that diagnostic, localization, and therapeutic methods based on TVM of the present invention are not limited to ovarian tumors, but rather are applicable to all solid tumors.

What is claimed is:

1. A method of localizing an ovarian tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 69; and
b. localizing said ligand,
thereby localizing an ovarian tumor vasculature in a subject.

2. A method of treating an ovarian tumor in a subject, the method comprising the steps of localizing said solid tumor by the method of claim 1 and irradiating said solid tumor, thereby treating an ovarian tumor in a subject.

3. A method of treating an ovarian tumor in a subject, the method comprising the steps of:
a. contacting said subject with a photoactivatable cytotoxic drug or pharmaceutical composition;
b. localizing said solid tumor by the method of claim 1; and
c. contacting said solid tumor with a concentrated light source,
thereby treating an ovarian tumor in a subject.

4. A method of localizing an ovarian tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 1; and
b. localizing said ligand,
thereby localizing an ovarian tumor vasculature in a subject.

5. A method of treating an ovarian tumor in a subject, the method comprising the steps of localizing said solid tumor by the method of claim 4 and irradiating said solid tumor, thereby treating an ovarian tumor in a subject.

6. A method of treating an ovarian tumor in a subject, the method comprising the steps of:
a. contacting said subject with a photoactivatable cytotoxic drug or pharmaceutical composition;
b. localizing said solid tumor by the method of claim 4; and
c. contacting said solid tumor with a concentrated light source,
thereby treating an ovarian tumor in a subject.

7. A method of localizing an ovarian tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 119; and
b. localizing said ligand,
thereby localizing an ovarian tumor vasculature in a subject.

8. A method of treating an ovarian tumor in a subject, the method comprising the steps of localizing said solid tumor by the method of claim 7 and irradiating said solid tumor, thereby treating an ovarian tumor in a subject.

9. A method of treating an ovarian tumor in a subject, the method comprising the steps of:
a. contacting said subject with a photoactivatable cytotoxic drug or pharmaceutical composition;
b. localizing said solid tumor by the method of claim 7; and
c. contacting said solid tumor with a concentrated light source,
thereby treating an ovarian tumor in a subject.

10. A method of localizing an ovarian tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 106; and
b. localizing said ligand,
thereby localizing an ovarian tumor vasculature in a subject.

11. A method of treating an ovarian tumor in a subject, the method comprising the steps of localizing said solid tumor by the method of claim 10 and irradiating said solid tumor, thereby treating an ovarian tumor in a subject.

12. A method of treating an ovarian tumor in a subject, the method comprising the steps of:
a. contacting said subject with a photoactivatable cytotoxic drug or pharmaceutical composition;
b. localizing said solid tumor by the method of claim 10; and
c. contacting said solid tumor with a concentrated light source,
thereby treating an ovarian tumor in a subject.

13. A method of localizing an ovarian tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 58; and
b. localizing said ligand,
thereby localizing an ovarian tumor vasculature in a subject.

14. A method of treating an ovarian tumor in a subject, the method comprising the steps of localizing said solid tumor by the method of claim 13 and irradiating said solid tumor, thereby treating an ovarian tumor in a subject.

15. A method of treating an ovarian tumor in a subject, the method comprising the steps of:
a. contacting said subject with a photoactivatable cytotoxic drug or pharmaceutical composition;
b. localizing said solid tumor by the method of claim 13; and
c. contacting said solid tumor with a concentrated light source,
thereby treating an ovarian tumor in a subject.

16. A method of localizing a renal tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 30; and
b. localizing said ligand,
thereby localizing a renal tumor vasculature in a subject.

17. A method of treating a renal tumor in a subject, the method comprising the steps of localizing said solid tumor by the method of claim 16 and irradiating said solid tumor, thereby treating a renal tumor in a subject.

18. A method of treating a renal tumor in a subject, the method comprising the steps of:
a. contacting said subject with a photoactivatable cytotoxic drug or pharmaceutical composition;
b. localizing said solid tumor by the method of claim 16; and
c. contacting said solid tumor with a concentrated light source,
thereby treating a renal tumor in a subject.

19. A method of localizing an ovarian tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 27; and
b. localizing said ligand,
thereby localizing an ovarian tumor vasculature in a subject.

20. A method of treating an ovarian tumor in a subject, the method comprising the steps of localizing said solid tumor by the method of claim 19 and irradiating said solid tumor, thereby treating an ovarian tumor in a subject.

21. A method of treating an ovarian tumor in a subject, the method comprising the steps of:
a. contacting said subject with a photoactivatable cytotoxic drug or pharmaceutical composition;
b. localizing said solid tumor by the method of claim 19; and
c. contacting said solid tumor with a concentrated light source,
thereby treating an ovarian tumor in a subject.

22. A method of localizing an ovarian tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 37; and
b. localizing said ligand,
thereby localizing an ovarian tumor vasculature in a subject.

23. A method of treating an ovarian tumor in a subject, the method comprising the steps of localizing said solid tumor by the method of claim 22 and irradiating said solid tumor, thereby treating an ovarian tumor in a subject.

24. A method of treating an ovarian tumor in a subject, the method comprising the steps of:
a. contacting said subject with a photoactivatable cytotoxic drug or pharmaceutical composition;
b. localizing said solid tumor by the method of claim 22; and
c. contacting said solid tumor with a concentrated light source,
thereby treating an ovarian tumor in a subject.

25. A method of localizing a breast cancer tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 21; and
b. localizing said ligand,
thereby localizing a breast cancer tumor vasculature in a subject.

26. A method of treating a breast cancer tumor in a subject, the method comprising the steps of localizing said solid tumor by the method of claim 25 and irradiating said solid tumor, thereby treating a breast cancer tumor in a subject.

27. A method of treating a breast cancer tumor in a subject, the method comprising the steps of:
a. contacting said subject with a photoactivatable cytotoxic drug or pharmaceutical composition;
b. localizing said solid tumor by the method of claim 25; and
c. contacting said solid tumor with a concentrated light source,
thereby treating a breast cancer tumor in a subject.

28. A method of localizing an ovarian tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 68; and
b. localizing said ligand,
thereby localizing an ovarian tumor vasculature in a subject.

29. A method of treating an ovarian tumor in a subject, the method comprising the steps of localizing said solid tumor by the method of claim 28 and irradiating said solid tumor, thereby treating an ovarian tumor in a subject.

30. A method of treating an ovarian tumor in a subject, the method comprising the steps of:
a. contacting said subject with a photoactivatable cytotoxic drug or pharmaceutical composition;
b. localizing said solid tumor by the method of claim 28; and
c. contacting said solid tumor with a concentrated light source,
thereby treating an ovarian tumor in a subject.

31. A method of localizing an ovarian tumor vasculature in a subject, the method comprising the steps of:
a. contacting said subject with a ligand that binds a nucleic acid molecule, or that binds a protein encoded by said nucleic acid molecule, wherein the sequence of said nucleic acid molecule is set forth in SEQ ID No: 2; and
b. localizing said ligand,
thereby localizing an ovarian tumor vasculature in a subject.

32. A method of treating an ovarian tumor in a subject, the method comprising the steps of localizing said solid tumor by the method of claim 31 and irradiating said solid tumor, thereby treating an ovarian tumor in a subject.

33. A method of treating an ovarian tumor in a subject, the method comprising the steps of:
a. contacting said subject with a photoactivatable cytotoxic drug or pharmaceutical composition;
b. localizing said solid tumor by the method of claim 31; and
c. contacting said solid tumor with a concentrated light source,
thereby treating an ovarian tumor in a subject.

34. A method of localizing an ovarian tumor vasculature in a subject, the method comprising the steps of:
a. contacting said subject with a ligand that binds a nucleic acid molecule, or that binds a protein encoded by said nucleic acid molecule, said nucleic acid molecule having a sequence selected from the sequences set forth in SEQ ID No: 13, 14, 26, 35, 39, 42, 43, 47, 104, 108, 111, 113, 116, 118, 121, 127, 130, 132, 134, 146, 152, 165, 169, 170, 179, and 181,
b. localizing said ligand,
thereby localizing an ovarian tumor vasculature in a subject.

35. A method of treating an ovarian tumor in a subject, the method comprising the steps of localizing said solid tumor by the method of claim 34 and irradiating said solid tumor, thereby treating an ovarian tumor in a subject.

36. A method of treating an ovarian tumor in a subject, the method comprising the steps of:
a. contacting said subject with a photoactivatable cytotoxic drug or pharmaceutical composition;
b. localizing said solid tumor by the method of claim 34; and
c. contacting said solid tumor with a concentrated light source,
thereby treating an ovarian tumor in a subject.

37. A method of treating an ovarian tumor in a subject, the method comprising the step of contacting said solid tumor with an antibody or ligand that is conjugated to a cytotoxic agent or cytotoxic drug and that binds a nucleic acid molecule or a protein encoded by said nucleic acid molecule, wherein the sequence of said nucleic acid molecule is selected from the sequences set forth in SEQ ID No: 1, 2, 13, 14, 26, 27, 35, 37, 39, 42, 43, 47, 58, 68, 69, 104, 106, 108, 111, 113, 116, 118, 119, 121, 127, 130, 132, 134, 146, 152, 165, 169, 170, 179, and 181, thereby treating an ovarian tumor in a subject.

38. The method of claim 37, wherein said cytotoxic agent is a radioactive isotope.

39. The method of claim 37, wherein said cytotoxic agent is a nucleic acid molecule.

40. The method of claim 37, wherein said cytotoxic agent is an antisense nucleotide, or RNA inhibitory molecule.

41. The method of claim 37, wherein said cytotoxic agent is a cytotoxic virus or pathogen.

42. The method of claim 37, wherein said cytotoxic agent is a nanoparticle.

43. The method of claim 37, wherein said cytotoxic agent is an engineered T cell or an engineered cytotoxic cell.

44. A method of treating a renal tumor in a subject, the method comprising the step of contacting said solid tumor with an antibody or ligand that is conjugated to a cytotoxic agent or cytotoxic drug and that binds a nucleic acid molecule or a protein encoded by said nucleic acid molecule, wherein the sequence of said nucleic acid molecule is set forth in SEQ ID No: 30, thereby treating a renal tumor in a subject.

45. The method of claim 44, wherein said cytotoxic agent is a radioactive isotope.

46. The method of claim 44, wherein said cytotoxic agent is a nucleic acid molecule.

47. The method of claim 44, wherein said cytotoxic agent is an antisense nucleotide, or RNA inhibitory molecule.

48. The method of claim 44, wherein said cytotoxic agent is a cytotoxic virus or pathogen.

49. The method of claim 44, wherein said cytotoxic agent is a nanoparticle.

50. The method of claim 44, wherein said cytotoxic agent is an engineered T cell or an engineered cytotoxic cell.

51. A method of treating a breast cancer tumor in a subject, the method comprising the step of contacting said solid tumor with an antibody or ligand that is conjugated to a cytotoxic agent or cytotoxic drug and that binds a nucleic acid molecule or a protein encoded by said nucleic acid molecule, wherein the sequence of said nucleic acid molecule is set forth in SEQ ID No: 21, thereby treating a breast cancer tumor in a subject.

52. The method of claim 51, wherein said cytotoxic agent is a radioactive isotope.

53. The method of claim 51, wherein said cytotoxic agent is a nucleic acid molecule.

54. The method of claim 51, wherein said cytotoxic agent is an antisense nucleotide, or RNA inhibitory molecule.

55. The method of claim 51, wherein said cytotoxic agent is a cytotoxic virus or pathogen.

56. The method of claim 51, wherein said cytotoxic agent is a nanoparticle.

57. The method of claim 51, wherein said cytotoxic agent is an engineered T cell or an engineered cytotoxic cell.

58. A method of impeding a vascularization of an ovarian tumor in a subject, the method comprising the step of contacting said subject with a ligand capable of inhibiting an activity of a nucleic acid molecule or a protein encoded by said nucleic acid molecule wherein the sequence of said nucleic acid molecule is selected from the sequences set forth in SEQ ID No: 1, 2, 13, 14, 26, 27, 35, 37, 39, 42, 43, 47, 58, 68, 69, 104, 106, 108, 111, 113, 116, 118, 119, 121, 127, 130, 132, 134, 146, 152, 165, 169, 170, 179, and 181, whereby said ligand is taken up by a vasculature cell of said ovarian tumor, thereby impeding a vascularization of a solid tumor in a subject.

59. The method of claim 58, wherein said ligand is an antisense or RNA inhibitory molecule.

60. A method of impeding a vascularization of a renal tumor in a subject, the method comprising the step of contacting said subject with a ligand capable of inhibiting an activity of a nucleic acid molecule or a protein encoded by said nucleic acid molecule wherein the sequence of said nucleic acid molecule is set forth in SEQ ID No: 30, whereby said ligand is taken up by a vasculature cell of said renal tumor, thereby impeding a vascularization of a solid tumor in a subject.

61. The method of claim 60, wherein said ligand is an antisense or RNA inhibitory molecule.

62. A method of impeding a vascularization of a breast cancer tumor in a subject, the method comprising the step of contacting said subject with a ligand capable of inhibiting an activity of a nucleic acid molecule or a protein encoded by said nucleic acid molecule wherein the sequence of said nucleic acid molecule is set forth in SEQ ID No: 21, whereby said ligand is taken up by a vasculature cell of said breast cancer tumor, thereby impeding a vascularization of a solid tumor in a subject.

63. The method of claim 62, wherein said ligand is an antisense or RNA inhibitory molecule.

64. A method of detecting a presence of an ovarian tumor in a subject, the method comprising the step of detecting a presence in a body fluid of said subject of a protein encoded by a nucleotide molecule, wherein the sequence of said nucleotide molecule is set forth in SEQ ID No: 2, whereby said presence in a body fluid of said protein indicates said presence of an ovarian tumor in said subject.

65. A method of detecting a presence of an ovarian tumor in a subject, the method comprising the step of detecting a presence in a body fluid of said subject of a nucleotide molecule, wherein the sequence of said nucleotide molecule is set forth in SEQ ID No: 2, whereby said presence in a body fluid of said nucleotide molecule indicates said presence of an ovarian tumor in said subject.

66. A method of detecting a presence of an ovarian tumor in a subject, the method comprising the step of detecting a presence in a body fluid of said subject of a protein encoded by a nucleotide molecule, wherein the sequence of said nucleotide molecule is set forth in SEQ ID No: 69, whereby said presence in a body fluid of said protein indicates said presence of an ovarian tumor in said subject.

67. A method of detecting a presence of an ovarian tumor in a subject, the method comprising the step of detecting a presence in a body fluid of said subject of a nucleotide molecule, wherein the sequence of said nucleotide molecule is set forth in SEQ ID No: 69, whereby said presence in a body fluid of said nucleotide molecule indicates said presence of an ovarian tumor in said subject.

68. A method of detecting a presence of an ovarian tumor vasculature cell in a subject, the method comprising the step of:
detecting a presence in a body fluid of said subject of a protein encoded by a nucleotide molecule selected from SEQ ID No: 14, 35, 42, 68, 121, 127, 169, 179, and 181,
whereby said presence in a body fluid of said protein indicates said presence of an ovarian tumor vasculature cell in said subject.

69. A method of detecting a presence of an ovarian tumor vasculature cell in a subject, the method comprising the step of:
detecting a presence in a body fluid of said subject of a nucleotide molecule selected from SEQ ID No: 14, 35, 42, 68, 121, 127, 169, 179, and 181,
whereby said presence in a body fluid of said nucleotide molecule indicates said presence of an ovarian tumor vasculature cell in said subject.

70. A method of detecting a presence of a breast cancer vasculature cell in a subject, the method comprising the step of:
detecting a presence in a body fluid of said subject of a protein encoded by a SEQ ID No: 21,
whereby said presence in a body fluid of said protein indicates said presence of a breast cancer vasculature cell in said subject.

71. A method of detecting a presence of a breast cancer vasculature cell in a subject, the method comprising the step of:
detecting a presence in a body fluid of said subject of a nucleotide molecule, wherein the sequence of said nucleotide molecule is set forth in SEQ ID No: 21,
whereby said presence in a body fluid of said nucleotide molecule indicates said presence of a breast cancer vasculature cell in said subject.

72. A peptide with the amino acid sequence CPGAKALSRVREDIVEDE (SEQ ID No: 88).

73. An antibody that recognizes the peptide of claim 72.

74. Use of the peptide of claim 72 in the detection of an ovarian tumor or tumor vasculature.

75. Use of the peptide of claim 72 in the localization of an ovarian tumor or tumor vasculature.

76. Use of the peptide of claim 72 in the treatment of an ovarian tumor or tumor vasculature.
